(19) United States
(12) Patent Application Publication
(10) Pub. No.: US 2004/0192771 A1
(43) Pub. Date: Sep. 30, 2004

(54) Ether compounds and compositions for cholesterol management and related uses

(76) Inventors: Jean-Louis Henri Dasseux, Brighton, MI (US); Carmen Daniela Oniciu, Ann Arbor, MI (US)

Correspondence Address: JONES DAY, 51 Louisiana Aveue, N.W, WASHINGTON, DC, 20001-2113, US

(21) Appl. No.: 10/743,951

(22) Filed: Dec. 24, 2003

Related U.S. Application Data
(63) Continuation-in-part of application No. 09/976,867, filed on Oct. 11, 2001, now Pat. No. 6,713,507.

(51) Int. Cl. A61K 31/275; A61K 31/20

(52) U.S. Cl. 514/526; 514/558; 554/111; 554/113

(57) ABSTRACT

The present invention relates to novel ether compounds, compositions comprising ether compounds, and methods useful for treating and preventing cardiovascular diseases, dyslipidemias, dysproteinemias, and glucose metabolism disorders comprising administering a composition comprising an ether compound. The compounds, compositions, and methods of the invention are also useful for treating and preventing Alzheimer's Disease, Syndrome X, peroxisome proliferator activated receptor-related disorders, septicemia, thrombotic disorders, obesity, pancreatitis, hypertension, renal disease, cancer, inflammation, and impotence. In certain embodiments, the compounds, compositions, and methods of the invention are useful in combination therapy with other therapeutics, such as hypocholesterolemic and hypoglycemic agents.

This application is a continuation-in-part application of pending U.S. application Ser. No. 09/976,867, filed Oct. 11, 2001, which is incorporated herein by reference in its entirety.

### 1. FIELD OF THE INVENTION

The present invention relates to ether compounds, and pharmaceutically acceptable salts, hydrates, solvates, and mixtures thereof; compositions comprising an ether compound or a pharmaceutically acceptable salt, hydrate, solvate, or a mixture thereof; and methods for treating or preventing a disease or disorder such as, but not limited to, aging, Alzheimer's Disease, cancer, cardiovascular disease, diabetic nephropathy, diabetic retinopathy, a disorder of glucose metabolism, dyslipidemia, dyslipoproteinemia, enhancing bile production, enhancing reverse lipid transport, hypertension, impotence, inflammation, insulin resistance, lipid elimination in bile, modulating C reactive protein, obesity, oxysterol elimination in bile, pancreatitis, Parkinson's disease, a peroxisome proliferator activated receptor-associated disorder, phospholipid elimination in bile, renal disease, septicemia, metabolic syndrome disorders (e.g., Syndrome X), and a thrombotic disorder, which method comprise administering an ether compound or composition of the invention. The compounds of the invention can also treat or prevent inflammatory processes and diseases like gastrointestinal disease, irritable bowel syndrome (IBS), inflammatory bowel disease (e.g., Crohn's Disease, ulcerative colitis), arthritis (e.g., rheumatoid arthritis, osteoarthritis), autoimmune disease (e.g., systemic lupus erythematosus), scleroderma, ankylosing spondylitis, gout and pseudogout, muscle pain: polymyositis/polymyalgia rheumatica/fibrositis; infection and arthritis, juvenile rheumatoid arthritis, tendonitis, bursitis and other soft tissue rheumatism.

### 2. BACKGROUND OF THE INVENTION

Obesity, hyperlipidemia, and diabetes have been shown to play a casual role in atherosclerotic cardiovascular diseases, which currently account for a considerable proportion of morbidity in Western society. Further, one human disease, termed “Syndrome X” or “Metabolic Syndrome”, is manifested by defective glucose metabolism (insulin resistance), elevated blood pressure (hypertension), and a blood lipid imbalance (dyslipidemia). See e.g. Reaven, 1993, Annu. Rev. Med. 44:121-131.

The evidence linking elevated serum cholesterol to coronary heart disease is overwhelming. Circulating cholesterol is carried by plasma lipoproteins, which are particles of complex lipid and protein composition that transport lipids in the blood. Low density lipoprotein (LDL) and high density lipoprotein (HDL) are the major cholesterol-carrier proteins. LDL is believed to be responsible for the delivery of cholesterol from the liver, where it is synthesized or obtained from dietary sources, to extrahepatic tissues in the body. The term “reverse cholesterol transport” describes the transport of cholesterol from extrahepatic tissues to the liver, where it is catabolized and eliminated. It is believed that plasma HDL particles play a major role in the reverse transport process, acting as scavengers of tissue cholesterol. HDL is also responsible for the removal of non-cholesterol lipid, oxidized cholesterol and other oxidized products from the bloodstream.

Atherosclerosis, for example, is a slowly progressive disease characterized by the accumulation of cholesterol within the arterial wall. Compelling evidence supports the belief that lipids deposited in atherosclerotic lesions are derived primarily from plasma apolipoprotein B (apo B)-containing lipoproteins, which include chylomicrons, VLDL, IDL and LDL. The apo B-containing lipoprotein, and in particular LDL, has popularly become known as the “bad” cholesterol. In contrast, HDL serum levels correlate inversely with coronary heart disease. Indeed, high serum levels of HDL is regarded as a negative risk factor. It is hypothesized that high levels of plasma HDL are not only protective against coronary artery disease, but may actually induce regression of atherosclerotic plaque (e.g., see Badimon et al., 1992, Circulation 86:(Suppl. III)86-94; Dansky and Fisher, 1999, Circulation 100:1762-3.). Thus, HDL has popularly become known as the “good” cholesterol.

### 2.1 Cholesterol Transport

The fat-transport system can be divided into two pathways: an exogenous one for cholesterol and triglycerides absorbed from the intestine and an endogenous one for cholesterol and triglycerides entering the bloodstream from the liver and other non-hepatic tissue.

In the exogenous pathway, dietary fats are packaged into lipoprotein particles called chylomicrons, which enter the bloodstream and deliver their triglycerides to adipose tissue for storage and to muscle for oxidation to supply energy. The remnant of the chylomicron, which contains cholesteryl esters, is removed from the circulation by a specific receptor found only on liver cells. This cholesterol then becomes available again for cellular metabolism or for recycling to extrahepatic tissues as plasma lipoproteins.

In the endogenous pathway, the liver secretes a large, very-low-density lipoprotein particle (VLDL) into the bloodstream. The core of VLDL consists mostly of triglycerides synthesized in the liver, with a smaller amount of cholesteryl esters either synthesized in the liver or recycled from chylomicrons. Two predominant proteins are displayed on the surface of VLDL, apolipoprotein B-100 (apo B-100) and apolipoprotein E (apo E), although other apolipoproteins are present, such as apolipoprotein CIII (apo CIII) and apolipoprotein CII (apo CII). When a VLDL reaches the capillaries of adipose tissue or of muscle, its triglyceride is extracted. This results in the formation of a new kind of particle called intermediate-density lipoprotein (IDL) or VLDL remnant, decreased in size and enriched in cholesteryl esters relative to a VLDL, but retaining its two apoproteins.

In human beings, about half of the IDL particles are removed from the circulation quickly, generally within two to six hours of their formation. This is because IDL particles bind tightly to liver cells, which extract IDL cholesterol to make new VLDL and bile acids. The IDL not taken up by the liver is catabolized by the hepatic lipase, an enzyme bound to the proteoglycan on liver cells. Apo E dissociates from IDL as it is transformed to LDL. Apo B-100 is the sole protein of LDL.

Primarily, the liver takes up and degrades circulating cholesterol to bile acids, which are the end products of cholesterol metabolism. The uptake of cholesterol-containing particles is mediated by LDL receptors, which are present in high concentrations on hepatocytes. The LDL receptor binds both apo E and apo B-100 and is responsible for binding and removing both IDL and LDL from the circulation. In addition, remnant receptors are responsible for clearing chylomicrons and VLDL remnants (i.e., IDL). However, the affinity of apo E for the LDL receptor is greater than that of apo B-100. As a result, the LDL particles have a much longer circulating life span than IDL particles; LDL circulates for an average of two and a half days before binding to the LDL receptors in the liver and other tissues. High serum levels of LDL, the “bad” cholesterol, are positively associated with coronary heart disease. For example, in atherosclerosis, cholesterol derived from circulating LDL accumulates in the walls of arteries. This accumulation forms bulky plaques that inhibit the flow of blood until a clot eventually forms, obstructing an artery and causing a heart attack or stroke.

Ultimately, the amount of intracellular cholesterol liberated from the LDL controls cellular cholesterol metabolism. The accumulation of cellular cholesterol derived from VLDL and LDL controls three processes. First, it reduces the cell's ability to make its own cholesterol by turning off the synthesis of HMGCoA reductase, a key enzyme in the cholesterol biosynthetic pathway. Second, the incoming LDL-derived cholesterol promotes storage of cholesterol by the action of ACAT, the cellular enzyme that converts cholesterol into cholesteryl esters that are deposited in storage droplets. Third, the accumulation of cholesterol within the cell drives a feedback mechanism that inhibits cellular synthesis of new LDL receptors. Cells, therefore, adjust their complement of LDL receptors so that enough cholesterol is brought in to meet their metabolic needs, without overloading (for a review, see Brown & Goldstein, In, The Pharmacological Basis Of Therapeutics, 8th Ed., Goodman & Gilman, Pergamon Press, New York, 1990, Ch. 36, pp. 874-896).

High levels of apo B-containing lipoproteins can be trapped in the subendothelial space of an artery and undergo oxidation. The oxidized lipoprotein is recognized by scavenger receptors on macrophages. Binding of oxidized lipoprotein to the scavenger receptors can enrich the macrophages with cholesterol and cholesteryl esters independently of the LDL receptor. Macrophages can also produce cholesteryl esters by the action of ACAT. LDL can also be complexed to a high molecular weight glycoprotein called apolipoprotein(a), also known as apo(a), through a disulfide bridge. The LDL-apo(a) complex is known as Lipoprotein(a) or Lp(a). Elevated levels of Lp(a) are detrimental, having been associated with atherosclerosis, coronary heart disease, myocardial infarction, stroke, cerebral infarction, and restenosis following angioplasty.

### 2.2 Reverse Cholesterol Transport

Peripheral (non-hepatic) cells predominantly obtain their cholesterol from a combination of local synthesis and uptake of preformed sterol from VLDL and LDL. Cells expressing scavenger receptors, such as macrophages and smooth muscle cells, can also obtain cholesterol from oxidized apo B-containing lipoproteins. In contrast, reverse cholesterol transport (RCT) is the pathway by which peripheral cell cholesterol can be returned to the liver for recycling to extrahepatic tissues, hepatic storage, or excretion into the intestine in bile. The RCT pathway represents the only means of eliminating cholesterol from most extrahepatic tissues and is crucial to maintenance of the structure and function of most cells in the body.

The enzyme in blood involved in the RCT pathway, lecithin:cholesterol acyltransferase (LCAT), converts cell-derived cholesterol to cholesteryl esters, which are sequestered in HDL destined for removal. LCAT is produced mainly in the liver and circulates in plasma associated with the HDL fraction. Cholesterol ester transfer protein (CETP) and another lipid transfer protein, phospholipid transfer protein (PLTP), contribute to further remodeling the circulating HDL population (see for example Bruce et al., 1998, Annu. Rev. Nutr. 18:297-330). PLTP supplies lecithin to HDL, and CETP can move cholesteryl ester made by LCAT to other lipoproteins, particularly apoB-containing lipoproteins, such as VLDL. HDL triglyceride can be catabolized by the extracellular hepatic triglyceride lipase, and lipoprotein cholesterol is removed by the liver via several mechanisms.

Each HDL particle contains at least one molecule, and usually two to four molecules, of apolipoprotein (apo A-I). Apo A-I is synthesized by the liver and small intestine as preproapolipoprotein which is secreted as a proprotein that is rapidly cleaved to generate a mature polypeptide having 243 amino acid residues. Apo A-I consists mainly of a 22 amino acid repeating segment, spaced with helix-breaking proline residues. Apo A-I forms three types of stable structures with lipids: small, lipid-poor complexes referred to as pre-beta-1 HDL; flattened discoidal particles, referred to as pre-beta-2 HDL, which contain only polar lipids (e.g., phospholipid and cholesterol); and spherical particles containing both polar and nonpolar lipids, referred to as spherical or mature HDL (HDL₃ and HDL₂). Most HDL in the circulating population contains both apo A-I and apo A-II, a second major HDL protein. This apo A-I- and apo A-II-containing fraction is referred to herein as the AI/AII-HDL fraction of HDL. But the fraction of HDL containing only apo A-I, referred to herein as the AI-HDL fraction, appears to be more effective in RCT. Certain epidemiologic studies support the hypothesis that the AI-HDL fraction is antiartherogenic (Parra et al., 1992, Arterioscler. Thromb. 12:701-707; Decossin et al., 1997, Eur. J. Clin. Invest. 27:299-307).

Although the mechanism for cholesterol transfer from the cell surface is unknown, it is believed that the lipid-poor complex, pre-beta-1 HDL, is the preferred acceptor for cholesterol transferred from peripheral tissue involved in RCT. Cholesterol newly transferred to pre-beta-1 HDL from the cell surface rapidly appears in the discoidal pre-beta-2 HDL. PLTP may increase the rate of disc formation (Lagrost et al., 1996, J. Biol. Chem. 271:19058-19065), but data indicating a role for PLTP in RCT is lacking. LCAT reacts preferentially with discoidal and spherical HDL, transferring the 2-acyl group of lecithin or phosphatidylethanolamine to the free hydroxyl residue of fatty alcohols, particularly cholesterol, to generate cholesteryl esters (retained in the HDL) and lysolecithin. The LCAT reaction requires an apolipoprotein such apo A-I or apo A-IV as an activator. ApoA-I is one of the natural cofactors for LCAT. The conversion of cholesterol to its HDL-sequestered ester prevents re-entry of cholesterol into the cell, resulting in the ultimate removal of cellular cholesterol. Cholesteryl esters in the mature HDL particles of the AI-HDL fraction are removed by the liver and processed into bile more effectively than those derived from the AI/AII-HDL fraction. This may be due, in part, to the more effective binding of AI-HDL to the hepatocyte membrane. Several HDL receptors have been identified, the most well characterized of which is the scavenger receptor class B, type I (SR-BI) (Acton et al., 1996, Science 271:518-520). The SR-BI is expressed most abundantly in steroidogenic tissues (e.g., the adrenals), andin the liver (Landshulz et al., 1996, J. Clin. Invest. 98:984-995; Rigotti et al., 1996, J. Biol. Chem. 271:33545-33549). Other proposed HDL receptors include HB1 and HB2 (Hidaka and Fidge, 1992, Biochem J. 15:161-7; Kurata et al., 1998, J. Atherosclerosis and Thrombosis 4:112-7).

While there is a consensus that CETP is involved in the metabolism of VLDL- and LDL-derived lipids, its role in RCT remains controversial. However, changes in CETP activity or its acceptors, VLDL and LDL, play a role in “remodeling” the HDL population. For example, in the absence of CETP, the HDL becomes enlarged particles that are poorly removed from the circulation (for reviews on RCT and HDL, see Fielding & Fielding, 1995, J. Lipid Res. 36:211-228; Barrans et al., 1996, Biochem. Biophys. Acta. 1300:73-85; Hirano et al., 1997, Arterioscler. Thromb. Vasc. Biol. 17:1053-1059).

### 2.3 Reverse Transport of Other Lipids

HDL is not only involved in the reverse transport of cholesterol, but also plays a role in the reverse transport of other lipids, i.e., the transport of lipids from cells, organs, and tissues to the liver for catabolism and excretion. Such lipids include sphingomyelin, oxidized lipids, and lysophophatidylcholine. For example, Robins and Fasulo (1997, J. Clin. Invest. 99:380-384) have shown that HDL stimulates the transport of plant sterol by the liver into bile secretions.

### 2.4 Peroxisome Proliferator Activated Receptor Pathway

Peroxisome proliferators are a structurally diverse group of compounds that, when administered to rodents, elicit dramatic increases in the size and number of hepatic and renal peroxisomes, as well as concomitant increases in the capacity of peroxisomes to metabolize fatty acids via increased expression of the enzymes required for the β-oxidation cycle (Lazarow and Fujiki, 1985, Ann. Rev. Cell Biol. 1:489-530; Vamecq and Draye, 1989, Essays Biochem. 24:1115-225; and Nelali et al., 1988, Cancer Res. 48:5316-5324). Chemicals included in this group are the fibrate class of hypolipidermic drugs, herbicides, and phthalate plasticizers (Reddy and Lalwani, 1983, Crit. Rev. Toxicol. 12:1-58). Peroxisome proliferation can also be elicited by dietary or physiological factors, such as a high-fat diet and cold acclimatization.

Insight into the mechanism whereby peroxisome proliferators exert their pleiotropic effects was provided by the identification of a member of the nuclear hormone receptor superfamily activated by these chemicals (Isseman and Green, 1990, Nature 347:645-650). This receptor, termed peroxisome proliferator activated receptor α (PPAR_{α}), was subsequently shown to be activated by a variety of medium and long-chain fatty acids. PPAR_{α} activates transcription by binding to DNA sequence elements, termed peroxisome proliferator response elements (PPRE), in the form of a heterodimer with the retinoid X receptor (RXR). RXR is activated by 9-cis retinoic acid (see Kliewer et al., 1992, Nature 358:771-774; Gearing et al., 1993, Proc. Natl. Acad. Sci. USA 90:1440-1444, Keller et al., 1993, Proc. Natl. Acad. Sci. USA 90:2160-2164; Heyman et al., 1992, Cell 68:397-406, and Levin et al., 1992, Nature 355:359-361). Since the discovery of PPAR_{α}, additional isoforms of PPAR have been identified, e.g., PPAR_{β} (also known as PPAR_{δ}) and PPAR_{γ} and, which have similar functions and are similarly regulated.

PPREs have been identified in the enhancers of a number of gene-encoding proteins that regulate lipid metabolism. These proteins include the three enzymes required for peroxisomal β-oxidation of fatty acids; apolipoprotein A-I; medium-chain acyl-CoA dehydrogenase, a key enzyme in mitochondrial β-oxidation; and aP2, a lipid binding protein expressed exclusively in adipocytes (reviewed in Keller and Whali, 1993, TEM, 4:291-296; see also Staels and Auwerx, 1998, Atherosclerosis 137 Suppl:S19-23). The nature of the PPAR target genes coupled with the activation of PPARs by fatty acids and hypolipidemic drugs suggests a physiological role for the PPARs in lipid homeostasis.

It is clear that none of the commercially available cholesterol management drugs has a general utility in regulating lipid, lipoprotein, insulin and glucose levels in the blood. Thus, compounds that have one or more of these utilities are clearly needed. Further, there is a clear need to develop safer drugs that are efficacious at lowering serum cholesterol, increasing HDL serum levels, preventing coronary heart disease, and/or treating existing disease such as atherosclerosis, obesity, diabetes, and other diseases that are affected by lipid metabolism and/or lipid levels. There is also a clear need to develop drugs that may be used with other lipid-altering treatment regimens in a synergistic manner. There is still a further need to provide useful therapeutic agents whose solubility and Hydrophile/Lipophile Balance (HLB) can be readily varied.

Citation or identification of any reference in Section 2 of this application is not an admission that such reference is available as prior art to the present invention.

### 3. SUMMARY OF THE INVENTION

The present invention relates to ether compounds and pharmaceutically acceptable salts, hydrates, solvates, clathrates, stereoisomers, diastereomers, geometric isomers, or mixtures thereof; compositions comprising the ether compounds, and methods for treating or preventing disorders in mammals, particularly in humans.

In one embodiment the invention encompasses compounds of formula I:

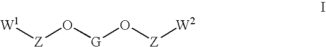

or a pharmaceutically acceptable salt, hydrate, solvate, or a mixture thereof, wherein:

(a) each occurrence of Z is independently (CH₂)ₘ, (CH═CH)ₜ, or phenyl, where each occurrence of m and t is an independent integer ranging from 1 to 9;

(b) G is (CH₂)ₓ, CH₂CH═CHCH₂, CH═CH, CH₂₋phenyl-CH₂, or phenyl, where x is 2, 3, or 4;

(c) W¹ and W² are independently C(R¹)(R²)(CH₂)ₙ₋Y, V, C(R³)(R⁴)—(CH₂)_{c-}C(R⁵)(R⁶)—(CH₂)ₙ₋Y, or C(R¹)(R²)—(CH₂)_{c-}V where c is 1 or 2 and n is an integer ranging from 0 to 4;

(d) each occurrence of R¹ and R² is independently (C₁₋C₆)alkyl, (C₂₋C₆)alkenyl, (C₂₋C₆)alkynyl, phenyl, benzyl, or R¹ and R² and the carbon to which they are both attached are taken together to form a (C₃-C₇)cycloakyl group;

(e) each occurrence of R³ and R⁴ is independently H, (C₁₋C₆)alkyl, (C₂₋C₆)alkenyl, (C₂₋C₆)alkynyl, phenyl, benzyl, R³ and R⁴ and the carbon to which they are both attached are taken together to form a (C₃-C₇)cycloakyl group;

(f) R⁵ is H, (C₁₋C₆)alkyl, (C₂₋C₆)alkenyl, (C₂₋C₆)alkynyl, (C₁₋C₆)alkoxy, phenyl, benzyl, Cl, Br, CN, NO₂, or CF₃;

(g) R⁶ is OH, (C₁₋C₆)alkyl, (C₂₋C₆)alkenyl, (C₂₋C₆)alkynyl, (C₁₋C₆)alkoxy, phenyl, benzyl, Cl, Br, CN, NO₂, or CF₃;

(h) V is

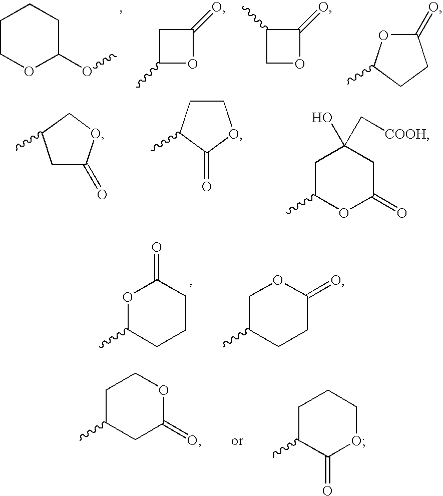

(i) each occurrence of Y is independently (C₁₋C₆)alkyl, OH, COOH, CHO, COOR⁷, SO₃H,

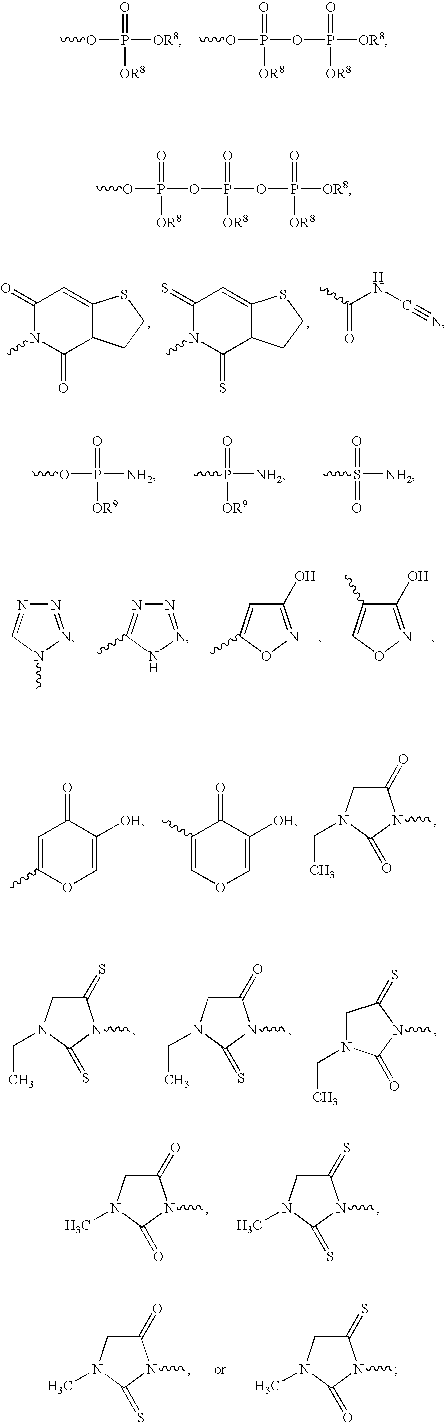

(j) R⁷ is (C₁₋C₆)alkyl, (C₂₋C₆)alkenyl, (C₂₋C₆)alkynyl, phenyl, or benzyl and is unsubstituted or substituted with one or more halo, OH, (C₁₋C₆)alkoxy, or phenyl groups;

(k) each occurrence of R⁸ is independently H, (C₁₋C₆)alkyl, (C₂₋C₆)alkenyl, or (C₂₋C₆)alkynyl and is unsubstituted or substituted with one or two halo, OH, (C₁₋C₆)alkoxy, or phenyl groups; and

(l) each occurrence of R⁹ is independently H, (C₁₋C₆)alkyl, (C₂₋C₆)alkenyl, or (C₂₋C₆)alkynyl.

Preferably, in the compounds of formula I:

(i) when G is (CH₂)ₓ, then W¹ and W² cannot both be C(R¹)(R²)—CHO or cannot both be

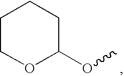

(ii) that when G is phenyl, then W¹ and W² cannot:

both be C(R¹)(R²)—COOH,

both be C(R¹)(R²)—CH₂OH,

both be C(R¹)(R²)—COOR⁷,

both be (CH₂)₃₋C(H)(OH)—CH₂OH,

both be (CH₂)₂₋C(H)(OH)—CH₂OH,

both be C(R¹)(R²)—CHO, or

both be

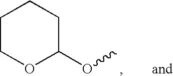

(iii) that when every occurrence of Z is phenyl, then W¹ and W² cannot both be C(R¹)(R²)—OH.

Preferably, in the compounds of formula I, W¹ and W² are independently C(R¹)(R²)(CH₂)ₙ₋Y, V, C(R³)(R⁴)—(CH₂)_{c-}C(R⁵)(R⁶)—Y, or C(R¹)(R²)—(CH₂)_{c-}V. More preferably, W¹ and W² are independent C(R¹)(R²)(CH₂)ₙ₋Y groups, where Y is independently OH, COOR⁷, or COOH.

It is also preferably in the compound of formula I, that m is an integer ranging from 1 to 4 and t is 1.

In another embodiment, the invention encompasses compounds of formula Ia:

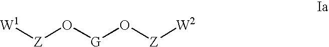

or a pharmaceutically acceptable salt, hydrate, solvate, or a mixture thereof, wherein:

(a) each occurrence of Z is independently (CH₂)ₘ or (CH═CH)ₜ, where each occurrence of m and t is an independent integer ranging from 1 to 9;

(b) G is (CH₂)ₓ, CH₂CH═CHCH₂, or CH═CH, where x is 2, 3, or 4;

(c) W¹ and W² are independently C(R¹)(R²)(CH₂)ₙ₋Y, V, or C(R¹)(R²)(CH₂)_{c-}V, where c is 1 or 2 and n is an integer ranging from 0 to 4;

(d) each occurrence of R¹ and R² is independently (C₁₋C₆)alkyl, (C₂₋C₆)alkenyl, (C₂₋C₆)alkynyl, phenyl, benzyl, or R¹ and R² and the carbon to which they are both attached are taken together to form a (C₃-C₇)cycloakyl group;

(e) V is

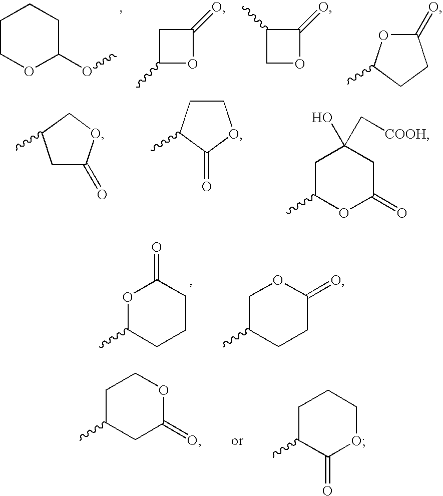

(f) each occurrence of Y is independently (C₁₋C₆)alkyl, OH, COOH, CHO, COOR⁷, SO₃H,

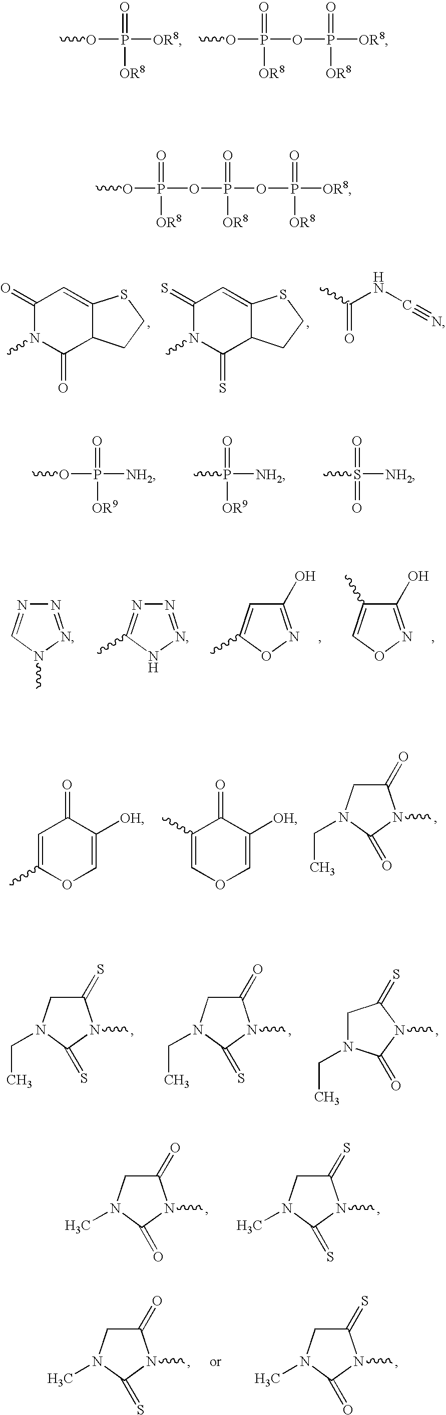

(g) R⁷ is (C₁₋C₆)alkyl, (C₂₋C₆)alkenyl, (C₂₋C₆)alkynyl, phenyl, or benzyl and is unsubstituted or substituted with one or more halo, OH, (C₁₋C₆)alkoxy, or phenyl groups;

(h) each occurrence of R⁸ is independently H, (C₁₋C₆)alkyl, (C₂₋C₆)alkenyl, or (C₂₋C₆)alkynyl and is unsubstituted or substituted with one or two halo, OH, (C₁₋C₆)alkoxy, or phenyl groups; and

(i) each occurrence of R⁹ is independently H, (C₁₋C₆)alkyl, (C₂₋C₆)alkenyl, or (C₂₋C₆)alkynyl.

Preferably, in the compounds of formula Ia, when G is (CH₂)ₓ, then W¹ and W² cannot both be C(R¹)(R²)—CHO or cannot both be

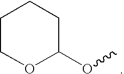

In still another embodiment, the invention encompasses compounds of formula Ib

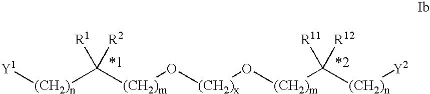

or a pharmaceutically acceptable salt, hydrate, solvate, or a mixture thereof, wherein:

(a) each occurrence of m is independently an integer ranging from 1 to 9;

(b) each occurrence of n is an independent integer ranging from 0 to 4;

(c) x is 2, 3, or 4;

(d) each occurrence of R¹ and R² is independently (C₁-C₆)alkyl, (C₂-C₆)alkenyl, (C₂-C₆)alkynyl, phenyl, benzyl, or R¹ and R² and the carbon to which they are both attached are taken together to form a (C₃-C₇)cycloakyl group;

(e) each occurrence of R¹¹ and R¹² is independently H, (C₁-C₆)alkyl, (C₂-C₆)alkenyl, (C₂-C₆)alkynyl, phenyl, benzyl, or R¹ and R² and the carbon to which they are both attached are taken together to form a (C₃-C₇)cycloakyl group;

(f) Y¹ and Y² are independently (C₁-C₆)alkyl, OH, COOH, CHO, COOR⁷, SO₃H,

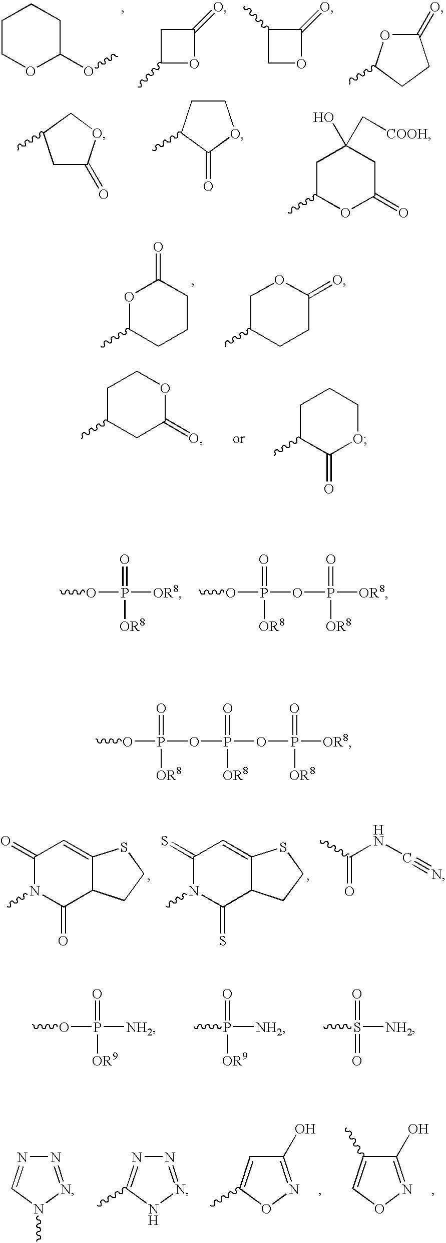

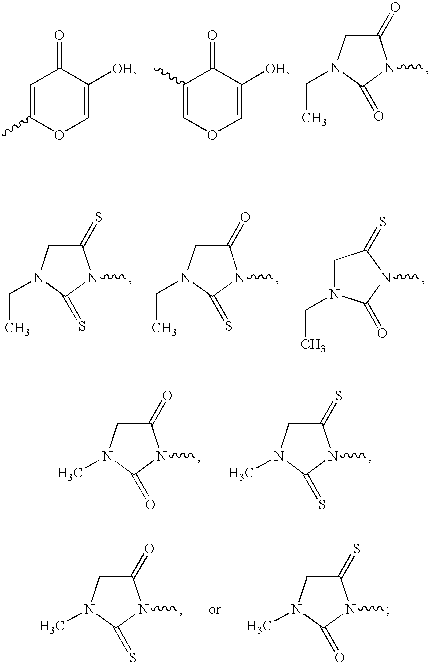

(g) R⁷ is (C₁₋C₆)alkyl, (C₂₋C₆)alkenyl, (C₂₋C₆)alkynyl, phenyl, or benzyl and is unsubstituted or substituted with one or more halo, OH, (C₁₋C₆)alkoxy, or phenyl groups;

(h) each occurrence of R⁸ is independently H, (C₁₋C₆)alkyl, (C₂₋C₆)alkenyl, or (C₂₋C₆)alkynyl and is unsubstituted or substituted with one or two halo, OH, (C₁₋C₆)alkoxy, or phenyl groups;

(i) each occurrence of R⁹ is independently H, (C₁₋C₆)alkyl, (C₂₋C₆)alkenyl, or (C₂₋C₆)alkynyl; and

(j) with the proviso that both occurrences of Y cannot both be CHO.

When R¹ and R² attached to the same carbon are different chemical groups, the symbols *¹ and *² represent independent chiral-carbon centers. Each chiral center is independent of the other and is racemic, substantially of configuration R, substantially of configuration S, or any mixture thereof. Thus in one embodiment, the compounds of formula Ib are optically active.

In a separate embodiment of compounds of formula Ib, the chiral center represented by *¹ is of the stereochemical configuration R or substantially R.

In another embodiment, the chiral center represented by *¹ is of the stereochemical configuration S or substantially S.

In still another embodiment, the chiral center represented by *² is of the stereochemical configuration R or substantially R.

In one more embodiment, the chiral center represented by *² is of the stereochemical configuration S or substantially S.

In yet another embodiment, the chiral centers represented by *¹ *² both have the same stereochemical configuration.

In another embodiment, the chiral centers represented by *¹ *² are of the stereochemical configuration (S¹,S²) or substantially (S¹,S²).

In yet another embodiment, the chiral centers represented by *¹ *² are of the stereochemical configuration (S¹,R²) or substantially (S¹,R²).

In still another embodiment, the chiral centers represented by *¹ *² are of the stereochemical configuration (R¹,R²) or substantially (R¹,R²).

In another embodiment, the chiral centers represented by *¹ *² are of the stereochemical configuration (R¹,S²) or substantially (R¹,S²).

In another embodiment, the invention encompasses compounds of formula Ic

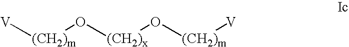

or a pharmaceutically acceptable salt, hydrate, solvate, or a mixture thereof, wherein:

(a) each occurrence of m is an independent integer ranging from 1 to 9;

(b) x is 2, 3, or 4;

(c) V is

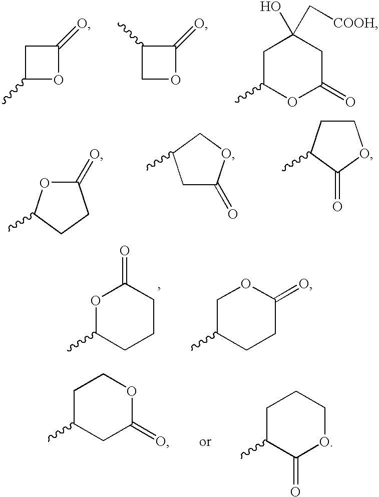

In yet another embodiment, the invention concerns compounds of the formula Id:

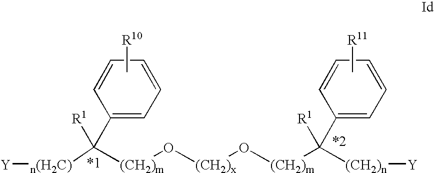

or a pharmaceutically acceptable salt, hydrate, solvate, or a mixture thereof, wherein

(a) each occurrence of m is independently an integer ranging from 1 to 9;

(b) each occurrence of n is an independent integer ranging from 0 to 4;

(c) x is 2, 3, or 4;

(d) each occurrence of R¹ is independently (C₁₋C₆)alkyl, (C₂₋C₆)alkenyl, (C₂₋C₆)alkynyl, phenyl, or benzyl;

(e) each occurrence of Y is (C₁₋C₆)alkyl, OH, COOH, CHO, COOR⁷, SO₃H,

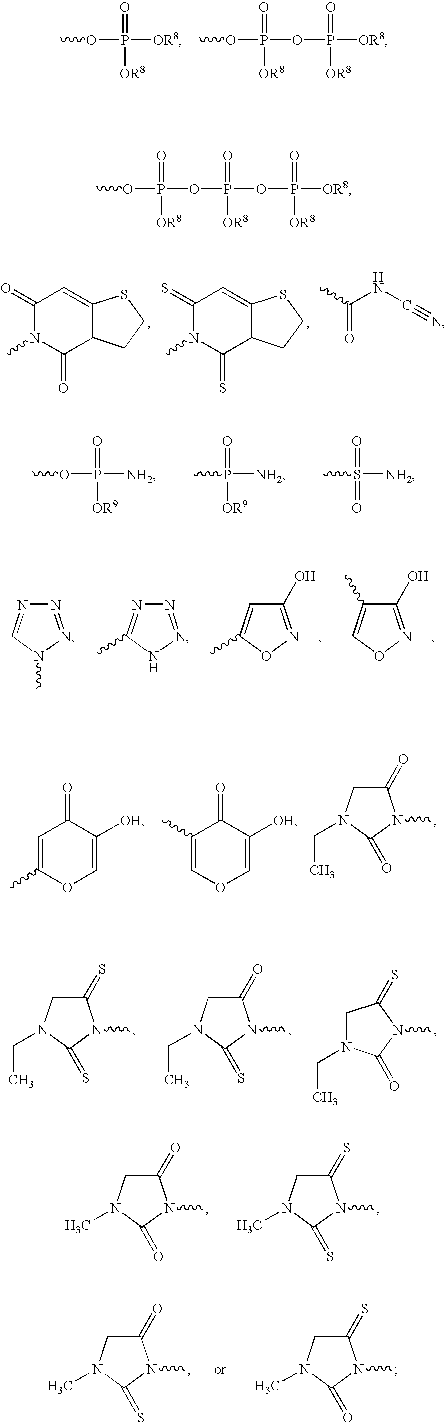

(f) R⁷ is H, (C₁₋C₄) alkyl, phenyl, or benzyl, and is substituted or unsubstituted with one or more halo, OH, (C₁-C₆)alkoxy, or phenyl groups;

(g) each occurrence of R⁸ is independently H, (C₁₋C₆)alkyl, (C₂₋C₆)alkenyl, or (C₂₋C₆)alkynyl and is unsubstituted or substituted with one or two halo, OH, (C₁₋C6)alkoxy, or phenyl groups;

(h) each occurrence of R⁹ is independently H, (C₁₋C₆)alkyl, (C₂₋C₆)alkenyl, or (C₂₋C₆)alkynyl;

(i) R¹⁰ and R¹¹ are independently H, halogen, (C₁₋C₄)alkyl, (C₁₋C₄)alkoxy, (C₆)aryl, (C₆)aryloxy, CN, or NO₂, N(R⁷)₂.

In formula Id, the symbols *¹ and *² represent chiral-carbon centers. Each chiral center is independent of the other and is racemic, substantially of configuration R, substantially of configuration S, or any mixture thereof. Thus in one embodiment, the compounds of formula Id are optically active.

In a separate embodiment of compounds of formula Id, the chiral center represented by *¹ is of the stereochemical configuration R or substantially R.

In another embodiment, the chiral center represented by *¹ is of the stereochemical configuration S or substantially S.

In still another embodiment, the chiral center represented by *² is of the stereochemical configuration R or substantially R.

In one more embodiment, the chiral center represented by *² is of the stereochemical configuration S or substantially S.

In yet another embodiment, the chiral centers represented by *¹ *² both have the same stereochemical configuration.

In another embodiment, the chiral centers represented by *¹ *² are of the stereochemical configuration (S¹,S²) or substantially (S¹,S²).

In yet another embodiment, the chiral centers represented by *¹ *² are of the stereochemical configuration (S¹,R²) or substantially (S¹,R²).

In still another embodiment, the chiral centers represented by *¹ *² are of the stereochemical configuration (R¹,R²) or substantially (R¹,R²).

In another embodiment, the chiral centers represented by *¹ *² are of the stereochemical configuration (R¹,S²) or substantially (R¹,S²).

In another embodiment, the invention encompasses compounds of formula II:

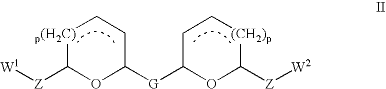

or a pharmaceutically acceptable salt, hydrate, solvate, or a mixture thereof, wherein:

(a) each occurrence of Z is independently (CH₂)ₘ, (CH═CH)ₜ, or phenyl, where each occurrence of m and t are independent integers ranging from 1 to 5;

(b) G is (CH₂)ₓ, CH₂CH═CHCH₂, CH═CH, CH₂₋phenyl-CH₂, or phenyl, where x is an integer ranging from 1 to 4;

(c) W¹ and W² are independently C(R¹)(R²)(CH₂)ₙ₋Y, V, or C(R¹)(R²)—(CH₂)_{c-}V where c is 1 or 2 and n is an integer ranging from 0 to 4;

(d) each occurrence of R¹ and R² is independently (C₁₋C₆)alkyl, (C₂₋C₆)alkenyl, (C₂₋C₆)alkynyl, phenyl, benzyl, or R¹ and R² and the carbon to which they are both attached are taken together to form a (C₃-C₇)cycloakyl group;;

(e) V is

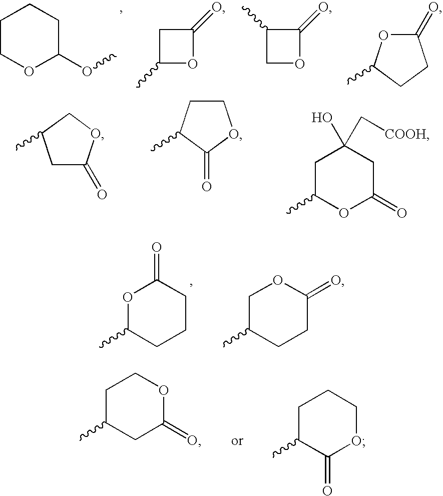

(f) each occurrence of Y is independently (C₁₋C₆)alkyl, OH, COOH, CHO, COOR⁷, SO₃H,

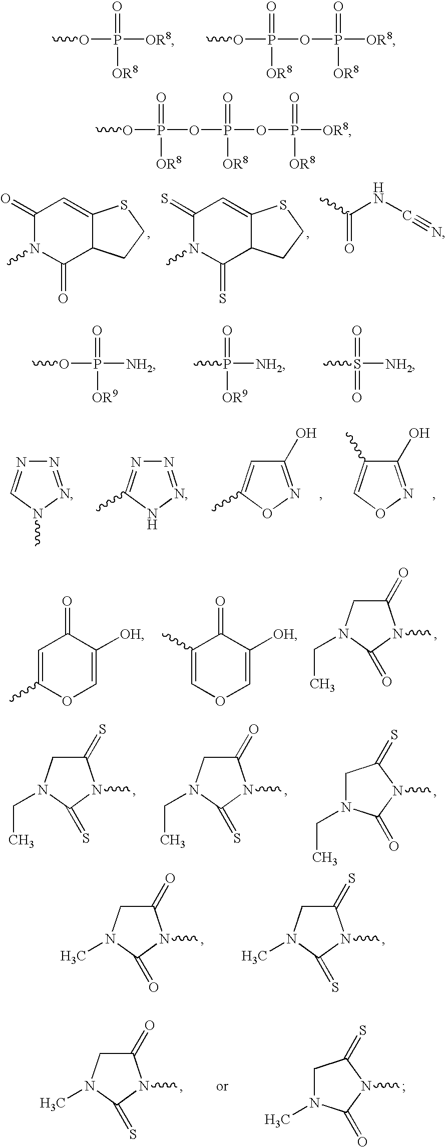

(g) R⁷ is (C₁₋C₆)alkyl, (C₂₋C₆)alkenyl, (C₂₋C₆)alkynyl, phenyl, or benzyl and is unsubstituted or substituted with one or more halo, OH, (C₁₋C₆)alkoxy, or phenyl groups;

(h) each occurrence of R⁸ is independently H, (C₁₋C₆)alkyl, (C₂₋C₆)alkenyl, or (C₂₋C₆)alkynyl and is unsubstituted or substituted with one or two halo, OH, (C₁₋C₆)alkoxy, or phenyl groups;

(i) each occurrence of R⁹ is independently H, (C₁₋C₆)alkyl, (C₂₋C₆)alkenyl, or (C₂₋C₆)alkynyl; and

(j) each occurrence of p is independently 0 or 1 where the broken line represents an optional presence of 1 or 2 additional carbon-carbon bonds that when present complete 1 or 2 carbon-carbon double bonds.

Preferably, in compounds of formula II, W¹ and W² are independent C(R¹)(R²)(CH₂)ₙ₋Y groups and each occurrence of Y is independently OH, COOR⁷, or COOH. In one embodiment of compounds of formula II that p is 0 in another p is 1. In still another embodiment of compounds of formula II, t is 1.

In another embodiment, the invention encompasses compounds of formula IIa:

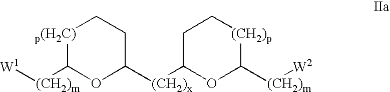

or a pharmaceutically acceptable salt, hydrate, solvate, or a mixture thereof, wherein:

(a) each occurrence of m is independently an integer ranging from 1 to 5;

(b) x is an integer ranging from 1 to 4;

(c) W¹ and W² are independently C(R¹)(R²)(CH₂)ₙ₋Y, V, or C(R¹)(R²)—(CH₂)_{c-}V where c is 1 or 2 and n is an integer ranging from 0 to 4;

(d) each occurrence of R¹ and R² is independently (C₁₋C₆)alkyl, (C₂₋C₆)alkenyl, (C₂₋C₆)alkynyl, phenyl, benzyl, or R¹ and R² and the carbon to which they are both attached are taken together to form a (C₃-C₇)cycloakyl group;

(e) V is

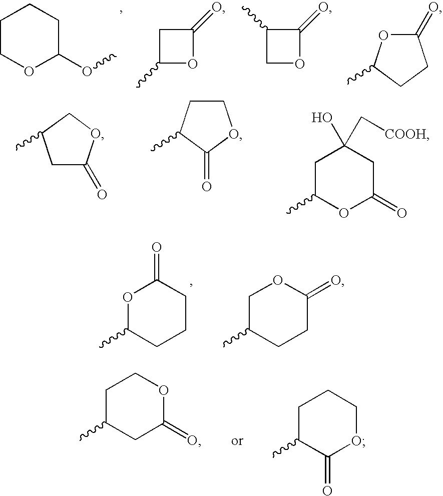

(f) Y is (C₁-C₆)alkyl, OH, COOH CHO, COOR⁷, SO₃H,

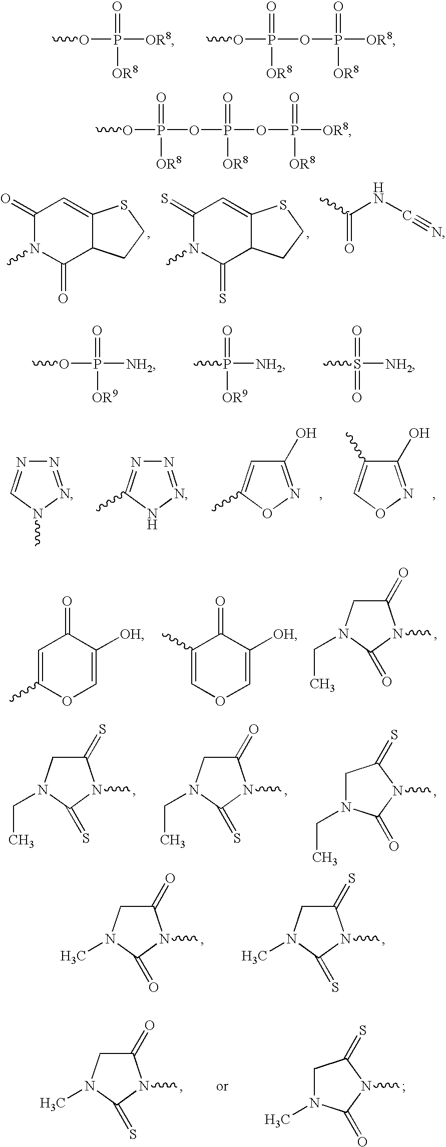

(g) R⁷ is (C₁₋C₆)alkyl, (C₂₋C₆)alkenyl, (C₂₋C₆)alkynyl, phenyl, or benzyl and is unsubstituted or substituted with one or more halo, OH, (C₁₋C₆)alkoxy, or phenyl groups;

(h) each occurrence of R⁸ is independently H, (C₁₋C₆)alkyl, (C₂₋C₆)alkenyl, or (C₂₋C₆)alkynyl and is unsubstituted or substituted with one or two halo, OH, (C₁₋C₆)alkoxy, or phenyl groups;

(i) each occurrence of R⁹ is independently H, (C₁₋C₆)alkyl, (C₂₋C₆)alkenyl, or (C₂₋C₆)alkynyl; and

(f) each occurrence of p is independently 0 or 1.

In another embodiment, the invention encompasses compounds of formula III:

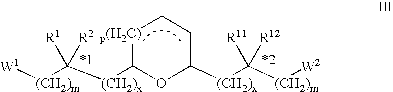

or a pharmaceutically acceptable salt, hydrate, solvate, or a mixture thereof, wherein:

(a) each occurrence of R¹ and R² is independently (C₁-C₆)alkyl, (C₂-C₆)alkenyl, (C₂₋C₆)alkynyl, phenyl, benzyl, or R¹ and R² and the carbon to which they are both attached are taken together to form a (C₃-C₇)cycloakyl group;

(b) each occurrence of R¹¹ and R¹² and the carbon to which they are both attached are taken together to form a (C₃-C₇)cycloakyl group;

(c) each occurrence of m is an independent integer ranging from 0 to 6;

(d) each occurrence of x is independently and integer from 2 to 5;

(e) W¹ and W² are independently (C₁₋C₆)alkyl, OH, C(O)OH, CHO, OC(O)R⁷, C(O)OR⁷, SO₃H,

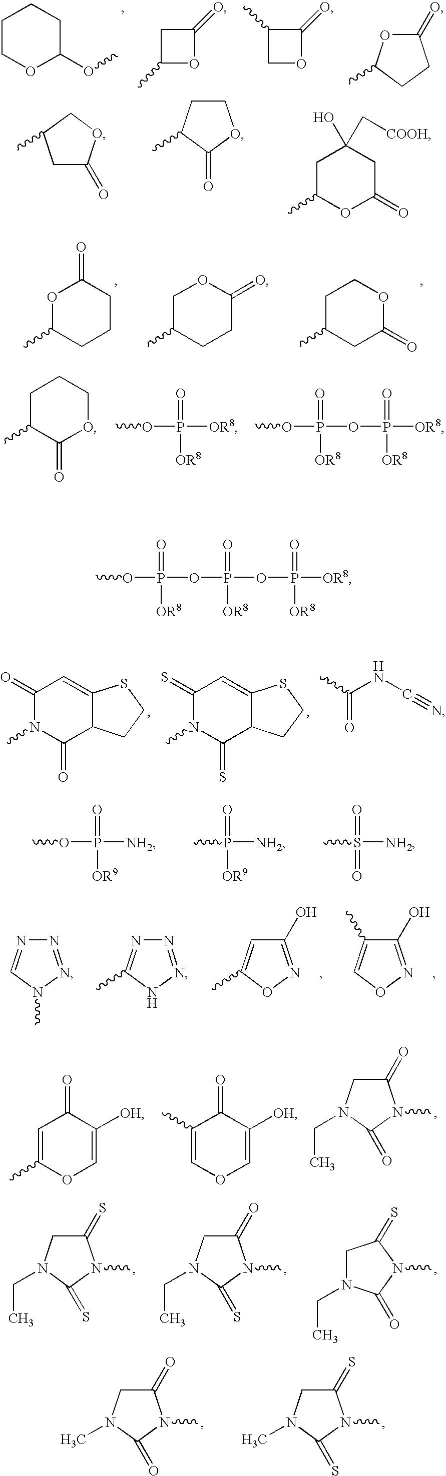

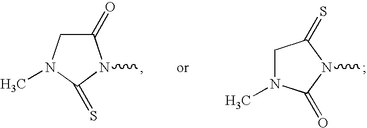

(e) R⁷ is (C₁₋C₆)alkyl, (C₂₋C₆)alkenyl, (C₂₋C₆)alkynyl, phenyl, or benzyl and is unsubstituted or substituted with one or more halo, OH, (C₁₋C₆)alkoxy, or phenyl groups;

(f) each occurrence of R⁸ is independently H, (C₁₋C₆)alkyl, (C₂₋C₆)alkenyl, or (C₂₋C₆)alkynyl and is unsubstituted or substituted with one or two halo, OH, (C₁₋C₆)alkoxy, or phenyl groups;

(g) each occurrence of R⁹ is independently H, (C₁₋C₆)alkyl, (C₂₋C₆)alkenyl, or (C₂₋C₆)alkynyl; and

(h) p is 0 or 1 where the broken line represents an optional presence of 1 or 2 additional carbon-carbon bonds that when present complete 1 or 2 carbon-carbon double bonds.

Preferably, in compound of formula III, W¹ and W² are independently OH, COOR⁷, or COOH.

The ring in formula III can be saturated or contain one or two double bonds. For example, the ring in compounds of formula III can be:

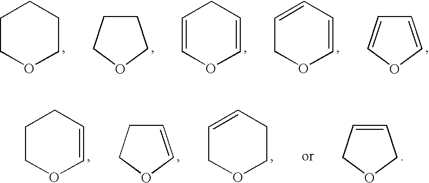

Preferably, in compound of formula III, W¹ and W² are independently OH, COOR⁷, or COOH.

In one more embodiment of compound of formula III, p is 0; in another, p is 1.

In still another embodiment of compounds of formula III, the broken line is absent.

In yet another embodiment of compounds of formula III, each occurrence of R¹ and R² is independently (C₁₋C₆)alkyl, (C₂₋C₆)alkenyl, (C₂₋C₆)alkynyl, phenyl, or benzyl.

In compounds of formula III, when R¹ and R² attached to the same carbon are different chemical groups, the symbols *¹ and *² represent chiral-carbon centers. Each chiral center is independent of the other and is racemic, substantially of configuration R, substantially of configuration S, or any mixture thereof. Thus in one embodiment, the compounds of formula III are optically active.

In a separate embodiment of compounds of formula III, the chiral center represented by *¹ is of the stereochemical configuration R or substantially R.

In another embodiment, the chiral center represented by *¹ is of the stereochemical configuration S or substantially S.

In still another embodiment, the chiral center represented by *² is of the stereochemical configuration R or substantially R.

In one more embodiment, the chiral center represented by *² is of the stereochemical configuration S or substantially S.

In yet another embodiment, the chiral centers represented by *¹ *² both have the same stereochemical configuration.

In another embodiment, the chiral centers represented by *¹ *² are of the stereochemical configuration (S¹,S²) or substantially (S¹,S²).

In yet another embodiment, the chiral centers represented by *¹ *² are of the stereochemical configuration (S¹,R²) or substantially (S¹,R²).

In still another embodiment, the chiral centers represented by *¹ *² are of the stereochemical configuration (R¹,R²) or substantially (R¹,R²).

In another embodiment, the chiral centers represented by *¹ *² are of the stereochemical configuration (R¹,S²) or substantially (R¹,S²).

In another embodiment, the invention encompasses compounds of formula IV:

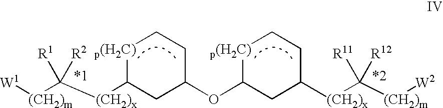

or a pharmaceutically acceptable salt, hydrate, solvate, or a mixture thereof, wherein:

(a) each occurrence of R¹ and R² is independently (C₁-C₆)alkyl, (C₂-C₆)alkenyl, (C₂-C₆)alkynyl, phenyl, benzyl, or R¹ and R² and the carbon to which they are both attached are taken together to form a (C₃-C₇)cycloakyl group;

(b) each occurrence of R¹¹ and R¹² is independently H, (C₁-C₆)alkyl, (C₂-C₆)alkenyl, (C₂-C₆)alkynyl, phenyl, benzyl, or R¹ and R² and the carbon to which they are both attached are taken together to form a (C₃-C₇)cycloakyl group;

(c) each occurrence of m is independently an integer ranging from 0 to 6;

(d) each occurrence of x is independently and integer from 0 to 4;

(e) W¹ and W² are independently (C₁-C₆)alkyl, OH, C(O)OH, CHO, OC(O)R⁷, C(O)OR⁷, SO₃H,

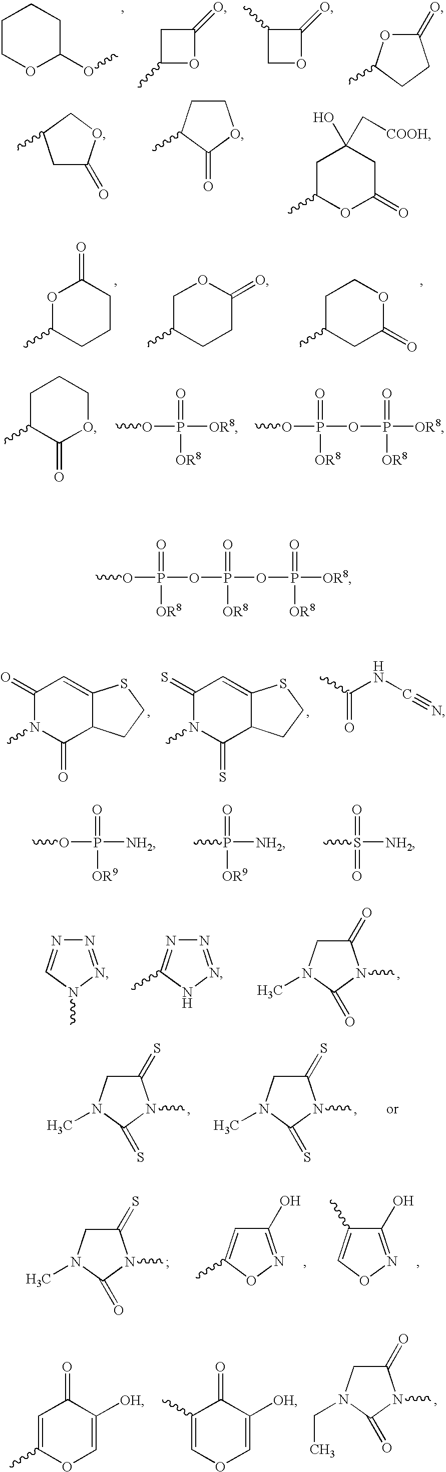

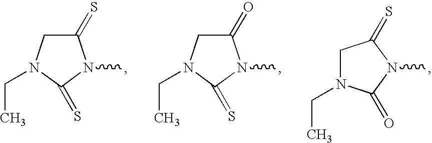

(f) R⁷ is (C₁₋C₆)alkyl, (C₂₋C₆)alkenyl, (C₂₋C₆)alkynyl, phenyl, or benzyl and is unsubstituted or substituted with one or more halo, OH, (C₁₋C₆)alkoxy, or phenyl groups;

(g) each occurrence of R⁸ is independently H, (C₁₋C₆)alkyl, (C₂₋C₆)alkenyl, or (C₂₋C₆)alkynyl and is unsubstituted or substituted with one or two halo, OH, (C₁₋C₆)alkoxy, or phenyl groups;

(h) each occurrence of R⁹ is independently H, (C₁₋C₆)alkyl, (C₂₋C₆)alkenyl, or (C₂₋C₆)alkynyl; and

(i) each occurrence of p is independently 0 or 1 where the broken line represents an optional presence of 1, 2, or 3 additional carbon-carbon bonds that when present form a cycloalkenyl group, a cyclodienyl group, or a phenyl group.

In compounds of formula IV, when R¹ and R² attached to the same carbon are different chemical groups, the symbols *¹ and *² represent independent chiral-carbon centers. Each chiral center is independent of the other and is racemic, substantially of configuration R, substantially of configuration S, or any mixture thereof. Thus in one embodiment, the compounds of formula IV are optically active.

In a separate embodiment of compounds of formula IV, the chiral center represented by *¹ is of the stereochemical configuration R or substantially R.

In another embodiment, the chiral center represented by *¹ is of the stereochemical configuration S or substantially S.

In still another embodiment, the chiral center represented by *² is of the stereochemical configuration R or substantially R.

In one more embodiment, the chiral center represented by *² is of the stereochemical configuration S or substantially S.

In yet another embodiment, the chiral centers represented by *¹ *² both have the same stereochemical configuration.

In another embodiment, the chiral centers represented by *¹ *² are of the stereochemical configuration (S¹,S²) or substantially (S¹,S²).

In yet another embodiment, the chiral centers represented by *¹ *² are of the stereochemical configuration (S¹,R²) or substantially (S¹,R²).

In still another embodiment, the chiral centers represented by *¹ *² are of the stereochemical configuration (R¹,R²) or substantially (R¹,R²).

In another embodiment, the chiral centers represented by *¹ *² are of the stereochemical configuration (R¹,S²) or substantially (R¹,S²).

The rings in compounds IV can be saturated or contain 1, 2, or 3 double bonds. Of course with 3 double bonds, the ring is a phenyl group. For example, the ring groups in compounds of formula IV can independently be:

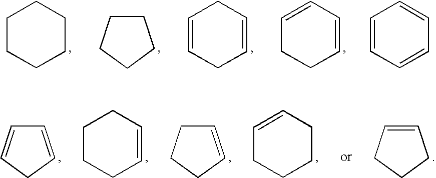

Preferably, the ring of compounds IV is a phenyl ring.

Preferably, in compounds of formula IV, W¹ and W² are independently OH, COOR⁷, or COOH.

In another embodiment of compounds of formula IV, each occurrence of R¹ and R² is independently (C₁₋C₆)alkyl, (C₂₋C₆)alkenyl, (C₂₋C₆)alkynyl, phenyl, or benzyl.

In still another embodiment of compounds of formula IV, p is 0, and in another, p is 1.

In one more embodiment of compounds of formula 4, the broken line is absent.

In another embodiment, compound of formula IV have the formula:

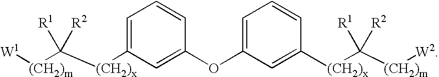

And in still another embodiment, compound of formula IV have the formula:

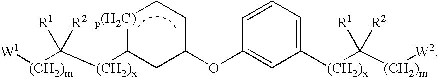

The compounds of the invention are useful for treating or preventing aging, Alzheimer's Disease, cancer, cardiovascular disease, diabetic nephropathy, diabetic retinopathy, a disorder of glucose metabolism, dyslipidemia, dyslipoproteinemia, enhancing bile production, enhancing reverse lipid transport, hypertension, impotence, inflammation, insulin resistance, lipid elimination in bile, modulating C reactive protein, obesity, oxysterol elimination in bile, pancreatitis, Parkinson's disease, a peroxisome proliferator activated receptor-associated disorder, phospholipid elimination in bile, renal disease, septicemia, metabolic syndrome disorders (e.g., Syndrome X), a thrombotic disorder, inflammatory processes and diseases like gastrointestinal disease, irritable bowel syndrome (IBS), inflammatory bowel disease (e.g., Crohn's Disease, ulcerative colitis), arthritis (e.g., rheumatoid arthritis, osteoarthritis), autoimmune disease (e.g., systemic lupus erythematosus), scleroderma, ankylosing spondylitis, gout and pseudogout, muscle pain: polymyositis/polymyalgia rheumatica/fibrositis; infection and arthritis, juvenile rheumatoid arthritis, tendonitis, bursitis and other soft tissue rheumatism.

The invention further provides pharmaceutical compositions comprising one or more compounds of the invention or a pharmaceutically acceptable salt, hydrate, solvate, clathrate, enantiomer, diastereomer, racemate, or a mixture of stereoisomers thereof and a pharmaceutically acceptable vehicle, excipient, or diluent and a pharmaceutically acceptable vehicle, excipient, or diluent.

These pharmaceutical compositions are useful for treating or preventing a disease or disorder such as, but not limited to, aging, Alzheimer's Disease, cancer, cardiovascular disease, diabetic nephropathy, diabetic retinopathy, a disorder of glucose metabolism, dyslipidemia, dyslipoproteinemia, enhancing bile production, enhancing reverse lipid transport, hypertension, impotence, inflammation, insulin resistance, lipid elimination in bile, modulating C reactive protein, obesity, oxysterol elimination in bile, pancreatitis, Parkinson's disease, a peroxisome proliferator activated receptor-associated disorder, phospholipid elimination in bile, renal disease, septicemia, metabolic syndrome disorders (e.g., Syndrome X), and a thrombotic disorder, inflammatory processes and diseases like gastrointestinal disease, irritable bowel syndrome (IBS), inflammatory bowel disease (e.g., Crohn's Disease, ulcerative colitis), arthritis (e.g., rheumatoid arthritis, osteoarthritis), autoimmune disease (e.g., systemic lupus erythematosus), scleroderma, ankylosing spondylitis, gout and pseudogout, muscle pain: polymyositis/polymyalgia rheumatica/fibrositis; infection and arthritis, juvenile rheumatoid arthritis, tendonitis, bursitis and other soft tissue rheumatism.

The present invention provides a method for treating or preventing a aging, Alzheimer's Disease, cancer, cardiovascular disease, diabetic nephropathy, diabetic retinopathy, a disorder of glucose metabolism, dyslipidemia, dyslipoproteinemia, enhancing bile production, enhancing reverse lipid transport, hypertension, impotence, inflammation, insulin resistance, lipid elimination in bile, modulating C reactive protein, obesity, oxysterol elimination in bile, pancreatitis, Parkinson's disease, a peroxisome proliferator activated receptor-associated disorder, phospholipid elimination in bile, renal disease, septicemia, metabolic syndrome disorders (e.g., Syndrome X), and a thrombotic disorder, inflammatory processes and diseases like gastrointestinal disease, irritable bowel syndrome (IBS), inflammatory bowel disease (e.g., Crohn's Disease, ulcerative colitis), arthritis (e.g., rheumatoid arthritis, osteoarthritis), autoimmune disease (e.g., systemic lupus erythematosus), scleroderma, ankylosing spondylitis, gout and pseudogout, muscle pain: polymyositis/polymyalgia rheumatica/fibrositis; infection and arthritis, juvenile rheumatoid arthritis, tendonitis, bursitis and other soft tissue rheumatism comprising administering to a patient in need of such treatment or prevention a therapeutically effective amount of a compound of the invention or a pharmaceutical composition comprising a compound of the invention and a pharmaceutically acceptable vehicle, excipient, or diluent.

The present invention provides a method for treating or preventing stroke, peripheral vascular disease, polymylagia rheumatica, polymyositis, fibrositis, gastrointestinal disease, irritable bowel syndrome, inflammatory bowel disease, asthma, vasculitis, ulcerative colitis, Crohn's disease, Kawasaki disease, Wegener's granulomatosis, systemic lupus erythematosus, multiple sclerosis, autoimmune chronic hepatitis, osteoporosis, rheumatoid arthritis, juvenile rheumatoid arthritis, osteoarthritis, tendonitis, bursitis, systemic lupus, erythematosus, scleroderma, ankylosing spondylitis, gout, pseudogout, non-insulin dependent diabetes mellitus, polycystic ovarian disease, hyperlipidemias, familial hypercholesterolemia, familial combined hyperlipidemia, lipoprotein lipase deficiencies, hypertriglyceridemia, hypoalphalipoproteinemia, hypercholesterolemia, lipoprotein abnormalities associated with diabetes, lipoprotein abnormalities associated with obesity, lipoprotein abnormalities associated with Alzheimer's Disease, high levels of blood triglycerides, high levels of low density lipopotein cholesterol, high levels of apolipoprotein B, high levels of lipoprotein Lp(a) cholesterol, high levels of very low density lipoprotein cholesterol, high levels of fibrinogen, high levels of insulin, high levels of glucose, low levels of high density lipoprotein cholesterol, or NIDDM in a patient comprising administering to a patient in need of such treatment or prevention a therapeutically effective amount of a compound of the invention or a pharmaceutical composition comprising a compound of the invention and a pharmaceutically acceptable vehicle, excipient, or diluent.

The present invention further provides a method for reducing the fat content of meat in livestock comprising administering to livestock in need of such fat-content reduction a therapeutically effective amount of a compound of the invention or a pharmaceutical composition comprising a compound of the invention and a pharmaceutically acceptable vehicle, excipient, or diluent.

The present invention provides a method for reducing the cholesterol content of a fowl egg comprising administering to a fowl species a therapeutically effective amount of a compound of the invention or a pharmaceutical composition comprising a compound of the invention and a pharmaceutically acceptable vehicle, excipient, or diluent.

The present invention may be understood by reference to the detailed description and examples, which are intended to exemplify non-limiting embodiments of the invention.

### 4. BRIEF DESCRIPTION OF THE FIGURE

FIG. 1. Shows the rate of lipid synthesis of saponified and non-saponified lipids in primary rat hepatocyte cells upon treatment with Compound A, Compound B, or lovastatin.

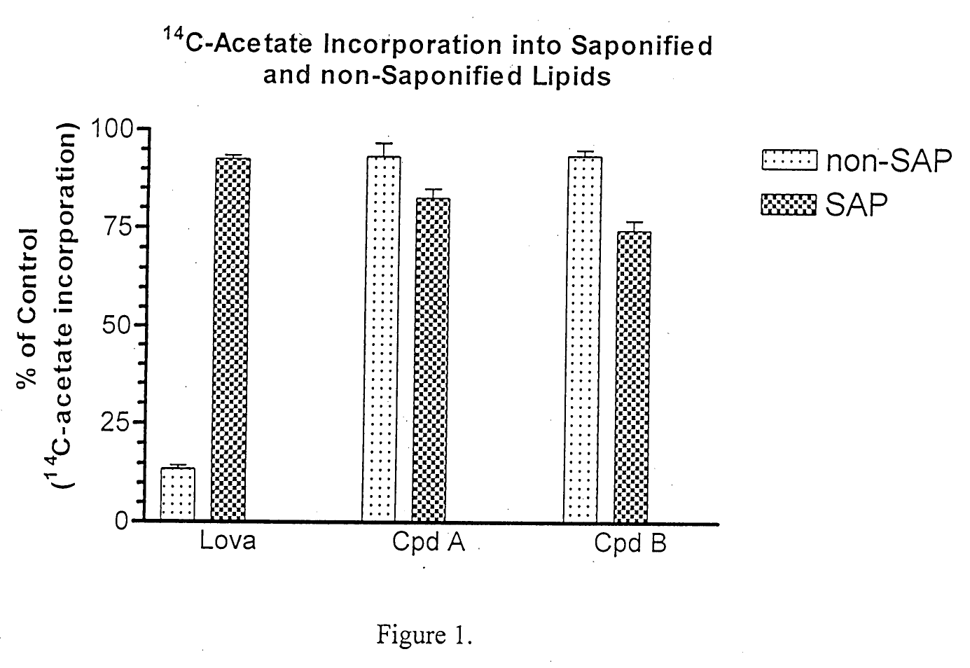

### 5. DETAILED DESCRIPTION OF THE INVENTION

The present invention provides novel compounds useful for treating or preventing aging, Alzheimer's Disease, cancer, cardiovascular disease, diabetic nephropathy, diabetic retinopathy, a disorder of glucose metabolism, dyslipidemia, dyslipoproteinemia, enhancing bile production, enhancing reverse lipid transport, hypertension, impotence, inflammation, insulin resistance, lipid elimination in bile, modulating C reactive protein, obesity, oxysterol elimination in bile, pancreatitis, Parkinson's disease, a peroxisome proliferator activated receptor-associated disorder, phospholipid elimination in bile, renal disease, septicemia, metabolic syndrome disorders (e.g., Syndrome X), a thrombotic disorder, inflammatory processes and diseases like gastrointestinal disease, irritable bowel syndrome (IBS), inflammatory bowel disease (e.g., Crohn's Disease, ulcerative colitis), arthritis (e.g., rheumatoid arthritis, osteoarthritis), autoimmune disease (e.g., systemic lupus erythematosus), scleroderma, ankylosing spondylitis, gout and pseudogout, muscle pain: polymyositis/polymyalgia rheumatica/fibrositis; infection and arthritis, juvenile rheumatoid arthritis, tendonitis, bursitis and other soft tissue rheumatism. In this regard, the compounds of the invention are particularly useful when incorporated in a pharmaceiutical composition having a carrier, excipient, diluent, or a mixture thereof. A composition of the invention need not contain additional ingredients, such as an excipient, other than a compound of the invention. Accordingly, in one embodiment, the compositions of the invention can omit pharmaceutically acceptable excipients and diluents and can be delivered in a gel cap or drug delivery device. Accordingly, the present invention provides methods for treating or preventing aging, Alzheimer's Disease, cancer, cardiovascular disease, diabetic nephropathy, diabetic retinopathy, a disorder of glucose metabolism, dyslipidemia, dyslipoproteinemia, enhancing bile production, enhancing reverse lipid transport, hypertension, impotence, inflammation, insulin resistance, lipid elimination in bile, modulating C reactive protein, obesity, oxysterol elimination in bile, pancreatitis, Parkinson's disease, a peroxisome proliferator activated receptor-associated disorder, phospholipid elimination in bile, renal disease, septicemia, metabolic syndrome disorders (e.g., Syndrome X), a thrombotic disorder, inflammatory processes and diseases like gastrointestinal disease, irritable bowel syndrome (IBS), inflammatory bowel disease (e.g., Crohn's Disease, ulcerative colitis), arthritis (e.g., rheumatoid arthritis, osteoarthritis), autoimmune disease (e.g., systemic lupus erythematosus), sclerodenna, ankylosing spondylitis, gout and pseudogout, muscle pain: polymyositis/polymyalgia rheumatica/fibrositis; infection and arthritis, juvenile rheumatoid arthritis, tendonitis, bursitis and other soft tissue rheumatism, comprising administering to a patient in need thereof a therapeutically effective amount of a compound or composition of the invention.

In certain embodiments of the invention, a compound of the invention is administered in combination with another therapeutic agent. The other therapeutic agent provides additive or synergistic value relative to the administration of a compound of the invention alone. The therapeutic agent can be a lovastatin; a thiazolidinedione or fibrate; a bile-acid-binding-resin; a phosphodiesteras-5-type inhibitor; niacin; an anti-obesity drug; a hormone; a tyrophostine; a sulfonylurea-based drug; a biguanide; an α-glucosidase inhibitor; an apolipoprotein A-I agonist; apolipoprotein E; a cardiovascular drug; an HDL-raising drug; an HDL enhancer; or a regulator of the apolipoprotein A-I, apolipoprotein A-IV and/or apolipoprotein genes.

TABLE 1
COMPOUNDS OF THE INVENTION

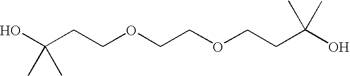

I-1:
4-[2-(3-Hydroxy-3-methyl-butoxy)-ethoxy]-2-methyl-butan-2-ol

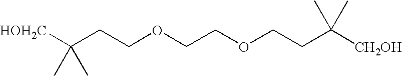

I-2:
4-[2-(4-Hydroxy-3,3-dimethyl-butoxy)-ethoxy]-2,2-dimethyl-butan-1-ol

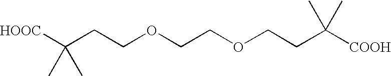

I-3:
4-[2-(3-Carboxy-3-methyl-butoxy)-ethoxy]-2,2-dimethyl-butyric acid

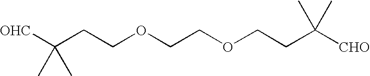

I-4:
4-[2-(3,3-Dimethyl-4-oxo-butoxy)-ethoxy]-2,2-dimethyl-butanal

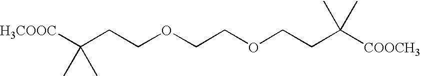

I-5:
4-[2-(3-Mehoxycarbonyl-3-methyl-butoxy)-ethoxy]-2,2-dimethyl-butyric acid methyl ester

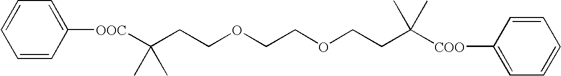

I-6:
2,2-Dimethyl-4-[2-(3-methyl-3-phenoxycarbonyl-butoxy)-ethoxy]-butyric acid phenyl ester

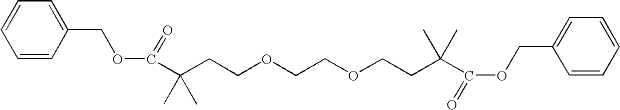

I-7:
Benzyl-2,2,2′,2′-tetramethyl-4,4′-[ethylenebis(oxadiyl)]dibutryrate

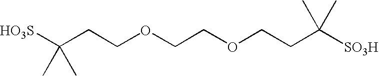

I-8:
2,2′-Dimethyl-4,4′-[ethylenebis(oxadiyl)]dibutane-2-sulfonic acid

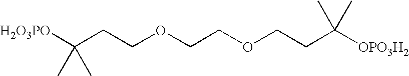

I-9:
Phosphoric acid mono-{3-[2-(3,3-dimethyl-butoxy)-ethoxy]-1,1-dimethyl-propyl} ester

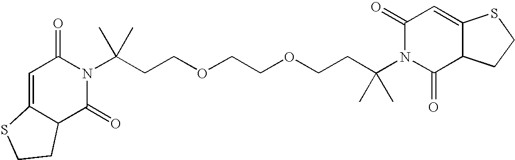

I-10:
1-Ethyl-3-(3-{2-[3-(4,6-dioxo-2,3,3a,6-tetrahydro-4H-thieno[3,2-c]pyridin-5-
yl))-3-methyl-butoxy]-ethoxy}-1,1-dimethyl-propyl)-4,6-dioxo-2,3,3a,6-tetrahydro-4H-
thieno[3,2-c]pyridin-5-yl-4,6-dione

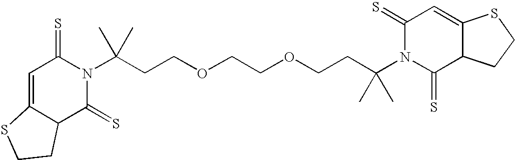

I-11:
1-Ethyl-3-(3-{2-[3-(4,6-dithioxo-2,3,3a,6-tetrahydro-4H-thieno[3,2-c]pyridin-5-
yl))-3-methyl-butoxy]-ethoxy}-1,1-dimethyl-propyl)-4,6-dioxo-2,3,3a,6-tetrahydro-4H-
thieno[3,2-c]pyridin-5-yl-4,6-dithione

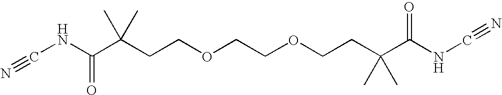

I-12:
2,2-Dimethyl-4-[2-(3-methyl-3-cyanocarbamoyl-butoxy)-ethoxy]-N-cyano-butyramide

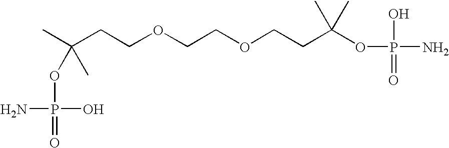

I-13:
Phosphoradimic acid mono-(3-{2-[3-(amino-hydroxy-phosphoryloxy)-3-methyl-butoxy]-
ethoxy}-1,1-dimethyl-propyl) ester

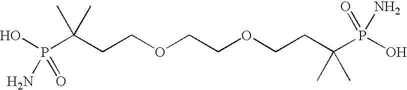

I-14:
{1,1-Dimethyl-3-[2-(3-methyl-3-phosphonamido-butoxy)-ethoxy]-
propyl}-phosphonic acid amide

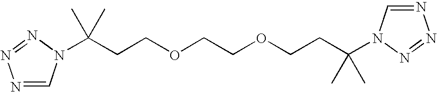

I-15:
1-{3-[2-(3-Methyl-3-{(1H)-tetrazol-1-yl}-butoxy)-ethoxy]-1,1-
dimethyl-propyl}-1H-tetrazole

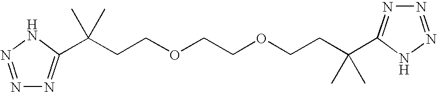

I-16:
5-{3-[2-(3-Methyl-3-{(1H)tetrazol-5-yl}-butoxy)-ethoxy]-1,1-dimethyl-
propyl}-1H-tetrazole

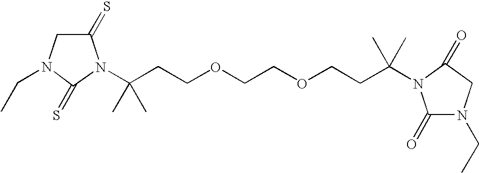

I-17:
1-Ethyl-3-(3-{2-[3-(3-ethyl-2,5-dithioxo-imidazolidin-1-yl)-3-methyl-butoxy]-
ethoxy}-1,1-dimethyl-propyl)-imidazolidine-2,4-dione

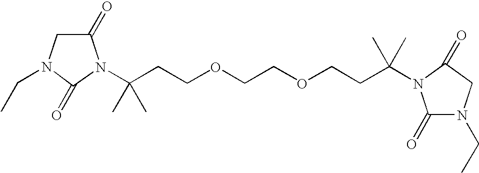

I-18:
1-Ethyl-3-(3-{2-[3-(3-ethyl-2,5-dioxo-imidazolidin-1-yl)-3-methyl-butoxy]-
ethoxy}-1,1-dimethyl-propyl)-imidazolidine-2,4-dione

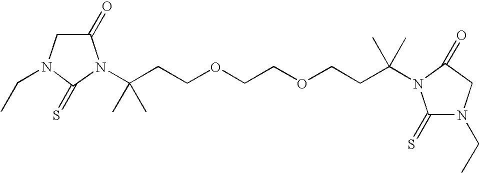

I:19:
1-Ethyl-3-(3-{2-[3-(3-ethyl-2-oxo-5-thioxo-imidazolidin-1-yl)-3-methyl-butoxy]-
ethoxy}-1,1-dimethyl-propyl)-imidazolidine-2-thioxo-4-one

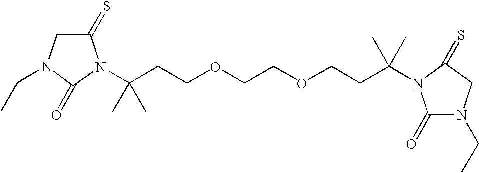

I-20:
1-Ethyl-3-(3-{2-[3-(3-ethyl-5-oxo-2-thioxo-imidazolidin-1-yl)-3-methyl-butoxy]-
ethoxy}-1,1-dimethyl-propyl)-imidazolidine-4-thioxo-2-one

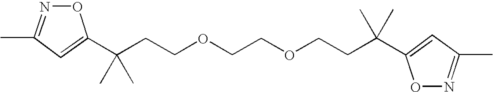

I-21:
1-{3-[2-(3-Methyl-3-(3-methyl-isoxazol-5-yl)-butoxy)-ethoxy]-1,1-dimethyl-
propyl}-5-isoxazole

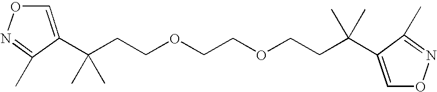

I-22:
1-{3-[2-(3-Methyl-3-(3-methyl-isoxazol-4-yl)-butoxy)-ethoxy]-1,1-dimethyl-
propyl}-4-isoxazole

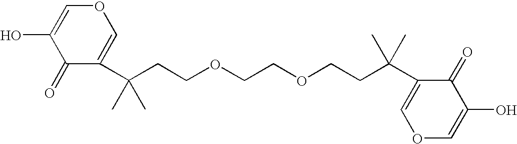

I-23:
3-{3-[2-(3-Methyl-3-(5-hydroxy-pyran-3-yl-4-one)-butoxy)-ethoxy]-1,1-dimethyl-
propyl}-5-hydroxy-pyran-4-one

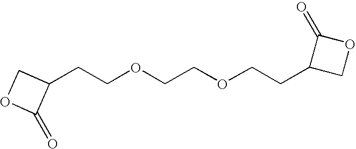

I-28:
3-{2-[2-(3-Oxetan-2-one)-propoxy-ethoxy]-ethyl}-oxetan-2-one

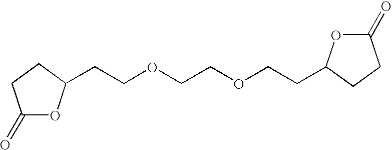

I-29:
5-{2-[2-(5-Dihydro-furan-2-one)-propoxy-ethoxy]-ethyl}-dihydro-furan-2-one

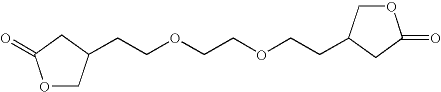

I-30:
4-{2-[2-(4-Dihydro-furan-2-one)-propoxy-ethoxy]-ethyl}-dihydro-furan-2-one

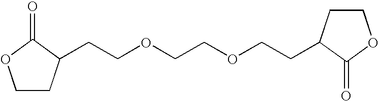

I-31:
3-{2-[2-(3-Dihydro-furan-2-one)-propoxy-ethoxy]-ethyl}-dihydro-furan-2-one

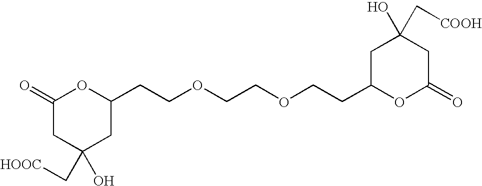

I-32:
2-{2-[2-(2-{2-[4-(Carboxy-methyl)-4-hydroxy-6-oxo-tetrahydro-pyran-2-yl]-
ethoxy}-ethoxy)-ethyl]-4-hydroxy-6-oxo-tetrahydro-pyran-4-yl}-acetic acid

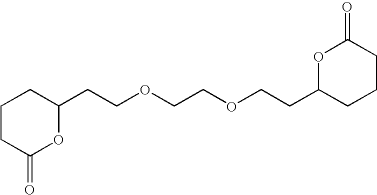

I-33:
2,2′-[Ethylenebis(oxadiyl)]diethane-6-δ-valerolactone

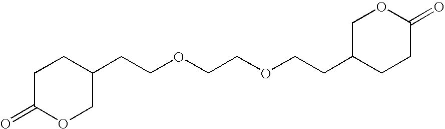

I-34:
2,2′-[Ethylenebis(oxadiyl)]diethane-5-δ-valerolactone

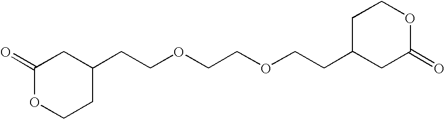

I-35:
2,2′-[Ethylenebis(oxadiyl)]diethane-4-δ-valerolactone

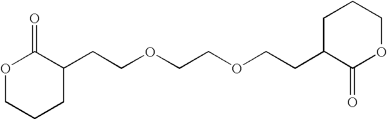

I-36:
2,2′-[Ethylenebis(oxadiyl)]diethane-3-δ-valerolactone

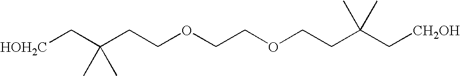

I-37:
3,3,3′,3′-Tetramethyl-5,5′-[ethylenebis(oxadiyl)]dipentanol

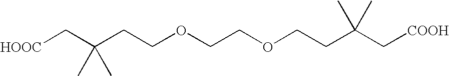

I-38:
3,3,3′,3′-Tetramethyl-5,5′-[ethylenebis(oxadiyl)]dipentanoic acid

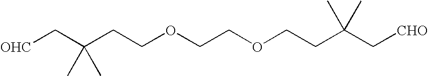

I-39:
3,3,3′,3′-Tetramethyl-5,5′-[ethylenebis(oxadiyl)]dipentanal

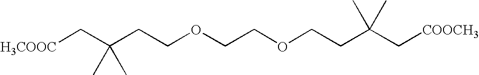

I-40:
Methyl-3,3,3′,3′-tetramethyl-5,5′-[ethylenebis(oxadiyl)]dipentanoate

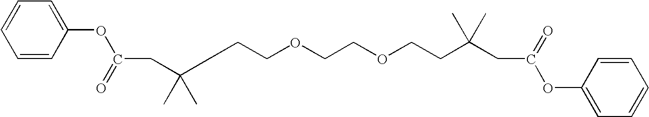

I-41:
Phenyl-3,3,3′,3′-tetramethyl-5,5′-[ethylenebis(oxadiyl)]dipentanoate

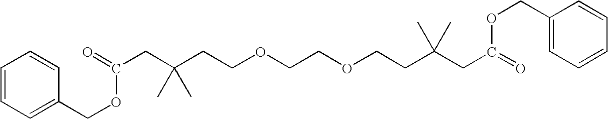

I-42:
Benzyl-3,3,3′,3′-tetramethyl-5,5′-[ethylenebis(oxadiyl)]dipentanoate

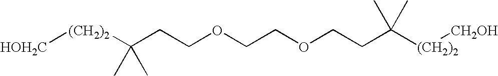

I-43:
4,4,4′,4′-Tetramethyl-6,6′-[ethylenebis(oxadiyl)]dihexanol

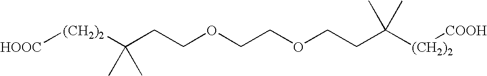

I-44:
4,4,4′,4′-Tetramethyl-6,6′-[ethylenebis(oxadiyl)]dihexanoic acid

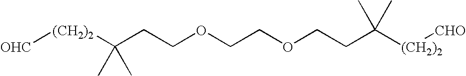

I-45:
4,4,4′,4′-Tetramethyl-6,6′-[ethylenebis(oxadiyl)]dihexanal

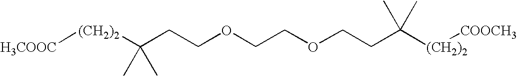

I-46:
Methyl-4,4,4′,4′-tetramethyl-6,6′-[ethylene-(oxadiyl)]-dihexanoate

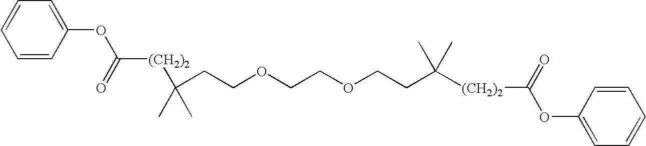

I-47:
Phenyl-4,4,4′,4′-tetramethyl-6,6′-[ethylenebis(oxadiyl)]dihexanoate

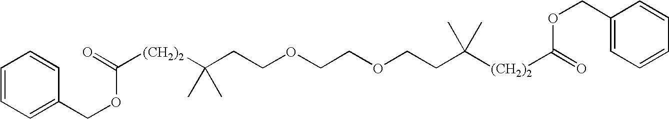

I-48:
Benzyl-4,4,4′,4′-tetramethyl-6,6′-[ethylenebis(oxadiyl)]dihexanoate

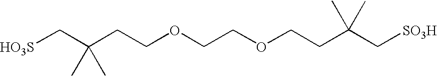

I-49:
2,2,2′,2′-Tetramethyl-4,4′-[ethylenebis(oxadiyl)]dibutane sulfonic acid

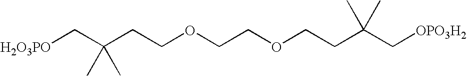

I-50:
Phosphoric acid mono-{4-[2-(3,3-dimethyl-4-phosphonooxy-butoxy)-ethoxy]-2,2-
dimethyl-butyl}ester

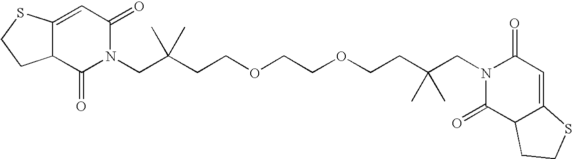

I-51:
5-{4-[2-(3,3-Dimethyl-4-(5-(3,3a-dihydro-2H-thieno-[3,2-c]pyridine-4,6-
dioxo)pentyloxy)-ethoxy]-2,2-dimethyl-butyl}-3,3a-dihydro 3,3a-dihydro-2H-thieno-
[3,2-c]pyridine-4,6-dione

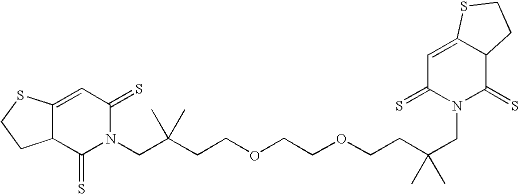

I-52:
5-{4-[2-(3,3-Dimethyl-4-(5-(3,3a-dihydro-2H-thieno-[3,2-c]pyridine-4,6-
dithioxo)pentyloxy)-ethoxy]-2,2-dimethyl-butyl}-3,3a-dihydro 3,3a-dihydro-2H-thieno-
[3,2-c]pyridine-4,6-dithione

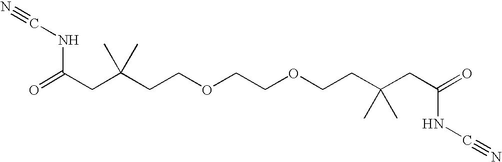

I-53:
5-[2-(3,3-Dimethyl-4-cyanocarbamoyl-butoxy)-ethoxy]-3,3-dimethyl-N-cyano-
pentanoic acid-amide

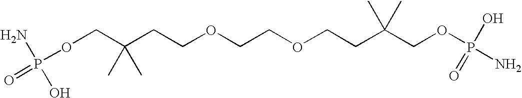

I-54:
Phosphoramidic acid mono-(4-{2-[4-(amino-hydroxy-phosphoryloxy)-
3,3-dimethyl-butoxy]-ethoxy}-2,2-dimethyl-butyl) ester

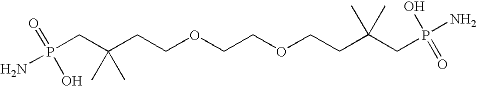

I-55:
{4-[2-(3,3-Dimethyl-4-phosphonamido-butoxy)-ethoxy]-2,2-dimethyl-butyl}-
phosphonamide

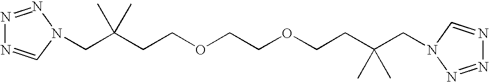

I-56:
1-{4-[2-(3,3-Dimethyl-5-{1H-tetrazol-1-yl}-butoxy)-ethoxy]-2,2-dimethyl-
butyl}-1H-tetrazole

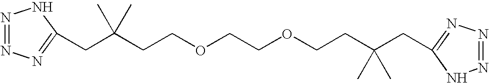

I-57:
5-{4-[2-(3,3-Dimethyl-5-(1H-tetrazol-5-yl}-butoxy)-ethoxy]-2,2-dimethyl-
butyl}-1H-tetrazole

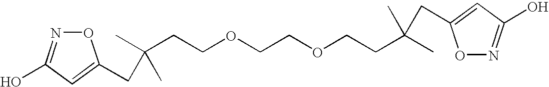

I-58:
5-{4-[2-(3,3-Dimethyl-5-{3-hydroxy-isoxazol-5-yl}-butoxy)-ethoxy]-2,2-dimethyl-
butyl}-3-hydroxy-isoxazole

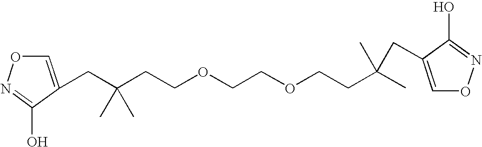

I-59:
4-{4-[2-(3,3-Dimethyl-5-{3-hydroxy-isoxazol-4-yl}-butoxy)-ethoxy]-2,2-dimethyl-
butyl}-3-hydroxy-isoxazole

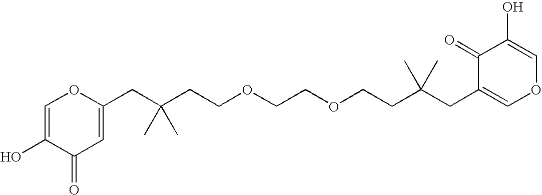

I-60:
2-{4-[2-(3,3-Dimethyl-5-{5-hydroxy-pyran-4-oxo-3-yl}-butyloxy)-ethoxy]-
2,2-dimethyl-butyl}-5-hydroxy-pyran-4-one

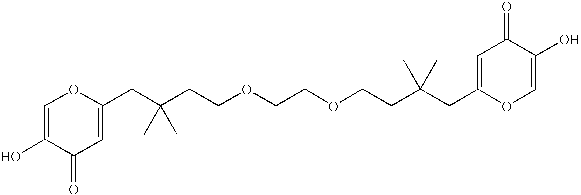

I-61:
2-{4-[2-(3,3-Dimethyl-5-{5-hydroxy-pyran-4-oxo-2-yl}-butyloxy)-ethoxy]-2,2-dimethyl-
butyl}-5-hydroxy-pyran-4-one

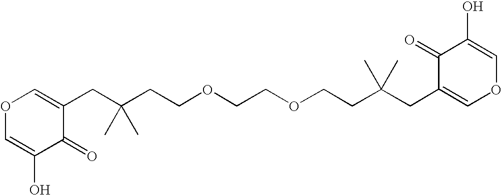

I-62:
3-{4-[2-(3,3-Dimethyl-5-{5-hydroxy-pyran-4-oxo-3-yl}-butyloxy)-ethoxy]-2,2-dimethyl-
butyl}-5-hydroxy-pyran-4-one

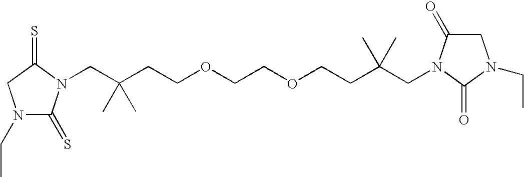

I-63:
1-Ethyl-3-(4-{2-[4-(3-ethyl-2,5-dithioxo-imidazolidin-1-yl)-3,3-dimethyl-butoxy]-
ethoxy}-2,2-dimethyl-butyl)-imidazolidine-2,4-dione

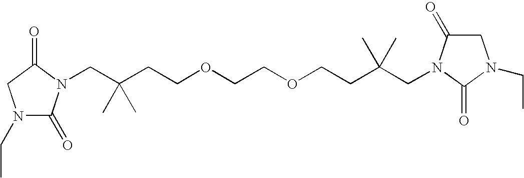

I-64:
1-Ethyl-3-(4-{2-[4-(3-ethyl-2,5-dioxo-imidazolidin-1-yl)-3,3-dimethyl-butoxy]-
ethoxy}-2,2-dimethyl-butyl)-imidazolidine-2,4-dione

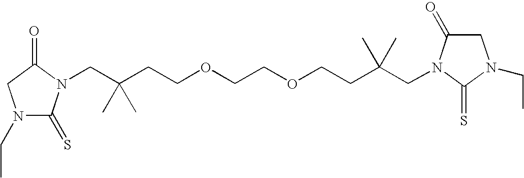

I-65:
1-Ethyl-3-(4-{2-[4-(3-ethyl-2-thioxo-4-oxo-imidazolidin-1-yl)-3,3-dimethyl-butoxy]-
ethoxy}-2,2-dimethyl-butyl)-imidazolidine-2-thioxo-4-one

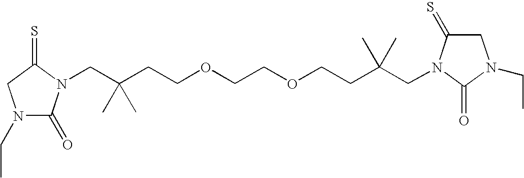

I-66:
1-Ethyl-3-(4-2-[4-(3-ethyl-2-oxo-4-thioxo-imidazolidin-1-yl)-3,3-dimethyl-butoxy]-
ethoxy}-2,2-dimethyl-btutyl)-imidazolidine-2-oxo-4-thione

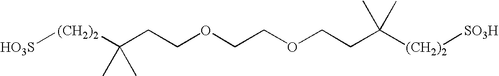

I-67:
3,3,3′,3′-tetramethyl-5,5′-[ethylenebis(oxadiyl)]dipentane sulfonic acid

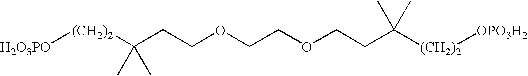

Phosphoric acid mono-{1,1-dimethyl-3-[2-(3-methyl-3-phosphonooxy-butoxy)-ethoxy]-
propyl}ester

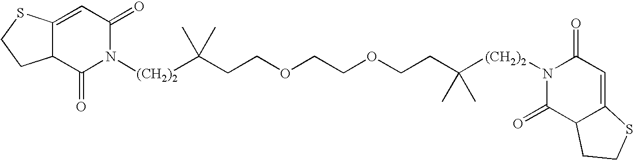

I-69:
5-(5-{2-[3,3-Dimethyl-5-(4,6-dioxo-2,3,3a,6-tetrahydro-4H-thieno[3,2-c]
pyridin-5-yl)-pentyloxy]-ethoxy}-3,3-dimethyl-pentyl)-3,3a-dihydro-2H-thieno[3,2-c]
pyridine-4,6-dione

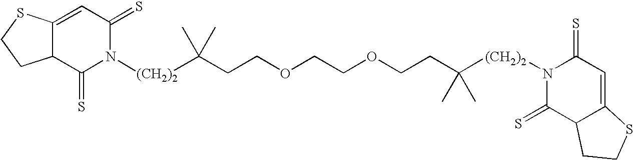

I-70:
5-(5-{2-[3,3-Dimethyl-5-(4,6-dithioxo-2,3,3a,6-tetrahydro-4H-thieno[3,2-c]
pyridin-5-yl)-pentyloxy]-ethoxy}-3,3-dimethyl-pentyl)-3,3a-dihydro-2H-thieno[3,2-c]
pyridine-4,6-dione

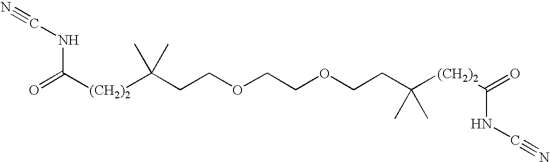

I-71:
6-[2-(3,3-Dimethyl-5-cyano-carbamoyl-butoxy)-ethoxy]-4,4-dimethyl-N-cyano-hexanoic
acid-amide

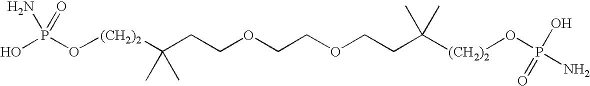

I-72:
Phosphoramidic acid mono-(5-{2-[5-(amino-hydroxy-phosphoryloxy)-
3,3-dimethyl-pentyloxy]-ethoxy}-3,3-dimethyl-pentyl) ester

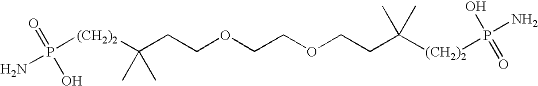

I-73:
{5-[2-(3,3-Dimethyl-5-phosphonamido-pentyloxy)-ethoxy]-3,3-dimethyl-pentyl}-
phosphonamide

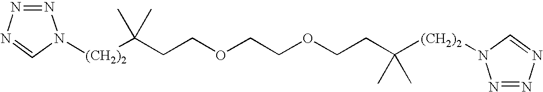

I-74:
1-{[2-(3,3-Dimethyl-5-tetrazol-1-yl-pentyloxy)-ethoxy]-3,3-dimethyl-pentyl}-1H-
tetrazole

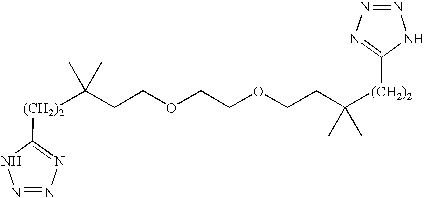

I-75:
5-{5-[2-(3,3-Dimethyl-5-tetrazol-1-yl-pentyloxy)-ethoxy]-3,3-dimethyl-pentyl}-1H-
tetrazole

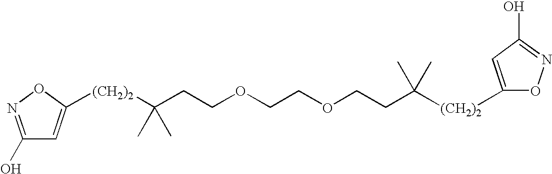

I-76:
5-{5-[2-(3,3-Dimethyl-5-{3-hydroxy-isoxazol-5-yl}-
pentyloxy)-ethoxy]-3,3-dimethyl-pentyl}-isoxazol-3-ol

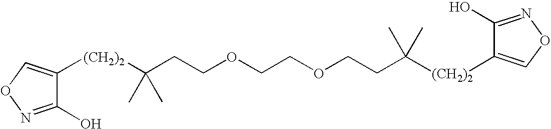

I-77:
4-{5-[2-(3,3-Dimethyl-5-{3-hydroxy-isoxazol-4-yl}-pentyloxy)-ethoxy]-
3,3-dimethyl-pentyl}-isoxazol-3-ol

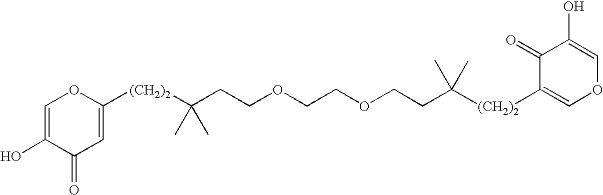

I-78:
3-{5-[2-(5-{5-Hydroxy-4-oxo-4H-pyran-2-yl}-3,3-dimethyl-pentyloxy)-3,3-dimethyl-
pentyl]-5-hydroxy-pyran-4-one

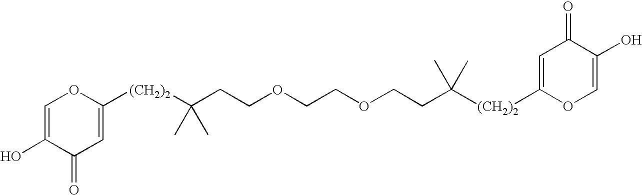

2-{5-[2-(5-{5-Hydroxy-4-oxo-4H-pyran-2-yl}-3,3-dimethyl-pentyloxy)-3,3-dimethyl-
pentyl]-5-hydroxy-pyran-4-one

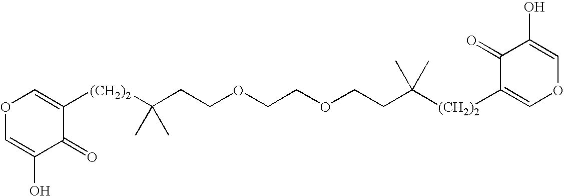

I-80:
3-{5-[2-(5-{5-Hydroxy-4-oxo-4H-pyran-3-yl}-3,3-dimethyl-pentyloxy)-3,3-dimethyl-
pentyl]-5-hydroxy-pyran-4-one

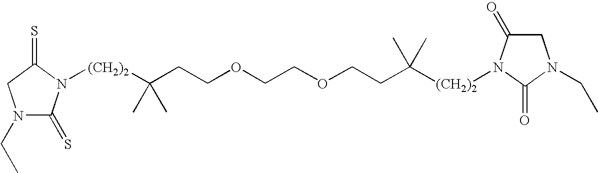

I-81:
1-Ethyl-3-(5-{2-[5-(3-ethyl-2,5-dithioxo-imidazolidin-1-yl)-3,3-dimethyl-pentyloxy]-
ethoxy}-3,3-dimethyl-pentyl)-imidazolidine-2,4-dione

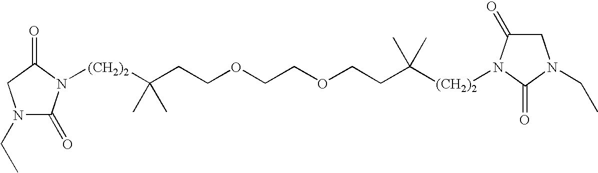

I-82:
1-Ethyl-3-(5-{2-[5-(3-ethyl-2,5-dioxo-imidazolidin-1-yl)-3,3-dimethyl-pentyloxy]-
ethoxy}-3,3-dimethyl-pentyl)-imidazolidine-2,4-dione

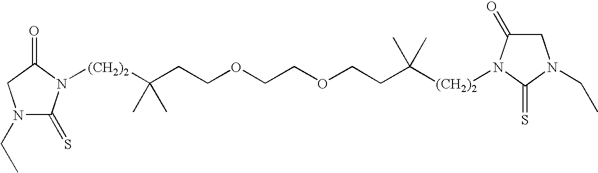

I-83:
1-Ethyl-3-(5-{2-[5-(1-ethyl-2-thioxo-5-oxo-imidazolidin-3-yl)-3,3-dimethyl-pentyloxy]-
ethoxy}-3,3-dimethyl-pentyl)-imidazolidine-2-thioxo-4-one

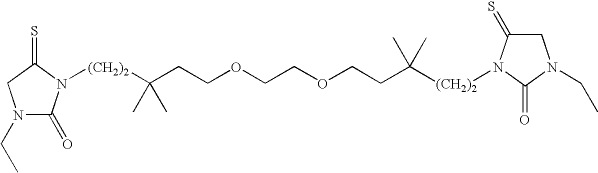

I-84:
1-Ethyl-3-(5-{2-[5-(1-ethyl-2-oxo-5-thioxo-imidazolidin-3-yl)-3,3-dimethyl-pentyloxy]-
ethoxy}-3,3-dimethyl-pentyl)-imidazolidine-2-oxo-4-thione

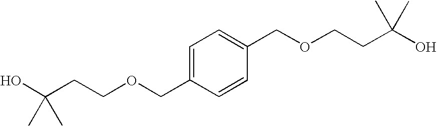

I-85:
4-[4-(3-Hydroxy-3-methyl-butoxymethyl)-benzyloxy]-2-methyl-butan-2-ol

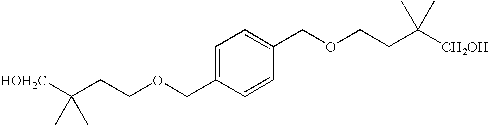

I-86:
4-[4-(4-Hydroxy-3,3-dimethyl-butoxymethyl)-benzyloxy]-2,2-dimethyl-butan-1-ol

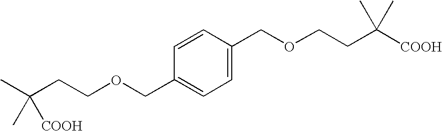

I-87:
4-[4-(3-Carboxyl-3,3-dimethyl-butoxymethyl)-benzyloxy]-2,2-dimethyl-butyric acid

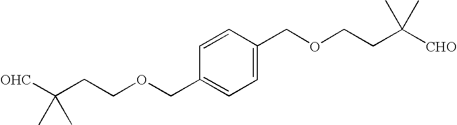

I-88:
4-[4-(4-Hydroxy-3,3-dimethyl-butoxymethyl)-benzyloxy]-2,2-dimethyl-butanal

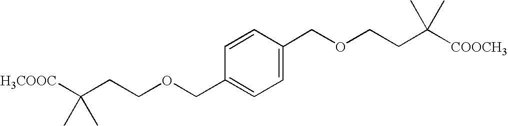

I-89:
4-[4-(3,3-Dimethyl-3-carboxymethyl-butoxymethyl)-benzyloxy]-2,2-dimethyl-butyric
acid methyl ester

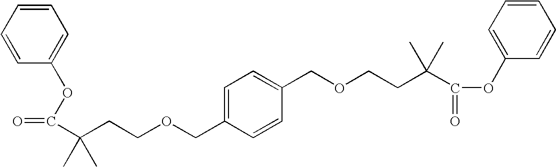

I-90:
2,2-Dimethyl-4-[4-(3-methyl-3-phenoxycarbonyl-butoxymethyl)-benzyloxy]-butyric acid
phenyl ester

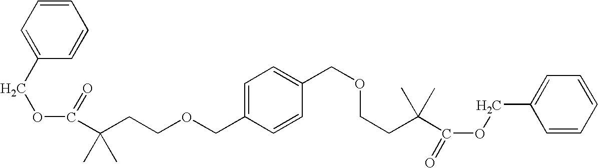

I-91:
4-[4-(3-Benzyloxycarbonyl-3-methyl-butoxymethyl)-benzyloxy]-2,2-dimethyl-butyric
acid benzyl ester

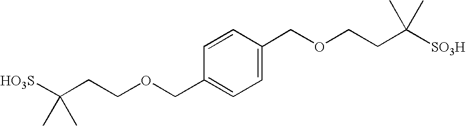

I-92:
2,2′-Dimethyl-4,4′-[vinylbis(oxadiyl)]dibutane-2-sulfonic acid

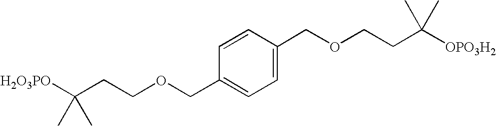

I-93:
Phosphoric acid mono-{1,1-dimethyl-3-[4-(3-methyl-3-phosphonooxy-butoxymethyl)-
benzyloxy]-propyl}ester

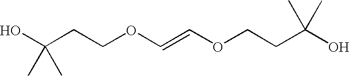

I-94:
2,2′-Dimethyl-4,4′-[vinylbis(oxadiyl)]dibutanol

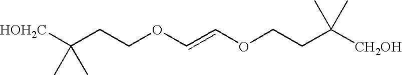

I-95:
4-[2-(4-Hydroxy-3,3-dimethyl-butoxy)-vinyloxy]-2,2-dimethyl-butan-1-ol

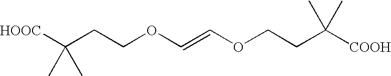

I-96:
4-[2-(3-Carboxyl-3,3-dimethyl-butoxy)-vinyloxy]-2,2-dimethyl-butyric acid

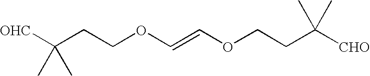

I-97:
4-[2-(4-Hydroxy-3,3-dimethyl-butoxy)-vinyloxy]-2,2-dimethyl-butanal

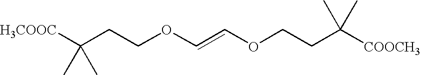

I-98:
4-[2-(3,3-Dimethyl-3-carboxymethyl-3-butoxy)-vinyloxy]-2,2-dimethyl-butyric acid
methyl ester

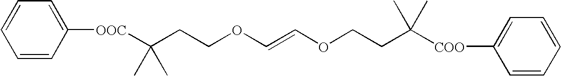

I-99:
2,2-Dimethyl-4-[2-(3-methyl-3-phenoxycarbonyl-butoxy)-vinyloxy]-butyric acid
phenyl ester

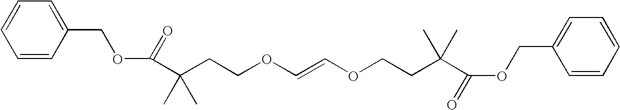

I-100:
2,2-Dimethyl-4-[2-(3-methyl-3-benzyloxycarbonyl-butoxy)-vinyloxy]-butyric acid
benzyl ester

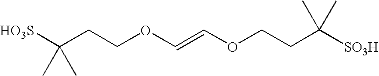

I-101:
4-[2-(3,3-Dimethyl-3-sulfono-butoxy)-vinyloxy]-2-methyl-butane-2-sulfonic acid

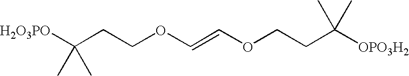

I-102:
Phosphoric acid mono-{3-[2-(3,3-dimethyl-butoxy)-vinyloxy]-1,1-dimethyl-propyl}
ester

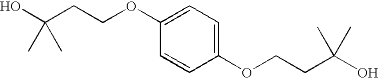

I-103:
4-[4-(3-Hydroxy-3-methyl-butoxy)-phenoxy]-2-methyl-butan-2-ol

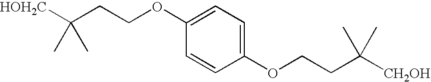

I-104:
4-[4-(4-Hydroxy-3,3-dimethyl-butoxy)-phenoxy]-2,2-dimethyl-butan-1-ol

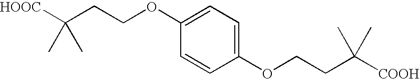

I-105:
4-[4-(3-Carboxyl-3,3-dimethyl-butoxy)-phenoxy]-2,2-dimethyl-butyric acid

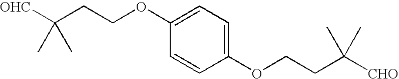

I-106:
4-[4-(4-Hydroxy-3,3-dimethyl-butoxy)-phenoxy]-2,2-dimethyl-butanal

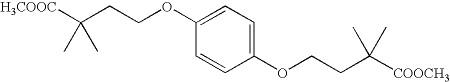

I-107:
4-[4-(3,3-Dimethyl-3-carboxymethyl-butoxy)-phenoxy]-2,2-dimethyl-
butyric acid methyl ester

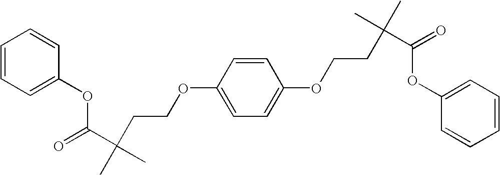

I-108:
2,2-Dimethyl-4-[4-(3-methyl-3-phenoxycarbonyl-butoxy)-phenoxy]-butyric
acid phenyl ester

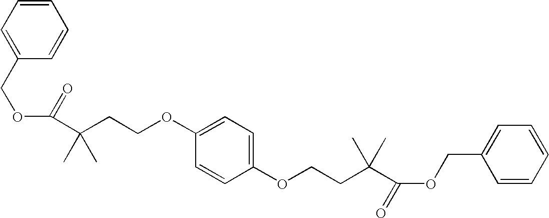

I-109:
4-[4-(3-Benzyloxycarbonyl-3-methyl-butoxy)-phenoxy]-2,2-dimethyl-butyric
acid benzyl ester

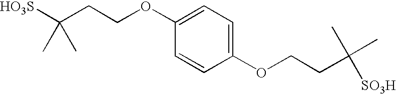

I-110:
4-[4-(3,3-Dimethyl-3-sulfono-butoxy)-phenoxy]-2-methyl-butane-2-sulfonic acid

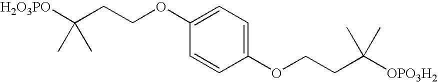

I-111:
4-[4-(3,3-Dimethyl-3-oxyphosphono-butoxy)-phenoxy]-2-methyl-butane-2-
oxyphosphoric acid

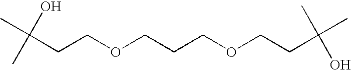

I-112:
4-[3-(3-Hydroxy-3-methyl-butoxy)-propoxy]-2-methyl-butan-2-ol

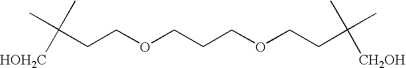

I-113:
4-[3-(4-Hydroxy-3,3-dimethyl-butoxy)-propoxy]-2,2-dimethyl-butan-1-ol

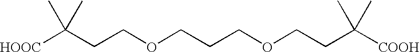

I-114:
4-[3-(3-Carboxy-3-methyl-butoxy)-propoxy]-2,2-dimethyl-butyric acid

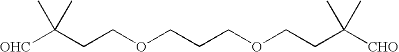

I-115:
4-[3-(3,3-Dimethyl-4-oxo-butoxy)-propoxy]-2,2-dimethyl-butanal

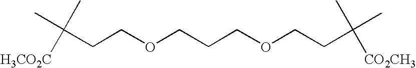

I:116:
4-[3-(3-Methoxycarbonyl-3-methyl-butoxy)-propoxy]-2,2-dimethyl-butyric acid methyl
ester

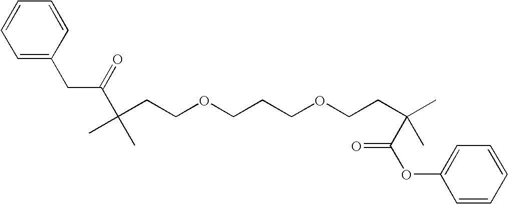

I-117:
4-[3-(3,3-Dimethyl-4-oxo-5-phenyl-pentyloxy)-propoxy]-2,2-dimethyl-butyric
acid phenyl ester

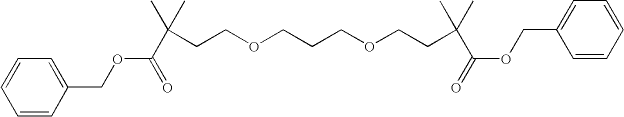

I-118:
4-[3-(3-Benzyloxycarbonyl-3-methyl-butoxy)-propoxy]-2,2-dimethyl-butyric acid benzyl
ester

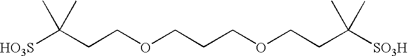

I-119:
2-Methyl-4-[3-(3-methyl-3-sulfo-butoxy)-propoxy]-butane-2-sulfonic acid

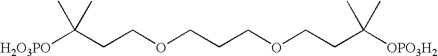

I-120:
Phosphoric acid mono-{1,1-dimethyl-3-[3-(3-methyl-3-
phosphonooxy-butoxy)-propoxy]-propyl}ester

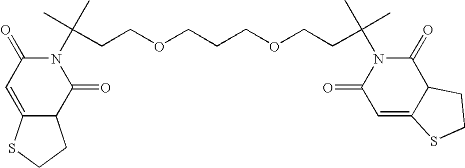

I-121:
1-Ethyl-3-(3-{3-[3-(4,6-dioxo-2,3,3a,6-tetrahydro-4H-thieno[3,2-c]pyridin-5-
yl))-3-methyl-butoxy]-propoxy}-1,1-dimethyl-propyl)-4,6-dioxo-2,3,3a,6-tetrahydro-4H-
thieno[3,2-c]pyridin-5-yl-4,6-dione

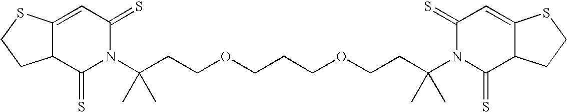

I-122:
1-Ethyl-3-(3-{3-[3-(4,6-dithioxo-2,3,3a,6-tetrahydro-4H-thieno[3,2-c]pyridin-5-
yl))-3-methyl-butoxy]-propoxy}-1,1-dimethyl-propyl)-4,6-dioxo-2,3,3a,6-tetrahydro-4H-
thieno[3,2-c]pyridin-5-yl-4,6-dithione

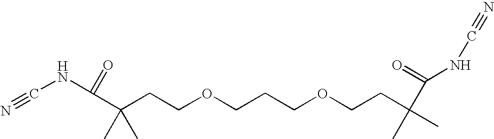

I-123:
2,2-Dimethyl-4-[3-(3-methyl-3-cyano-carbamoyl-butoxy)-propoxy]-N-cyano-butyric
acid-amide

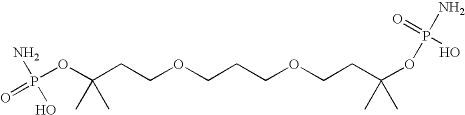

I-124:
Phosphoramidic acid mono-(3-{3-[3-(amino-hydroxy-phosphoryloxy)-
3-methyl-butoxy]-propoxy}-1,1-dimethyl-propyl) ester

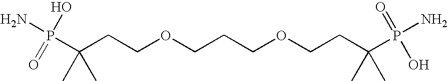

I-125:
{1,1-Dimethyl-3-[3-(3-(methyl-3-phosponamido-butoxy)-propoxy]-propyl}-
phosphonamide

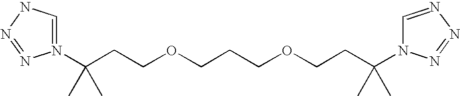

I-126:
1-{3-[3-(3-Methyl-3-tetrazol-1-yl-butoxy)-propoxy]-1,1-dimethyl-propyl}-1H-tetrazole

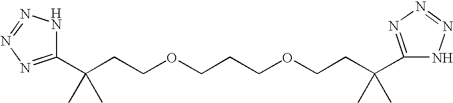

I-127:
5-{3-[3-(3-Methyl-3-tetrazol-5-yl-butoxy)-propoxy]-1,1-dimethyl-propyl}-(1H)-tetrazole

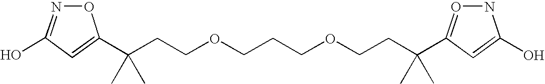

I-128:
5-{3-[3-(3-Methyl-3-(3-methyl-isoxazol-5-yl)-butoxy)-propoxy]-1,1-dimethyl-propyl}-3-
methyl-isoxazole

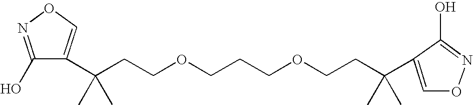

I-129:
4-{3-[3-(3-Methyl-3-(3-methyl-isoxazol-4-yl)-butoxy)-propoxy]-1,1-dimethyl-propyl}-3-
methyl-isoxazole

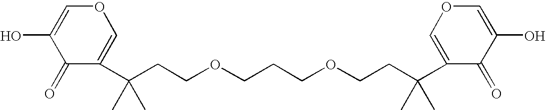

I-130:
3-{3-[3-(3-Methyl-3-(5-hydroxy-pyran-3-yl-4-one)-butoxy)-propoxy]-1,1-dimethyl-
propyl}-5-hydroxy-pyran-4-one

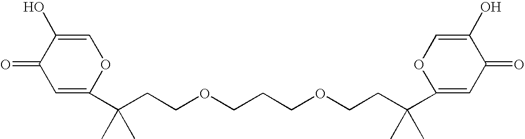

I-131:
2-{3-[3-(3-Methyl-3-(5-hydroxy-pyran-2-yl-4-one)-butoxy)-propoxy]-1,1-dimethyl-
propyl}-5-hydroxy-pyran-4-one

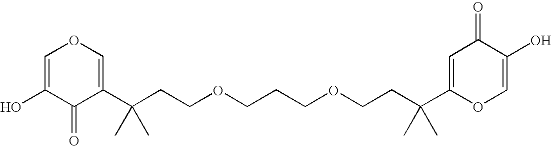

I-132:
3-{3-[3-(3-Methyl-3-(5-hydroxy-pyran-2-yl-4-one)-butoxy)-propoxy]-1,1-dimethyl-
propyl}-5-hydroxy-pyran-4-one

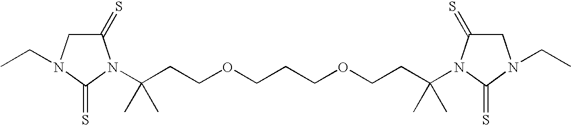

I-133:
1-Ethyl-3-(3-{3-[3-(3-ethyl-2,5-dithioxo-imidazolidin-1-yl)-3-methyl-butoxy]-
propoxy}-1,1-dimethyl-propyl)-imidazolidine-2,4-dithione

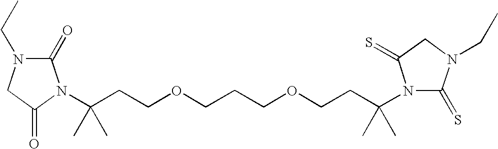

I-134:
1-Ethyl-3-(3-{3-[3-(3-ethyl-2,5-dioxo-imidazolidin-1-yl)-3-methyl-butoxy]-propoxy}-1,1-
dimethyl-propyl)-imidazolidine-2,4-dithione

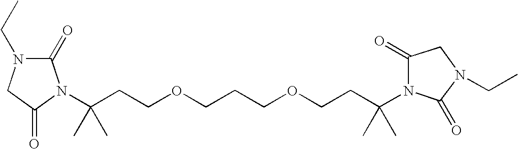

I-135:
1-Ethyl-3-(3-{3-[3-(3-ethyl-2,5-dioxo-imidazolidin-1-yl)-3-methyl-butoxy]-propoxy}-
1,1-dimethyl-propyl)-imidazolidine-2,4-dione

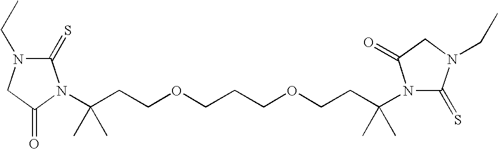

I-136:
1-Ethyl-3-(3-{3-[3-(3-ethyl-2-thioxo-5-oxo-imidazolidin-1-yl)-3-
methyl-butoxy]-propoxy}-1,1-dimethyl-propyl)-imidazolidine-2-thioxo-4-one

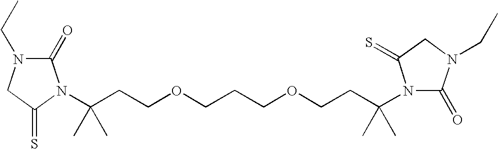

I-137:
1-Ethyl-3-(3-{3-[3-(3-ethyl-2-oxo-5-thioxo-imidazolidin-1-yl)-
3-methyl-butoxy]-propoxy}-1,1-dimethyl-propyl)-imidazolidine-2-oxo-4-thione

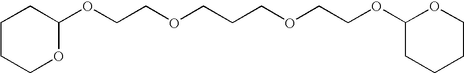

I-138:
1-(2-Tetrahydropyranyloxy)-2-{2-[2-(2-tetrahydropyranyloxy)-ethoxy]-propoxy}ethane

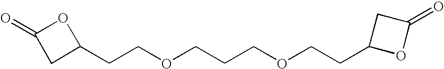

I-139:
4-{2-[3-(Oxetan-4-yl-2-one)-propoxy-propoxy]-ethyl}-oxetan-2-one

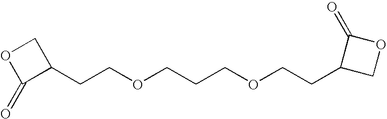

I-140:
3-{2-[3-(Oxetan-3-yl--2-one)-propoxy-propoxy]-ethyl}-oxetan-2-one

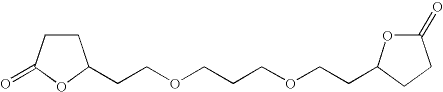

I-141:
5-{2-[3-(Dihydro-furan-5-yl-2-one)-propoxy-propoxy]-ethyl}-dihydro-furan-2-one

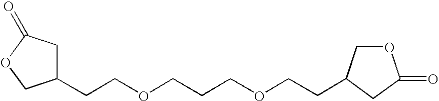

I-142:
4-{2-[3-(Dihydro-furan-4-yl-2-one)-propoxy-propoxy]-ethyl}-dihydro-furan-2-one

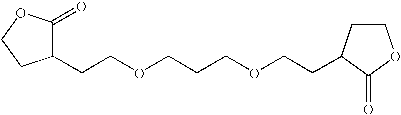

I-143:
3-{2-[3-(Dihydro-furan-3-yl-2-one)-propoxy-propoxy]-ethyl}-dihydro-furan-2-one

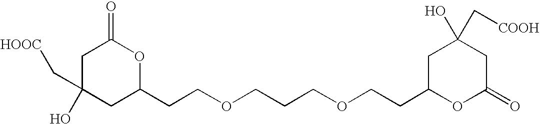

I-144:
[2-(2-{3-{2-(4-Carboxymethyl-4-hydroxy-6-oxo-tetrahydro-pyran-2-yl)-ethoxy]-
propoxy}-ethyl)-4-hydroxy-6-oxo-tetrahydro-pyran-4-yl]-acetic acid

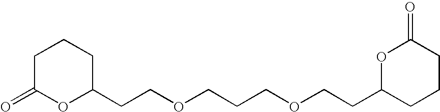

I-145:
2,2′-[Propylenebis(oxadiyl)]diethane-6-δ-valerolactone

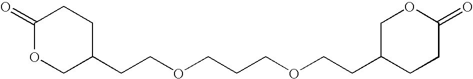

I-146:
2,2′-[Propylenebis(oxadiyl)]diethane-5-δ-valerolactone

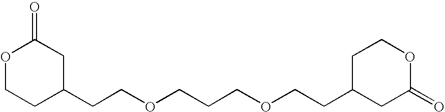

I-147:
2,2′-[Propylenebis(oxadiyl)]diethane-4-δ-valerolactone

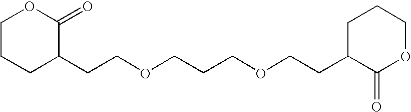

I-148:
2,2′-[Propylenebis(oxadiyl)]diethane-3-δ-valerolactone

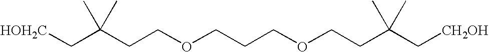

I-149:
5-[3-(5-Hydroxy-3,3-dimethyl-pentyloxy)-propoxy]-3,3-dimethyl-pentan-1-ol

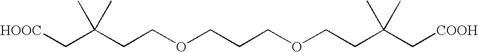

I-150:
5-[3-(4-Carboxy-3,3-dimethyl-butoxy)-propoxy]-3,3-dimethyl-pentanoic acid

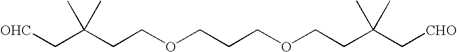

I-151:
5-[3-(3,3-Dimethyl-5-oxo-pentyloxy)-propoxy]-3,3-dimethyl-pentanal

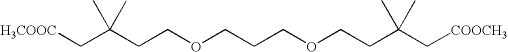

I-152:
5-[3-(4-Methoxycarbonyl-3,3-dimethyl-butoxy)-propoxy]-3,3-dimethyl-pentanoic acid
methyl ester

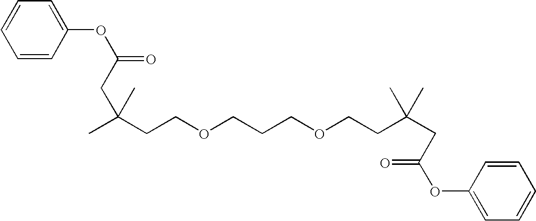

I-153:
5-[3-(3,3-Dimethyl-4-phenoxycarbonyl-butoxy)-propoxy]-3,3-dimethyl-pentanoic acid
phenyl ester

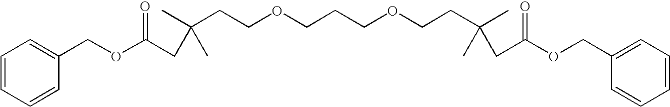

I-154:
5-[3-(4-Benzyloxycarbonyl-3,3-dimethyl-butoxy)-propoxy]-3,3-dimethyl-pentanoic acid
benzyl ester

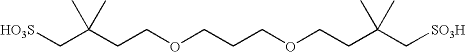

I-155:
4-[3-(3,3-Dimethyl-pentyloxy)-propoxy]-2,2-dimethyl-butane-1-sulfonic acid

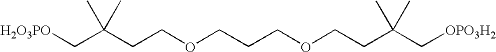

I-156:
Phosphoric acid mono-{4-[3-(3,3-dimethyl-4-phosphonooxy-butoxy)-propoxy]-2,2-
dimethyl-butyl}ester

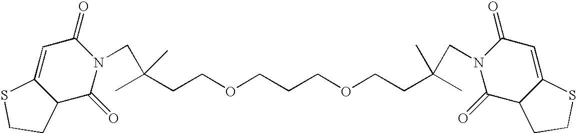

I-157:
5-{4-[3-(3,3-Dimethyl-4-(5-(3,3a-dihydro-2H-thieno-[3,2-c]pyridine-4,6-
dioxo)pentyloxy)-propoxy]-2,2-dimethyl-butyl}-3,3a-dihydro 3,3a-dihydro-2H-thieno-
[3,2-c]pyridine-4,6-dione

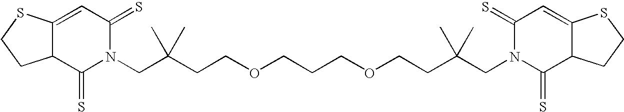

I-158:
5-{4-[3-(3,3-Dimethyl-4-(5-(3,3a-dihydro-2H-thieno-[3,2-c]pyridine-4,6-
dithioxo)pentyloxy)-propoxy]-2,2-dimethyl-butyl}-3,3a-dihydro 3,3a-dihydro-2H-
thieno-[3,2-c]pyridine-4,6-dithione

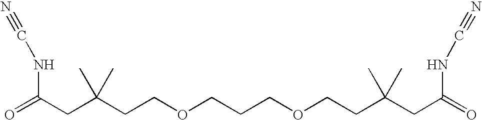

I-159:
5-[3-(3,3-Dimethyl-4-cyano-carbamoyl-butoxy)-propoxy]-3,3-dimethyl-N-cyano-
pentanoic acid-amide

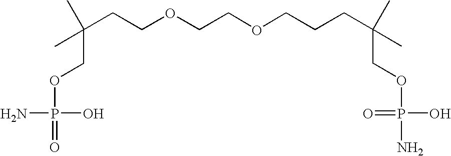

I-160:
Phosphoramidic acid mono-(5-{2-[4-(amino-hydroxy-phosphoryloxy)-
3,3-dimethyl-butoxy]-ethoxy}-2,2-dimethyl-pentyl) ester

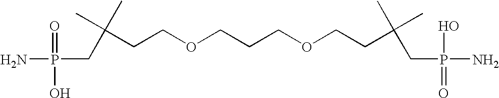

I-161:
{4-[3-(3,3-Dimethyl-4-phosponamido-butoxy)-propoxy]-2,2-dimethyl-butyl}-
phosphonamide

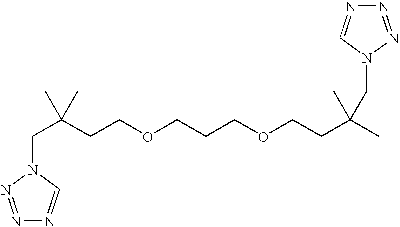

I-162:
I-{4-[3-(3,3-Dimethyl-5-(1H-tetrazol-1-yl)-butoxy)-propoxy]-2,2-dimethyl-butyl}-1H-
tetrazole

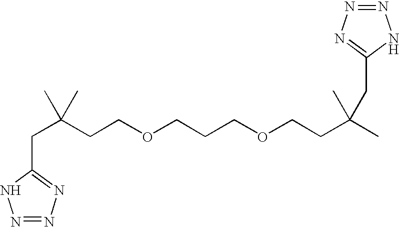

I-163:
5-{4-[3-(3,3-Dimethyl-5-(1H-tetrazol-5-yl)-butoxy)-propoxy]-2,2-dimethyl-butyl}-1H-
tetrazole

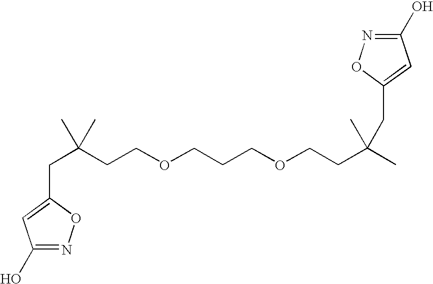

I-164:
5-{4-[3-(3,3-Dimethyl-5-(3-hydroxy-isoxazol-5-yl)-butoxy)-propoxy]-2,2-dimethyl-
butyl}-3-hydroxy-isoxazole

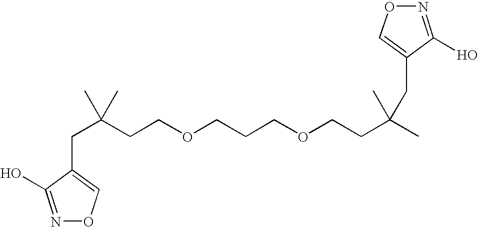

I-165:
4-{4-[3-(3,3-Dimethyl-5-(3-hydroxy-isoxazol-4-yl)-butoxy)-propoxy]-2,2-dimethyl-
butyl}-3-hydroxy-isoxazole

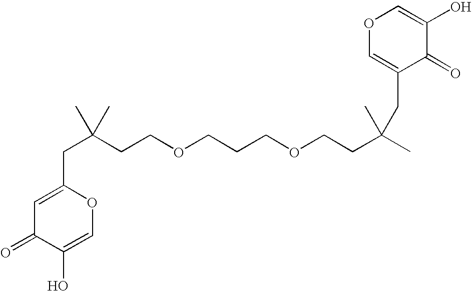

I-166:
2-{4-[3-(3,3-Dimethyl-4-{5-hydroxy-pyran-4-oxo-3-yl}-butyloxy)-propoxy]-2,2-
dimethyl-butyl}-5-hydroxy-pyran-4-one

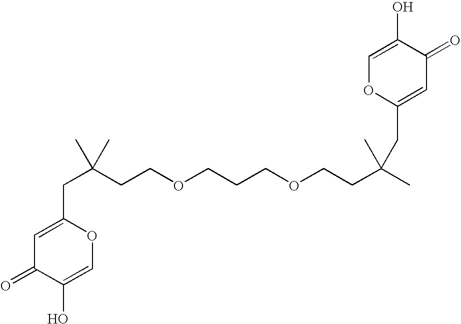

I-167:
2-{4-[3-(3,3-Dimethyl-4-{5-hydroxy-pyran-4-oxo-2-yl}-butyloxy)-propoxy]-2,2-
dimethyl-butyl}-5-hydroxy-pyran-4-one

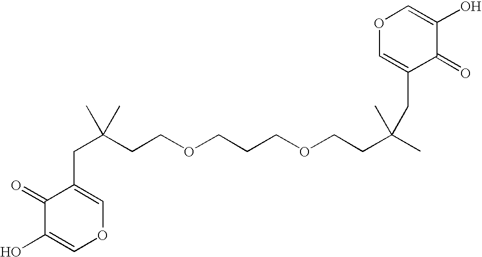

I-168:
3-{4-[3-(3,3-Dimethyl-4-{5-hydroxy-pyran-4-oxo-3-yl}-butyloxy)-propoxy]-2,2-
dimethyl-butyl}-5-hydroxy-pyran-4-one

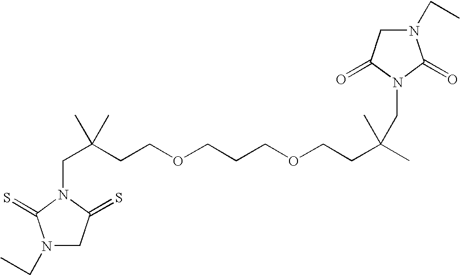

I-169:
1-Ethyl-3-(4-{3-[4-(3-ethyl-2,5-dithioxo-imidazolidin-1-yl)-3,3-dimethyl-butoxy]-
propoxy}-2,2-dimethyl-butyl)-imidazolidine-2,4-dione

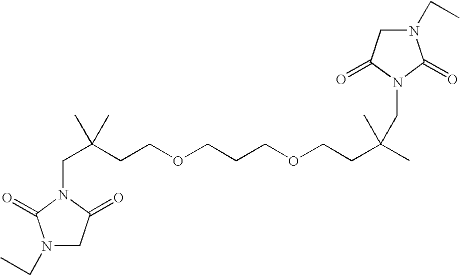

I-170:
1-Ethyl-3-(4-{3-[4-(3-ethyl-2,5-oxo-imidazolidin-1-yl)-3,3-dimethyl-butoxy]-
propoxy}-2,2-dimethyl-butyl)-imidazolidine-2,4-dio

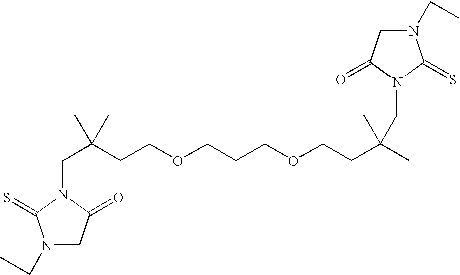

I-171:
1-Ethyl-3-(4-{3-[4-(3-ethyl-2-thioxo-5-oxo-imidazolidin-1-yl)-
3,3-dimethyl-butoxy]-propoxy}-2,2-dimethyl-butyl)-imidazolidine-2-thioxo-4-one

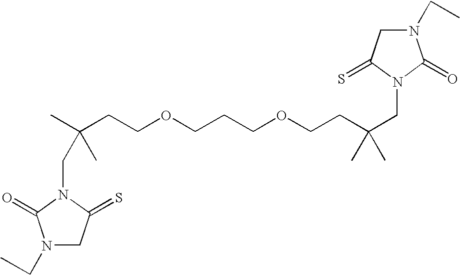

I-172:
1-Ethyl-3-(4-{3-[4-(3-ethyl-2-oxo-5-thioxo-imidazolidin-1-yl)-3,3-
dimethyl-butoxy]-propoxy}-2,2-dimethyl-butyl)-imidazolidine-2-oxo-4-thione

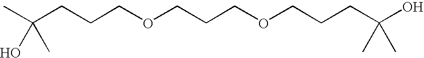

I-173:
5-[3-(4-Hydroxy-4-methyl-pentyloxy)-propoxy]-2-methyl-pentan-2-ol

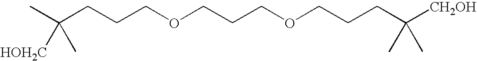

I-174:
5-[3-(5-Hydroxy-4,4-dimethyl-pentyloxy)-propoxy]-2,2-dimethyl-pentan-1-ol

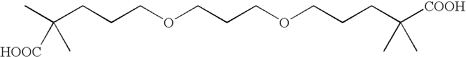

I-175:
5-[3-(4-Carboxy-4-methyl-pentyloxy)-propoxy]-2,2-dimethyl-pentanoic acid

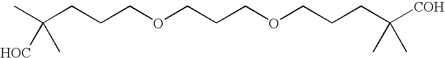

I-176:
5-[3-(4,4-Dimethyl-5-oxo-pentyloxy)-propoxy]-2,2-dimethyl-pentanal

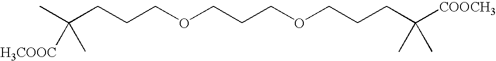

I-177:
5-[3-(4-Methoxycarbonyl-4-methyl-pentyloxy)-propoxy]-2,2-dimethyl-pentanoic
acid methyl ester

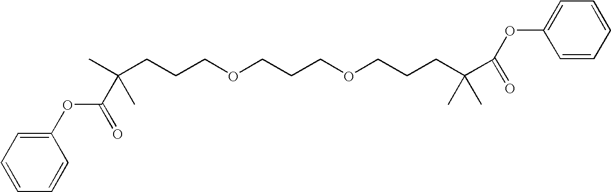

I-178:
5-[3-(4,4-Dimethyl-5-oxo-6-phenyl-hexyloxy)-propoxy]-2,2-dimethyl-pentanoic
acid phenyl ester

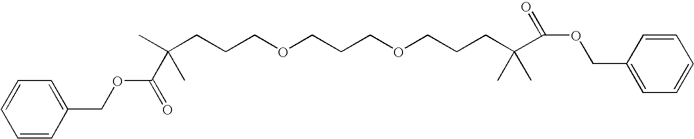

I-179:
4-{3-[1-(2-Benzyloxycarbonyl-2-methyl-propyl)-vinyloxy]-propoxy}-
2,2-dimethyl-pent-4-enoic acid benzyl ester

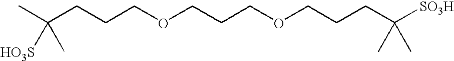

I-180:
2-Methyl-5-[3-(4-methyl-4-sulfo-pentyloxy)-propoxy]-pentane-2-sulfonic acid

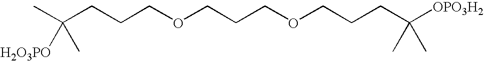

I-181:
Phosphoric acid mono-{1,1-dimethyl-4-[3-(4-methyl-4-phosphonooxy-
pentyloxy)-propoxy]-butyl}ester

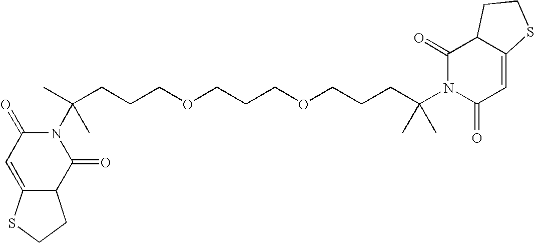

I-182:
5-(5-{3-[3,3-Dimethyl-5-(4,6-dioxo-2,3,3a,6-tetrahydro-4H-thieno[3,2-c]
pyridin-5-yl)-pentyloxy]-propoxy}-3,3-dimethyl-pentyl)-3,3a-dihydro-2H-thieno[3,2-c]
pyridine-4,6-dione

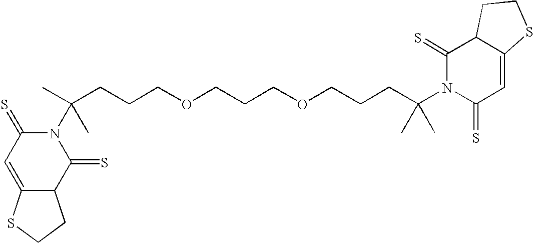

I-183:
5-(5-{3-[3,3-Dimethyl-5-(4,6-dithioxo-2,3,3a,6-tetrahydro-4H-thieno[3,2-c]
pyridin-5-yl)-pentyloxy]-propoxy}-3,3-dimethyl-pentyl)-3,3a-dihydro-2H-thieno[3,2-c]
pyridine-4,6-dithione

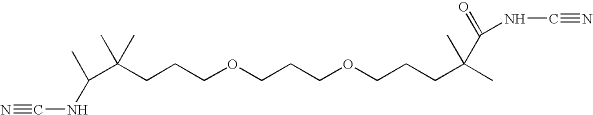

I-184:
5-{3-[4-N-Cyano-carbamoyl-4-methyl-pentyloxy)-propoxy]-2,2-dimethyl-N-cyano-
pentanoic acid-amide

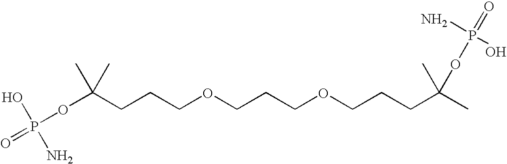

I-185:
Phosphoramidic acid mono-[3-(3-{1-[2-(amino-hydroxy-phosphoryloxy)-2-methyl-
propyl]-vinyloxy}-propoxy)-1,1-dimethyl-but-3-enyl]ester

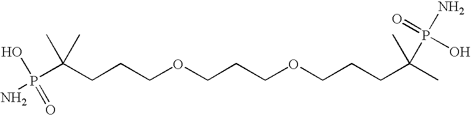

I-186:
{1,1-Dimethyl-4-[3-(4-methyl-4-phosphoriamido-pentyloxy)-propoxy]-butyl}-
phosphonamide

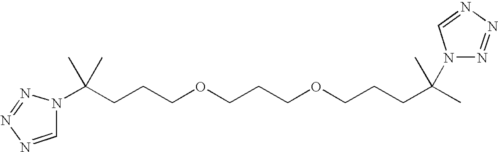

I-187:
1-{4-[3-(4-{1H-Tetrazol-1-yl}-4-methyl-pentyloxy)-propoxy]-1,1-dimethyl-
butyl}-1H-tetrazol

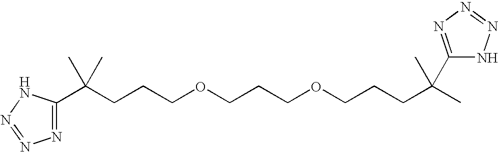

I-188:
5-{4-[3-(4-{1H-Tetrazol-5-yl}-4-methyl-pentyloxy)-propoxy]-1,1-dimethyl-
butyl}-1H-tetrazole

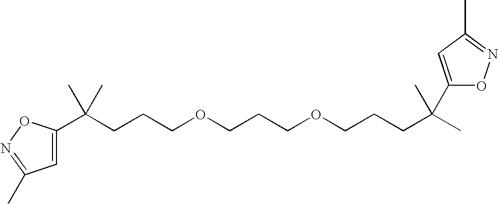

I-189:
5-{4-[3-(4-{3-Methyl-isoxazol-5-yl}-4-methyl-pentyloxy)-propoxy]-1,1-
dimethyl-butyl}-3-methyl-isoxazole

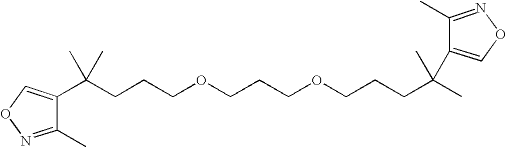

I-190:
4-{4-[3-(4-{3-Methyl-isoxazol-4-yl}-4-methyl-pentyloxy)-propoxy]-1,1-dimethyl-
butyl}-3-methyl-isoxazole

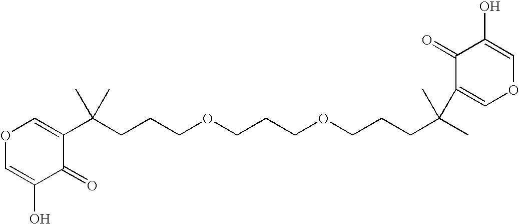

I-191:
3-{4-[3-(4-{5-Hydroxy-4-oxo-pyran-3-yl}-4-methyl-pentyloxy)-propoxy]-
1,1-dimethyl-butyl}-5-hydroxy-pyran-4-one

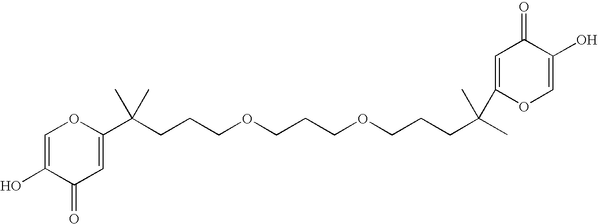

I-192:
2-{4-[3-(4-{5-Hydroxy-4-oxo-pyran-2-yl}-4-methyl-pentyloxy)-propoxy]-1,1-dimethyl-
butyl}-5-hydroxy-pyran-4-one

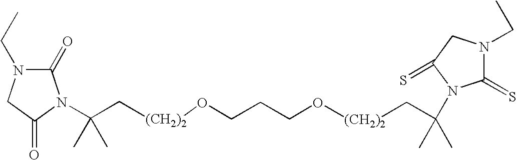

I-193:
1-Ethyl-3-(4-{3-[4-(3-ethyl-2,5-dithioxo-imidazolidin-1-yl)-4-methyl-pentyloxy]-
propoxy}-1,1-dimethyl-butyl)-imidazolidine-2,4-dione

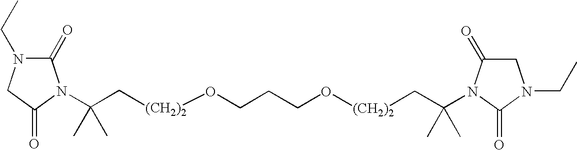

I-194:
1-Ethyl-3-(4-{3-[4-(3-ethyl-2,5-dioxo-imidazolidin-1-yl)-4-methyl-pentyloxy]-propoxy}-
1,1-dimethyl-butyl)-imidazolidine-2,4-dione

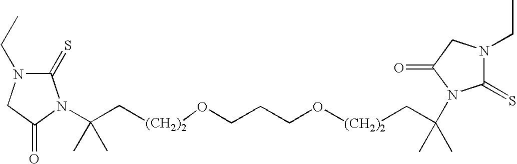

I-195:
1-Ethyl-3-(4-{3-[4-(3-ethyl-5-oxo-2-thioxo-imidazolidin-1-yl)-4-methyl-pentyloxy]-
propoxy}-1,1-dimethyl-butyl)-imidazolidine-4-oxo-2-thione

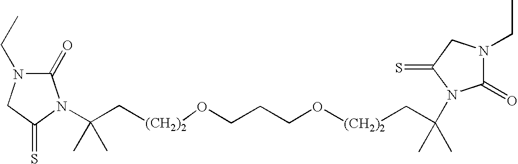

I-196:
1-Ethyl-3-(4-{3-[4-(3-ethyl-2-oxo-5-thioxo-imidazolidin-1-yl)-4-methyl-pentyloxy]-
propoxy}-1,1-dimethyl-butyl)-imidazolidine-2-oxo-4-thione

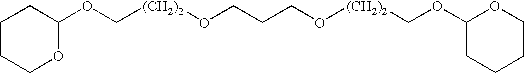

I-197:
2-{3-[3-(3-{Tetrahydro-pyran-2-yl}-propoxy)-propoxy]-propoxy}-tetrahydro-pyran

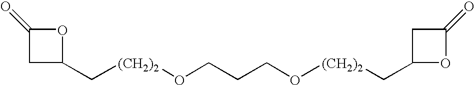

I-198:
4-{3-[3-(3-{Oxetan-2-one-4-yl}propoxy)-propoxy]-propyl}-oxetan-2-one

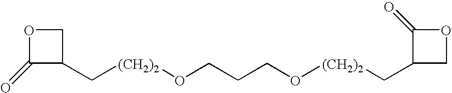

I-199:
3-{3-[3-(3-{Oxetan-2-one-3-yl}propoxy)-propoxy]-propyl}-oxetan-2-one

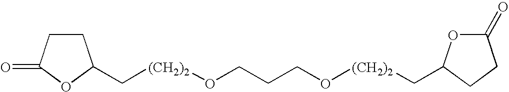

I-200:
5-{3-[3-(3-{Dihydro-furan-2-one-5-yl}-propoxy)-propoxy]-propyl}-dihydro-furan-2-one

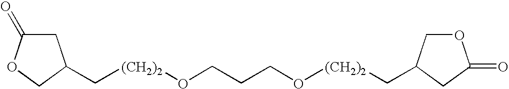

I-201:
4-{3-[3-(3-{Dihydro-furan-2-one-4-yl}-propoxy)-propoxy]-propyl}-dihydro-furan-2-one

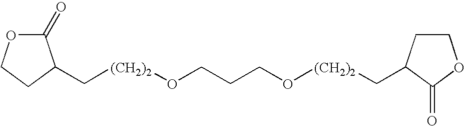

I-202:
3-{3-[3-(3-{Dihydro-furan-2-one-3-yl}-propoxy)-propoxy]-propyl}-dihydro-furan-2-one

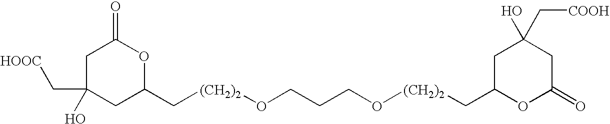

I-203:
{2-[3-(3-{3-[4-Carboxymethyl-4-hydroxy-6-oxo-tetrahydro-pyran-2-yl)-propoxy}-
propoxy)-propyl]-4-hydroxy-6-oxo-tetrahydro-pyran-4-yl}-acetic acid

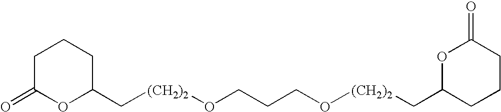

I-204:
6-{3-[3-(3-{Dihydro-pyran-2-one-6-yl}-propoxy)-propoxy]-propyl}-dihydro-pyran-2-one

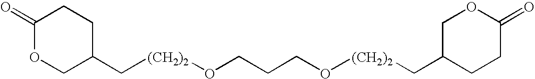

I-205:
5-{3-[3-(3-{Dihydro-pyran-2-one-5-yl}-propoxy)-propoxy]-propyl}-dihydro-pyran-2-one

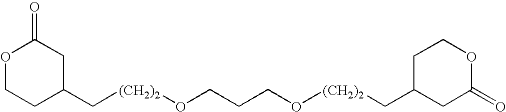

I-206:
4-{3-[3-(3-{Dihydro-pyran-2-one-4-yl-propoxy)-propoxy]-propyl}-dihydro-pyran-2-one

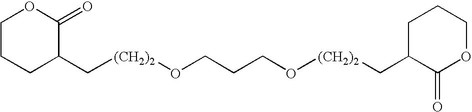

I-207:
3-{3-[3-(3-{Dihydro-pyran-2-one-3-yl}-propoxy)-propoxy]-propyl}-dihydro-pyran-2-one

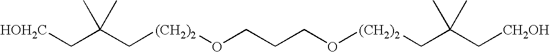

I-208:
6-[3-(6-Hydroxy-4,4-dimethyl-hexyloxy)-propoxy]-3,3-dimethyl-hexan-1-ol

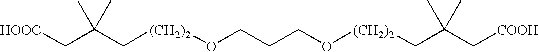

I-209:
6-[3-(5-Carboxy-4,4-dimethyl-pentyloxy)-propoxy]-3,3-dimethyl-hexanoic acid

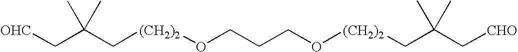

I-210:
6-[3-(4,4-Dimethyl-6-oxo-hexyloxy)-propoxy]-3,3-dimethyl-hexanal

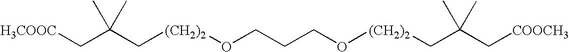

I-211:
6-[3-(5-Methoxycarbonyl-4,4-dimethyl-pentyloxy)-propoxy]-3,3-dimethyl-hexanoic acid
methyl ester

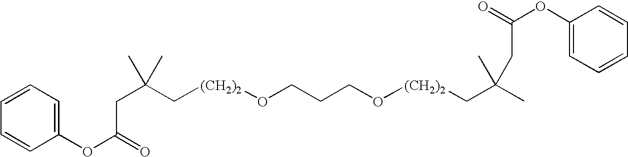

I-212:
6-[3-(4,4-Dimethyl-5-phenoxycarbonyl-pentyloxy)-propoxy]-3,3-dimethyl-hexanoic acid
cyclohexyl ester

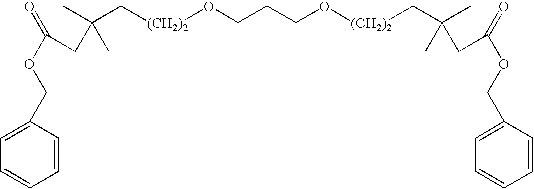

I-213:
6-[3-(5-Benzyloxycarbonyl-4,4-dimethyl-pentyloxy)-propoxy]-3,3-dimethyl-hexanoic
acid benzyl ester

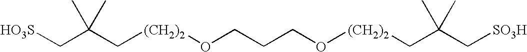

I-214:
5-[3-(4,4-Dimethyl-5-sulfo-pentyloxy)-propoxy]-2,2-dimethyl-pentane-1-sulfonic acid

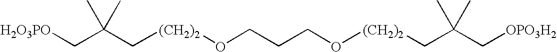

I-215:
5-[3-(4,4-Dimethyl-5-phospho-pentyloxy)-propoxy]-2,2-dimethyl-pentane-1-phosphonic acid

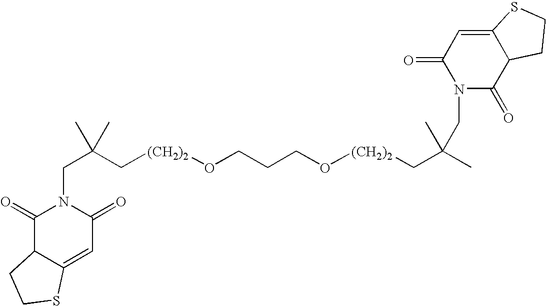

I-216:
5-{5-[3-(5-(3,3a-Dihydro-2H-thieno[3,2-c]pyridine-4,6-dione-5-yl}-4,4-dimethyl-
pentyloxy)-propoxy]-2,2-dimethyl-3-pentyl}-3,3a-dihydro-2H-thieno[3,2-c]pyridine-4,6-dione

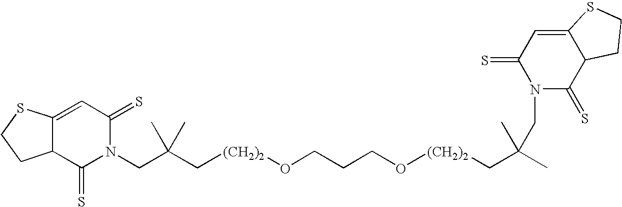

I-217:
5-{5-[3-(5-{3,3a-Dihydro-2H-thieno[3,2-c]pyridine-4,6-dithione-5-yl}-4,4-dimethyl-
pentyloxy)-propoxy]-2,2-dimethyl-3-pentyl}-3,3a-dihydro-2H-thieno[3,2-c]pyridine-4,6-
dithione

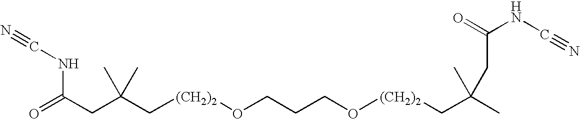

I-218:
6-[3-(5-Cyano-carbamoyl-4,4-dimethyl-pentyloxy)-propoxy]-3,3-dimethyl-N-cyano-
hexanoic acid-amide

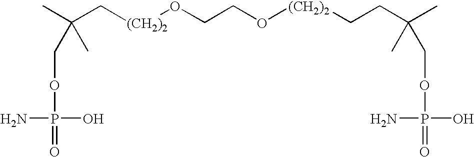

I-219:
Phosphoramidic acid mono-(6-{2-[5-(amino-hydroxy-phosphoryloxy)-4,4-dimethyl-
pentyloxy]-ethoxy}-2,2-dimethyl-hexyl) ester

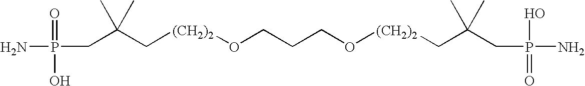

I-220:
{5-[3-(4,4-Dimethyl-5-phosphonamido-pentyloxy)-propoxy]-2,2-dimethyl-pentyl}-
phosphonamide

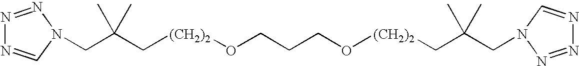

I-221:
1-{[3-(5-{1H-Tetrazol-1-yl}-4,4-dimethyl-pentyloxy)-propoxy]-2,2-dimethyl-pentyl}-
1H-tetrazole

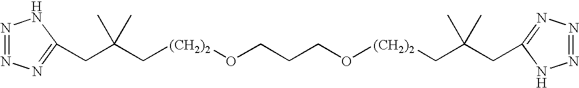

I-222:
5-{5-[3-(5-(1H-Tetrazol-5-yl}-4,4-dimethyl-pentyloxy)-propoxy]-2,2-dimethyl-pentyl}-
1H-tetrazole

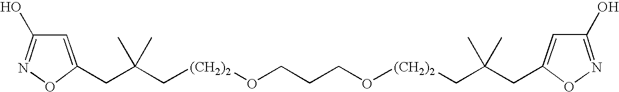

I-223:
5-{5-[3-(5-{3-Hydroxy-isoxazol-5-yl}-4,4-dimethyl-pentyloxy)-propoxy]-2,2-dimethyl-
pentyl}-3-hydroxy-isoxazole

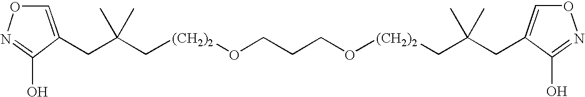

I-224:
4-{5-[3-(5-{3-Hydroxy-isoxazol-4-yl}-4,4-dimethyl-pentyloxy)-propoxy]-2,2-dimethyl-
pentyl}-3-hydroxy-isoxazole

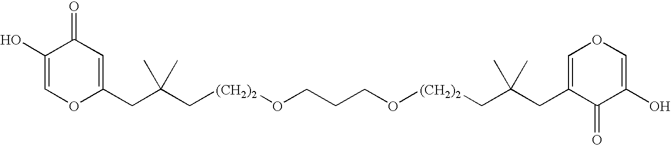

I-225:
2-{5-[3-(5-{5-Hydroxy-4-oxo-pyran-3-yl}-4,4-dimethyl-pentyloxy)-propoxy]-2,2-
dimethyl-pentyl}-5-hydroxy-pyran-4-one

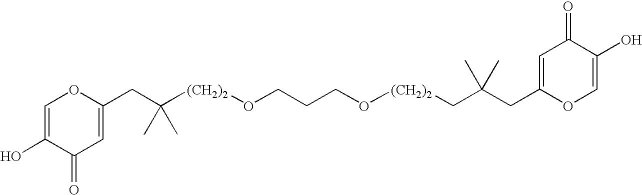

I-226:
2-{5-[3-(5-{-Hydroxy-4-oxo-pyran-2-yl}-4,4-dimethyl-pentyloxy)-propoxy]-2,2-
dimethyl-pentyl}-5-hydroxy-pyran-4-one

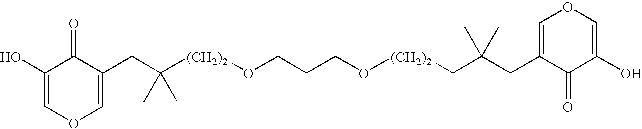

I-227:
3-{5-[3-(5-{5-Hydroxy-4-oxo-pyran-3-yl}-4,4-dimethyl-pentyloxy)-propoxy]-2,2-
dimethyl-pentyl}-5-hydroxy-pyran-4-one

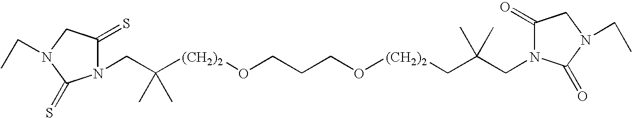

I-228:
3-{4-[3-(5-{3-Ethyl-2,5-dithioxo-imidazolidin-1-yl}-4,4-dimethyl-pentyloxy)-propoxy]-
2,2-dimethyl-butyl}-1-ethyl-imidazolidine-2,4-dithione

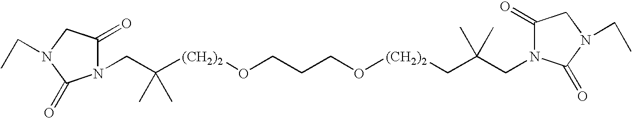

I-229:
3-{4-[3-(5-{3-Ethyl-2,5-dioxo-imidazolidin-1-yl}-4,4-dimethyl-pentyloxy)-propoxy]-2,2-
dimethyl-butyl}-1-ethyl-imidazolidine-2,4-dione

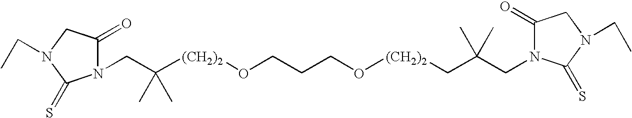

I-230:
3-{4-[3-(5-{3-Ethyl-5-oxo-2-thioxo-imidazolidin-1-yl}-4,4-dimethyl-pentyloxy)-
propoxy]-2,2-dimethyl-butyl}-1-ethyl-imidazolidine-4-oxo-2-thione

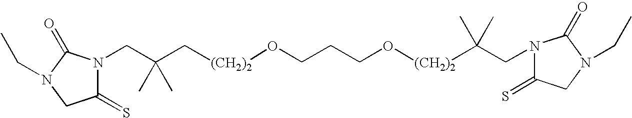

I-231:
3-{4-[3-(5-(3-Ethyl-5-oxo-2-thioxo-imidazolidin-1-yl}-4,4-dimethyl-pentyloxy)-
propoxy]-2,2-dimethyl-butyl}-1-ethyl-imidazolidine-2-oxo-4-thione

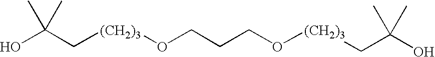

I-232:
6-[3-(5-Hydroxy-5-methyl-hexyloxy)-propoxy]-2-methyl-hexan-2-ol

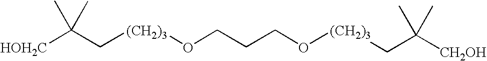

I-233:
6-[3-(6-Hydroxy-5,5-dimethyl-hexyloxy)-propoxy]-2,2-dimethyl-hexan-1-ol

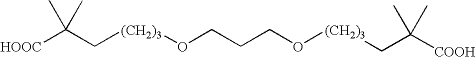

I-234:
6-[3-(5-Carboxy-5-methyl-hexyloxy)-propoxy]-2,2-dimethyl-hexanoic acid

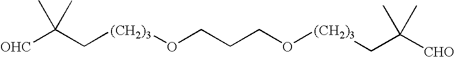

I-235:
6-[3-(5,5-Dimethyl-6-oxo-hexyloxy)-propoxy]-2,2-dimethyl-hexanal

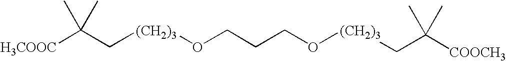

I-236:
6-[3-(5-Methoxycarbonyl-5-methyl-hexyloxy)-propoxy]-2,2-dimethyl-hexanoic acid
methyl ester

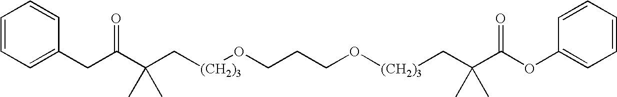

I-237:
6-[3-(5,5-Dimethyl-6-oxo-7-phenyl-heptyloxy)-propoxy]-2,2-dimethyl-hexanoic acid
phenyl ester

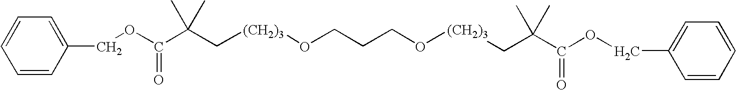

I-238:
6-[3-(5-Benzyloxycarbonyl-5-methyl-hexyloxy)-propoxy]-2,2-dimethyl-hexanoic acid
benzyl ester

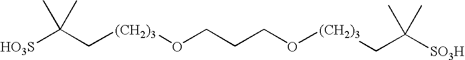

I-239:
2-Methyl-6-[3-(5-methyl-5-sulfo-hexyloxy)-propoxy]-hexane-2-sulfonic acid

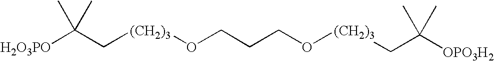

I-240:
Phosphoric acid mono-{1,1-dimethyl-5-[3-(5-methyl-5-phosphonooxy
hexyloxy)-propoxy]-pentyl}ester

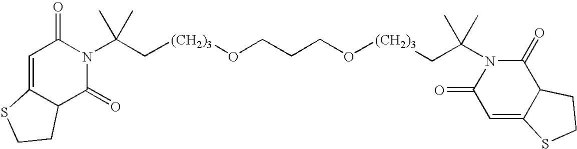

I-241:
5-(5-{3-[4-(4,6-Dioxo-hexahydro-thieno[3,2-c]pyridi n-5-yl)-4-methyl-pentyloxy]-
propoxy}-1,1-dimethyl-pentyl)-3,3a-dihydro-2H-thieno[3,2-c]pyridine-4,6-dione

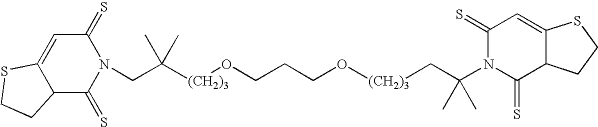

I-242:
5-(5-{3-[4-(4,6-Dithioxo-hexahydro-thieno[3,2-c]pyridin-5-yl)-4-methyl-pentyloxy]-
propoxy}-1,1-dimethyl-pentyl)-3,3a-dihydro-2H-thieno[3,2-c]pyridine-4,6-dithione

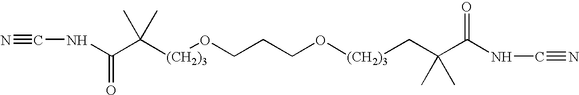

I-243:
6-[3-(4-N-Cyano-carbamoyl-4-methyl-pentyloxy)-propoxy]-2,2-dimethyl-N-cyano-
hexanoic acid-amide

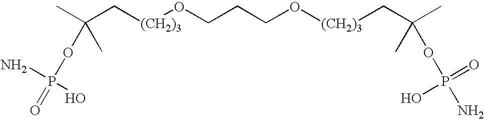

I-244:
Phosphoramidic acid mono-(5-{3-[5-(amino-hydroxy-phosphoryloxy)-
5-methyl-hexyloxy]-propoxy}-1,1-dimethyl-pentyl) ester

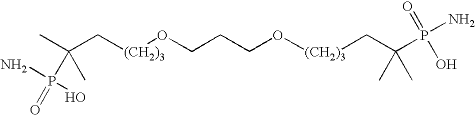

I-245:
{1,1-Dimethyl-5-[3-(5-methyl-5-phosphonamido-hexyloxy)-propoxy]-pentyl}-
phosphonamide

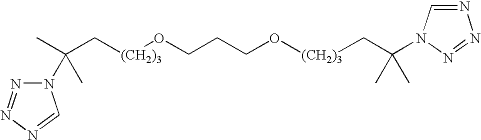

I-246:
I-{5-[3-(5-{1H-Tetrazol-1-yl}-5-methyl-hexyloxy)-propoxy]-1,1-dimethyl-pentyl}-1H-
tetrazole

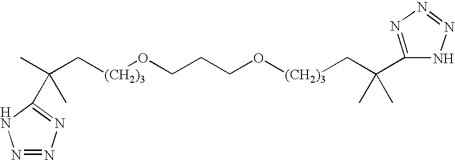

I-247:
5-{5-[3-(5-{1H-Tetrazol-5-yl}-5-methyl-hexyloxy)-propoxy]-1,1-dimethyl-pentyl}-1H-
tetrazole

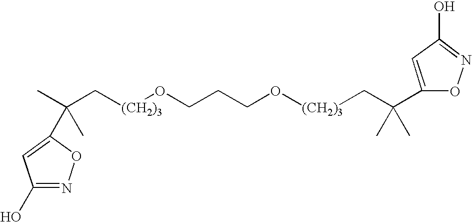

I-248:
5-{5-[3-(5-{3-Hydroxy-isoxazol-5-yl}-5-methyl-hexyloxy)-propoxy]-1,1-dimethyl-
pentyl}-3-hydroxy-isoxazole

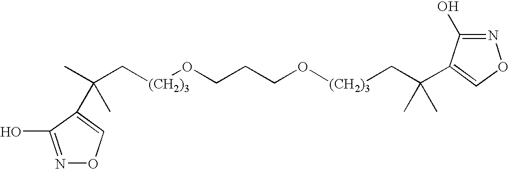

I-249:
4-{5-[3-(5-{3-Hydroxy-isoxazol-4yl}-5-methyl-hexyloxy)-propoxy]-1,1-dimethyl-
pentyl}-3-hydroxy-isoxazole

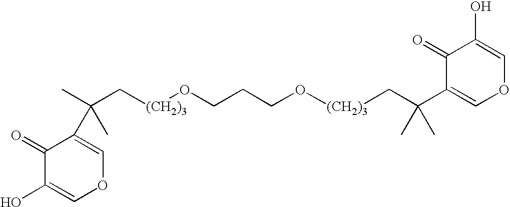

I-250:
3-{5-[3-(5-{5-Hydroxy-4-oxo-pyran-3-yl}-5-methyl-hexyloxy)-propoxy]-1,1-dimethyl-
pentyl}-5-hydroxy-pyran-4-one

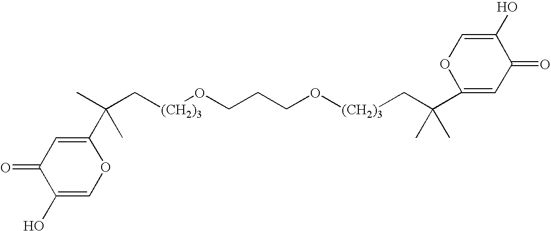

I-251:
2-{5-[3-(5-{5-Hydroxy-4-oxo-pyran-2-yl}-5-methyl-hexyloxy)-propoxy]-1,1-dimethyl-
pentyl}-5-hydroxy-pyran-4-one

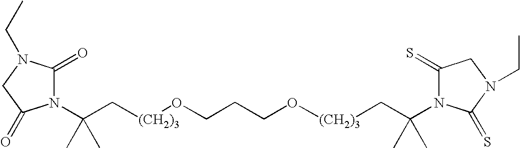

I-252:
1-Ethyl-3-(5-{3-[5-(3-ethyl-2,5-dithioxo-imidazolidin-1-yl)-5-methyl-hexyloxy]-
propoxy}-2,2-dimethyl-hexyl)-imidazolidine-2,4-dione

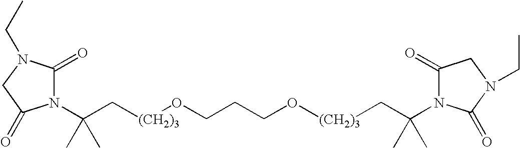

I-253:
1-Ethyl-3-(5-{3-[5-(3-ethyl-2,5-dioxo-imidazolidin-1-yl)-5-methyl-hexyloxy]-
propoxy}-2,2-dimethyl-hexyl)-imidazolidine-2,4-dione

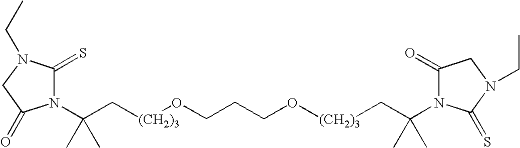

I-254:
1-Ethyl-3-(5-{3-[5-(3-ethyl-2-thioxo-5-oxo-imidazolidin-1-yl)-5-methyl-hexyloxy]-
propoxy}-2,2-dimethyl-hexyl)-imidazolidine-4-oxo-2-thione

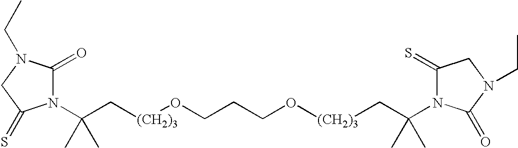

I-255:
1-Ethyl-3-(5-{3-[5-(3-ethyl-5-thioxo-2-oxo-imidazolidin-1-yl)-5-methyl-hexyloxy]-
propoxy}-2,2-dimethyl-hexyl)-imidazolidine-2-oxo-4-thione

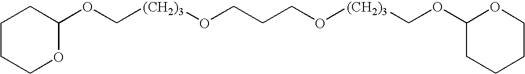

I-256:
2-{4-[3-(4-{Tetrahydro-pyran-2-yl}-butoxy)-propoxy]-butoxy}-tetrahydro-pyran

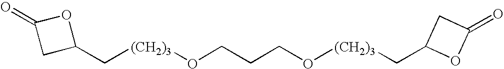

I-257:
4-{4-[3-(4-{Oxetan-2-one-4-yl}-butoxy)-propoxy]-butyl}-oxetan-2-one

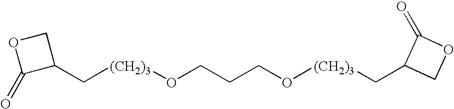

I-258:
3-{4-[3-(4-{Oxetan-2-one-3-yl}-butoxy)-propoxy]-butyl}-oxetan-2-one

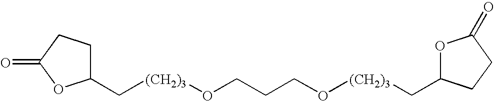

5-{4-[3-(4-{Tetrahydro-furan-2-one-5-yl}-butoxy)-propoxy]-butyl}-tetrahydro-
furan-2-one

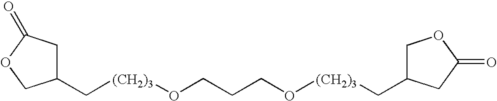

I-260:
4-{4-[3-(4-{Tetrahydro-furan-2-one-4-yl}-butoxy)-propoxy]-butyl}-tetrahydro-furan-2-one

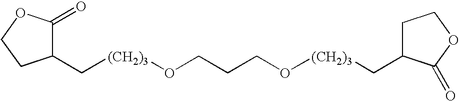

I-261:
3-{4-[3-(4-{Tetrahydro-furan-2-one-3-yl}-butoxy)-propoxy]-butyl}-tetrahydro-furan-2-one

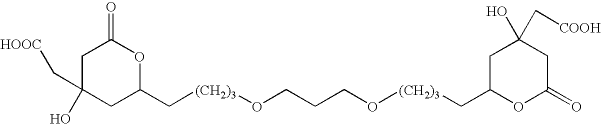

I-262:
[2-(4-{3-[4-(4-Carboxymethyl-4-hydroxy-6-oxo-tetrahydro-pyran-2-yl)-butoxy]-
propoxy}-butyl)-4-hydroxy-6-oxo-tetrahydro-pyran-4-yl)-acetic acid

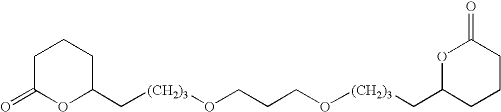

6-{4-[3-(4-{Tetrahydro-pyran-2-one-6-yl}-butoxy)-propoxy]-butyl}-tetrahydro-pyran-2-one

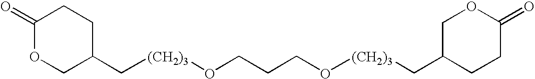

I-264:
5-{4-[3-(4-{Tetrahydro-pyran-2-one-5-yl}-butoxy)-propoxy]-butyl}-tetrahydro-pyran-2-one

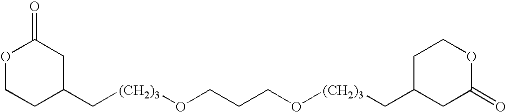

I-265:
4-{4-[3-(4-{Tetrahydro-pyran-2-one-4-yl}-butoxy)-propoxy]-butyl}-tetrahydro-pyran-2-one

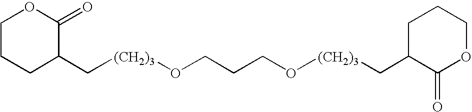

I-266:
3-{4-[3-(4-{Tetrahydro-pyran-2-one-3-yl}-butoxy)-propoxy]-butyl}-tetrahydro-pyran-2-one

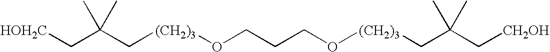

I-267:
7-[3-(7-Hydroxy-5,5-dimethyl-heptyloxy)-propoxy]-3,3-dimethyl-heptan-1-ol

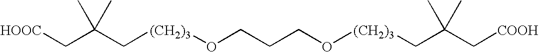

I-268:
7-[3-(6-Carboxy-5,5-dimethyl-hexyloxy)-propoxy]-3,3-dimethyl-heptanoic acid

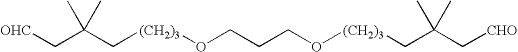

I-269:
7-[3-(5,5-dimethyl-6-oxo-hexyloxy)-propoxy]-3,3-dimethyl-heptanal

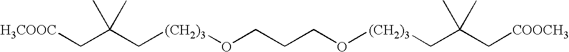

I-270:
7-[3-(6-Methoxycarbonyl-5,5-dimethyl-hexyloxy)-propoxy]-3,3-dimethyl-heptanoic acid
methyl ester

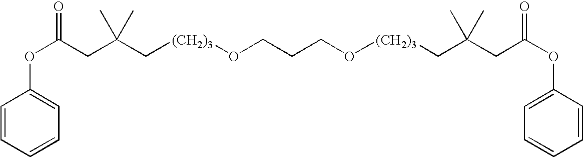

I-271:
7-[3-(5,5-Dimethyl-6-phenoxycarbonyl-hexyloxy)-propoxy]-3,3-dimethyl-heptanoic acid
phenyl ester

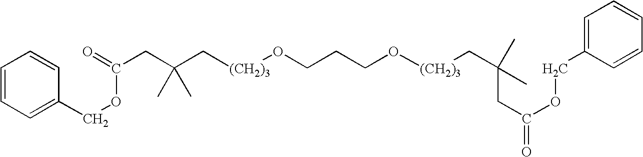

I-272:
7-[3-(6-Benzyloxycarbonyl-5,5-dimethyl-hexyloxy)-propoxyl-3,3-dimethyl-heptanoic
acid benzyl ester

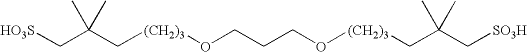

I-273:
6-[3-(5,5-Dimethyl-6-sulfo-hexyloxy)-propoxy]-2,2-dimethyl-hexane-1-sulfonic acid

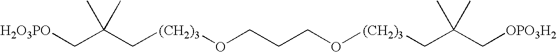

I-274:
Phosphoric acid mono-{6-[3-(5,5-dimethyl-6-phosphonooxy-hexyloxy)-propoxy]-2,2-
dimethyl-hexyl}-ester

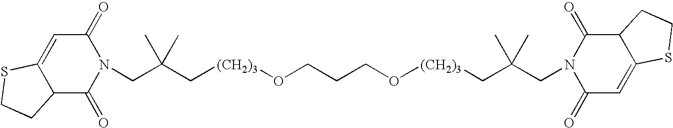

I-275:
5-(6-{3-[6-(4,6-Dioxo-hexahydro-thieno[3,2-c]pyridin-5-yl)-5,5-dimethyl-hexyloxy]-
propoxy}-2,2-dimethyl-hexyl)-3,3a-dihydro-2H-thieno[3,2-c]pyridine-4,6-dione

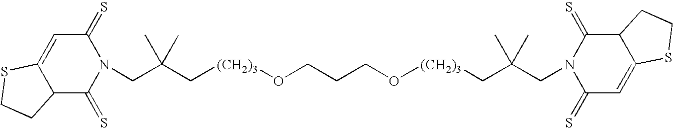

I-276:
5-(5-{3-[4-(4,6-Dithioxo-hexahydro-thieno[3,2-c]pyridin-5-yl)-4-methyl-pentyloxy]-
propoxy}-1,1-dimethyl-pentyl)-3,3a-dihydro-2H-thieno[3,2-c]pyridine-4,6-dithione

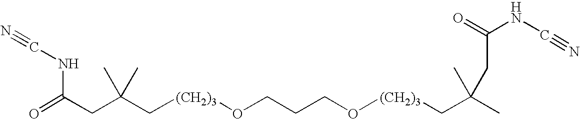

I-277:
7-[3-(6-N-Cyano-carbamoyl-5,5-dimethyl-hexyloxy)-propoxy]-3,3-dimethyl-N-cyano-
heptanoic acid-amide

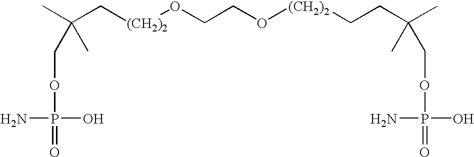

I-278:
Phosphoramidic acid mono-{7-[2-(6-{amino-hydroxy-phosphoryloxy}-5,5-dimethyl-
hexyloxy)-ethoxy]-2,2-dimethyl-heptyl}ester

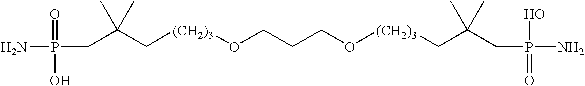

I-279:
{6-[3-(5,5-Dimethyl-6-phosphonamido-hexyloxy)-propoxy]-2,2,-dimethyl-hexyl}-
phosphonamide

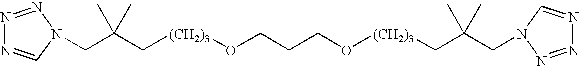

I-280:
1-{6-[3-(6-{1H-Tetrazol-1-yl}-5,5-dimethyl-hexyloxy)-propoxy]-2,2-dimethyl-hexyl}-
1H-tetrazole

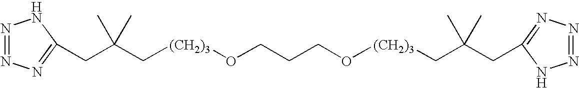

I-281:
5-{6-[3-(6-{1H-Tetrazol-5-yl}-5,5-dimethyl-hexyloxy)-propoxy]-2,2-dimethyl-hexyl}-
1H-tetrazole

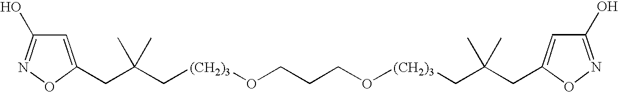

I-282:
5-{6-[3-(6-{3-Hydroxy-isoxazol-5-yl}-5,5-dimethyl-hexyloxy)-propoxy]-2,2-dimethyl-
hexyl}-3-hydroxy-isoxazole

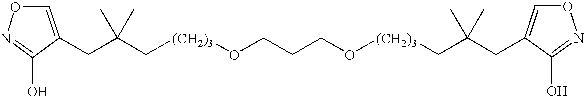

I-283:
4-{6-[3-(6-{3-Hydroxy-isoxazol-4-yl}-5,5-dimethyl-hexyloxy)-propoxy]-2,2-dimethyl-
hexyl}-3-hydroxy-isoxazole

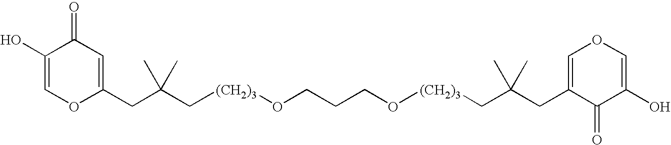

I-284:
2-{6-[3-(6-{5-Hydroxy-4-oxo-pyran-3-yl}-5,5-dimethyl-hexyloxy)-propoxy]-2,2-
dimethyl-hexyl}-5-hydroxy-pyran-4-one

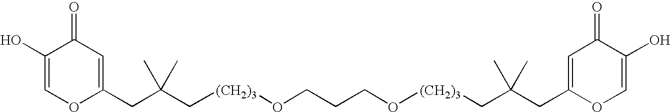

I-285:
2-{6-[3-(6-{5-Hydroxy-4-oxo-pyran-2-yl}-5,5-dimethyl-hexyloxy)-propoxy]-2,2-
dimethyl-hexyl}-5-hydroxy-pyran-4-one

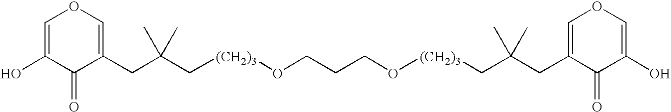

I-286:
3-{6-[3-(6-{5-Hydroxy-4-oxo-pyran-3-yl}-5,5-dimethyl-hexyloxy)-propoxy]-2,2-
dimethyl-hexyl}-5-hydroxy-pyran-4-one

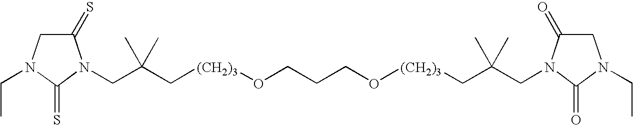

I-287:
1-Ethyl-3-(6-{3-[6-(3-ethyl-2,5-dithioxo-imidazolidin-1-yl)-5,5-dimethyl-hexyloxy]-
propoxy}-2,2-dimethyl-hexyl)-imidazolidine-2,4-dione

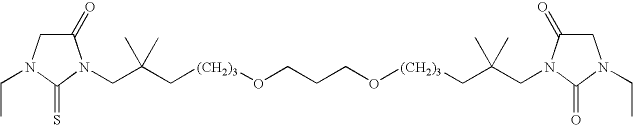

I-288:
1-Ethyl-3-(6-{3-[6-(3-ethyl-2,5-dioxo-imidazolidin-1-yl)-5,5-dimethyl-hexyloxy]-
propoxy}-2,2-dimethyl-hexyl)-imidazolidine-2,4-dione

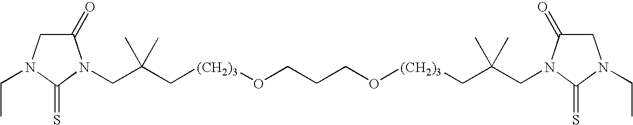

I-289:
1-Ethyl-3-(6-{3-[6-(3-ethyl-5-oxo-2-thioxo-imidazolidin-1-yl)-5,5-dimethyl-
hexyloxy]-propoxy}-2,2-dimethyl-hexyl)-imidazolidine-4-oxo-2-thione

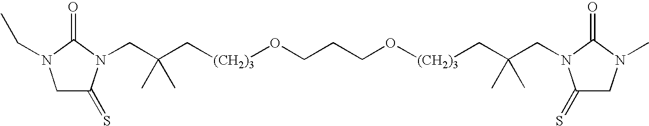

I-290:
1-Ethyl-3-(6-{3-[6-(3-ethyl-2-oxo-5-thioxo-imidazolidin-1-yl)-5,5-dimethyl-
hexyloxy]-propoxy}-2,2-dimethyl-hexyl)-imidazolidine-2-oxo-4-thione

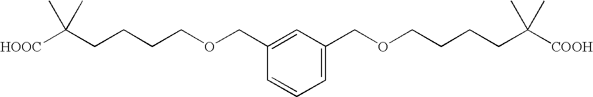

I-291:
6-[3-(5-Carboxy-5-methyl-hexyloxymethyl)-benzyloxy]-2,2-dimethyl-hexanoic acid

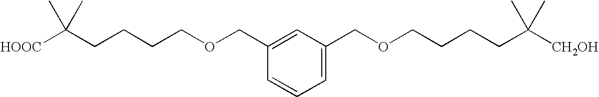

6-[3-(5-Carboxy-5-methyl-hexyloxymethyl)-benzyloxy]-2,2-dimethyl-hexan-1-ol

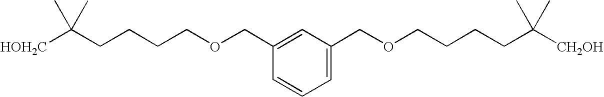

I-293:
6-[3-(6-Hydroxy-5,5-dimethyl-hexyloxymethyl)-benzyloxy]-2,2-dimethyl-hexan-1-ol

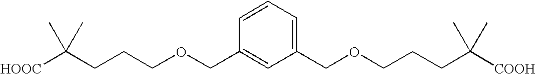

I-294:
5-[3-(4-Carboxy-4-methyl-pentyloxymethyl)-benzyloxy]-2,2-dimethyl-pentanoic acid

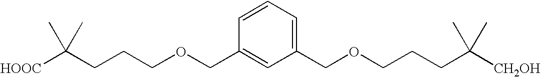

I-295:
5-[3-(4-Carboxy-4-methyl-pentyloxymethyl)-benzyloxy]-2,2-dimethyl-hexan-1-ol

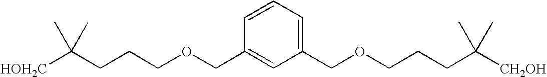

I-296:
5-[3-(5-Hydroxy-4,4-dimethyl-pentyloxymethyl)-benzyloxy]-2,2-dimethyl-pentan-1-ol

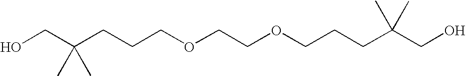

I-297
5-[2-(5-hydroxy-4,4-dimethyl-pentyloxy)-ethoxy]-2,2-dimethyl-pentan-1-ol

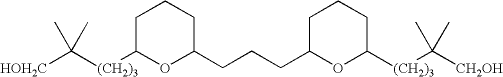

II-1:
5-(6-{3-[6-(5-Hydroxy-4,4-dimethyl-pentyl)-tetrahydro-pyran-2-yl]-propyl}-
tetrahydro-pyran-2-yl)-2,2-dimethyl-pentan-1-ol

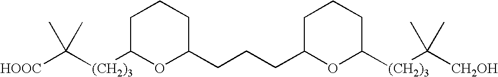

II-2:
5-(6-{3-[6-(5-Hydroxy-4,4-dimethyl-pentyl)-tetrahydro-pyran-2-yl]-propyl}-tetrahydro-
pyran-2-yl)-2,2-dimethyl-pentanoic acid

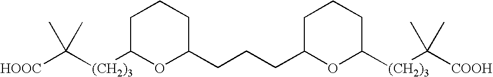

II-3:
5-(6-{3-[6-(4-Carboxyl-4,4-dimethyl-pentyl)-tetrahydro-pyran-2-yl]-propyl}-
tetrahydro-pyran-2-yl)-2,2-dimethyl-pentanoic acid

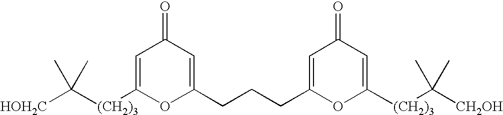

II-4:
5-(6-{3-[6-(5-Hydroxy-4,4-dimethyl-pentyl)-4-oxo-pyran-2-yl]-propyl}-4-
oxo-pyran-2-yl)-2,2-dimethyl-pentan-1-ol

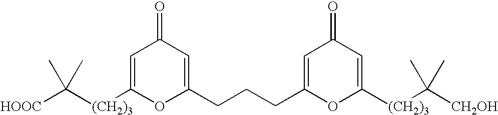

II-5:
5-(6-{3-[6-(5-Hydroxy-4,4-dimethyl-pentyl)-4-oxo-pyran-2-yl]-propyl}-4-
oxo-pyran-2-yl)-2,2-dimethyl-pentanoic acid

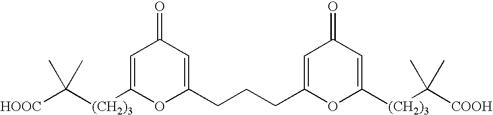

II-6:
5-(6-{3-[6-(4-Carboxyl-4,4-dimethyl-pentyl)-4-oxo-pyran-2-yl]-propyl}-
4-oxo-pyran-2-yl)-2,2-dimethyl-pentanoic acid

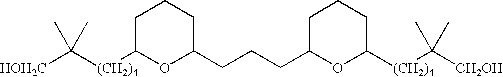

II-7:
6-(6-{3-[6-(6-Hydroxy-5,5-dimethyl-hexyl)-tetrahydro-pyran-2-yl]-propyl}-
tetrahydro-pyran-2-yl)-2,2-dimethyl-hexan-1-ol

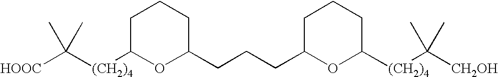

II-8:
6-(6-{3-[6-(6-Hydroxy-5,5-dimethyl-hexyl)-tetrahydro-pyran-2-yl]-propyl}-tetrahydro-
pyran-2-yl)-2,2-dimethyl-hexanoic acid

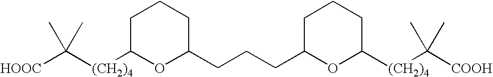

II-9:
6-(6-{3-[6-(5-Carboxyl-5,5-dimethyl-hexyl)-tetrahydro-pyran-2-yl]-propyl}-
tetrahydro-pyran-2-yl)-2,2-dimethyl-hexanoic acid

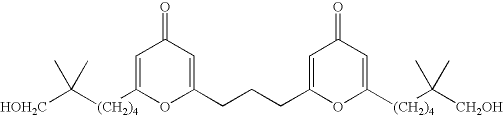

II-10:
6-(6-{3-[6-(6-Hydroxy-5,5-dimethyl-hexyl)-4-oxo-pyran-2-yl]-propyl}-4-
oxo-pyran-2-yl)-2,2-dimethyl-hexan-1-ol

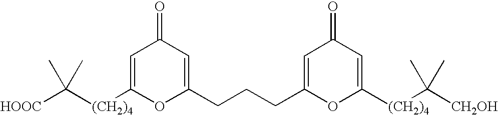

II-11:
6-(6-{3-[6-(6-Hydroxy-5,5-dimethyl-hexyl)-4-oxo-pyran-2-yl]-propyl}-4-
oxo-pyran-2-yl)-2,2-dimethyl-hexanoic acid

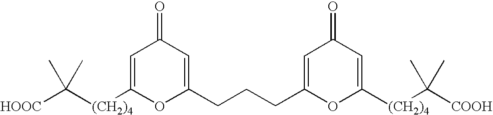

II-12:
6-(6-{3-[6-(5-Carboxyl-5,5-dimethyl-hexyl)-4-oxo-pyran-2-yl]-propyl}-4-
oxo-pyran-2-yl)-2,2-dimethyl-hexanoic acid

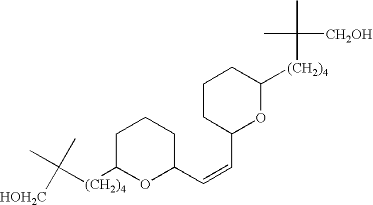

II-13:
6-(6-{2-[6-(6-Hydroxy-5,5-dimethyl-hexyl)-tetrahydro-pyran-2-yl]-vinyl}-tetrahydro-
pyran-2-yl)-2,2-dimethyl-hexan-1-ol

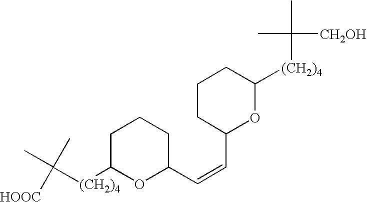

II-14:
6-(6-{2-[6-(6-Hydroxy-5,5-dimethyl-hexyl)-tetrahydro-pyran-2-yl]-vinyl}-tetrahydro-
pyran-2-yl)-2,2-dimethyl-hexanoic acid

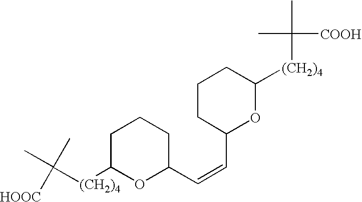

II-15:
6-(6-{2-[6-(5-Carboxyl-5,5-dimethyl-hexyl)-tetrahydro-pyran-2-yl]-vinyl}-tetrahydro-
pyran-2-yl)-2,2-dimethyl-hexanoic acid

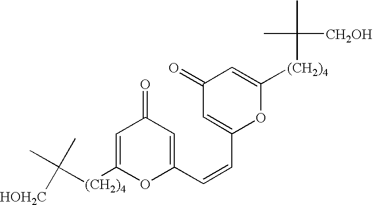

II-16:
6-(6-{2-[6-(6-Hydroxy-5,5-dimethyl-hexyl)-4-oxo-pyran-2-yl]-vinyl}-4-oxo-
pyran-2-yl)-2,2-dimethyl-hexan-1-ol

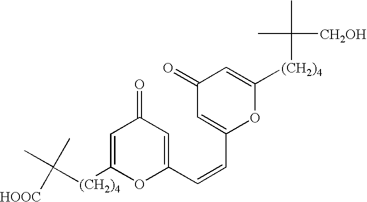

II-17:
6-(6-{2-[6-(6-Hydroxy-5,5-dimethyl-hexyl)-4-oxo-pyran-2-yl]-vinyl}-4-oxo-
pyran-2-yl)-2,2-dimethyl-hexanoic acid

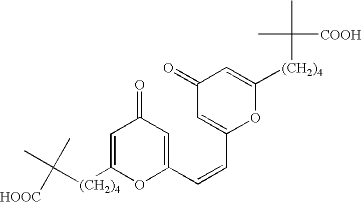

II-18:
6-(6-{2-[6-(5-Carboxyl-5,5-dimethyl-hexyl)-4-oxo-pyran-2-yl]-vinyl}-4-oxo-
pyran-2-yl)-2,2-dimethyl-hexanoic acid

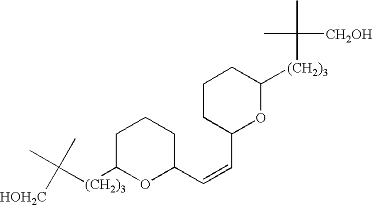

II-19:
5-(6-{2-[6-(5-Hydroxy-4,4-dimethyl-pentyl)-tetrahydro-pyran-2-yl]-vinyl}-tetrahydro-
pyran-2-yl)-2,2-dimethyl-pentan-1-ol

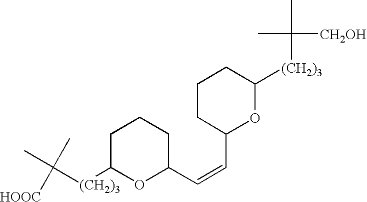

II-20:
5-(6-{2-[6-(5-Hydroxy-4,4-dimethyl-pentyl)-tetrahydro-pyran-2-yl]-vinyl}-tetrahydro-
pyran-2-yl)-2,2-dimethyl-pentanoic acid

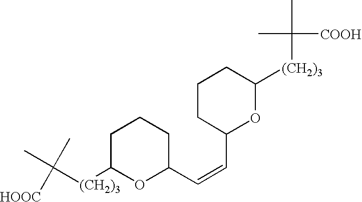

II-21:
5-(6-{2-[6-(4-Carboxyl-4,4-dimethyl-pentyl)-tetrahydro-pyran-2-yl]-vinyl}-
tetrahydro-pyran-2-yl)-2,2-dimethyl-pentanoic acid

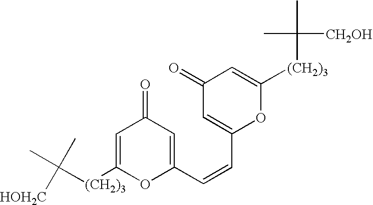

II-22:
5-(6-{2-[6-(5-Hydroxy-4,4-dimethyl-pentyl)-4-oxo--pyran-2-yl]-vinyl}-4-
oxo-pyran-2-yl)-2,2-dimethyl-pentan-1-ol

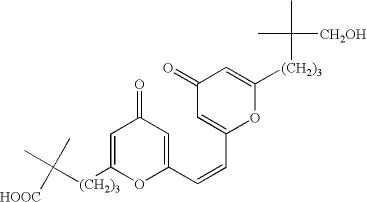

II-23:
5-(6-{2-[6-(5-Hydroxy-4,4-dimethyl-pentyl)-4-oxo--pyran-2-yl]-vinyl}-4-
oxo-pyran-2-yl)-2,2-dimethyl-pentanoic acid

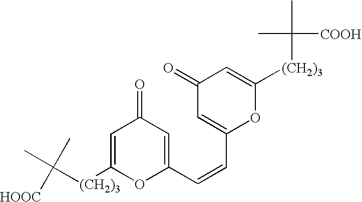

II-24:
5-(6-{2-[6-(4-Carboxyl-4,4-dimethyl-pentyl)-4-oxo-pyran-2-yl]-vinyl}-4-
oxo-pyran-2-yl)-2,2-dimethyl-pentanoic acid

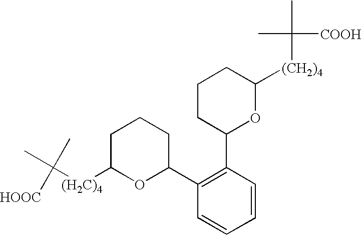

II-27:
6-(6-{2-[6-(5-Carboxyl-5,5-dimethyl-hexyl)-tetrahydro-pyran-2-yl]-phenyl}-tetrahydro-
pyran-2-yl)-2,2-dimethyl-hexanoic acid

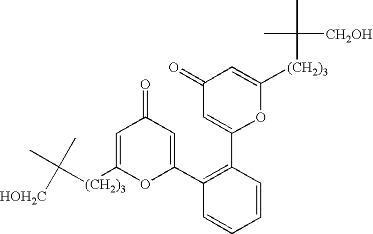

II-28:
5-(6-{2-[6-(5-Hydroxy-4,4-dimethyl-pentyl)-4-oxo-pyran-2-yl]-phenyl}-4-oxo-
pyran-2-yl)-2,2-dimethyl-pentan-1-ol

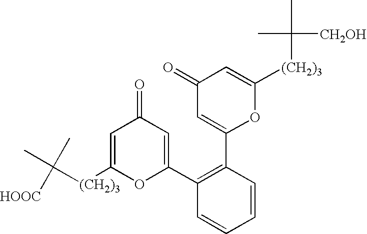

II-29:
5-(6-{2-[6-(5-Hydroxy-4,4-dimethyl-pentyl)-4-oxo-pyran-2-yl]-phenyl}-4-oxo-
pyran-2-yl)-2,2-dimethyl-pentanoic acid

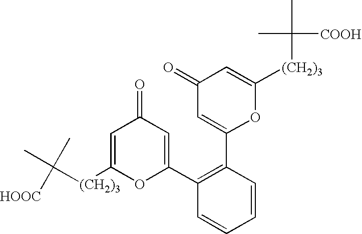

II-30:
5-(6-{2-[6-(4-Carboxyl-4,4-dimethyl-pentyl)-4-oxo-pyran-2-yl]-phenyl}-4-oxo-
pyran-2-yl)-2,2-dimethyl-pentan-1-ol

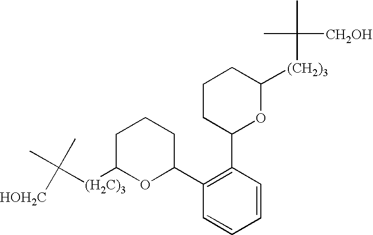

II-31:
5-(6-{2-[6-(5-Hydroxy-4,4-dimethyl-pentyl)-tetrahydro-pyran-2-yl]-phenyl}-tetrahydro-
pyran-2-yl)-2,2-dimethyl-pentan-1-ol

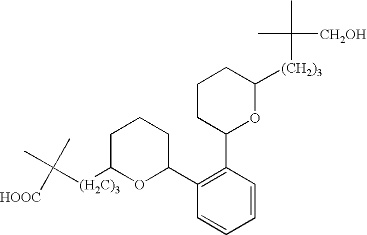

II-32:
5-(6-{2-[6-(5-Hydroxy-4,4-dimethyl-pentyl)-4-oxo-pyran-2-yl]-phenyl}-4-oxo-
pyran-2-yl)-2,2-dimethyl-pentanoic acid

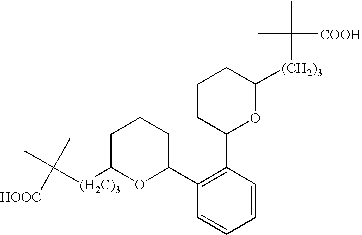

II-33:
5-(6-{2-[6-(4-Carboxyl-4,4-dimethyl-pentyl)-4-oxo-pyran-2-yl]-phenyl}-4-oxo-
pyran-2-yl)-2,2-dimethyl-pentanoic acid

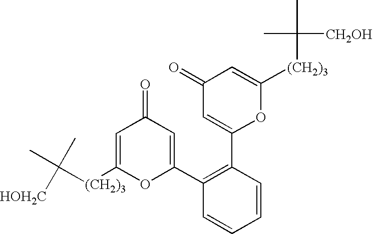

II-34:
6-(6-{2-[6-(6-Hydroxy-5,5-dimethyl-hexyl)-4-oxo-pyran-2-yl]-phenyl}-4-oxo-
pyran-2-yl)-2,2-dimethyl-hexan-1-ol

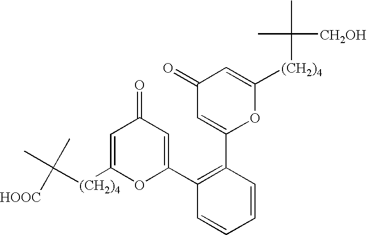

II-35:
6-(6-{2-[6-(6-Hydroxy-5,5-dimethyl-hexyl)-4-oxo-pyran-2-yl]-phenyl}-4-oxo-
pyran-2-yl)-2,2-dimethyl-hexanoic acid

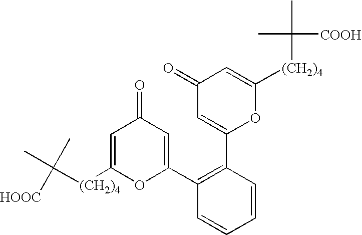

II-36:
6-(6-{2-[6-(5-Carboxyl-5,5-dimethyl-hexyl)-4-oxo-pyran-2-yl]-phenyl}-4-oxo-
pyran-2-yl)-2,2-dimethyl-hexanoic acid

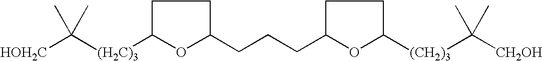

5-(5-{3-[5-(5-Hydroxy-4,4-dimethyl-pentyl)-tetrahydro-furan-2-yl]-propyl}-
tetrahydro-furan-2-yl)-2,2-dimethyl-pentan-1-ol

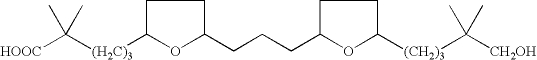

II-38:
5-(5-{3-[5-(5-Hydroxy-4,4-dimethyl-pentyl)-tetrahydro-furan-2-yl]-propyl}-
tetrahydro-furan-2-yl)-2,2-dimethyl-pentanoic acid

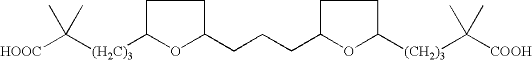

5-(5-{3-[5-(4-Carboxyl-4,4-dimethyl-pentyl)-
tetrahydro-furan-2-yl]-propyl}-tetrahydro-furan-2-yl)-2,2-dimethyl-pentanoic acid

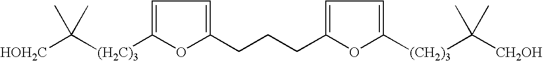

II-40:
5-(5-{3-[5-(5-Hydroxy-4,4-dimethyl-pentyl)-furan-2-yl]-propyl}-furan-2-yl)-2,2-
dimethyl-pentan-1-ol

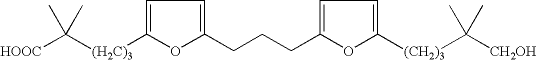

II-41:
5-(5-{3-[5-(5-Hydroxy-4,4-dimethyl-pentyl)-furan-2-yl]-propyl}-furan-2-yl)-2,2-dimethyl-
pentanoic acid

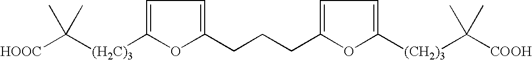

II-42:
5-(5-{3-[5-(4-Carboxyl-4,4-dimethyl-pentyl)-furan-2-yl]-propyl}-furan-2-yl)-
2,2-dimethyl-pentanoic acid

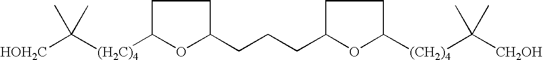

II-43:
6-(5-{3-[5-(6-Hydroxy-5,5-dimethyl-hexyl)-tetrahydro-furan-2-yl]-propyl}-
tetrahydro-furan-2-yl)-2,2-dimethyl-hexan-1-ol

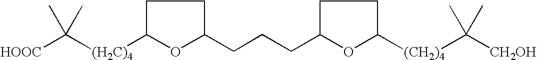

II-44:
6-(5-{3-[5-(6-Hydroxy-5,5-dimethyl-hexyl)-tetrahydro-furan-2-yl]-propyl}-tetrahydro-
furan-2-yl)-2,2-dimethyl-hexanoic acid

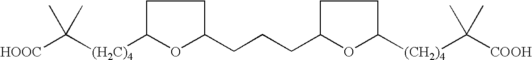

II-45:
6-(5-{3-[5-(5-Carboxyl-5,5-dimethyl-hexyl)-tetrahydro-furan-2-yl]-propyl}-
tetrahydro-furan-2-yl)-2,2-dimethyl-hexanoic acid

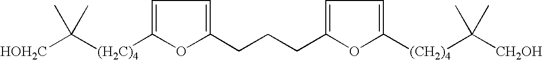

II-46:
6-(5-{3-[5-(6-Hydroxy-5,5-dimethyl-hexyl)-furan-2-yl]-propyl}-furan-2-yl)-2,2-dimethyl-
hexan-1-ol

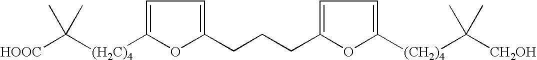

II-47:
6-(5-{3-[5-(6-Hydroxy-5,5-dimethyl-hexyl)-furan-2-yl]-propyl}-furan-2-yl)-2,2-dimethyl-
hexanoic acid

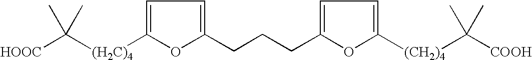

II-48:
6-(5-{3-[5-(5-Carboxyl-5,5-dimethyl-hexyl)-furan-2-yl]-propyl}-furan-2-yl)-
2,2-dimethyl-hexanoic acid

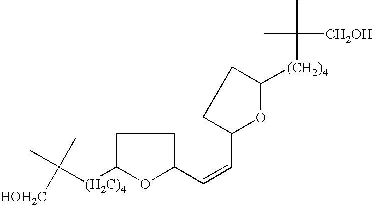

II-49:
6-(5-{2-[5-(6-Hydroxy-5,5-dimethyl-hexyl)-tetrahydro-furan-2-yl]-vinyl}-tetrahydro-
furan-2-yl)-2,2-dimethyl-hexan-1-ol

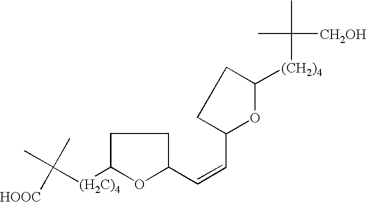

II-50:
6-(5-{2-[5-(6-Hydroxy-5,5-dimethyl-hexyl)-tetrahydro-furan-2-yl]-vinyl}-tetrahydro-
furan-2-yl)-2,2-dimethyl-hexanoic acid

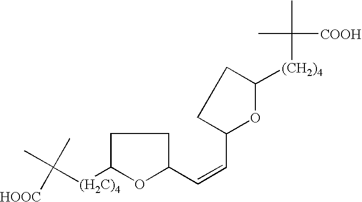

II-51:
6-(5-{2-[5-(5-Carboxyl-5,5-dimethyl-hexyl)-tetrahydro-furan-2-yl]-vinyl}-tetrahydro-
furan-2-yl)-2,2-dimethyl-hexanoic acid

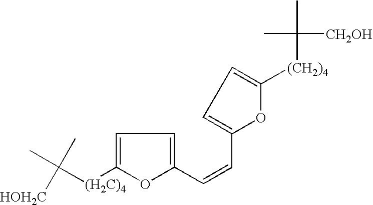

II-52:
6-(5-{2-[5-(6-Hydroxy-5,5-dimethyl-hexyl)-furan-2-yl]-vinyl}-furan-2-yl)-2,2-dimethyl-
hexan-1-ol

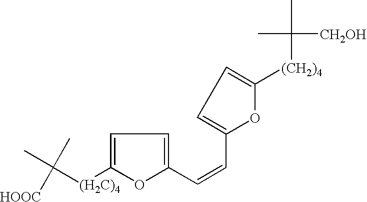

II-53:
6-(5-{2-[5-(6-Hydroxy-5,5-dimethyl-hexyl)-furan-2-yl]-vinyl}-furan-2-yl)-2,2-dimethyl-
hexanoic acid

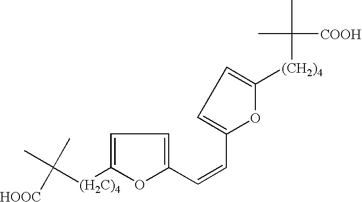

II-54:
6-(5-{2-[5-(5-Carboxyl-5,5-dimethyl-hexyl)-furan-2-yl]-vinyl}-furan-2-yl)-2,2-dimethyl-
hexanoic acid

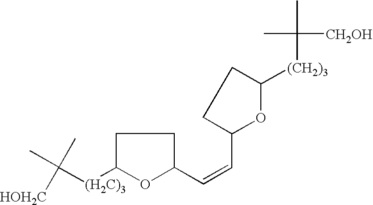

II-55:
5-(5-{2-[5-(5-Hydroxy-4,4-dimethyl-pentyl)-tetrahydro-furan-2-yl]-vinyl}-tetrahydro-
furan-2-yl)-2,2-dimethyl-pentan-1-ol

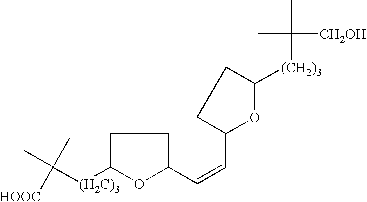

II-56:
5-(5-{2-[5-(5-Hydroxy-4,4-dimethyl-pentyl)-tetrahydro-furan-2-yl]-vinyl}-tetrahydro-
furan-2-yl)-2,2-dimethyl-pentanoic acid

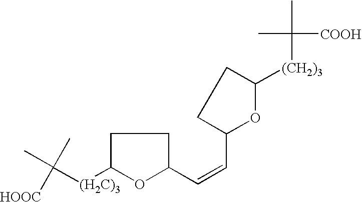

II-57:
5-(5-{2-[5-(4-Carboxyl-4,4-dimethyl-pentyl)-tetrahydro-furan-2-yl]-vinyl}-tetrahydro-
furan-2-yl)-2,2-dimethyl-pentanoic acid

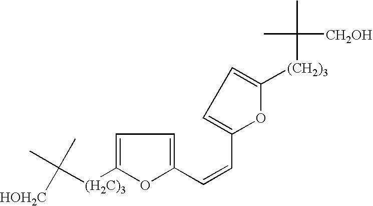

II-58:
5-(5-{2-[5-(5-Hydroxy-4,4-dimethyl-pentyl)-furan-2-yl]-vinyl}-furan-2-yl)-2,2-dimethyl-
pentan-1-ol

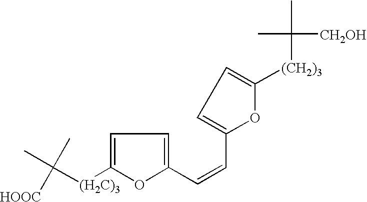

II-59:
5-(5-{2-[5-(5-Hydroxy-4,4-dimethyl-pentyl)-furan-2-yl]-vinyl}-furan-2-yl)-2,2-dimethyl-
pentanoic acid

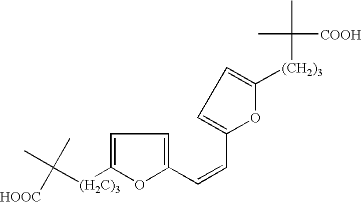

II-60:
5-(5-{2-[5-(4-Carboxyl-4,4-dimethyl-pentyl)-furan-2-yl]-vinyl}-furan-2-yl)-2,2-dimethyl-
pentanoic acid

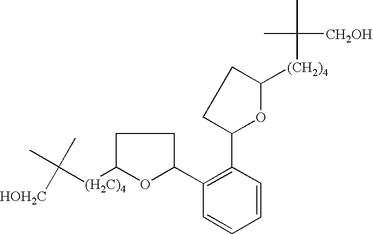

II-61:
6-(5-{2-[5-(6-Hydroxy-5,5-dimethyl-hexyl)-tetrahydro-furan-2-yl]-phenyl}-tetrahydro-
furan-2-yl)-2,2-dimethyl-hexan-1-ol

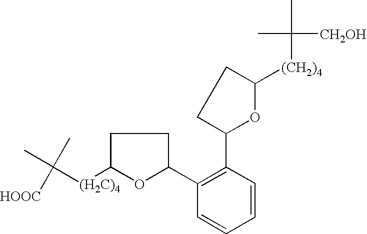

II-62:
6-(5-{2-[5-(6-Hydroxy-5,5-dimethyl-hexyl)-tetrahydro-furan-2-yl]-phenyl}-tetrahydro-
furan-2-yl)-2,2-dimethyl-hexanoic acid

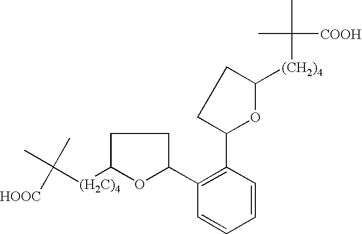

II-63:
6-(5-{2-[5-(5-Carboxyl-5,5-dimethyl-hexyl)-tetrahydro-furan-2-yl]-phenyl}-tetrahydro-
furan-2-yl)-2,2-dimethyl-hexanoic acid

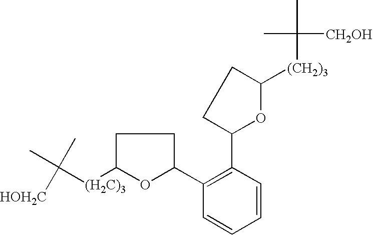

II-64:
5-(5-{2-[5-(5-Hydroxy-4,4-dimethyl-pentyl)-tetrahydro-furan-2-yl]-phenyl}-tetrahydro-
furan-2-yl)-2,2-dimethyl-pentan-1-ol

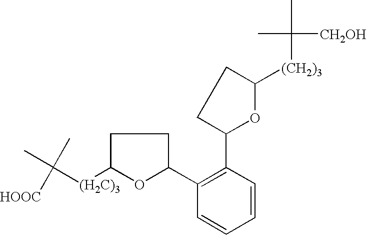

II-65:
5-(5-{2-[5-(5-Hydroxy-4,4-dimethyl-pentyl)-tetrahydro-furan-2-yl]-phenyl}-tetrahydro-
furan-2-yl)-2,2-dimethyl-pentanoic acid

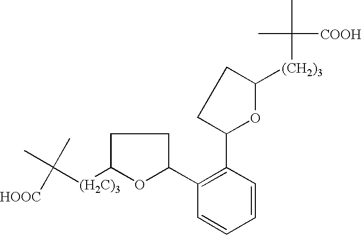

II-66:
5-(5-{2-[5-(4-Carboxyl-4,4-dimethyl-pentyl)-tetrahydro-furan-2-yl]-phenyl}-tetrahydro-
furan-2-yl)-2,2-dimethyl-pentanoic acid

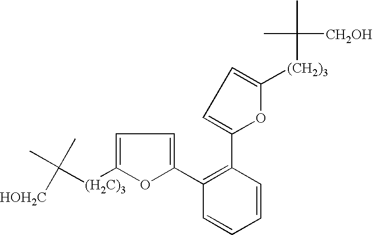

II-67:
5-(5-{2-[5-(5-Hydroxy-4,4-dimethyl-pentyl)-furan-2-yl]-phenyl}-furan-2-yl)-2,2-
dimethyl-pentan-1-ol

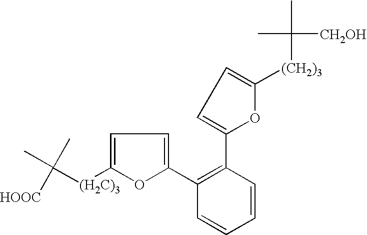

II-68:
5-(5-{2-[5-(5-Hydroxy-4,4-dimethyl-penyl)-furan-2-yl]-phenyl}-furan-2-yl)-2,2-
dimethyl-pentanoic acid

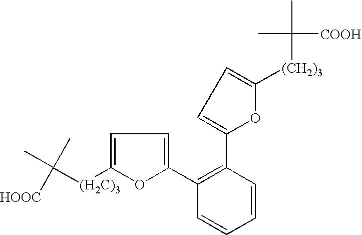

II-69:
5-(5-{2-[5-(4-Carboxyl-4,4-dimethyl-pentyl)-furan-2-yl]-phenyl}-furan-2-yl)-2,2-
dimethyl-pentanoic acid

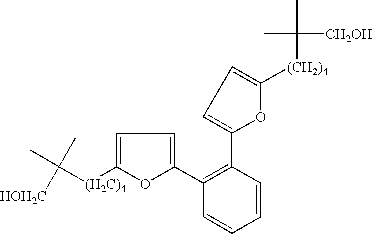

II-70:
6-(5-{2-[5-(6-Hydroxy-5,5-dimethyl-hexyl)-furan-2-yl]-phenyl}-furan-2-yl)-2,2-
dimethyl-hexan-1-ol

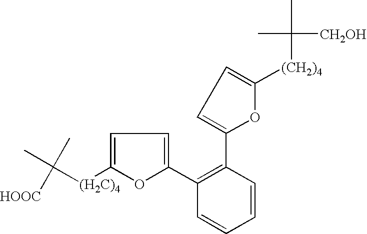

II-71:
6-(5-{2-[5-(6-Hydroxy-5,5-dimethyl-hexyl)-furan-2-yl]-phenyl}-furan-2-yl)-2,2-dimethyl-
hexanoic acid

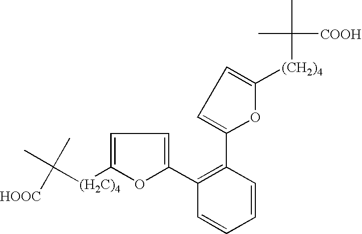

II-72:
6-(5-{2-[5-(5-Carboxyl-5,5-dimethyl-hexyl)-furan-2-yl]-phenyl}-furan-2-yl)-2,2-dimethyl-
hexanoic acid

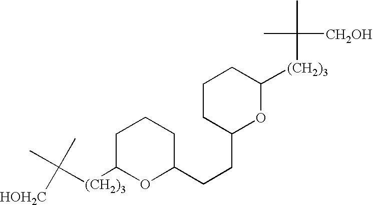

II-73:
5-(6-{2-[6-(5-Hydroxy-4,4-dimethyl-pentyl)-tetrahydro-pyran-2-yl]-ethyl}-tetrahydro-
pyran-2-yl)-2,2-dimethyl-pentan-1-ol

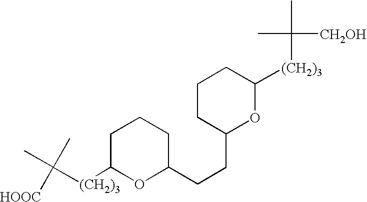

II-74:
5-(6-{2-{6-(5-Hydroxy-4,4-dimethyl-pentyl)-tetrahydro-pyran-2-yl]-ethyl}-tetrahydro-
pyran-2-yl)-2,2-dimethyl-pentanoic acid

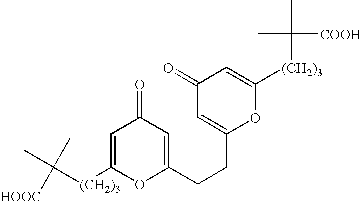

II-78:
5-(6-{2-[6-(4-Carboxyl-4,4-dimethyl-pentyl)-4-oxo-pyran-2-yl]-ethyl}-4-
oxo-pyran-2-yl)-2,2-dimethyl-pentanoic acid

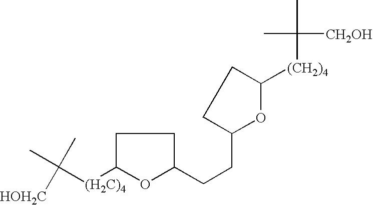

II-79:
6-(5-{2-[5-(6-Hydroxy-5,5-dimethyl-hexyl)-tetrahydro-furan-2-yl]-ethyl}-tetrahydro-
furan-2-yl)-2,2-dimethyl-hexan-1-ol

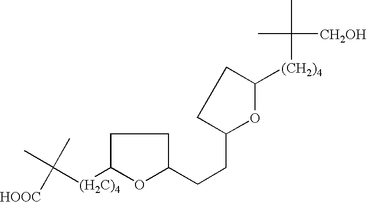

II-80:
6-(5-{2-[5-(6-Hydroxy-5,5-dimethyl-hexyl)-tetrahydro-furan-2-yl]-ethyl}-tetrahydro-
furan-2-yl)-2,2-dimethyl-hexanoic acid

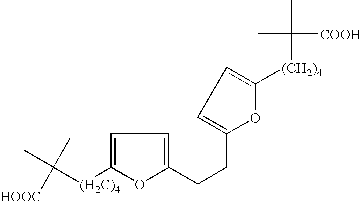

II-84:
6-(5-{2-[5-(5-Carboxyl-5,5-dimethyl-hexyl)-furan-2-yl]-ethyl}-furan-2-yl)-2,2-dimethyl-
hexanoic acid

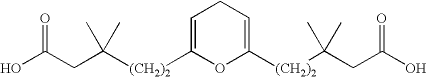

III-1
5-[6-(4-Carboxy-3,3-dimethyl-butyl)-4H-pyran-2-yl]-3,3-dimethyl-pentanoic acid

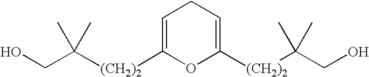

III-2
4-[6-(4-Hydroxy-3,3-dimethyl-butyl)-4H-pyran-2-yl]-2,2-dimethyl-butan-1-ol

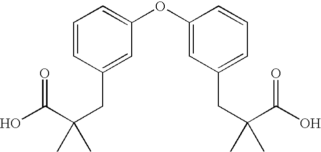

(IV-1)
3-{3-[3-(2-Carboxy-2-methyl-propyl)-phenoxy]-phenyl}-2,2-dimethyl-propionic acid

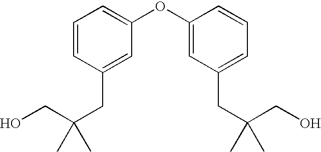

IV-2
1-{3-[3-(2-Hydroxy-2-methyl-propyl)-phenoxy]-phenyl}-2-methyl-propan-2-ol

A few examples of preferred compounds of the invention are listed in Table 2 below.

TABLE 2
Preferred Compounds of the Invention

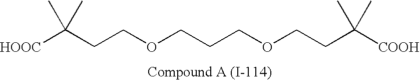

4-[3-(3-Carboxy-3-methyl-butoxy)-propoxy]-2,2-dimethyl-butyric acid

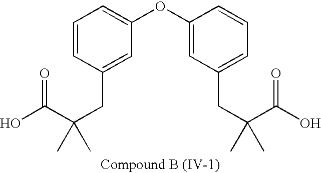

3-{3-[3-(2-Carboxy-2-methyl-propyl)-phenoxy]-phenyl}-2,2-dimethyl-pro-
pionic acid

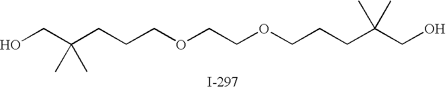

5-[2-(5-hydroxy-4,4-dimethyl-pentyloxy)-ethoxy]-2,2-dimethyl-pen-
tan-1-ol

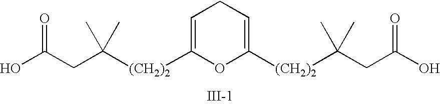

5-[6-(4-Carboxy-3,3-dimethyl-butyl)-4H-pyran-2-yl]-3,3-dimethyl-pen-
tanoic acid

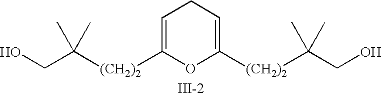

4-[6-(4-Hydroxy-3,3-dimethyl-butyl)-4H-pyran-2-yl]-2,2-dimeth-
yl-butan-ol

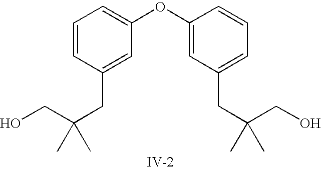

1-{3-[3-(2-Hydroxy-2-methyl-propyl)-phenoxy]-phenyl}-2-methyl-pro-
pan-2-ol

A few examples of illustrative compounds of the invention are listed in Table 2 below.

TABLE 2
Illustrative compounds of the invention

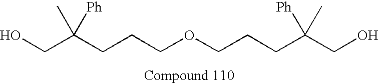

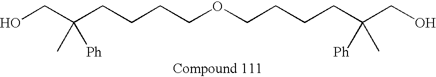

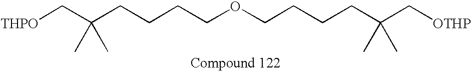

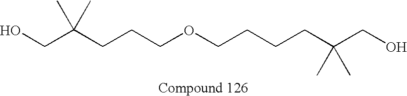

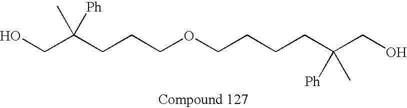

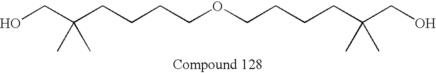

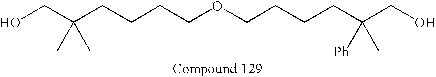

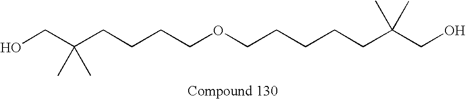

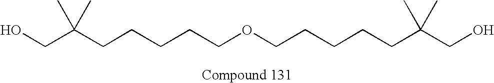

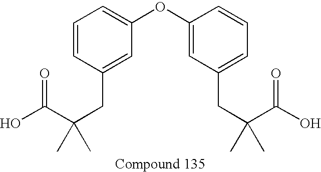

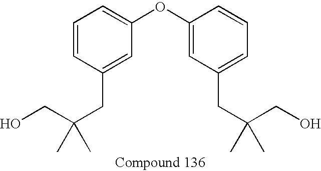

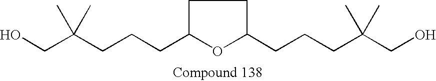

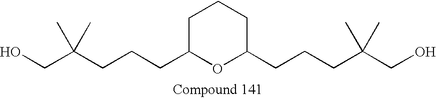

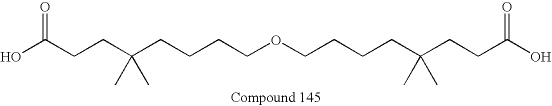

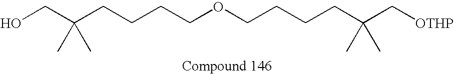

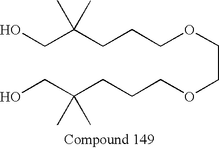

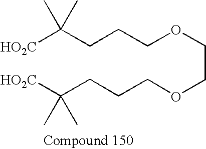

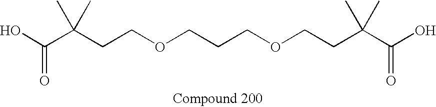

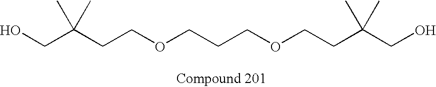

### 5.1 Definitions and Abbreviations

Apo(a): apolipoprotein(a)

Apo A-I: apolipoprotein A-I

Apo B: apolipoprotein B

Apo E: apolipoprotein E

FH: Familial hypercholesterolemia

FCH: Familial combined hyperlipidemia

GDM: Gestational diabetes mellitus

HDL: High density lipoprotein

IDL: Intermediate density lipoprotein

IDDM: Insulin dependent diabetes mellitus

LDH: Lactate dehdyrogenase

LDL: Low density lipoprotein

Lp(a): Lipoprotein (a)

MODY: Maturity onset diabetes of the young

NIDDM: Non-insulin dependent diabetes mellitus

PPAR: Peroxisome proliferator activated receptor

RXR: Retinoid X receptor

VLDL: Very low density lipoprotein

As used herein, the phrase “compounds of the invention” means, collectively, the compounds of formulas I, Ia-Id, II, Ia, III, and IV and pharmaceutically acceptable salts, hydrates, solvates, clathrates, enantiomers, diastereomers, racemates, or mixtures of stereoisomers thereof.

The compounds of the invention can contain one or more chiral centers and/or double bonds and, therefore, exist as stereoisomers, such as double-bond isomers (i.e., geometric isomers), enantiomers, or diastereomers. According to the invention, the chemical structures depicted herein, and therefore the compounds of the invention, encompass the racemic form of compounds of the invention as well as all enantiomers and stereoisomers, that is, both the stereomerically pure form (e.g., geometrically pure, enantiomerically pure, or diastereomerically pure) and enantiomeric and stereoisomeric mixtures.

A compound of the invention is considered optically active or enantiomerically pure (i.e., substantially the R-form or substantially the S-form) with respect to a chiral center when the compound is about 90% ee (enantiomeric excess) or greater, preferably, equal to or greater than 95% ee with respect to a particular chiral center. A compound of the invention is considered to be in enantiomerically enriched form when the compound has an enantiomeric excess of greater than about 80% ee, preferably greater than about. As used herein, a racemic mixture means about 50% of one enantiomer and about 50% of is corresponding enantiomer relative to all chiral centers in the molecule. Thus, the invention encompasses all enantiomerically pure, enantiomerically enriched, and racemic mixtures of compounds of formulas I, Ia-Id, II, Ia, III, and IV.

Enantiomeric and stereoisomeric mixtures can be resolved into their component enantiomers or stereoisomers by well known methods, such as chiral-phase gas chromatography, chiral-phase high performance liquid chromatography, crystallizing the compound as a chiral salt complex, or crystallizing the compound in a chiral solvent. Enantiomers and stereoisomers can also be obtained from stereomerically- or enantiomerically-pure intermediates, reagents, and catalysts by well known asymmetric synthetic methods.

When administered to a patient, e.g., to an animal for veterinary use or for improvement of livestock, or to a human for clinical use, the compounds of the invention are administered in isolated form or as the isolated form in a pharmaceutical composition. As used herein, “isolated” means that the compounds of the invention are separated from other components of either (a) a natural source, such as a plant or cell, preferably bacterial culture, or (b) a synthetic organic chemical reaction mixture. Preferably, via conventional techniques, the compounds of the invention are purified. As used herein, “purified” means that when isolated, the isolate contains at least 95%, preferably at least 98%, of a single ether compound of the invention by weight of the isolate.

The term “therapeutically effective amount” means the amount of a compound of the invention that will elicit the biological or medical response in a mammal that is being that is being treated by the veterinarian, medical doctor, or other clinician.

The term “prophylactically effective” or “preventive” means the amount of a compound of the invention that will prevent or inhibit affliction or mitigate affliction of a mammal with a medical condition that a veterinarian, medical doctor, or other clinician is trying to prevent, inhibit, or mitigate.

The phrase “pharmaceutically acceptable salt(s),” as used herein includes but are not limited to salts of acidic or basic groups that may be present in the compounds of the invention. Compounds that are basic in nature are capable of forming a wide variety of salts with various inorganic and organic acids. The acids that may be used to prepare pharmaceutically acceptable acid addition salts of such basic compounds are those that form non-toxic acid addition salts, i.e., salts containing pharmacologically acceptable anions, including but not limited to sulfuric, citric, maleic, acetic, oxalic, hydrochloride, hydrobromide, hydroiodide, nitrate, sulfate, bisulfate, phosphate, acid phosphate, isonicotinate, acetate, lactate, salicylate, citrate, acid citrate, tartrate, oleate, tannate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, p-toluenesulfonate and pamoate (i.e., 1,1′-methylene-bis-(2-hydroxy-3-naphthoate)) salts. Compounds of the invention that include an amino moiety also can form pharmaceutically acceptable salts with various amino acids, in addition to the acids mentioned above. Compounds of the invention that are acidic in nature are capable of forming base salts with various pharmacologically acceptable cations. Examples of such salts include alkali metal or alkaline earth metal salts and, particularly, calcium, magnesium, sodium lithium, zinc, potassium, and iron salts.

As used herein, the term “solvate” means a compound of the invention or a salt thereof, that further includes a stoichiometric or non-stoichiometric amount of a solvent bound by non-covalent intermolecular forces. Preferred solvents are volatile, non-toxic, and/or acceptable for administration to humans in trace amounts.

As used herein, the term “hydrate” means a compound of the invention or a salt thereof, that further includes a stoichiometric or non-stoichiometric amount of water bound by non-covalent intermolecular forces.

The term “clathrate” means a compound of the invention or a salt thereof in the form of a crystal lattice that contains spaces (e.g., channels) that have a guest molecule (e.g., a solvent or water) trapped within.

“Altering lipid metabolism” indicates an observable (measurable) change in at least one aspect of lipid metabolism, including but not limited to total blood lipid content, blood HDL cholesterol, blood LDL cholesterol, blood VLDL cholesterol, blood triglyceride, blood Lp(a), blood apo A-I, blood apo E and blood non-esterified fatty acids.

“Altering glucose metabolism” indicates an observable (measurable) change in at least one aspect of glucose metabolism, including but not limited to total blood glucose content, blood insulin, the blood insulin to blood glucose ratio, insulin sensitivity, and oxygen consumption.

As used herein, the term “alkyl group” means a saturated, monovalent, unbranched or branched hydrocarbon chain. Examples of alkyl groups include, but are not limited to, (C₁₋C₆)alkyl groups, such as methyl, ethyl, propyl, isopropyl, 2-methyl-1-propyl, 2-methyl-2-propyl, 2-methyl-1-butyl, 3-methyl-1-butyl, 2-methyl-3-butyl, 2,2-dimethyl-1-propyl, 2-methyl-1-pentyl, 3-methyl-1-pentyl, 4-methyl-1-pentyl, 2-methyl-2-pentyl, 3-methyl-2-pentyl, 4-methyl-2-pentyl, 2,2-dimethyl-1-butyl, 3,3-dimethyl-1-butyl, 2-ethyl-1-butyl, butyl, isobutyl, t-butyl, pentyl, isopentyl, neopentyl, and hexyl, and longer alkyl groups, such as heptyl, and octyl. An alkyl group can be unsubstituted or substituted with one or two suitable substituents.

An “alkenyl group” means a monovalent, unbranched or branched hydrocarbon chain having one or more double bonds therein. The double bond of an alkenyl group can be unconjugated or conjugated to another unsaturated group. Suitable alkenyl groups include, but are not limited to (C₂₋C₆)alkenyl groups, such as vinyl, allyl, butenyl, pentenyl, hexenyl, butadienyl, pentadienyl, hexadienyl, 2-ethylhexenyl, 2-propyl-2-butenyl, 4-(2-methyl-3-butene)-pentenyl. An alkenyl group can be unsubstituted or substituted with one or two suitable substituents.

An “alkynyl group” means monovalent, unbranched or branched hydrocarbon chain having one or more triple bonds therein. The triple bond of an alkynyl group can be unconjugated or conjugated to another unsaturated group. Suitable alkynyl groups include, but are not limited to, (C₂₋C₆)alkynyl groups, such as ethynyl, propynyl, butynyl, pentynyl, hexynyl, methylpropynyl, 4-methyl-I -butynyl, 4-propyl-2-pentynyl, and 4-butyl-2-hexynyl. An alkynyl group can be unsubstituted or substituted with one or two suitable substituents.

An “aryl group” means a monocyclic or polycyclic-aromatic radical comprising carbon and hydrogen atoms. Examples of suitable aryl groups include, but are not limited to, phenyl, tolyl, anthacenyl, fluorenyl, indenyl, azulenyl, and naphthyl, as well as benzo-fused carbocyclic moieties such as 5,6,7,8-tetrahydronaphthyl. An aryl group can be unsubstituted or substituted with one or two suitable substituents. Preferably, the aryl group is a monocyclic ring, wherein the ring comprises 6 carbon atoms, referred to herein as “(C₆)aryl”.

A “heteroaryl group” means a monocyclic- or polycyclic aromatic ring comprising carbon atoms, hydrogen atoms, and one or more heteroatoms, preferably 1 to 3 heteroatoms, independently selected from nitrogen, oxygen, and sulfur. Illustrative examples of heteroaryl groups include, but are not limited to, pyridinyl, pyridazinyl, pyrimidyl, pyrazyl, triazinyl, pyrrolyl, pyrazolyl, imidazolyl, (1,2,3,)- and (1,2,4)-triazolyl, pyrazinyl, pyrimidinyl, tetrazolyl, furyl, thienyl, isoxazolyl, thiazolyl, furyl, phienyl, isoxazolyl, and oxazolyl. A heteroaryl group can be unsubstituted or substituted with one or two suitable substituents. Preferably, a heteroaryl group is a monocyclic ring, wherein the ring comprises 2 to 5 carbon atoms and 1 to 3 heteroatoms, referred to herein as “(C₂₋C₅)heteroaryl”.

A “cycloalkyl group” means a monocyclic or polycyclic saturated ring comprising carbon and hydrogen atoms and having no carbon-carbon multiple bonds. Examples of cycloalkyl groups include, but are not limited to, (C₃₋C₇)cycloalkyl groups, such as cyclopropyl, cyclobutyl, cyclopentyl, cyclohexyl, and cycloheptyl, and saturated cyclic and bicyclic terpenes. A cycloalkyl group can be unsubstituted or substituted by one or two suitable substituents. Preferably, the cycloalkyl group is a monocyclic ring or bicyclic ring.

A “heterocycloalkyl group” means a monocyclic or polycyclic ring comprising carbon and hydrogen atoms and at least one heteroatom, preferably, 1 to 3 heteroatoms selected from nitrogen, oxygen, and sulfur, and having no unsaturation. Examples of heterocycloalkyl groups include pyrrolidinyl, pyrrolidino, piperidinyl, piperidino, piperazinyl, piperazino, morpholinyl, morpholino, thiomorpholinyl, thiomorpholino, and pyranyl. A heterocycloalkyl group can be unsubstituted or substituted with one or two suitable substituents. Preferably, the heterocycloalkyl group is a monocyclic or bicyclic ring, more preferably, a monocyclic ring, wherein the ring comprises from 3 to 6 carbon atoms and form 1 to 3 heteroatoms, referred to herein as (C₁₋C₆)heterocycloalkyl.

As used herein a “heterocyclic radical” or “heterocyclic ring” means a heterocycloalkyl group or a heteroaryl group.

The term “alkoxy group” means an —O-alkyl group, wherein alkyl is as defined above. An alkoxy group can be unsubstituted or substituted with one or two suitable substituents. Preferably, the alkyl chain of an alkyloxy group is from 1 to 6 carbon atoms in length, referred to herein as “(C₁₋C₆)alkoxy”.

The term “aryloxy group” means an —O-aryl group, wherein aryl is as defined above. An aryloxy group can be unsubstituted or substituted with one or two suitable substituents. Preferably, the aryl ring of an aryloxy group is a monocyclic ring, wherein the ring comprises 6 carbon atoms, referred to herein as “(C₆)aryloxy”.

The term “benzyl” means —CH₂₋phenyl.

The term “phenyl” means —C₆H₅. A phenyl group can be unsubstituted or substituted with one or two suitable substituents.

The term “phenylene” means a divalent —C₆H₅₋ group. A phenylene group can be unsubstituted or substituted with one or two suitable substituents.

A “hydrocarbyl” group means a monovalent group selected from (C₁-C₈)alkyl, (C₂₋C₈)alkenyl, and (C₂₋C₈)alkynyl, optionally substituted with one or two suitable substituents. Preferably, the hydrocarbon chain of a hydrocarbyl group is from 1 to 6 carbon atoms in length, referred to herein as “(C₁₋C₆)hydrocarbyl”.

A “carbonyl” group is a divalent group of the formula —C(O)—.

An “alkoxycarbonyl” group means a monovalent group of the formula —C(O)— alkoxy. Preferably, the hydrocarbon chain of an alkoxycarbonyl group is from 1 to 8 carbon atoms in length, referred to herein as a “lower alkoxycarbonyl” group.

A “carbamoyl” group means the radical —C(O)N(R′)₂, wherein R′ is chosen from the group consisting of hydrogen, alkyl, and aryl.

As used herein, “halogen” means fluorine, chlorine, bromine, or iodine. Correspondingly, the meaning of the terms “halo” and “Hal” encompass fluoro, chloro, bromo, and iodo.

As used herein, a “suitable substituent” means a group that does not nullify the synthetic or pharmaceutical utility of the compounds of the invention or the intermediates useful for preparing them. Examples of suitable substituents include, but are not limited to: (C₁₋C₈)alkyl; (C₁₋C₈)alkenyl; (C₁₋C₈)alkynyl; aryl; (C₂₋C₅)heteroaryl; (C₁₋C₆)heterocycloalkyl; (C₃₋C₇)cycloalkyl; O—(C₁₋C₈)alkyl; O—(C₁₋C₈)alkenyl; O—(C₁₋C₈)alkynyl; O-aryl; CN; OH; oxo; halo; C(O)OH; COhalo; O(CO)halo; CF₃; N₃; NO₂; NH₂; NH((C₁₋C₈)alkyl); N((C₁₋C₈)alkyl)₂; NH(aryl); N(aryl)₂; (CO)NH₂; (CO)NH((C₁₋C₈)alkyl); (CO)N((C₁₋C₈)alkyl)₂; (CO)NH(aryl); (CO)N(aryl)₂; O(CO)NH₂; NHOH; NOH((C₁₋C₈)alkyl); NOH(aryl); O(CO)NH((C₁₋C₈)alkyl); O(CO)N((C₁₋C₈)alkyl)₂; O(CO)NH(aryl); O(CO)N(aryl)₂; CHO; CO((C₁₋C₈)alkyl); CO(aryl); C(O)O((C₁₋C₈)alkyl); C(O)O(aryl); O(CO)((C₁₋C₈)alkyl); O(CO)(aryl); O(CO)O((C₁₋C₈)alkyl); O(CO)O(aryl); S—(C₁₋C₈)alkyl; S—(C₁₋C₈)alkenyl; S—(C₁₋C₈)alkynyl; and S-aryl. One of skill in art can readily choose a suitable substituent based on the stability and pharmacological and synthetic activity of the compound of the invention.

The compounds of the invention are identified herein by their chemical structure and/or chemical name. Where a compound is referred to by both a chemical structure and a chemical name, and the chemical structure and chemical name conflict, the chemical structure is determinative of the compound's identity.

### 5.2 Synthesis

The compounds of the invention can be obtained via standard, synthetic methodology. Some convenient methods are illustrated in Schemes 1-9. Starting materials useful for preparing the compounds of the invention and intermediates therefor, are commercially available or can be prepared from commercially available materials using known synthetic methods and reagents.

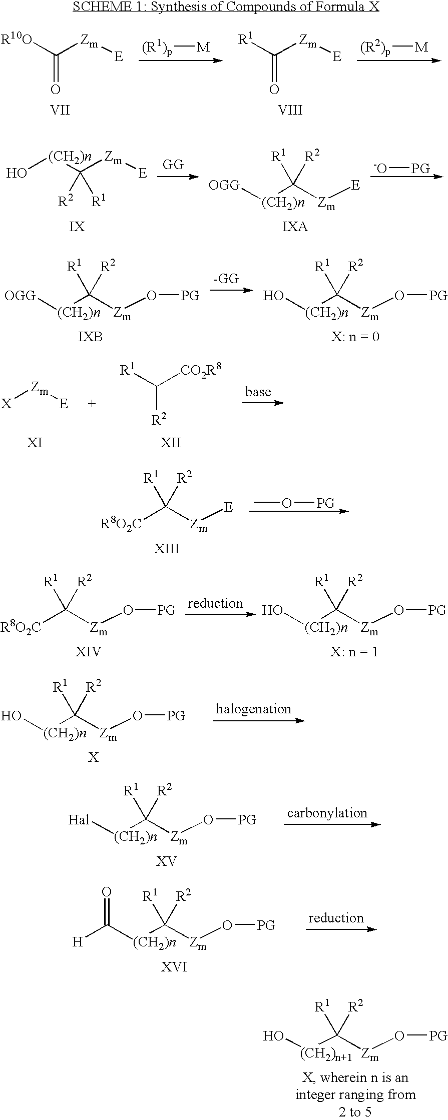

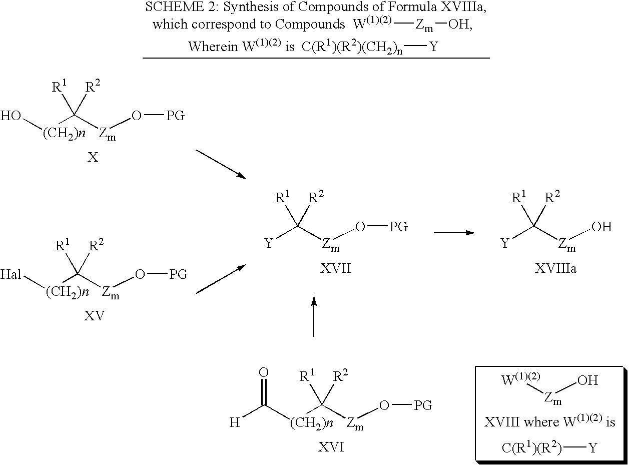

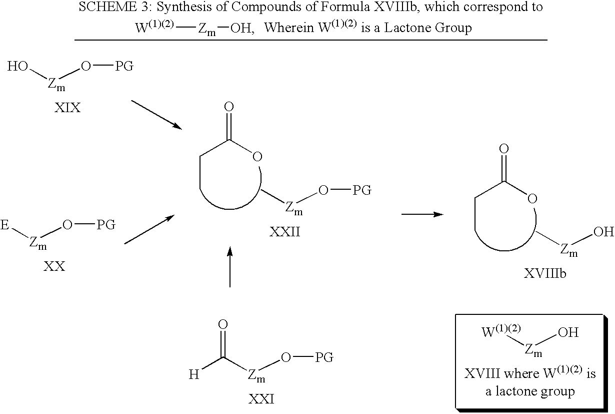

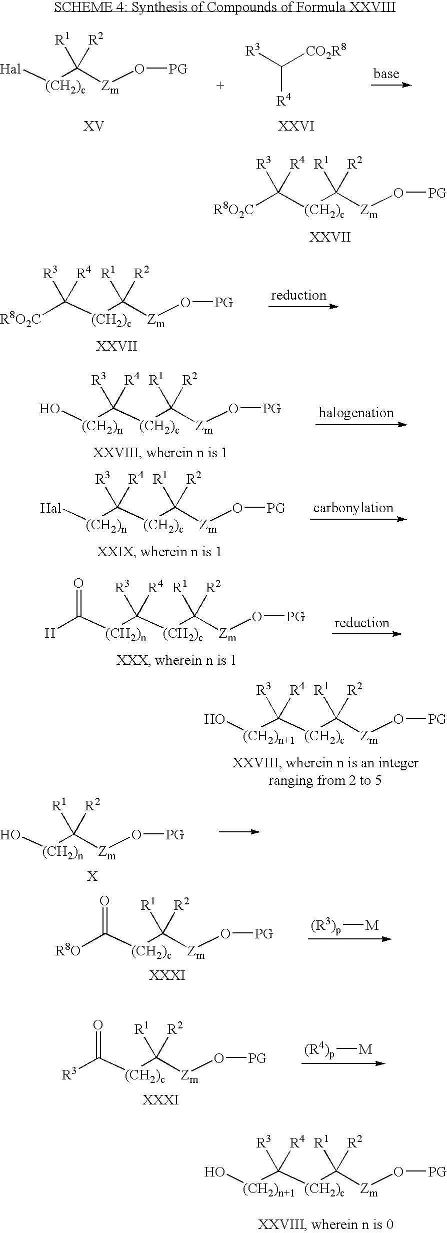

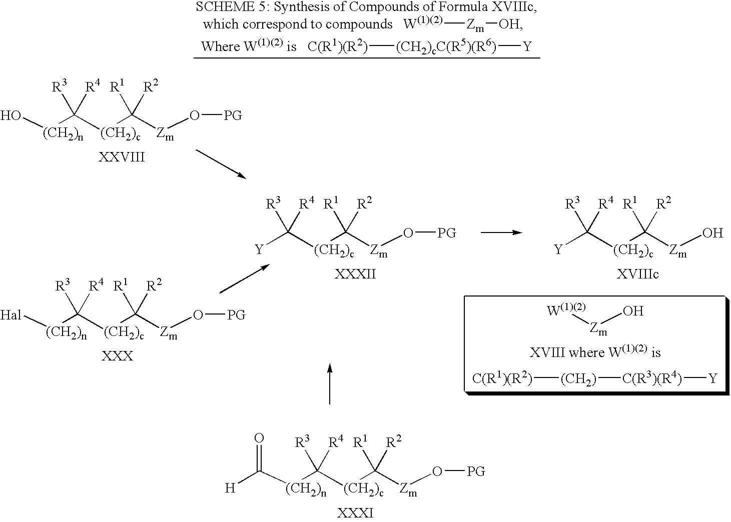

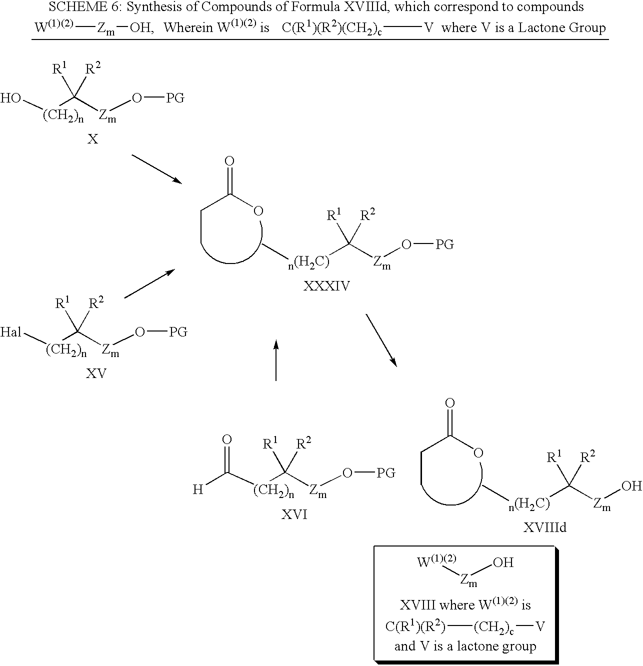

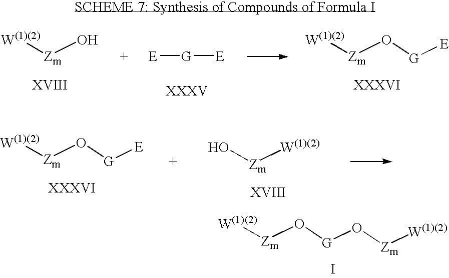

Scheme 1 illustrates the synthesis of mono-protected diols of the formula X, wherein n is an integer ranging from 0 to 4 and R¹ and R² are as defined above. Scheme 1 first outlines the synthesis of mono-protected diols X, wherein n is 0, where esters VII are successively reacted with a first ((R¹)ₚ₋M) then a second ((R²)ₚ₋M) organometallic reagent providing ketones VIII and alcohols IX, respectively. M is a metal group and p is the metal's valency value (e.g., the valency of Li is 1 and that of Zn is 2). Suitable metals include, but are not limited to, Zn, Na, Li, and —Mg-Hal, wherein Hal is a halide selected from iodo, bromo, or chloro. Preferably, M is —Mg-Hal, in which case the organometallic reagents, (R¹)ₚ₋Mg-Hal and (R²)ₚ₋Mg-Hal, are known in the art as Grignard reagents. Esters VII are available commercially (e.g., Aldrich Chemical Co., Milwaukee, Wis.) or can be prepared by well-known synthetic methods, for example, via esterification of the appropriate 5-halovaleric acid (commercially available, e.g., Aldrich Chemical Co., Milwaukee, Wis.). Both (R¹)ₚ₋M and (R²)ₚ₋M are available commercially (e.g., Aldrich Chemical Co., Milwaukee, Wis.) or can be prepared by well-known methods (see e.g., Kharasch et al., Grignard Reactions of Non-Metallic Substances; Prentice-Hall, Englewood Cliffs, N.J., pp. 138-528 (1954) and Hartley; Patai, The Chemistry of the Metal-Carbon Bond, Vol. 4, Wiley: New York, pp. 159-306 and pp. 162-175 (1989), both citations are incorporated by reference herein). The reaction of a first ((R¹)ₚ₋M) then a second ((R²)ₚ₋M) organometallic reagent with esters VII can be performed using the general procedures referenced in March, J. Advanced Organic Chemistry; Reactions Mechanisms, and Structure, 4th ed., 1992, pp. 920-929 and Eicher, Patai, The Chemistry of the Carbonyl Group, pt. 1, pp. 621-693; Wiley: New York, (1966), incorporated by reference herein. For example, the synthetic procedure described in Comins et al., 1981,Tetrahedron Lett. 22:1085, incorporated by reference herein, can be used. As one example, the reaction can be performed by adding an organic solution of (R¹)ₚ₋M (about 0.5 to about 1 equivalents) to a stirred, cooled (about 0° C. to about −80° C.) solution comprising esters VII, under an inert atmosphere (e.g., nitrogen) to give a reaction mixture comprising ketones VIII. Preferably, (R¹)ₚ₋M is added at a rate such that the reaction-mixture temperature remains within about one to two degrees of the initial reaction-mixture temperature. The progress of the reaction can be followed by using an appropriate analytical method, such as thin-layer chromatography or high-performance-liquid chromatography. Next, an organic solution of (R²)ₚ₋M (about 0.5 to about 1 equivalent) is added to the reaction mixture comprising ketones VIII in the same manner used to add (R¹)ₚ₋M. After the reaction providing alcohols IX is substantially complete, the reaction mixture can be quenched and the product can be isolated by workup. Suitable solvents for obtaining alcohols IX include, but are not limited to, dichloromethane, diethyl ether, tetrahydrofuran, benzene, toluene, xylene, hydrocarbon solvents (e.g., pentane, hexane, and heptane), and mixtures thereof. Preferably, the organic solvent is diethyl ether or tetrahydrofuran. Next, alcohols IX are converted to mono-protected diols X, wherein n is 0, using the well-known Williamson ether synthesis. This involves reacting alcohols IX with —O-PG, wherein -PG is a hydroxy-protecting group. For a general discussion of the Williamson ether synthesis, see March, J. Advanced Organic Chemistry; Reactions Mechanisms, and Structure, 4th ed., 1992, pp. 386-387, and for a list of procedures and reagents useful in the Williamson ether synthesis see Larock Comprehensive Organic Transformations; VCH: New York, 1989, pp. 446-448, both of which references are incorporated herein by reference. As used herein, a “hydroxy-protecting group” means a group that is reversibly attached to a hydroxy moiety that renders the hydroxy moiety unreactive during a subsequent reaction(s) and that can be selectively cleaved to regenerate the hydroxy moiety once its protecting purpose has been served. Examples of hydroxy-protecting groups are found in Greene et al., Protective Groups in Organic Synthesis, 3rd ed., John Wiley & Sons, Inc., pp. 17-237 (1999), incorporated herein by reference. Preferably, the hydroxy-protecting group is stable in a basic reaction medium, but can be cleaved by acid. Examples of suitable base-stable acid-labile hydroxy-protecting groups suitable for use with the invention include, but are not limited to, ethers, such as methyl, methoxy methyl, methylthiomethyl, methoxyethoxymethyl, bis(2-chloroethoxy)methyl, tetrahydropyranyl, tetrahydrothiopyranyl, tetrahyrofuranyl, tetrahydrothiofuranyl; 1-ethoxyethyl, 1-methyl-1-methoxyethyl, t-butyl, allyl, benzyl, o-nitrobenzyl, triphenylmethyl, α-naphthyldiphenylmethyl, p-methoxyphenyldiphenylmethyl, 9-(9-phenyl-10-oxo)anthranyl, trimethylsilyl, isopropyldimethylsilyl, t-butyldimethylsilyl, t-butyldiphenylsilyl, tribenzylsilyl, and triisopropylsilyl; and esters, such as pivaloate, adamantoate, and 2,4,6-trimethylbenzoate. Ethers are preferred, particularly straight chain ethers, such as methyl ether, methoxymethyl ether, methylthiomethyl ether, methoxyethoxymethyl ether, bis(2-chloroethoxy)methyl ether. Preferably -PG is methoxymethyl (CH₃OCH₂). Reaction of alcohols IX with —O-PG under the conditions of the Williamson ether synthesis require the protection of the hydroxy group. Alcohols IX are protected with a base-labile protecting group, but stable in the presence of nucleophiles of NaH, Na or other metals used in the next step. Protecting groups recommended for this step are: pivaloate, 2,4,6-trimethylbenzoate (mesitoate), alkylmethyl carbonate, or other similar reagents described in Greene et al., Protective Groups in Organic Synthesis, 3rd ed., John Wiley & Sons, Inc., pp.170-187 (1999). In a typical experiment, the alcohol IX is protected with a hydroxy-protecting group GG, by treating IX with an acid chloride or an anhydride in the presence of a suitable base preferably pyridine or dimethylamino-pyridine in a temperature range of −20 to 100° C., preferably at 0° C., for various periods of time, from a few hours to a few days. The reaction may occur with or without the presence of a solvent, with the base catalyst acting as one, or if a solvent is required dichloromethane, tetrachloroethylene, and toluene are preferred. The protected alcohols IXA are then subjected to the Williamson ether synthesis, which involves adding a base to a stirred organic solution comprising HO-PG (e.g., methoxymethanol), maintained at a constant temperature within the range of about 0° C. to about 80° C., preferably at about room temperature. Preferably, the base is added at a rate such that the reaction-mixture temperature remains within about one to two degrees of the initial reaction-mixture temperature. The base can be added as an organic solution or in undiluted form. Preferably, the base will have a base strength sufficient to deprotonate a proton, wherein the proton has a pKₐ of greater than about 15, preferably greater than about 20. As is well known in the art, the pKₐ is a measure of the acidity of an acid H-A, according to the equation pKₐ=−log Kₐ, wherein Kₐ is the equilibrium constant for the proton transfer. The acidity of an acid H-A is proportional to the stability of its conjugate base -A. For tables listing pKₐ values for various organic acids and a discussion on pKₐ measurement, see March, J. Advanced Organic Chemistry; Reactions Mechanisms, and Structure, 4th ed., 1992, pp. 248-272, incorporated herein by reference. Suitable bases include, but are not limited to, alkylmetal bases such as methyllithium, n-butyllithium, tert-butyllithium, sec-butyllithium, phenyllithium, phenyl sodium, and phenyl potassium; metal arnide bases such as lithium amide, sodium amide, potassium amide, lithium tetramethylpiperidide, lithium diisopropylamide, lithium diethylamide, lithium dicyclohexylamide, sodium hexamethyldisilazide, and lithium hexamethyldisilazide; and hydride bases such as sodium hydride and potassium hydride. The preferred base is sodium hydride. Solvents suitable for reacting alcohols IXA with —OPG include, but are not limited, to dimethyl sulfoxide, dichloromethane, ethers, and mixtures thereof, preferably tetrahydrofuran. After addition of the base, the reaction mixture can be adjusted to within a temperature range of about 0° C. to about room temperature and alcohols IXA can be added, preferably at a rate such that the reaction-mixture temperature remains within about one to two degrees of the initial reaction-mixture temperature. Alcohols IXA can be diluted in an organic solvent or added in their undiluted form. The resulting reaction mixture is stirred until the reaction is substantially complete as determined by using an appropriate analytical method, preferably by gas chromatography, then the bis-protected diols IXB can be isolated by workup and purification. Bis-protected diols IXB are further treated with a suitable base or nucleophile to remove the GG protection. The preferred reagent for this purpose is lithium aluminum hydride, using as solvent THF, diethyl ether, diisopropyl either, t-butyl-methyl ether or mixtures of solvents, at temperatures ranging from −20 to 50° C. and reaction times from 1 hr to 24 hr. Such procedures are extensively describes in Greene et al., Protective Groups in Organic Synthesis, 3rd ed., John Wiley & Sons, Inc., pp.170-187 (1999). The workup of the resulting reaction mixture is performed when the deprotection is complete, which is determined by using the appropriate analytical method, such as thin-layer chromatography or HPLC. Alcohols IX are isolated from the reaction mixture by methods well-known in the art.

Next, Scheme 1 outlines a method useful for synthesizing mono-protected diols X, wherein n is 1. First, compounds XI, wherein E is a suitable leaving group, are reacted with compounds XII, wherein R¹ and R² are as defined above and R⁸ is H, (C₁₋C₆)alkyl or (C₆)aryl, providing compounds XIII. Suitable leaving groups are well known in the art, for example, but not limited to halides, such as chloride, bromide, and iodide; aryl- or alkyl-sulfonyloxy, substituted arylsulfonyloxy (e.g., tosyloxy or mesyloxy); substituted alkyl-sulfonyloxy (e.g., haloalkylsulfonyloxy); phenoxy or subsituted phenoxy; and acyloxy groups. Compounds XI are available commercially (e.g., Aldrich Chemical Co., Milwaukee, Wis.) or can be prepared by well-known methods such as halogenation or sulfonation of butanediol. Compounds XII are also available commercially (e.g., Aldrich Chemical Co., Milwaukee, Wis.) or can be prepared by well-known methods, such as those listed in Larock Comprehensive Organic Transformations; Wiley-VCH: New York, 1999, pp. 1754-1755 and 1765. A review on alkylation of esters of type XII is given in J. Mulzer in Comprehensive Organic Functional Transformations, Pergamon, Oxford 1995, pp. 148-151 and exemplary synthetic procedures for reacting compounds XI with compounds XII are described in U.S. Pat. No. 5,648,387, column 6 and Ackerly, et al., 1995, J. Med. Chem. 1608, all of which citations are incorporated by reference herein. The reaction requires the presence of a suitable base. Preferably, a suitable base will have a pKₐ of greater than about 25, more preferably greater than about 30. Suitable bases include, but are not limited to, alkylmetal bases such as methyllithium, n-butyllithium, tert-butyllithium, sec-butyllithium, phenyllithium, phenyl sodium, and phenyl potassium; metal amide bases such as lithium amide, sodium amide, potassium amide, lithium tetramethylpiperidide, lithium diisopropylamide, lithium diethylamide, lithium dicyclohexylamide, sodium hexamethyldisilazide, and lithium hexamethyldisilazide; hydride bases such as sodium hydride and potassium hydride. Metal amide bases, such as lithium diisopropylamide are preferred. Preferably, to react compounds XI with compounds XII, a solution of about 1 to about 1.2 equivalents of a suitable base is added to a stirred solution comprising esters XII and a suitable organic solvent, under an inert atmosphere, the solution maintained at a constant temperature within the range of about −95° C. to about room temperature, preferably at about −78° C. to about −20° C. Preferably, the base is diluted in a suitable organic solvent before addition. Preferably, the base is added at a rate of about 1.5 moles per hour. Organic solvents suitable for the reaction of compounds XI with the compounds XII include, but are not limited to, diethyl ether, tetrahydrofliran, benzene, toluene, xylene, hydrocarbon solvents (e.g., pentane, hexane, and heptane), and mixtures thereof. After addition of the base, the reaction mixture is allowed to stir for about 1 to about 4 hours, and a compound XI, preferably dissolved in a suitable organic solvent, is added, preferably at a rate such that the reaction-mixture temperature remains within about one to two degrees of the initial reaction-mixture temperature. After addition of compounds XI, the reaction-mixture temperature can be adjusted to within a temperature range of about −20° C. to about room temperature, preferably to about room temperature, and the reaction mixture is allowed to stir until the reaction is substantially complete as determined by using an appropriated analytical method, preferably thin-layer chromatography or high-performance liquid chromatography. Then the reaction mixture is quenched and compounds XIII, wherein n is 1 can be isolated by workup. Compounds XIV are then synthesized by reacting compounds XIII with —O-PG according to the protocol described above for reacting alcohols IX with —O-PG. Next, compounds XIV can be converted to mono-protected diols X, wherein n is 1, by reduction of the ester group of compounds XIV to an alcohol group with a suitable reducing agent. A wide variety of reagents are available for reduction of such esters to alcohols, e.g., see M. Hudlicky, Reductions in Organic Chemistry, 2nd ed., 1996 pp. 212-217, incorporated by reference herein. Preferably, the reduction is effected with a hydride type reducing agent, for example, lithium aluminum hydride, lithium borohydride, lithium triethyl borohydride, diisobutylaluminum hydride, lithium trimethoxyaluminum hydride, or sodium bis(2-methoxy)aluminum hydride. For exemplary procedures for reducing esters to alcohols, see Nystrom et al., 1947, J. Am. Chem. Soc. 69:1197; and Moffet et al., 1963, Org. Synth., Collect. 834(4), lithium aluminum hydride; Brown et al., 1965, J. Am. Chem. Soc. 87:5614, lithium trimethoxyaluminum hydride; Cerny et al., 1969, Collect. Czech. Chem. Commun. 34:1025, sodium bis(2-methoxy)aluminum hydride; Nystrom et al., 1949, J. Am. Chem. 71:245, lithium borohydride; and Brown et al., 1980, J. Org. Chem. 45:1, lithium triethyl borohydride, all of which citations are incorporated herein by reference. Preferably, the reduction is conducted by adding an organic solution of compounds XIV to a stirred mixture comprising a reducing agent, preferably lithium aluminum hydride, and an organic solvent. During the addition, the reaction mixture is maintained at a constant temperature within the range of about −20° C. to about 80° C., preferably at about room temperature. Organic solvents suitable for reacting XIII with -OPG include, but are not limited to, dichloromethane, diethyl ether, tetrahydrofuran or mixtures thereof, preferably tetrahydrofuran. After the addition, the reaction mixture is stirred at a constant temperature within the range of about room temperature to about 60° C., until the reaction is substantially complete as determined by using an appropriate analytical method, preferably thin-layer chromatography or high-performance-liquid chromatography. Then the reaction mixture can be quenched and mono-protected diols X, wherein n is 1, can be isolated by workup and purification.

Scheme 1 next illustrates a three step synthetic sequence for homologating mono-protected diols X comprising: (a) halogenation ( converting —CH₂OH to —CH₂₋Hal); (b) carbonylation (replacing -Hal with —CHO); and (c) reduction (converting —CHO to —CH₂OH), wherein a reaction sequence of (a), (b), and (c) increases the value of n by 1. In step (a) protected halo-alcohols XV, wherein Hal is a halide selected from the group of chloro, bromo, or iodo, preferably iodo, can be prepared by halogenating mono-protected diols X, by using well-known methods (for a discussion of various methods for conversion of alcohols to halides see March, J. Advanced Organic Chemistry; Reactions Mechanisms, and Structure, 4th ed., 1992, pp. 431-433, incorporated herein by reference). For example, protected iodo-alcohols XV can be synthesized starting from mono-protected diols X by treatment with Ph₃/I₂/imidazole (Garegg et al., 1980, J.C.S Perkin I 2866 ); 1,2-dipheneylene phosphorochloridite/I₂ (Corey et al., 1967, J. Org. Chem. 82:4160); or preferably with Me₃SiCl/NaI (Olah et al., 1979, J. Org. Chem. 44:8, 1247), all of which citations are incorporated by reference herein. Step (b); carbonylation of alkyl halides, such as protected halo-alcohols XV, is reviewed in Olah et al., 1987, Chem Rev. 87:4, 671; and March, J., Advanced Organic Chemistry; Reactions Mechanisms, and Structure, 4th ed., 1992, pp. 483-484, both of which are incorporated by reference herein). Protected halo-alcohols XV can be carbonylated with Li(BF₃.Et₂O)/HCONMe₂ using the procedure described in Maddaford et al., 1993, J. Org. Chem. 58:4132; Becker et al., 1982, J. Org. Chem. 3297; or Myers et al., 1992, J. Am. Chem. Soc. 114:9369 or, alternatively, with an organometallic/N-formylmorpholine using the procedure described in Olah et al., 1984, J. Org. Chem. 49:3856 or Vogtle et al., 1987, J. Org. Chem. 52:5560, all of which citations are incorporated by reference herein. The method described in Olah et al., 1984, J. Org. Chem. 49:3856 is preferred. Reduction step (c) useful for synthesizing mono-protected diols X from aldehydes XVI, can be accomplished by well-known methods in the art for reduction of aldehydes to the corresponding alcohols (for a discussion see M. Hudlicky, Reductions in Organic Chemistry, 2nd ed., 1996 pp 137-139), for example, by catalytic hydrogenation (see e.g., Carothers, 1949, J. Am. Chem. Soc. 46:1675) or, preferably by reacting aldehydes XVI with a hydride reducing agent, such as lithium aluminum hydride, lithium borohydride, sodium borohydride (see e.g., the procedures described in Chaikin et al., 1949, J. Am. Chem. Soc. 71:3245; Nystrom et al., 1947, J. Am. Chem. Soc. 69:1197; and Nystrom et al., 1949, J. Am. Chem. 71:3245, all of which are incorporated by reference herein). Reduction with lithium aluminum hydride is preferred.

Scheme 2 outlines methodology for the synthesis of protected alcohols XVIIIa wherein Y, R¹, R², Z, and m are defined as above. Protected alcohols XVIIIa correspond to compounds of the formula W⁽¹⁾⁽²⁾⁻Zm-OPG, wherein W⁽¹⁾⁽²⁾ is C(R¹)(R²)(CH₂)ₙ₋Y.

O-Protected alcohols XVII, wherein Y comprises a —C(O)OH group, can be synthesized by oxidizing mono-protected diols X with an agent suitable for oxidizing a primary alcohol to a carboxylic acid (for a discussion see M. Hudlicky, Oxidations in Organic Chemistry, ACS Monograph 186, 1990, pp. 127-130, incorporated by reference herein). Suitable oxidizing agents include, but are not limited to, pyridinium dichromate (Corey et al., 1979, Tetrahedron Lett. 399); manganese dioxide (Ahrens et al., 1967, J. Heterocycl. Chem. 4:625); sodium permanganate monohydrate (Menger et al., 1981, Tetrahedron Lett. 22:1655); and potassium permanganate (Sam et al., 1972, J. Am. Chem. Soc. 94:4024), all of which citations are incorporated by reference herein. The preferred oxidizing reagent is pyridinium dichromate. In an alternative synthetic procedure, protected alcohols XVII, wherein Y comprises a —C(O)OH group, can be synthesized by treatment of O-protected halo-alcohols XV, wherein X is iodo, with CO or CO₂, as described in Bailey et al., 1990, J. Org. Chem. 55:5404 and Yanagisawa et al., 1994, J. Am. Chem. Soc. 116:6130, the two of which citations are incorporated by reference herein. Protected alcohols XVII, wherein Y comprises —C(O)OR⁷, wherein R⁷ is as defined above, can be synthesized by oxidation of mono-protected diols X in the presence of R⁷OH (see generally, March, J. Advanced Organic Chemistry; Reactions Mechanisms, and Structure, 4th ed., 1992, p. 1196). An exemplary procedure for such an oxidation is described in Stevens et al., 1982, Tetrahedron Lett. 23:4647 (HOCl); Sundararaman et al., 1978, Tetrahedron Lett. 1627 (O₃/KOH); Wilson et al., 1982, J. Org. Chem. 47:1360 (t-BuOOH/Et₃N); and Williams et al., 1988, Tetrahedron Lett. 29:5087 (Br₂), the four of which citations are incorporated by reference herein. Preferably, O-protected alcohols XVII, wherein Y comprises a —C(O)OR⁷ group are synthesized from the corresponding carboxylic acid (i.e., XVII, wherein Y comprises —C(O)OH) by esterification with R⁷OH (e.g., see March, J., Advanced Organic Chemistry; Reactions Mechanisms, and Structure, 4th ed., 1992, p. 393-394, incorporated by reference herein). In another alternative synthesis, protected alcohols XVII, wherein Y comprises —C(O)OR⁷, can be prepared from protected halo-alcohols XV by carbonylation with transition metal complexes (see e.g., March, J. Advanced Organic Chemistry; Reactions Mechanisms, and Structure, 4th ed., 1992, p. 484-486; Urata et al., 1991, Tetrahedron Lett. 32:36, 4733); and Ogata et al., 1969, J. Org. Chem. 3985, the three of which citations are incorporated by reference herein).

O-Protected alcohols XVII, wherein Y comprises —OC(O)R⁷, wherein R⁷ is as defined above, can be prepared by acylation of mono-protected diols X with a carboxylate equivalent such as an acyl halide (i.e., R⁷C(O)-Hal, wherein Hal is iodo, bromo, or chloro, see e.g., March, J. Advanced Organic Chemistry; Reactions Mechanisms, and Structure, 4th ed., 1992, p. 392 and Org. Synth. Coll. Vol. III, Wiley, NY, pp. 142, 144, 167, and 187 (1955)) or an anhydride (i.e., R⁷C(O)—O—(O)CR⁷, see e.g., March, J. Advanced Organic Chemistry; Reactions Mechanisms, and Structure, 4th ed., 1992, p. 392-393 and Org. Synth. Coll. Vol. III, Wiley, NY, pp. 11, 127, 141, 169, 237, 281, 428, 432, 690, and 833 (1955), all of which citations are incorporated herein by reference). Preferably, the reaction is conducted by adding a base to a solution comprising mono-protected diols X, a carboxylate equivalent, and an organic solvent, which solution is preferably maintained at a constant temperature within the range of 0° C. to about room temperature. Solvents suitable for reacting mono-protected diols X with a carboxylate equivalent include, but are not limited to, dichloromethane, toluene, and ether, preferably dichloromethane. Suitable bases include, but are not limited to, hydroxide sources, such as sodium hydroxide, potassium hydroxide, sodium carbonate, or potassium carbonate; or an amine such as triethylamine, pyridine, or dimethylaminopyridine. The progress of the reaction can be followed by using an appropriate analytical technique, such as thin layer chromatography or high performance liquid chromatography and when substantially complete, the product can be isolated by workup and purified if desired.

Protected alcohols XVII, wherein Y comprises one of the following phosphate ester groups

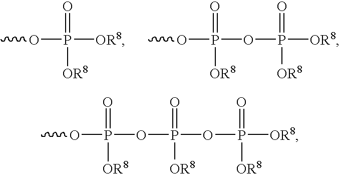

wherein R⁸ is defined as above, can be prepared by phosphorylation of mono-protected diols X according to well-known methods (for a general reviews, see Corbridge Phosphorus: An Outline of its Chemistry, Biochemistry, and Uses, Studies in Inorganic Chemistry, 3rd ed., pp. 357-395 (1985); Ramirez et al., 1978, Acc. Chem. Res. 11:239; and Kalckare Biological Phosphorylations, Prentice-Hall, New York (1969); J. B. Sweeny in Comprehensive Organic Functional Group Transformations, A. R. Katritzky, O. Meth-Cohn and C. W. Rees, Eds. Pergamon: Oxford, 1995, vol 2, pp. 104-109, the four of which are incorporated herein by reference). Protected alcohols XVII wherein Y comprises a monophosphate group of the formula:

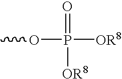

wherein R⁸ is defined as above, can be prepared by treatment of mono-protected diol X with phosphorous oxychloride in a suitable solvent, such as xylene or toluene, at a constant temperature within the range of about 100NC to about 150NC for about 2 hours to about 24 hours. After the reaction is deemed substantially complete, by using an appropriate analytical method, the reaction mixture is hydrolyzed with R⁸⁻OH. Suitable procedures are referenced in Houben-Weyl, Methoden der Organische Chemie, Georg Thieme Verlag Stuttgart 1964, vol. XII/2, pp. 143-210 and 872-879, incorporated by reference herein. Alternatively, when both R⁸ are hydrogen, can be synthesized by reacting mono-protected diols X with silyl polyphosphate (Okamoto et al., 1985, Bull Chem. Soc. Jpn. 58:3393, incorporated herein by reference) or by hydrogenolysis of their benzyl or phenyl esters (Chen et al., 1998, J. Org. Chem. 63:6511, incorporated herein by reference). In another alternative procedure, when R⁸ is (C₁₋C₆)alkyl, (C₂₋C₆)alkenyl, or (C₂₋C₆)alkynyl, the monophosphate esters can be prepared by reacting mono-protected diols X with appropriately substituted phophoramidites followed by oxidation of the intermediate with m-chloroperbenzoic acid (Yu et al., 1988, Tetrahedron Lett. 29:979, incorporated herein by reference) or by reacting mono-protected diols X with dialkyl or diaryl substituted phosphorochloridates (Pop et al, 1997, Org. Prep. and Proc. Int. 29:341, incorporated herein by reference). The phosphoramidites are commercially available (e.g., Aldrich Chemical Co., Milwaukee, Wis.) or readily prepared according to literature procedures (see e.g., Uhlmann et al. 1986, Tetrahedron Lett. 27:1023 and Tanaka et al., 1988, Tetrahedron Lett. 29:199, both of which are incorporated herein by reference). The phosphorochloridates are also commercially available (e.g., Aldrich Chemical Co., Milwaukee, Wis.) or prepared according to literature methods (e.g., Gajda et al, 1995, Synthesis 25:4099. In still another alternative synthesis, protected alcohols XVII, wherein Y comprises a monophosphate group and R⁸ is alkyl or aryl, can be prepared by reacting IP⁺(OR⁸)₃ with mono-protected diols X according to the procedure described in Stowell et al., 1995, Tetrahedron Lett. 36:11, 1825 or by alkylation of protected halo alcohols XV with the appropriate dialkyl or diaryl phosphates (see e.g., Okamoto, 1985, Bull Chem. Soc. Jpn. 58:3393, incorporated herein by reference).

Protected alcohols XVII wherein Y comprises a diphosphate group of the formula

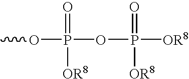

wherein R⁸ is defined as above, can be synthesized by reacting the above-discussed monophosphates of the formula:

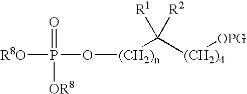

with a phosphate of the formula

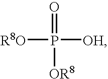

(commercially available, e.g., Aldrich Chemical Co., Milwaukee, Wis.), in the presence of carbodiimide such as dicyclohexylcarbodiimide, as described in Houben-Weyl, Methoden der Organische Chemie, Georg Thieme Verlag Stuttgart 1964, vol. XII/2, pp. 881-885. In the same fashion, protected alcohols XVII, wherein Y comprises a triphosphate group of the formula:

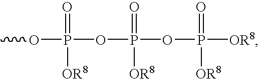

can be synthesized by reacting the above-discussed diphosphate protected alcohols, of the formula:

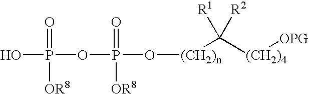

with a phosphate of the formula:

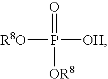

as described above. Alternatively, when R⁸ is H, protected alcohols XVII wherein Y comprises the triphosphate group, can be prepared by reacting mono-protected diols X with salicyl phosphorochloridite and then pyrophosphate and subsequent cleavage of the adduct thus obtained with iodine in pyridine as described in Ludwig et al., 1989, J. Org. Chem. 54:631, incorporated herein by reference.

Protected alcohols XVII, wherein Y is -SO₃H or a heterocyclic group selected from the group consisting of:

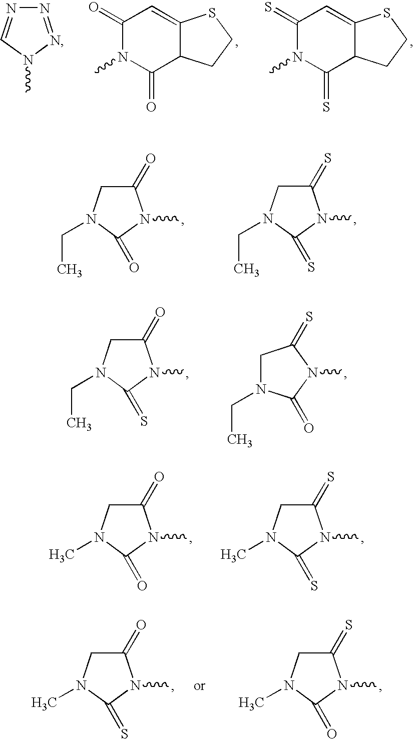

can be prepared by halide displacement from protected halo-alcohols XV. Thus, when Y is -SO₃H, protected alcohols XVII can by synthesized by reacting protected halo-alcohols XV with sodium sulfite as described in Gilbert Sulfonation and Related Reactions; Wiley: New York, 1965, pp. 136-148 and pp. 161-163; Org. Synth. Coll. Vol. II, Wiley, NY, 558, 564 (1943); and Org. Synth. Coll. Vol. IV, Wiley, NY, 529 (1963), all three of which are incorporated herein by reference. When Y is one of the above-mentioned heterocycles, protected alcohols XVII can be prepared by reacting protected halo-alcohols XV with the corresponding heterocycle in the presence of a base. The heterocycles are available commercially (e.g., Aldrich Chemical Co., Milwaukee, Wis.) or prepared by well-known synthetic methods (see the procedures described in Ware, 1950, Chem. Rev. 46:403-470, incorporated herein by reference). Preferably, the reaction is conducted by stirring a mixture comprising XV, the heterocycle, and a solvent at a constant temperature within the range of about room temperature to about 100NC, preferably within the range of about 50NC to about 70NC for about 10 to about 48 hours. Suitable bases include hydroxide bases such as sodium hydroxide, potassium hydroxide, sodium carbonate, or potassium carbonate. Preferably, the solvent used in forming protected alcohols XVII is selected from dimethylformamide; formamide; dimethyl sulfoxide; alcohols, such as methanol or ethanol; and mixtures thereof. The progress of the reaction can be followed by using an appropriate analytical technique, such as thin layer chromatography or high performance liquid chromatography and when substantially complete, the product can be isolated by workup and purified if desired.

Protected alcohols XVII, wherein Y is a heteroaryl ring selected from

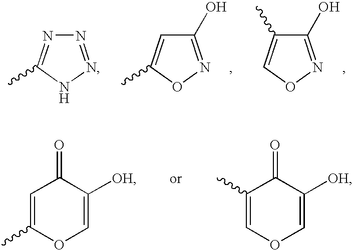

can be prepared by metallating the suitable heteroaryl ring then reacting the resulting metallated heteroaryl ring with protected halo-alcohols XV (for a review, see Katritzky Handbook of Heterocyclic Chemistry, Pergamon Press: Oxford 1985). The heteroaryl rings are available commercially or prepared by well-known synthetic methods (see e.g., Joule et al., Heterocyclic Chemistry, 3rd ed., 1995; De Sarlo et al., 1971, J. Chem. Soc. (C) 86; Oster et al., 1983, J. Org. Chem. 48:4307; Iwai et al., 1966, Chem. Pharm. Bull. 14:1277; and U.S. Pat. No. 3,152,148, all of which citations are incorporated herein by reference). As used herein, the term “metallating” means the forming of a carbon-metal bond, which bond may be substantially ionic in character. Metallation can be accomplished by adding about 2 equivalents of strong organometallic base, preferably with a pKₐ of about 25 or more, more preferably with a pKₐ of greater than about 35, to a mixture comprising a suitable organic solvent and the heterocycle. Two equivalents of base are required: one equivalent of the base deprotonates the —OH group or the —NH group, and the second equivalent metallates the heteroaryl ring. Alternatively, the hydroxy group of the heteroaryl ring can be protected with a base-stable, acid-labile protecting group as described in Greene, T. W., Protective Groups in Organic Synthesis, 3rd edition 17-237 (1999), incorporated herein by reference. Where the hydroxy group is protected, only one equivalent of base is required. Examples of suitable base-stable, acid-labile hydroxyl-protecting groups, include but are not limited to, ethers, such as methyl, methoxy methyl, methylthiomethyl, methoxyethoxymethyl, bis(2-chloroethoxy)methyl, tetrahydropyranyl, tetrahydrothiopyranyl, tetrahyrofuranyl, tetrahydrothiofuranyl, 1-ethoxyethyl, 1-methyl-1-methoxyethyl, t-butyl, allyl, benzyl, o-nitrobenzyl, triphenylmethyl, α-naphthyldiphenylmethyl, p-methoxyphenyldiphenylmethyl, 9-(9-phenyl-10-oxo)anthranyl, trimethylsilyl, isopropyldimethylsilyl, t-butyldimethylsilyl, t-butyldiphenylsilyl, tribenzylsilyl, triisopropylsilyl; and esters, such as pivaloate, adamantoate, and 2,4,6-trimethylbenzoate. Ethers are preferred, particularly straight chain ethers, such as methyl ether, methoxymethyl ether, methylthiomethyl ether, methoxyethoxymethyl ether, bis(2-chloroethoxy)methyl ether. Preferably, the pKₐ of the base is higher than the pKₐ of the proton of the heterocycle to be deprotonated. For a listing of pKₐs for various heteroaryl rings, see Fraser et al., 1985, Can. J. Chem. 63:3505, incorporated herein by reference. Suitable bases include, but are not limited to, alkylmetal bases such as methyllithium, n-butyllithium, tert-butyllithium, sec-butyllithium, phenyllithium, phenyl sodium, and phenyl potassium; metal amide bases such as lithium amide, sodium amide, potassium amide, lithium tetramethylpiperidide, lithium diisopropylamide, lithium diethylamide, lithium dicyclohexylamide, sodium hexamethyldisilazide, and lithium hexamethyldisilazide; and hydride bases such as sodium hydride and potassium hydride. If desired, the organometallic base can be activated with a complexing agent, such as N,N,N′,N′-tetramethylethylenediamine or hexamethylphosphoramide (1970, J. Am. Chem. Soc. 92:4664, incorporated by reference herein). Solvents suitable for synthesizing protected alcohols XVII, wherein Y is a heteroaryl ring include, but are not limited to, diethyl ether; tetrahydrofuran; and hydrocarbons, such as pentane. Generally, metallation occurs alpha to the heteroatom due to the inductive effect of the heteroatom, however, modification of conditions, such as the identity of the base and solvents, order of reagent addition, reagent addition times, and reaction and addition temperatures can be modified by one of skill in the art to achieve the desired metallation position (see e.g., Joule et al., Heterocyclic Chemistry, 3rd ed., 1995, pp. 30-42, incorporated by reference herein) Alternatively, the position of metallation can be controlled by use of a halogenated heteroaryl group, wherein the halogen is located on the position of the heteroaryl ring where metallation is desired (see e.g., Joule et al., Heterocyclic Chemistry, 3rd ed., 1995, p. 33 and Saulnier et al., 1982, J. Org. Chem. 47:757, the two of which citations are incorporated by reference herein). Halogenated heteroaryl groups are available commercially (e.g., Aldrich Chemical Co., Milwaukee, Wis.) or can be prepared by well-known synthetic methods (see e.g., Joule et al., Heterocyclic Chemistry, 3rd ed., 1995, pp. 78, 85, 122, 193, 234, 261, 280, 308, incorporated by reference herein). After metallation, the reaction mixture comprising the metallated heteroaryl ring is adjusted to within a temperature range of about [symbol] to about room temperature and protected halo-alcohols XV (diluted with a solvent or in undiluted form) are added, preferably at a rate such that the reaction-mixture temperature remains within about one to two degrees of the initial reaction-mixture temperature. After addition of protected halo-alcohols XV, the reaction mixture, is stirred at a constant temperature within the range of about room temperature and about the solvent's boiling temperature and the reaction's progress can be monitored by the appropriate analytical technique, preferably thin-layer chromatography or high-performance liquid chromatography. After the reaction is substantially complete, protected alcohols XVII can be isolated by workup and purification. It is to be understood that conditions, such as the identity of protected halo-alcohol XV, the base, solvents, orders of reagent addition, times, and temperatures, can be modified by one of skill in the art to optimize the yield and selectivity. Exemplary procedures that can be used in such a transformation are described in Shirley et al., 1995, J. Org. Chem. 20:225; Chadwick et al., 1979, J. Chem. Soc., Perkin Trans. 1 2845; Rewcastle, 1993, Adv. Het. Chem. 56:208; Katritzky et al., 1993, Adv. Het. Chem. 56:155; and Kessar et al., 1997, Chem. Rev. 97:721.

When Y is

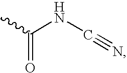

protected alcohols XVII can be prepared from their corresponding carboxylic acid derivatives (XVII, wherein Y is —CO₂H) as described in Belletire et al, 1988, Synthetic Commun. 18:2063 or from the corresponding acylchlorides (XVII, wherein Y is —CO-halo) as described in Skinner et al., 1995, J. Am. Chem. Soc. 77:5440, both citations are incorporated herein by reference. The acylhalides can be prepared from the carboxylic acids by well known procedures such as those described in March, J., Advanced Organic Chemistry; Reactions Mechanisms, and Structure, 4th ed., 1992, pp. 437-438, incorporated by reference herein. When Y is

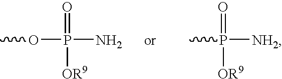

wherein R⁹ is as defined above, protected alcohols XVII can be prepared by first reacting protected halo-alcohols XV with a trialkyl phosphite according to the procedure described in Kosolapoff, 1951, Org. React. 6:273 followed by reacting the derived phosphonic diester with ammonia according to the procedure described in Smith et al., 1957, J. Org. Chem. 22:265, incorporated herein by reference. When Y is

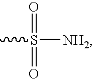

protected alcohols XVII can be prepared by reacting their sulphonic acid derivatives (i.e., XVII, wherein Y is —SO₃H) with ammonia as described in Sianesi et al., 1971, Chem. Ber. 104:1880 and Campagna et al., 1994, Farmaco, Ed. Sci. 49:653, both of which citations are incorporated herein by reference).

As further illustrated in Scheme 2, protected alcohols XVII can be deprotected providing alcohols XVIIIa. The deprotection method depends on the identity of the alcohol-protecting group, see e.g., the procedures listed in Greene, T. W., Protective Groups in Organic Synthesis, 3rd edition 17-237 (1999), particularly see pages 48-49, incorporated herein by reference. One of skill in the art will readily be able to choose the appropriate deprotection procedure. When the alcohol is protected as an ether function (e.g., methoxymethyl ether), the alcohol is preferably deprotected with aqueous or alcoholic acid. Suitable deprotection reagents include, but are not limited to, aqueous hydrochloric acid, p-toluenesulfonic acid in methanol, pyridinium-p-toluenesulfonate in ethanol, Amberlyst H-15 in methanol, boric acid in ethylene-glycol-monoethylether, acetic acid in a water-tetrahydrofuran mixture, aqueous hydrochloric acid is preferred. Examples of such procedures are described, respectively, in Bemady et al., 1979, J. Org. Chem. 44:1438; Miyashita et al., 1977, J. Org. Chem. 42:3772; Johnston et al., 1988, Synthesis 393; Bongini et al., 1979, Synthesis 618; and Hoyer et al., 1986, Synthesis 655; Gigg et al., 1967, J. Chem. Soc. C, 431; and Corey et al., 1978, J. Am. Chem. Soc. 100:1942, all of which are incorporated herein by reference.

Scheme 3 depicts the synthesis of protected lactone alcohols XXII and lactone alcohols XVIIIb. Compounds XXII and XVIIIb correspond to compounds of the formula W⁽¹⁾⁽²⁾⁻Zm-OPG and W⁽¹⁾⁽²⁾⁻Zm-OH respectively, wherein W⁽¹⁾⁽²⁾ is a lactone group selected from:

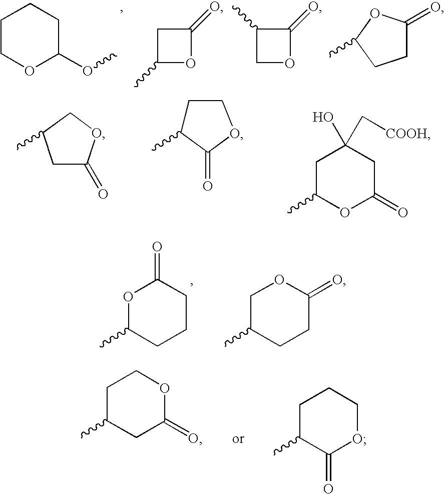

Protected lactone alcohols XXII can be prepared from compounds of the formula XIX, XX, or XXI by using well-known condensation reactions and variations of the Michael reaction. Methods for the synthesis of lactones are disclosed in Multzer in Comprehensive Organic Functional Group Transformations, A. R. Katritzky, O. Meth-Cohn and C. W. Rees, Eds. Pergamon: Oxford, 1995, vol 5, pp. 161-173, incorporated herein by reference. Mono-protected diols XIX, electrophilic protected alcohols XX, and aldehydes XXI are readily available ether commercially (e.g., Aldrich Chemical Co., Milwaukee, Wis.) or by well known synthetic procedures.

When W⁽¹⁾⁽²⁾ is a beta-lactone group of the formula:

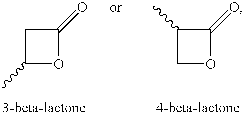

protected lactone alcohols XXII can be prepared from aldehydes XXI and electrophilic protected alcohols XX, respectively, by a one-pot-addition-lactonization according to the procedure of Masamune et al., 1976, J. Am. Chem. Soc. 98:7874 and Danheiser et al., 1991, J. Org. Chem. 56:1176, both of which are incorporated herein by reference. This one-pot-addition-lactonization methodology has been reviewed by Multzer in Comprehensive Organic Functional Group Transformations, A. R. Katritzky, O. Meth-Cohn and C. W. Rees, Eds. Pergamon: Oxford, 1995, vol 5, pp. 161, incorporated herein by reference When W⁽¹⁾⁽²⁾ is a gamma- or delta-lactone group of the formula:

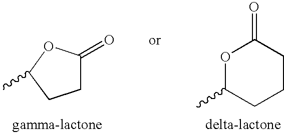

protected lactone alcohols XXII can be prepared from aldehydes XXI according to well known synthetic methodology. For example, the methodology described in Masuyama et al., 2000, J. Org. Chem. 65:494; Eisch et al.,1978, J. Organometall. Chem. C8 160; Eaton et al., 1947, J. Org. Chem. 37:1947; Yunker et al., 1978, Tetrahedron Lett. 4651; Bhanot et al.,1977, J. Org. Chem. 42:1623; Ehlinger et al., 1980, J. Am. Chem. Soc. 102:5004; and Raunio et al., 1957, J. Org. Chem. 22:570, all of which citations are incorporated herein by reference. For instance, as described in Masuyama et al., 2000, J. Org. Chem. 65:494, aldehydes XXI can be treated with about 1 equivalent of a strong organometallic base, preferably with a pKₐ of about 25 or more, more preferably with a pKₐ of greater than about 35, in a suitable organic solvent to give a reaction mixture. Suitable bases include, but are not limited to, alkylmetal bases such as methyllithium, n-butyllithium, tert-butyllithium, sec-butyllithium, phenyllithium, phenyl sodium, and phenyl potassium; metal amide bases such as lithium amide, sodium amide, potassium amide, lithium tetramethylpiperidide, lithium diisopropylamide, lithium diethylamide, lithium dicyclohexylamide, sodium hexamethyldisilazide, and lithium hexamethyldisilazide; and hydride bases such as sodium hydride and potassium hydride, preferably lithium tetramethylpiperidide. Suitable solvents include, but are not limited to, diethyl ether and tetrahydrofuran.

The reaction-mixture temperature is adjusted to within the range of about 0NC to about 100NC, preferably about room temperature to about 50NC, and a halide of the formula:

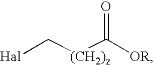

wherein z is 1 or 2 (diluted with a solvent or in undiluted form) is added. The reaction mixture is stirred for a period of about 2 hours to about 48 hours, preferably about 5 to about 10 hours, during which time the reaction's progress can be followed by using an appropriate analytical technique, such as thin layer chromatography or high performance liquid chromatography. When the reaction is deemed substantially complete, protected lactone alcohols XXII can be isolated by workup and purified if desired. When W⁽¹⁾⁽²⁾ is a gamma- or delta-lactone group of the formula:

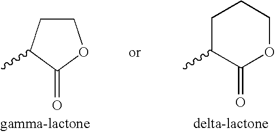

protected lactone alcohols XXII can be synthesized by deprotonating the corresponding lactone with a strong base providing the lactone enolate and reacting the enolate with electrophilic protected alcohols XX (for a detailed discussion of enolate formation of active methylene compounds such as lactones, see House Modern Synthetic Reactions; W.A. Benjamin, Inc. Philippines 1972 pp. 492-570, and for a discussion of reaction of lactone enolates with electrophiles such as carbonyl compounds, see March, J. Advanced Organic Chemistry; Reactions Mechanisms, and Structure, 4th ed., 1992, pp. 944-945, both of which are incorporated herein by reference). Lactone-enolate formation can be accomplished by adding about 1 equivalent of a strong organometallic base, preferably with a pKₐ of about 25 or more, more preferably with a pKₐ of greater than about 35, to a mixture comprising a suitable organic solvent and the lactone. Suitable bases include, but are not limited to, alkylmetal bases such as methyllithium, n-butyllithium, tert-butyllithium, sec-butyllithium, phenyllithium, phenyl sodium, and phenyl potassium; metal amide bases such as lithium amide, sodium amide, potassium amide, lithium tetramethylpiperidide, lithium diisopropylamide, lithium diethylamide, lithium dicyclohexylamide, sodium hexamethyldisilazide, and lithium hexamethyldisilazide; and hydride bases such as sodium hydride and potassium hydride, preferably lithium tetramethylpiperidide. Solvents suitable for lactone-enolate formation include, but are not limited to, diethyl ether and tetrahydrofuran. After enolate formation, the reaction-mixture temperature is adjusted to within the range of about −78NC to about room temperature, preferably about −50NC to about 0NC, and electrophilic protected alcohols XX (diluted with a solvent or in undiluted form) are added, preferably at a rate such that the reaction-mixture temperature remains within about one to two degrees of the initial reaction-mixture temperature. The reaction mixture is stirred for a period of about 15 minutes to about 5 hours, during which time the reaction's progress can be followed by using an appropriate analytical technique, such as thin layer chromatography or high performance liquid chromatography. When the reaction is deemed substantially complete, protected lactone alcohols XXII can be isolated by workup and purified if desired. When W⁽¹⁾⁽²⁾ is a lactone group of the formula:

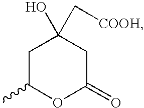

protected lactone alcohols XXII can be prepared from aldehydes XXI according to the procedure described in U.S. Pat. No. 4,622,338, incorporated by reference herein.

When W⁽¹⁾⁽²⁾ is a gamma- or delta-lactone group of the formula:

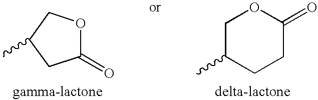

protected lactone alcohols XXII can be prepared according to a three step sequence. The first step comprises base-mediated reaction of electrophilic protected alcohols XX with succinic acid esters (i.e., R⁹O₂CCH₂CH₂CO₂R⁹, wherein R⁹ is alkyl) or glutaric acid esters (i.e., R⁹O₂CCH₂CH₂CH₂CO₂R⁹, wherein R⁹ is alkyl) providing a diester intermediate of the formula XXIV:

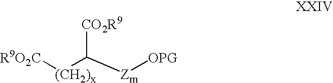

wherein x is 1 or 2 depending on whether the ganma or delta lactone group is desired. The reaction can be performed by adding about 1 equivalent of a strong organometallic base, preferably with a pKₐ of about 25 or more, more preferably with a pKₐ of greater than about 35, to a mixture comprising a suitable organic solvent and the succinic or glutaric acid ester. Suitable bases include, but are not limited to, alkylmetal bases such as methyllithium, n-butyllithium, tert-butyllithium, sec-butyllithium, phenyllithium, phenyl sodium, and phenyl potassium; metal amide bases such as lithium amide, sodium amide, potassium amide, lithium tetramethylpiperidide, lithium diisopropylamide, lithium diethylamide, lithium dicyclohexylamide, sodium hexamethyldisilazide, and lithium hexamethyldisilazide; and hydride bases such as sodium hydride and potassium hydride, preferably lithium tetramethylpiperidide. Suitable solvents include, but are not limited to, diethyl ether and tetrahydrofuran. After enolate formation, the reaction-mixture temperature is adjusted to within the range of about −78° C. to about room temperature, preferably about −50° C. to about 0° C., and electrophilic protected alcohols XX (diluted with a solvent or in undiluted form) are added, preferably at a rate such that the reaction-mixture temperature remains within about one to two degrees of the initial reaction-mixture temperature. The reaction mixture is stirred for a period of about 15 minutes to about 5 hours, during which time the reaction's progress can be followed by using an appropriate analytical technique, such as thin layer chromatography or high performance liquid chromatography. When the reaction is deemed substantially complete, the diester intermediate be isolated by workup and purified if desired. In the second step, the intermediate diester can be reduced, with a hydride reducing agent, to yield a diol of the formula XXV:

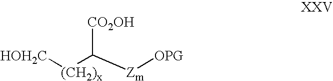

The reduction can be performed according to the procedures referenced in March, J. Advanced Organic Chemistry; Reactions Mechanisms, and Structure, 4th ed., 1992, p. 1214, incorporated herein by reference). Suitable reducing agents include, but are not limited to, lithium aluminum hydride, diisobutylaluminum hydride, sodium borohydride, and lithium borohydride). In the third step, the diol can be oxidatively cyclized with RuH₂(PPh₃)₄ to the product protected lactone alcohols XXII according to the procedure of Yoshikawa et al., 1986, J. Org. Chem. 51:2034 and Yoshikawa et al., 1983, Tetrahedron Lett. 26:2677, both of which citations are incorporated herein by reference. When W⁽¹⁾⁽²⁾ is a lactone group of the formula:

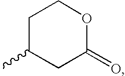

protected lactone alcohols XXII can be synthesized by reacting the Grignard salts of electrophilic protected alcohols XX, where E is a halide, with 5,6-dihydro-2H-pyran-2-one, commercially available (e.g., Aldrich Chemical Co., Milwaukee, Wis.), in the presence of catalytic amounts of a 1-dimethylaminoacetyl)pyrolidine-2yl)methyl-diarylphosphine-copper (I) iodide complex as described in Tomioka et al., 1995, Tetrahedron Lett. 36:4275, incorporated herein by reference.

Scheme 4 outlines methodology for the synthesis of protected alcohols XXVIII. Compounds XXVIII, wherein n is an integer ranging from 1 to 4 can be prepared from compounds XV using general synthetic strategy depicted and adapting the synthetic protocols from those discussed for Scheme 1.

Next, Scheme 4 depicts the general strategy for the synthesis of compounds XXVIII wherein n is 0. First, Esters XXXI, wherein R⁸ is as defined above, are synthesized by oxidation of mono-protected diols X in the presence of R⁸OH (see generally, March, J. Advanced Organic Chemistry; Reactions Mechanisms, and Structure, 4th ed., 1992, p. 1196). An exemplary procedure for such an oxidation is described in Stevens et al., 1982, Tetrahedron Lett. 23:4647 (HOCl); Sundararaman et al., 1978, Tetrahedron Lett. 1627 (O₃/KOH); Wilson et al., 1982, J. Org. Chem. 47:1360 (t-BuOOH/Et₃N); and Williams et al., 1988, Tetrahedron Lett. 29:5087 (Br₂), the four of which citations are incorporated by reference herein. Compounds XXXI are converted to compounds XXVIII wherein n is 0 by adapting the synthetic procedures depicted in Scheme 1.

Scheme 5 outlines methodology for the synthesis of protected alcohols XXXII and alcohols XVIIIc, which correspond to W⁽¹⁾⁽²⁾⁻Zₘ₋OPG and W⁽¹⁾⁽²⁾⁻Zₘ₋OH, respectively, wherein W⁽¹⁾⁽²⁾ is C(R¹)(R²)—(CH₂)_{c}C(R⁵)(R⁶)—Y. The synthesis of starting materials XXVIII, XXX and XXXI are depicted in Scheme 4 and the synthetic methods and procedures can be adapted from those described for Scheme 2.

Scheme 6 depicts the synthesis of protected lactone alcohols XXXIV and lactone alcohols XVIIId. Compounds XXXIV and XVIIId correspond to compounds of the formula, which correspond to compounds W⁽¹⁾⁽²⁾⁻Zₘ₋OH, Wherein W⁽¹⁾⁽²⁾ is C(R¹)(R²)(CH₂)_{c-}V and V is a Group selected from:

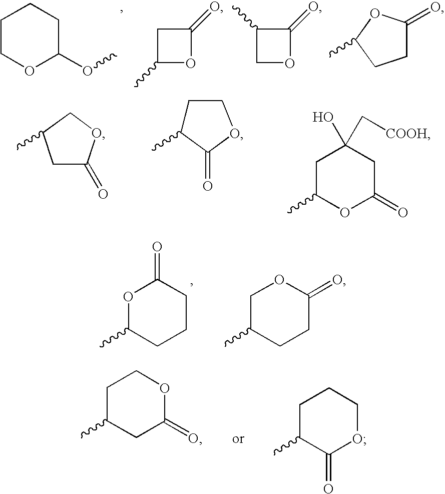

As shown in Scheme 6, protected lactone alcohols XXXIV and lactone alcohols XVIIId can be synthesized from compounds of the formula X, XV, or XVI by adaptation of the methods and procedures discussed above for Scheme 3.

Scheme 7 outlines the synthesis of compounds I. In the first step, compounds XXXVI are synthesized by reacting compounds XVIII (compounds XVIII a,b,c, and d are encompassed by XVIII) with compounds XXXV under the conditions of the Williamson ether synthesis. The conditions and methods discussed in Scheme 1 above for the synthesis of mono-protected diols X from alcohols IX can be adapted for the synthesis of compounds XXXVI. Compounds XXXV, wherein E is a suitable leaving group as defined above, preferably chloride or bromide, are readily obtained commercially (e.g., Aldrich Chemical Co. Milwaukee Wis.) or by well known synthetic methods. Compounds I are obtained by reacting compounds XXXVI with compounds XVII under the conditions of the Williamson ether synthesis. In a preferred Williamson procedure, first, a base is added to a stirred organic solution comprising alcohols XVIII, maintained at a constant temperature within the range of about 0° C. to about 80° C., preferably at about room temperature. Preferably, the base is added at a rate such that the reaction-mixture temperature remains within about one to two degrees of the initial reaction-mixture temperature. The base can be added as an organic solution or in undiluted form. Preferably, the base has a pKₐ of about 15 or greater. Suitable bases include, but are not limited to, alkylmetal bases such as methyllithium, n-butyllithium, tert-butyllithium, sec-butyllithium, phenyllithium, phenyl sodium, and phenyl potassium; metal amide bases such as lithium amide, sodium amide, potassium amide, lithium tetramethylpiperidide, lithium diisopropylamide, lithium diethylamide, lithium dicyclohexylamide, sodium hexamethyldisilazide, and lithium hexamethyldisilazide; and hydride bases such as sodium hydride and potassium hydride. The preferred base is sodium hydride. Suitable solvents include, but are not limited, to dimethyl sulfoxide, dichloromethane, ethers, and mixtures thereof, preferably tetrahydrofuran. After addition of the base, the reaction mixture is adjusted to within a temperature range of about 0° C. to about room temperature and compounds XXXV are added, preferably at a rate such that the reaction-mixture temperature remains within about one to two degrees of the initial reaction-mixture temperature. Compounds XXXV can be diluted in an organic solvent or added in undiluted form. The resulting reaction mixture is heated at a constant temperature within the range of about room temperature to about the solvent's boiling temperature until the reaction is substantially complete as determined by using an appropriate analytical method, preferably by gas chromatography. The product I can be isolated by workup and purification.

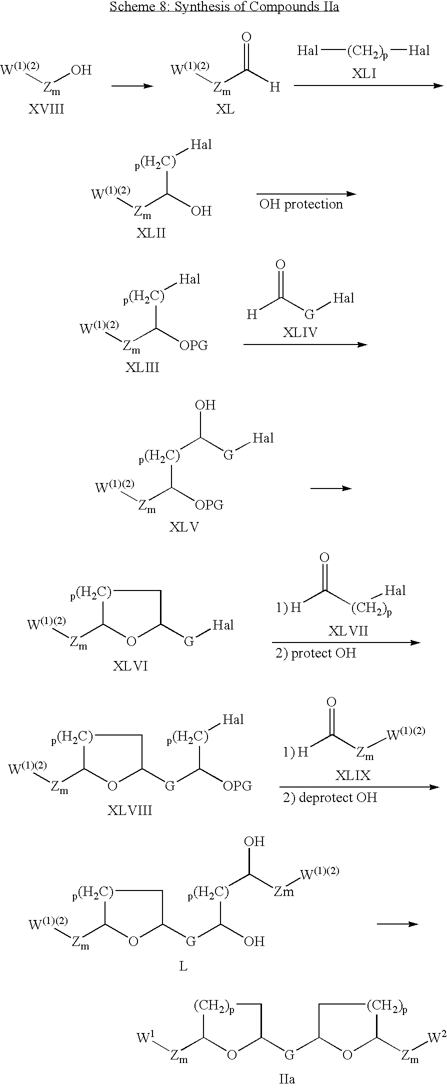

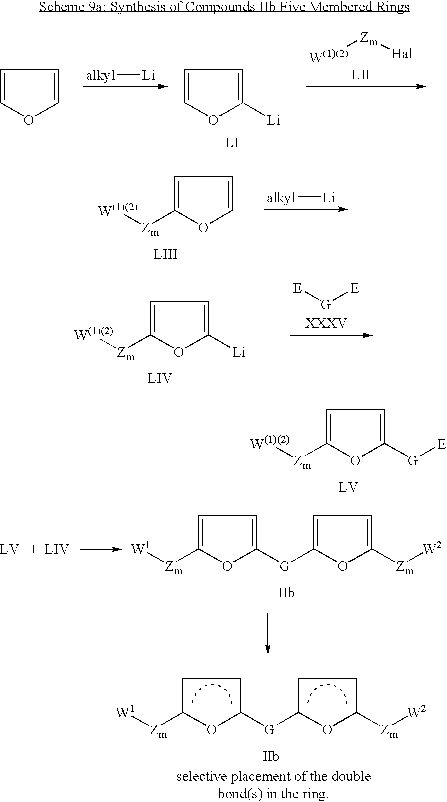

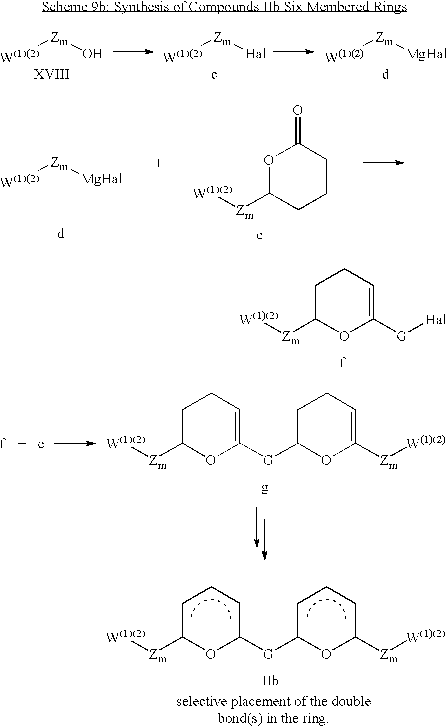

Scheme 8 depicts the synthesis of compounds IIa, that is, compounds of formula II where a double bond is not present in the ring. In the first step, compounds XVIII, prepared as discussed in Schemes 1 to 6 above, can be converted to compounds XL by standard oxidation of the primary alcohol to an aldehyde group. Such oxidations are described in M. Hudlicky, Oxidations in Organic Chemistry, ACS Monograph 186, 1990, pp. 114-127, incorporated by reference herein. In the next step Grignard reaction of XL with XLI followed by standard OH protection gives XLIII. Compounds XLI are commercially available (e.g., from Aldrich Chemical Co. Milwakee, Wis.) or readily prepared by standard synthetic methodology. For exemplary procedures for Grignard reaction see March, J. Advanced Organic Chemistry; Reactions Mechanisms, and Structure, 4th ed., 1992, pp. 920-929, incorporated herein by reference. Similarly, in the next step, the Grignard salt of XLIII is condensed with XLIV to provide XLV. Next XLV is cyclized to XLVI. When p is one, exemplary cyclization procedures are found in Friedrichsen, W. in Comprehensive Heterocyclic Chemistry II; Katritzky, A. R.; Rees, W. C.; Scriven, E. F. V. Eds.; Pergamon Press: Oxford, 1996; Vol.2, p 351, and Comprehensive Heterocyclic Chemistry; Katritzky, A. R.; Rees, W. C. Eds.; Pergamon Press: Oxford, 1986; Vol.3. When p is 0, cyclization procedures are found in Hepworth, J. D. in Comprehensive Heterocyclic Chemistry II; Katritzky, A. R.; Rees, W. C.; Scriven, E. F. V. Eds.; Pergamon Press: Oxford, 1996; Vol.5, p 351 and Comprehensive Heterocyclic Chemistry; Katritzky, A. R.; Rees, W. C. Eds.; Pergamon Press: Oxford, 1986; Vol.3, all of which citations are incorporated by reference herein.

Intermediates XLV and L can be cyclized as diols, or the newly introduced alcohol moiety is first oxidized to a ketone, then the hydroxy-protected ketone is subjected to cyclization, as described in the above Hepworth, J. D. in Comprehensive Heterocyclic Chemistry II; Katritzky, A. R.; Rees, W. C.; Scriven, E. F. V. Eds.; Pergamon Press: Oxford, 1996; Vol.5, p 386. For compounds II where W⁽¹⁾⁽²⁾ is HO(CH₂)ₙ—R¹R², the hydroxy group is first protected as described in Greene, T. W., Protective Groups in Organic Synthesis, 3rd edition(1999). For other structures, whereY is a group such as an acid, aldehydes, etc., protection is needed (acids as esters, preferably pivaloyl, aldehydes as silyl derivatives such as TIPS, stable in both basic and acidic conditions). When W⁽¹⁾⁽²⁾ is a lactone it can be introduced as discussed in Scheme 3 above.

Scheme 9a depicts the synthesis of compounds IIb, that is, compounds II where a double bond is present in the five membered ring. In the first step, the appropriate heterocycle is lithiated with an alkyl lithium base (alkyl-Li, e.g., butyl lithium or mixtures of alkyl lithiums with potassium t-butoxide, Wakefield, B. J., Organolithium Methods, Academic Press:London, 1998) by well known synthetic methods (for a review, see Katritzky Handbook of Heterocyclic Chemistry, Pergamon Press: Oxford 1985). Furan-type heterocycles are exclusively lithiated in the 2-position to provide compounds LI, which in turn are then reacted with electrophiles LII to produce derivatives LIII (Benkeser, R. A. et al., J. Amer. Chem. Soc. 1948, 70, 1780; Ramanathan, V. et al., J. Amer. Chem. Soc. 1962, 27, 1216; Chadwick, D. J. et al., J. Chem. Soc. Perkin 1 1977, 887; Feringa, B. L. et al., Synthesis 1988, 316, all of which citations are incorporated herein by reference). Lithiation is performed according to the literature methods, by reacting the heterocycles with alkyl-lithium derivatives such as methyl-lithium, n-,s-, or t-butyl-lithium in solvents such as ether, glyme or tetrahydrofuran, preferably ether. Preferably, ligands, such as TMEDA, DMPU or HMPA or another strong base, such as potassium t-butoxide are included in the reaction medium. Preferably, the reaction temperature is between −40° C. to +60° C., and the reaction time is about 1 to 5 hr. The heterocycles are available commercially or prepared by well-known synthetic methods. Next, in a similar fashion, LV is condensed with LIV to give IIb, wherein each ring has two double bonds. The reactions are performed under similar conditions for substituted heterocycles (for a review on lithiation of 2-substituted furans and thiophenes see Comprehensive Heterocyclic Chemistry; Katritzky, A. R.; Rees, W. C. Eds.; Pergamon Press: Oxford, 1986; Vol.3, p 771). After the formation of the metallated heterocylces, they are in situ reacted with electrophiles (e.g., LV) at temperatures between −40° C. to +60° C., for a reaction time of 1 hr to 5 days. The ring double bonds can be selectively reduced or otherwise manipulated by well known synthetic methods to give compounds IIb having only one selectively-placed double bond (i.e., the double bond can be positioned in the desired location within the ring), for example, the methods disclosed in March, J. Advanced Organic Chemistry; Reactions Mechanisms, and Structure, 4th ed., 1992, pp. 771-780, incorporated herein by reference.

Scheme 9b depicts the synthesis of compounds IIb, that is, compounds II where a double bond is present in the six membered ring. In the first step, compounds XVIII are converted to compounds c according to the halogenation procedure discussed for Scheme 1. Compound e, readily available by adaptation of the synthetic methods presented in Scheme 1 is reacted with the Grignard salt d to give f. For exemplary procedures for Grignard reactions see March, J. Advanced Organic Chemistry; Reactions Mechanisms, and Structure, 4th ed., 1992, pp. 920-929. In a similar fashion, Grignard reaction of f and e gives compounds g. The ring double bonds of g can be selectively reduced or otherwise manipulated by well known synthetic methods to give compounds IIb having only one selectively-placed double bond in the six-membered ring (i.e., the double bond can be positioned in the desired location within the ring), for example, the methods disclosed in March, J. Advanced Organic Chemistry; Reactions Mechanisms, and Structure, 4th ed., 1992, pp. 771-780, incorporated herein by reference.

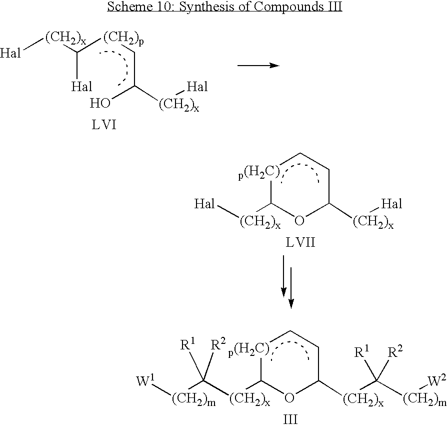

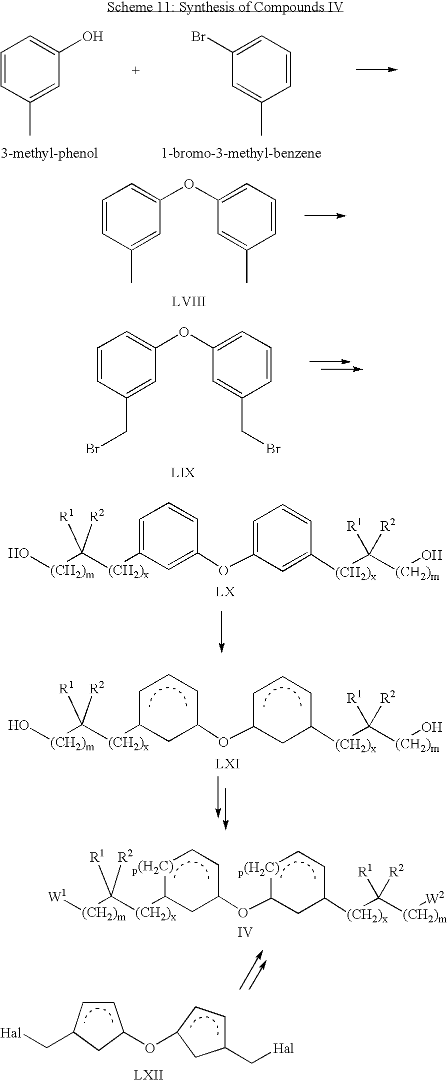

Scheme 10 depicts the general synthesis of compounds III. Compounds LVI where p is 1 or 2, are readily available either commercially (e.g., Aldrich Chemical Co. Milwaukee, Wis.) or by well known synthetic methods from readily available starting materials. Compounds LVI are cyclized to compounds LVII by well known cyclization methods. For example Hamonet, 1918, Ann. Chim. (Paris) 10:19, incorporated herein by reference. This cyclization can also be performed under the conditions of the Williamson ether synthesis discussed in detail for Scheme 7 and relying on the kinetic drive for 5 and 6-membered ring closure. Once general synthon LVII is obtained, it is a routine matter to convert it to the compounds III by adapting the chemistry discussed for Schemes 1 and 2.

Scheme 11 depicts the general synthesis of compounds IV. When p of compounds IV is 1, the first step involves Ullmann type coupling between 3-methyl-phenol and 1-bromo-3-methyl-benzene to give LVIII. The Ullmann reaction is well known, for example, see the procedures in March, J. Advanced Organic Chemistry; Reactions Mechanisms, and Structure, 4th ed., 1992, pp. 665, incorporated herein by reference. Next LVIII is oxidatively brominated in the benzylic position using well known methods, e.g., N-bromosuccinimide and benzoyl peroxide. Compounds LIX can then be converted to LX by adapting the methods discussed for Scheme 1. If desired, compounds LX can be selectively reduced or partially reduced to provide compounds LXI having mono and dienyl rings, according to well known procedures, see e.g., M. Hudlicky, Reductions in Organic Chemistry, ACS Monograph 188, 2nd ed., 1996, pp. 61-68 and 308-309, incorporated herein by reference. Compounds LX and LXI can be converted to compounds IV according to the methods discussed for Schemes 1 and 2. In a similar fashion, compounds LXII, available by well known synthetic methods, can be converted to compounds IV where p is 0.

### 5.3 Therapeutic Uses

In accordance with the invention, a compound of the invention or a composition of the invention, comprising a compound of the invention and a pharmaceutically acceptable vehicle, is administered to a patient, preferably a human, with or at risk of aging, Alzheimer's Disease, cancer, cardiovascular disease, diabetic nephropathy, diabetic retinopathy, a disorder of glucose metabolism, dyslipidemia, dyslipoproteinemia, enhancing bile production, enhancing reverse lipid transport, hypertension, impotence, inflammation, insulin resistance, lipid elimination in bile, modulating C reactive protein, obesity, oxysterol elimination in bile, pancreatitis, Parkinson's disease, a peroxisome proliferator activated receptor-associated disorder, phospholipid elimination in bile, renal disease, septicemia, metabolic syndrome disorders (e.g., Syndrome X), a thrombotic disorder, gastrointestinal disease, irritable bowel syndrome (IBS), inflammatory bowel disease (e.g., Crohn's Disease, ulcerative colitis), arthritis (e.g., rheumatoid arthritis, osteoarthritis), autoimmune disease (e.g., systemic lupus erythematosus), scleroderma, ankylosing spondylitis, gout and pseudogout, muscle pain: polymyositis/polymyalgia rheumatica/fibrositis; infection and arthritis, juvenile rheumatoid arthritis, tendonitis, bursitis and other soft tissue rheumatism. In one embodiment, “treatment” or “treating” refers to an amelioration of a disease or disorder, or at least one discernible symptom thereof. In another embodiment, “treatment” or “treating” refers to inhibiting the progression of a disease or disorder, either physically, e.g., stabilization of a discernible symptom, physiologically, e.g., stabilization of a physical parameter, or both.

In certain embodiments, the compounds of the invention or the compositions of the invention are administered to a patient, preferably a human, as a preventative measure against such diseases. As used herein, “prevention” or “preventing” refers to a reduction of the risk of acquiring a given disease or disorder. In a preferred mode of the embodiment, the compositions of the present invention are administered as a preventative measure to a patient, preferably a human having a genetic predisposition to a aging, Alzheimer's Disease, cancer, cardiovascular disease, diabetic nephropathy, diabetic retinopathy, a disorder of glucose metabolism, dyslipidemia, dyslipoproteinemia, enhancing bile production, enhancing reverse lipid transport, hypertension, impotence, inflammation, insulin resistance, lipid elimination in bile, modulating C reactive protein, obesity, oxysterol elimination in bile, pancreatitis, Parkinson's disease, a peroxisome proliferator activated receptor-associated disorder, phospholipid elimination in bile, renal disease, septicemia, metabolic syndrome disorders (e.g., Syndrome X), a thrombotic disorder, inflammatory processes and diseases like gastrointestinal disease, irritable bowel syndrome (IBS), inflammatory bowel disease (e.g., Crohn's Disease, ulcerative colitis), arthritis (e.g., rheumatoid arthritis, osteoarthritis), autoimmune disease (e.g., systemic lupus erythematosus), scleroderma, ankylosing spondylitis, gout and pseudogout, muscle pain: polymyositis/polymyalgia rheumatica/fibrositis; infection and arthritis, juvenile rheumatoid arthritis, tendonitis, bursitis and other soft tissue rheumatism. Examples of such genetic predispositions include but are not limited to the ε4 allele of apolipoprotein E, which increases the likelihood of Alzheimer's Disease; a loss of function or null mutation in the lipoprotein lipase gene coding region or promoter (e.g., mutations in the coding regions resulting in the substitutions D9N and N291S; for a review of genetic mutations in the lipoprotein lipase gene that increase the risk of cardiovascular diseases, dyslipidemias and dyslipoproteinemias, see Hayden and Ma, 1992, Mol. Cell Biochem. 113:171-176); and familial combined hyperlipidemia and familial hypercholesterolemia.

In another preferred mode of the embodiment, the compounds of the invention or compositions of the invention are administered as a preventative measure to a patient having a non-genetic predisposition to a aging, Alzheimer's Disease, cancer, cardiovascular disease, diabetic nephropathy, diabetic retinopathy, a disorder of glucose metabolism, dyslipidemia, dyslipoproteinemia, enhancing bile production, enhancing reverse lipid transport, hypertension, impotence, inflammation, insulin resistance, lipid elimination in bile, modulating C reactive protein, obesity, oxysterol elimination in bile, pancreatitis, Parkinson's disease, a peroxisome proliferator activated receptor-associated-disorder, phospholipid elimination in bile, renal disease, septicemia, metabolic syndrome disorders (e.g., Syndrome X), a thrombotic disorder, inflammatory processes and diseases like gastrointestinal disease, irritable bowel syndrome (IBS), inflammatory bowel disease (e.g., Crohn's Disease, ulcerative colitis), arthritis (e.g., rheumatoid arthritis, osteoarthritis), autoimmune disease (e.g., systemic lupus erythematosus), scleroderma, ankylosing spondylitis, gout and pseudogout, muscle pain: polymyositis/polymyalgia rheumatica/fibrositis; infection and arthritis, juvenile rheumatoid arthritis, tendonitis, bursitis and other soft tissue rheumatism. Examples of such non-genetic predispositions include but are not limited to cardiac bypass surgery and percutaneous transluminal coronary angioplasty, which often lead to restenosis, an accelerated form of atherosclerosis; diabetes in women, which often leads to polycystic ovarian disease; and cardiovascular disease, which often leads to impotence. Accordingly, the compositions of the invention may be used for the prevention of one disease or disorder and concurrently treating another (e.g., prevention of polycystic ovarian disease while treating diabetes; prevention of impotence while treating a cardiovascular disease).

5.3.1 Treatment of Cardiovascular Diseases

The present invention provides methods for the treatment or prevention of a cardiovascular disease, comprising administering to a patient a therapeutically effective amount of a compound or a composition comprising a compound of the invention and a pharmaceutically acceptable vehicle. As used herein, the term “cardiovascular diseases” refers to diseases of the heart and circulatory system. These diseases are often associated with dyslipoproteinemias and/or dyslipidemias. Cardiovascular diseases which the compositions of the present invention are useful for preventing or treating include but are not limited to arteriosclerosis; atherosclerosis; stroke; ischemia; endothelium dysfunctions, in particular those dysfunctions affecting blood vessel elasticity; peripheral vascular disease; coronary heart disease; myocardial infarcation; cerebral infarction and restenosis.

5.3.2 Treatment of Dyslipidemias

The present invention provides methods for the treatment or prevention of a dyslipidemia comprising administering to a patient a therapeutically effective amount of a compound or a composition comprising a compound of the invention and a pharmaceutically acceptable vehicle.

As used herein, the term “dyslipidemias” refers to disorders that lead to or are manifested by aberrant levels of circulating lipids. To the extent that levels of lipids in the blood are too high, the compositions of the invention are administered to a patient to restore normal levels. Normal levels of lipids are reported in medical treatises known to those of skill in the art. For example, recommended blood levels of LDL, HDL, free triglycerides and others parameters relating to lipid metabolism can be found at the web site of the American Heart Association and that of the National Cholesterol Education Program of the National Heart, Lung and Blood Institute (http://www.americanheart.org/cholesterol/about_level.html and http://www.nhlbi.nih.gov/health/public/heart/chol/hbc_what.html, respectively). At the present time, the recommended level of HDL cholesterol in the blood is above 35 mg/dL; the recommended level of LDL cholesterol in the blood is below 130 mg/dL; the recommended LDL:HDL cholesterol ratio in the blood is below 5:1, ideally 3.5:1; and the recommended level of free triglycerides in the blood is less than 200 mg/dL.

Dyslipidemias which the compositions of the present invention are useful for preventing or treating include but are not limited to hyperlipidemia and low blood levels of high density lipoprotein (HDL) cholesterol. In certain embodiments, the hyperlipidemia for prevention or treatment by the compounds of the present invention is familial hypercholesterolemia; familial combined hyperlipidemia; reduced or deficient lipoprotein lipase levels or activity, including reductions or deficiencies resulting from lipoprotein lipase mutations; hypertriglyceridemia; hypercholesterolemia; high blood levels of urea bodies (e.g. β-OH butyric acid); high blood levels of Lp(a) cholesterol; high blood levels of low density lipoprotein (LDL) cholesterol; high blood levels of very low density lipoprotein (VLDL) cholesterol and high blood levels of non-esterified fatty acids.

The present invention further provides methods for altering lipid metabolism in a patient, e.g., reducing LDL in the blood of a patient, reducing free triglycerides in the blood of a patient, increasing the ratio of HDL to LDL in the blood of a patient, and inhibiting saponified and/or non-saponified fatty acid synthesis, said methods comprising administering to the patient a compound or a composition comprising a compound of the invention in an amount effective alter lipid metabolism.

5.3.3 Treatment of Dyslipoproteinemias

The present invention provides methods for the treatment or prevention of a dyslipoproteinemia comprising administering to a patient a therapeutically effective amount of a compound or a composition comprising a compound of the invention and a pharmaceutically acceptable vehicle.

As used herein, the term “dyslipoproteinemias” refers to disorders that lead to or are manifested by aberrant levels of circulating lipoproteins. To the extent that levels of lipoproteins in the blood are too high, the compositions of the invention are administered to a patient to restore normal levels. Conversely, to the extent that levels of lipoproteins in the blood are too low, the compositions of the invention are administered to a patient to restore normal levels. Normal levels of lipoproteins are reported in medical treatises known to those of skill in the art.

Dyslipoproteinemias which the compositions of the present invention are useful for preventing or treating include but are not limited to high blood levels of LDL; high blood levels of apolipoprotein B (apo B); high blood levels of Lp(a); high blood levels of apo(a); high blood levels of VLDL; low blood levels of HDL; reduced or deficient lipoprotein lipase levels or activity, including reductions or deficiencies resulting from lipoprotein lipase mutations; hypoalphalipoproteinemia; lipoprotein abnormalities associated with diabetes; lipoprotein abnormalities associated with obesity; lipoprotein abnormalities associated with Alzheimer's Disease; and familial combined hyperlipidemia.

The present invention further provides methods for reducing apo C-II levels in the blood of a patient; reducing apo C-III levels in the blood of a patient; elevating the levels of HDL associated proteins, including but not limited to apo A-I, apo A-II, apo A-IV and apo E in the blood of a patient; elevating the levels of apo E in the blood of a patient, and promoting clearance of triglycerides from the blood of a patient, said methods comprising administering to the patient a compound or a composition comprising a compound of the invention in an amount effective to bring about said reduction, elevation or promotion, respectively.

5.3.4 Treatment of Glucose Metabolism Disorders

The present invention provides methods for the treatment or prevention of a glucose metabolism disorder, comprising administering to a patient a therapeutically effective amount of a compound or a composition comprising a compound of the invention and a pharmaceutically acceptable vehicle. As used herein, the term “glucose metabolism disorders” refers to disorders that lead to or are manifested by aberrant glucose storage and/or utilization. To the extent that indicia of glucose metabolism (i.e., blood insulin, blood glucose) are too high, the compositions of the invention are administered to a patient to restore normal levels. Conversely, to the extent that indicia of glucose metabolism are too low, the compositions of the invention are administered to a patient to restore normal levels. Normal indicia of glucose metabolism are reported in medical treatises known to those of skill in the art.

Glucose metabolism disorders which the compositions of the present invention are useful for preventing or treating include but are not limited to impaired glucose tolerance; insulin resistance; insulin resistance related breast, colon or prostate cancer; diabetes, including but not limited to non-insulin dependent diabetes mellitus (NIDDM), insulin dependent diabetes mellitus (IDDM), gestational diabetes mellitus (GDM), and maturity onset diabetes of the young (MODY); pancreatitis; hypertension; polycystic ovarian disease; and high levels of blood insulin and/or glucose.

The present invention further provides methods for altering glucose metabolism in a patient, for example to increase insulin sensitivity and/or oxygen consumption of a patient, said methods comprising administering to the patient a compound or a composition comprising a compound of the invention in an amount effective to alter glucose metabolism.

5.3.5 Treatment of PPAR-Associated Disorders

The present invention provides methods for the treatment or prevention of a PPAR-associated disorder, comprising administering to a patient a therapeutically effective amount of a compound or a composition comprising a compound of the invention and a pharmaceutically acceptable vehicle. As used herein, “treatment or prevention of PPAR associated disorders” encompasses treatment or prevention of rheumatoid arthritis; multiple sclerosis; psoriasis; inflammatory bowel diseases; breast; colon or prostate cancer; low levels of blood HDL; low levels of blood, lymph and/or cerebrospinal fluid apo E; low blood, lymph and/or cerebrospinal fluid levels of apo A-I; high levels of blood VLDL; high levels of blood LDL; high levels of blood triglyceride; high levels of blood apo B; high levels of blood apo C-III and reduced ratio of post-heparin hepatic lipase to lipoprotein lipase activity. HDL may be elevated in lymph and/or cerebral fluid.

5.3.6 Treatment of Renal Diseases

The present invention provides methods for the treatment or prevention of a renal disease, comprising administering to a patient a therapeutically effective amount of a compound or a composition comprising a compound of the invention and a pharmaceutically acceptable vehicle. Renal diseases that can be treated by the compounds of the present invention include glomerular diseases (including but not limited to acute and chronic glomerulonephritis, rapidly progressive glomerulonephritis, nephrotic syndrome, focal proliferative glomerulonephritis, glomerular lesions associated with systemic disease, such as systemic lupus erythematosus, Goodpasture's syndrome, multiple myeloma, diabetes, neoplasia, sickle cell disease, and chronic inflammatory diseases), tubular diseases (including but not limited to acute tubular necrosis and acute renal failure, polycystic renal diseasemedullary sponge kidney, medullary cystic disease, nephrogenic diabetes, and renal tubular acidosis), tubulointerstitial diseases (including but not limited to pyelonephritis, drug and toxin induced tubulointerstitial nephritis, hypercalcemic nephropathy, and hypokalemic nephropathy) acute and rapidly progressive renal failure, chronic renal failure, nephrolithiasis, or tumors (including but not limited to renal cell carcinoma and nephroblastoma). In a most preferred embodiment, renal diseases that are treated by the compounds of the present invention are vascular diseases, including but not limited to hypertension, nephrosclerosis, microangiopathic hemolytic anemia, atheroembolic renal disease, diffuse cortical necrosis, and renal infarcts.

5.3.7 Treatment of Cancer

The present invention provides methods for the treatment or prevention of cancer, comprising administering to a patient a therapeutically effective amount of a compound or a composition comprising a compound of the invention and a pharmaceutically acceptable vehicle. Types of cancer that can be treated using a Compound of the Invention include, but are not limited to, those listed in Table 2.

TABLE 2

Solid tumors, including but not limited to

fibrosarcoma
myxosarcoma
liposarcoma
chondrosarcoma
osteogenic sarcoma
chordoma
angiosarcoma
endotheliosarcoma
lymphangiosarcoma
lymphangioendotheliosarcoma
synovioma
mesothelioma
Ewing's tumor
leiomyosarcoma
rhabdomyosarcoma
colon cancer
colorectal cancer
kidney cancer
pancreatic cancer
bone cancer
breast cancer
ovarian cancer
prostate cancer
esophogeal cancer
stomach cancer
oral cancer
nasal cancer
throat cancer
squamous cell carcinoma
basal cell carcinoma
adenocarcinoma
sweat gland carcinoma
sebaceous gland carcinoma
papillary carcinoma
papillary adenocarcinomas
cystadenocarcinoma
medullary carcinoma
bronchogenic carcinoma
renal cell carcinoma
hepatoma
bile duct carcinoma
choriocarcinoma
seminoma
embryonal carcinoma
Wilms' tumor
cervical cancer
uterine cancer
testicular cancer
small cell lung carcinoma
bladder carcinoma
lung cancer
epithelial carcinoma
glioma
glioblastoma multiforme
astrocytoma
medulloblastoma
craniopharyngioma
ependymoma
pinealoma
hemangioblastoma
acoustic neuroma
oligodendroglioma
meningioma
skin cancer
melanoma
neuroblastoma
retinoblastoma

Blood-borne cancers, including but not limited to:

acute lymphoblastic B-cell leukemia
acute lymphoblastic T-cell leukemia
acute myeloblastic leukemia “AML”
acute promyelocytic leukemia “APL”
acute monoblastic leukemia
acute erythroleukemic leukemia
acute megakaryoblastic leukemia
acute myelomonocytic leukemia
acute nonlymphocyctic leukemia
acute undifferentiated leukemia
chronic myelocytic leukemia “CML”
chronic lymphocytic leukemia “CLL”
hairy cell leukemia
multiple myeloma

Acute and chronic leukemias

Lymphoblastic
myelogenous
lymphocytic
myelocytic leukemias

Lymphomas:

Hodgkin's disease
non-Hodgkin's Lymphoma
Multiple myeloma
Waldenstrom's macroglobulinemia
Heavy chain disease
Polycythemia vera

Cancer, including, but not limited to, a tumor, metastasis, or any disease or disorder characterized by uncontrolled cell growth, can be treated or prevented by administration of a Compound of the Invention.

5.3.8 Treatment of Other Diseases

The present invention provides methods for the treatment or prevention of Alzheimer's Disease, Syndrome X, septicemia, thrombotic disorders, obesity, pancreatitis, hypertension, inflammation, and impotence, comprising administering to a patient a therapeutically effective amount of a compound or a composition comprising a compound of the invention and a pharmaceutically acceptable vehicle.

As used herein, “treatment or prevention of Alzheimer's Disease” encompasses treatment or prevention of lipoprotein abnormalities associated with Alzheimer's Disease.

As used herein, “treatment or prevention of Syndrome X or Metabolic Syndrome” encompasses treatment or prevention of a symptom thereof, including but not limited to impaired glucose tolerance, hypertension and dyslipidemia/dyslipoproteinemia.

As used herein, “treatment or prevention of septicemia” encompasses treatment or prevention of septic shock.

As used herein, “treatment or prevention of thrombotic disorders” encompasses treatment or prevention of high blood levels of fibrinogen and promotion of fibrinolysis.

In addition to treating or preventing obesity, the compositions of the invention can be administered to an individual to promote weight reduction of the individual.

As used herein, “treatment or prevention of diabetic nephropathy” encompasses treating or preventing kidney disease that develops as a result of diabetes mellitus (DM). Diabetes mellitus is a disorder in which the body is unable to metabolize carbohydrates (e.g., food starches, sugars, cellulose) properly. The disease is characterized by excessive amounts of sugar in the blood (hyperglycemia) and urine; inadequate production and/or utilization of insulin; and by thirst, hunger, and loss of weight. Thus, the compounds of the invention can also be used to treat or prevent diabetes mellitus.

As used herein, “treatment or prevention of diabetic retinopathy” encompasses treating or preventing complications of diabetes that lead to or cause blindness. Diabetic retinopathy occurs when diabetes damages the tiny blood vessels inside the retina, the light-sensitive tissue at the back of the eye.

As used herein, “treatment or prevention of impotence” includes treating or preventing erectile dysfunction, which encompasses the repeated inability to get or keep an erection firm enough for sexual intercourse. The word “impotence” may also be used to describe other problems that interfere with sexual intercourse and reproduction, such as lack of sexual desire and problems with ejaculation or orgasm. The term “treatment or prevention of impotence includes, but is not limited to impotence that results as a result of damage to nerves, arteries, smooth muscles, and fibrous tissues, or as a result of disease, such as, but not limited to, diabetes, kidney disease, chronic alcoholism, multiple sclerosis, atherosclerosis, vascular disease, and neurologic disease.

As used herein, “treatment or prevention of hypertension” encompasses treating or preventing blood flow through the vessels at a greater than normal force, which strains the heart; harms the arteries; and increases the risk of heart attack, stroke, and kidney problems. The term hypertension includes, but is not limited to, cardiovascular disease, essential hypertension, hyperpiesia, hyperpiesis, malignant hypertension, secondary hypertension, or white-coat hypertension.

As used herein, “treatment or prevention of inflammation” encompasses treating or preventing inflammation diseases including, but not limited to, chronic inflammatory disorders of the joints including arthritis, e.g., rheumatoid arthritis and osteoarthritis; respiratory distress syndrome, inflammatory bowel diseases such as ileitis, ulcerative colitis and Crohn's disease; and inflammatory lung disorders such as asthma and chronic obstructive airway disease, inflammatory disorders of the eye such as corneal dystrophy, trachoma, onchocerciasis, uveitis, sympathetic ophthalmitis, and endophthalmitis; inflammatory disorders of the gum, e.g., periodontitis and gingivitis; tuberculosis; leprosy; inflammatory diseases of the kidney including glomerulonephritis and nephrosis; inflammatory disorders of the skin including acne, sclerodermatitis, psoriasis, eczema, photoaging and wrinkles; inflammatory diseases of the central nervous system, including AIDS-related neurodegeneration, stroke, neurotrauma, Alzheimer's disease, encephalomyelitis and viral or autoimmune encephalitis; autoimmune diseases including immune-complex vasculitis, systemic lupus and erythematodes; systemic lupus erythematosus (SLE); and inflammatory diseases of the heart such as cardiomyopathy.

### 5.4 Combination Therapy

In certain embodiments of the present invention, the compounds and compositions of the invention can be used in combination therapy with at least one other therapeutic agent. The compound of the invention and the therapeutic agent can act additively or, more preferably, synergistically. In a preferred embodiment, a compound or a composition comprising a compound of the invention is administered concurrently with the administration of another therapeutic agent, which can be part of the same composition as the compound of the invention or a different composition. In another embodiment, a compound or a composition comprising a compound of the invention is administered prior or subsequent to administration of another therapeutic agent. As many of the disorders for which the compounds and compositions of the invention are useful in treating are chronic disorders, in one embodiment combination therapy involves alternating between administering a compound or a composition comprising a compound of the invention and a composition comprising another therapeutic agent, e.g., to minimize the toxicity associated with a particular drug. The duration of administration of each drug or therapeutic agent can be, e.g., one month, three months, six months, or a year. In certain embodiments, when a composition of the invention is administered concurrently with another therapeutic agent that potentially produces adverse side effects including but not limited to toxicity, the therapeutic agent can advantageously be administered at a dose that falls below the threshold at which the adverse side is elicited.

The present compositions can be administered together with a statin. Statins for use in combination with the compounds and compositions of the invention include but are not limited to atorvastatin, pravastatin, fluvastatin, lovastatin, simvastatin, and cerivastatin.

The present compositions can also be administered together with a PPAR agonist, for example a thiazolidinedione or a fibrate. Thiazolidinediones for use in combination with the compounds and compositions of the invention include but are not limited to 5 ((4(2(methyl2pyridinylamino)ethoxy)phenyl)methyl)2,4thiazolidinedione, troglitazone, pioglitazone, ciglitazone, WAY 120,744, englitazone, AD 5075, darglitazone, and rosiglitazone. Fibrates for use in combination with the compounds and compositions of the invention include but are not limited to gemfibrozil, fenofibrate, clofibrate, or ciprofibrate. As mentioned previously, a therapeutically effective amount of a fibrate or thiazolidinedione often has toxic side effects. Accordingly, in a preferred embodiment of the present invention, when a composition of the invention is administered in combination with a PPAR agonist, the dosage of the PPAR agonist is below that which is accompanied by toxic side effects.

The present compositions can also be administered together with a bile acid binding resin. Bile acid binding resins for use in combination with the compounds and compositions of the invention include but are not limited to cholestyramine and colestipol hydrochloride. The present compositions can also be administered together with niacin or nicotinic acid. The present compositions can also be administered together with a RXR agonist. RXR agonists for use in combination with the compounds of the invention include but are not limited to LG 100268, LGD 1069, 9-cis retinoic acid, 2(1(3,5,5,8,8pentamethyl 5,6,7,8 tetrahydro 2 naphthyl) cyclopropyl) pyridine 5 carboxylic acid, or 4((3,5,5,8,8pentamethyl5,6,7,8tetrahydro2naphthyl)2carbonyl) benzoic acid. The present compositions can also be administered together with an anti-obesity drug. Anti-obesity drugs for use in combination with the compounds of the invention include but are not limited to β-adrenergic receptor agonists, preferably β-3 receptor agonists, fenfluramine, dexfenfluramine, sibutramine, bupropion, fluoxetine, and phentermine. The present compositions can also be administered together with a hormone. Hormones for use in combination with the compounds of the invention include but are not limited to thyroid hormone, estrogen and insulin. Preferred insulins include but are not limited to injectable insulin, transdermal insulin, inhaled insulin, or any combination thereof. As an alternative to insulin, an insulin derivative, secretagogue, sensitizer or mimetic may be used. Insulin secretagogues for use in combination with the compounds of the invention include but are not limited to forskolin, dibutryl cAMP or isobutylmethylxanthine (IBMX).

The present compositions can also be administered together with a phosphodiesterase type 5 (“PDE5”) inhibitor to treat or prevent disorders, such as but not limited to, impotence. In a particular, embodiment the combination is a synergistic combination of a composition of the invention and a PDE5 inhibitor.

The present compositions can also be administered together with a tyrophostine or an analog thereof. Tyrophostines for use in combination with the compounds of the invention include but are not limited to tryophostine 51.

The present compositions can also be administered together with sulfonylurea-based drugs. Sulfonylurea-based drugs for use in combination with the compounds of the invention include, but are not limited to, glisoxepid, glyburide, acetohexamide, chlorpropamide, glibornuride, tolbutamide, tolazamide, glipizide, gliclazide, gliquidone, glyhexamide, phenbutamide, and tolcyclamide. The present compositions can also be administered together with a biguanide. Biguanides for use in combination with the compounds of the invention include but are not limited to metformin, phenformin and buformin.

The present compositions can also be administered together with an α-glucosidase inhibitor. α-glucosidase inhibitors for use in combination with the compounds of the invention include but are not limited to acarbose and miglitol.

The present compositions can also be administered together with an apo A-I agonist. In one embodiment, the apo A-I agonist is the Milano form of apo A-I (apo A-IM). In a preferred mode of the embodiment, the apo A-IM for administration in conjunction with the compounds of the invention is produced by the method of U.S. Pat. No. 5,721,114 to Abrahamsen. In a more preferred embodiment, the apo A-I agonist is a peptide agonist. In a preferred mode of the embodiment, the apo A-I peptide agonist for administration in conjunction with the compounds of the invention is a peptide of U.S. Pat. No. 6,004,925 or U. S. Pat. No. 6,037,323 to Dasseux.

The present compositions can also be administered together with apolipoprotein E (apo E). In a preferred mode of the embodiment, the apoE for administration in conjunction with the compounds of the invention is produced by the method of U.S. Pat. No. 5,834,596 to Ageland.

In yet other embodiments, the present compositions can be administered together with an HDL-raising drug; an HDL enhancer; or a regulator of the apolipoprotein A-I, apolipoprotein A-IV and/or apolipoprotein genes.

In one embodiment, the other therapeutic agent can be an antiemetic agent. Suitable antiemetic agents include, but are not limited to, metoclopromide, domperidone, prochlorperazine, promethazine, chlorpromazine, trimethobenzamide, ondansetron, granisetron, hydroxyzine, acethylleucine monoethanolamine, alizapride, azasetron, benzquinamide, bietanautine, bromopride, buclizine, clebopride, cyclizine, dimenhydrinate, diphenidol, dolasetron, meclizine, methallatal, metopimazine, nabilone, oxyperndyl, pipamazine, scopolamine, sulpiride, tetrahydrocannabinols, thiethylperazine, thioproperazine andtropisetron.

In another embodiment, the other therapeutic agent can be an hematopoietic colony stimulating factor. Suitable hematopoietic colony stimulating factors include, but are not limited to, filgrastim, sargramostim, molgramostim and erythropoietin alfa.

In still another embodiment, the other therapeutic agent can be an opioid or non-opioid analgesic agent. Suitable opioid analgesic agents include, but are not limited to, morphine, heroin, hydromorphone, hydrocodone, oxymorphone, oxycodone, metopon, apomorphine, normorphine, etorphine, buprenorphine, meperidine, lopermide, anileridine, ethoheptazine, piminidine, betaprodine, diphenoxylate, fentanil, sufentanil, alfentanil, remifentanil, levorphanol, dextromethorphan, phenazocine, pentazocine, cyclazocine, methadone, isomethadone and propoxyphene. Suitable non-opioid analgesic agents include, but are not limited to, aspirin, celecoxib, rofecoxib, diclofinac, diflusinal, etodolac, fenoprofen, flurbiprofen, ibuprofen, ketoprofen, indomethacin, ketorolac, meclofenamate, mefanamic acid, nabumetone, naproxen, piroxicam and sulindac.

5.4.1 Combination Therapy for Cardiovascular Diseases

The present compositions can be administered together with a known cardiovascular drug. Cardiovascular drugs for use in combination with the compounds of the invention to prevent or treat cardiovascular diseases include but are not limited to peripheral antiadrenergic drugs, centrally acting antihypertensive drugs (e.g., methyldopa, methyldopa HCl), antihypertensive direct vasodilators (e.g., diazoxide, hydralazine HCl), drugs affecting renin-angiotensin system, peripheral vasodilators, phentolamine, antianginal drugs, cardiac glycosides, inodilators (e.g., amrinone, milrinone, enoximone, fenoximone, imazodan, sulmazole), antidysrhythmic drugs, calcium entry blockers, ranitine, bosentan, and rezulin.

5.4.2 Combination Therapy for Cancer

The present invention includes methods for treating cancer, comprising administering to an animal in need thereof an effective amount of a Compound of the Invention and another therapeutic agent that is an anti-cancer agent. Suitable anticancer agents include, but are not limited to, those listed in Table 3.

TABLE 3

| | |
|---|---|
| Alkylating agents | |
| Nitrogen mustards: | Cyclophosphamide |
| | Ifosfamide |
| | trofosfamide |
| | Chlorambucil |
| | Treos |
| Nitrosoureas: | carbustine (BCNU) |
| | Lomustine (CCNU) |
| Alkylsulphonates | Busulfan |
| | Treosulfan |
| Triazenes: | Dacarbazine |
| Platinum containing compounds: | Cisplatin |
| | carboplatin |
| Plant Alkaloids | |
| Vinca alkaloids: | Vicristine |
| | Vinblastine |
| | Vindesine |
| | Vinorelbine |
| Taxoids: | paclitaxel |
| | Docetaxol |
| DNA Topoisomerase Inhibitors | |
| Epipodophyllins: | Etoposide |
| | Teniposide |
| | Topotecan |
| | 9-aminocamptothecin |
| | camptothecin |
| | crisnatol |
| mitomycins: | Mitomycin C |
| Anti-metabolites | |
| Anti-folates: | |
| DHFR inhibitors: | METHOTREXATE |
| | Trimetrexate |
| IMP dehydrogenase Inhibitors: | Mycophenolic acid |
| | Tiazofurin |
| | Ribavirin |
| | EICAR |
| Ribonuclotide reductase Inhibitors: | Hydroxyurea |
| | deferoxamine |
| Pyrimidine analogs: | |
| Uracil analogs | 5-Fluorouracil |
| | Floxuridine |
| | Doxifluridine |
| | Ratitrexed |
| Cytosine analogs | cytarabine (ara C) |
| | Cytosine arabinoside |
| | fludarabine |
| Purine analogs: | mercaptopurine |
| | Thioguanine |
| Hormonal therapies: | |
| Receptor antagonists: | |
| Anti-estrogen | Tamoxifen |
| | Raloxifene |
| | megestrol |
| | goscrclin |
| | Leuprolide acetate |
| LHRH agonists: | flutamide |
| | bicalutamide |
| Retinoids/Deltoids | |
| Vitamin D3 analogs: | EB 1089 |
| | CB 1093 |
| | KH 1060 |
| Photodynamic therapies: | vertoporfin (BPD-MA) |
| | Phthalocyanine |
| | photosensitizer Pc4 |
| | Demethoxy-hypocrellin A |
| | (2BA-2-DMHA) |
| Cytokines: | Interferon-α |
| | Interferon-γ |
| | Tumor necrosis factor |
| Others: | |
| Isoprenylation inhibitors: | Lovastatin |
| Dopaminergic neurotoxins: | 1-methyl-4-phenylpyridinium ion |
| Cell cycle inhibitors: | staurosporine |
| Actinomycines: | Actinomycin D |
| | Dactinomycin |
| Bleomycins: | bleomycin A2 |
| | Bleomycin B2 |
| | Peplomycin |
| Anthracyclines: | daunorubicin |
| | Doxorubicin (adriamycin) |
| | Idarubicin |
| | Epirubicin |
| | Pirarubicin |
| | Zorubicin |
| | Mitoxantrone |
| MDR inhibitors | verapamil |
| Ca²⁺ ATPase inhibitors: | thapsigargin |

In a specific embodiment, a composition of the invention further comprises one or more chemotherapeutic agents and/or is administered concurrently with radiation therapy. In another specific embodiment, chemotherapy or radiation therapy is administered prior or subsequent to administration of a present composition, preferably at least an hour, five hours, 12 hours, a day, a week, a month, more preferably several months (e.g., up to three months), subsequent to administration of a composition of the invention.

In other embodiments, the invention provides methods for treating or preventing cancer, comprising administering to an animal in need thereof an effective amount of a Compound of the Invention and a chemotherapeutic agent. In one embodiment the chemotherapeutic agent is that with which treatment of the cancer has not been found to be refractory. In another embodiment, the chemotherapeutic agent is that with which the treatment of cancer has been found to be refractory. The Compounds of the Invention can be administered to an animal that has also undergone surgery as treatment for the cancer.

In one embodiment, the additional method of treatment is radiation therapy.

In a specific embodiment, the Compound of the Invention is administered concurrently with the chemotherapeutic agent or with radiation therapy. In another specific embodiment, the chemotherapeutic agent or radiation therapy is administered prior or subsequent to administration of a Compound of the Invention, preferably at least an hour, five hours, 12 hours, a day, a week, a month, more preferably several months (e.g., up to three months), prior or subsequent to administration of a Compound of the Invention.

A chemotherapeutic agent can be administered over a series of sessions, any one or a combination of the chemotherapeutic agents listed in Table 3 can be administered. With respect to radiation, any radiation therapy protocol can be used depending upon the type of cancer to be treated. For example, but not by way of limitation, x-ray radiation can be administered; in particular, high-energy megavoltage (radiation of greater that 1 MeV energy) can be used for deep tumors, and electron beam and orthovoltage x-ray radiation can be used for skin cancers. Gamma-ray emitting radioisotopes, such as radioactive isotopes of radium, cobalt and other elements, can also be administered.

Additionally, the invention provides methods of treatment of cancer with a Compound of the Invention as an alternative to chemotherapy or radiation therapy where the chemotherapy or the radiation therapy has proven or can prove too toxic, e.g., results in unacceptable or unbearable side effects, for the subject being treated. The animal being treated can, optionally, be treated with another cancer treatment such as surgery, radiation therapy or chemotherapy, depending on which treatment is found to be acceptable or bearable.

The Compounds of the Invention can also be used in an in vitro or ex vivo fashion, such as for the treatment of certain cancers, including, but not limited to leukemias and lymphomas, such treatment involving autologous stem cell transplants. This can involve a multi-step process in which the animal's autologous hematopoietic stem cells are harvested and purged of all cancer cells, the patient's remaining bone-marrow cell population is then eradicated via the administration of a high dose of a Compound of the Invention with or without accompanying high dose radiation therapy, and the stem cell graft is infused back into the animal. Supportive care is then provided while bone marrow function is restored and the animal recovers.

### 5.5 Surgical Uses

Cardiovascular diseases such as atherosclerosis often require surgical procedures such as angioplasty. Angioplasty is often accompanied by the placement of a reinforcing a metallic tube shaped structure known as a “stent” into a damaged coronary artery. For more serious conditions, open heart surgery such as coronary bypass surgery may be required. These surgical procedures entail using invasive surgical devices and/or implants, and are associated with a high risk of restenosis and thrombosis. Accordingly, the compounds and compositions of the invention may be used as coatings on surgical devices (e.g., catheters) and implants (e.g., stents) to reduce the risk of restenosis and thrombosis associated with invasive procedures used in the treatment of cardiovascular diseases.

### 5.6 Veterinary and Livestock Uses

A composition of the invention can be administered to a non-human animal for a veterinary use for treating or preventing a disease or disorder disclosed herein.

In a specific embodiment, the non-human animal is a household pet. In another specific embodiment, the non-human animal is a livestock animal. In a preferred embodiment, the non-human animal is a mammal, most preferably a cow, horse, sheep, pig, cat, dog, mouse, rat, rabbit, or guinea pig. In another preferred embodiment, the non-human animal is a fowl species, most preferably a chicken, turkey, duck, goose, or quail.

In addition to veterinary uses, the compounds and compositions of the invention can be used to reduce the fat content of livestock to produce leaner meats. Alternatively, the compounds and compositions of the invention can be used to reduce the cholesterol content of eggs by administering the compounds to a chicken, quail, or duck hen. For non-human animal uses, the compounds and compositions of the invention can be administered via the animals' feed or orally as a drench composition.

5.7 Therapeutic/Prophylactic Administration and Compositions

Due to the activity of the compounds and compositions of the invention, they are useful in veterinary and human medicine. As described above, the compounds and compositions of the invention are useful for the treatment or prevention of aging, Alzheimer's Disease, cancer, cardiovascular disease, diabetic nephropathy, diabetic retinopathy, a disorder of glucose metabolism, dyslipidemia, dyslipoproteinemia, hypertension, impotence, inflammation, insulin resistance, lipid elimination in bile, modulating C reactive protein, obesity, oxysterol elimination in bile, pancreatitis, Parkinson's disease, a peroxisome proliferator activated receptor-associated disorder, phospholipid elimination in bile, renal disease, septicemia, metabolic syndrome disorders (e.g., Syndrome X), a thrombotic disorder, enhancing bile production, enhancing reverse lipid transport, inflammatory processes and diseases like gastrointestinal disease, irritable bowel syndrome (IBS), inflammatory bowel disease (e.g., Crohn's Disease, ulcerative colitis), arthritis (e.g., rheumatoid arthritis, osteoarthritis), autoimmune disease (e.g., systemic lupus erythematosus), scleroderma, ankylosing spondylitis, gout and pseudogout, muscle pain: polymyositis/polymyalgia rheumatica/fibrositis; infection and arthritis, juvenile rheumatoid arthritis, tendonitis, bursitis and other soft tissue rheumatism.

The invention provides methods of treatment and prophylaxis by administration to a patient of a therapeutically effective amount of a compound or a composition comprising a compound of the invention. The patient is an animal, including, but not limited, to an animal such a cow, horse, sheep, pig, chicken, turkey, quail, cat, dog, mouse, rat, rabbit, guinea pig, etc., and is more preferably a mammal, and most preferably a human.

The compounds and compositions of the invention, are preferably administered orally. The compounds and compositions of the invention may also be administered by any other convenient route, for example, by intravenous infusion or bolus injection, by absorption through epithelial or mucocutaneous linings (e.g., oral mucosa, rectal and intestinal mucosa, etc.) and may be administered together with another biologically active agent. Administration can be systemic or local. Various delivery systems are known, e.g., encapsulation in liposomes, microparticles, microcapsules, capsules, etc., and can be used to administer a compound of the invention. In certain embodiments, more than one compound of the invention is administered to a patient. Methods of administration include but are not limited to intradermal, intramuscular, intraperitoneal, intravenous, subcutaneous, intranasal, epidural, oral, sublingual, intranasal, intracerebral, intravaginal, transdermal, rectally, by inhalation, or topically, particularly to the ears, nose, eyes, or skin. The preferred mode of administration is left to the discretion of the practitioner, and will depend in-part upon the site of the medical condition. In most instances, administration will result in the release of the compounds of the invention into the bloodstream.

In specific embodiments, it may be desirable to administer one or more compounds of the invention locally to the area in need of treatment. This may be achieved, for example, and not by way of limitation, by local infusion during surgery, topical application, e.g., in conjunction with a wound dressing after surgery, by injection, by means of a catheter, by means of a suppository, or by means of an implant, said implant being of a porous, non-porous, or gelatinous material, including membranes, such as sialastic membranes, or fibers. In one embodiment, administration can be by direct injection at the site (or former site) of an atherosclerotic plaque tissue.

In certain embodiments, for example, for the treatment of Alzheimer's Disease, it may be desirable to introduce one or more compounds of the invention into the central nervous system by any suitable route, including intraventricular, intrathecal and epidural injection. Intraventricular injection may be facilitated by an intraventricular catheter, for example, attached to a reservoir, such as an Ommaya reservoir.

Pulmonary administration can also be employed, e.g., by use of an inhaler or nebulizer, and formulation with an aerosolizing agent, or via perfusion in a fluorocarbon or synthetic pulmonary surfactant. In certain embodiments, the compounds of the invention can be formulated as a suppository, with traditional binders and vehicles such as triglycerides.

In another embodiment, the compounds and compositions of the invention can be delivered in a vesicle, in particular a liposome (see Langer, 1990, Science 249:1527 1533; Treat et al., in Liposomes in the Therapy of Infectious Disease and Cancer, Lopez-Berestein and Fidler (eds.), Liss, New York, pp. 353 365 (1989); Lopez Berestein, ibid., pp. 317 327; see generally ibid.).

In yet another embodiment, the compounds and compositions of the invention can be delivered in a controlled release system. In one embodiment, a pump may be used (see Langer, supra; Sefton, 1987, CRC Crit. Ref. Biomed. Eng. 14:201; Buchwald et al., 1980, Surgery 88:507 Saudek et al., 1989, N. Engl. J. Med. 321:574). In another embodiment, polymeric materials can be used (see Medical Applications of Controlled Release, Langer and Wise (eds.), CRC Pres., Boca Raton, Fla. (1974); Controlled Drug Bioavailability, Drug Product Design and Performance, Smolen and Ball (eds.), Wiley, New York (1984); Ranger and Peppas, 1983, J. Macromol. Sci. Rev. Macromol. Chem. 23:61; see also Levy et al., 1985, Science 228:190; During et al., 1989, Ann. Neurol. 25:351; Howard et al., 1989, J. Neurosurg. 71:105). In yet another embodiment, a controlled-release system can be placed in proximity of the target area to be treated, e.g., the liver, thus requiring only a fraction of the systemic dose (see, e.g., Goodson, in Medical Applications of Controlled Release, supra, vol. 2, pp. 115 138 (1984)). Other controlled-release systems discussed in the review by Langer, 1990, Science 249:1527 1533) may be used.

The present compositions will contain a therapeutically effective amount of a compound of the invention, optionally more than one compound of the invention, preferably in purified form, together with a suitable amount of a pharmaceutically acceptable vehicle so as to provide the form for proper administration to the patient.

In a specific embodiment, the term “pharmaceutically acceptable” means approved by a regulatory agency of the Federal or a state government or listed in the U.S. Pharmacopeia or other generally recognized pharmacopeia for use in animals, and more particularly in humans. The term “vehicle” refers to a diluent, adjuvant, excipient, or carrier with which a compound of the invention is administered. Such pharmaceutical vehicles can be liquids, such as water and oils, including those of petroleum, animal, vegetable or synthetic origin, such as peanut oil, soybean oil, mineral oil, sesame oil and the like. The pharmaceutical vehicles can be saline, gum acacia, gelatin, starch paste, talc, keratin, colloidal silica, urea, and the like. In addition, auxiliary, stabilizing, thickening, lubricating and coloring agents may be used. When administered to a patient, the compounds and compositions of the invention and pharmaceutically acceptable vehicles are preferably sterile. Water is a preferred vehicle when the compound of the invention is administered intravenously. Saline solutions and aqueous dextrose and glycerol solutions can also be employed as liquid vehicles, particularly for injectable solutions. Suitable pharmaceutical vehicles also include excipients such as starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The present compositions, if desired, can also contain minor amounts of wetting or emulsifying agents, or pH buffering agents.

The present compositions can take the form of solutions, suspensions, emulsion, tablets, pills, pellets, capsules, capsules containing liquids, powders, sustained-release formulations, suppositories, emulsions, aerosols, sprays, suspensions, or any other form suitable for use. In one embodiment, the pharmaceutically acceptable vehicle is a capsule (see e.g., U.S. Pat. No. 5,698,155). Other examples of suitable pharmaceutical vehicles are described in “Remington's Pharmaceutical Sciences” by E. W. Martin.

In a preferred embodiment, the compounds and compositions of the invention are formulated in accordance with routine procedures as a pharmaceutical composition adapted for intravenous administration to human beings. Typically, compounds and compositions of the invention for intravenous administration are solutions in sterile isotonic aqueous buffer. Where necessary, the compositions may also include a solubilizing agent. Compositions for intravenous administration may optionally include a local anesthetic such as lignocaine to ease pain at the site of the injection. Generally, the ingredients are supplied either separately or mixed together in unit dosage form, for example, as a dry lyophilized powder or water free concentrate in a hermetically sealed container such as an ampoule or sachette indicating the quantity of active agent. Where the compound of the invention is to be administered by intravenous infusion, it can be dispensed, for example, with an infusion bottle containing sterile pharmaceutical grade water or saline. Where the compound of the invention is administered by injection, an ampoule of sterile water for injection or saline can be provided so that the ingredients may be mixed prior to administration.

Compounds and compositions of the invention for oral delivery may be in the form of tablets, lozenges, aqueous or oily suspensions, granules, powders, emulsions, capsules, syrups, or elixirs. Compounds and compositions of the invention for oral delivery can also be formulated in foods and food mixes. Orally administered compositions may contain one or more optionally agents, for example, sweetening agents such as fructose, aspartame or saccharin; flavoring agents such as peppermint, oil of wintergreen, or cherry; coloring agents; and preserving agents, to provide a pharmaceutically palatable preparation. Moreover, where in tablet or pill form, the compositions may be coated to delay disintegration and absorption in the gastrointestinal tract thereby providing a sustained action over an extended period of time. Selectively permeable membranes surrounding an osmotically active driving compound are also suitable for orally administered compounds and compositions of the invention. In these later platforms, fluid from the environment surrounding the capsule is imbibed by the driving compound, which swells to displace the agent or agent composition through an aperture. These delivery platforms can provide an essentially zero order delivery profile as opposed to the spiked profiles of immediate release formulations. A time delay material such as glycerol monostearate or glycerol stearate may also be used. Oral compositions can include standard vehicles such as mannitol, lactose, starch, magnesium stearate, sodium saccharine, cellulose, magnesium carbonate, etc. Such vehicles are preferably of pharmaceutical grade.

The amount of a compound of the invention that will be effective in the treatment of a particular disorder or condition disclosed herein will depend on the nature of the disorder or condition, and can be determined by standard clinical techniques. In addition, in vitro or in vivo assays may optionally be employed to help identify optimal dosage ranges. The precise dose to be employed in the compositions will also depend on the route of administration, and the seriousness of the disease or disorder, and should be decided according to the judgment of the practitioner and each patient's circumstances. However, suitable dosage ranges for oral administration are generally about 0.001 milligram to 2000 milligrams of a compound of the invention per kilogram body weight. In specific preferred embodiments of the invention, the oral dose is 0.01 milligram to 1000 milligrams per kilogram body weight, more preferably 0.1 milligram to 100 milligrams per kilogram body weight, more preferably 0.5 milligram to 25 milligrams per kilogram body weight, and yet more preferably 1 milligram to 10 milligrams per kilogram body weight. In a most preferred embodiment, the oral dose is 5 milligrams of a compound of the invention per kilogram body weight. The dosage amounts described herein refer to total amounts administered; that is, if more than one compound of the invention is administered, the preferred dosages correspond to the total amount of the compounds of the invention administered. Oral compositions preferably contain 10% to 95% active ingredient by weight.

Suitable dosage ranges for intravenous (i.v.) administration are 0.01 milligram to 1000 milligrams per kilogram body weight, 0.1 milligram to 350 milligrams per kilogram body weight, and 1 milligram to 100 milligrams per kilogram body weight. Suitable dosage ranges for intranasal administration are generally about 0.01 pg/kg body weight to 1 mg/kg body weight. Suppositories generally contain 0.01 milligram to 50 milligrams of a compound of the invention per kilogram body weight and comprise active ingredient in the range of 0.5% to 10% by weight. Recommended dosages for intradermal, intramuscular, intraperitoneal, subcutaneous, epidural, sublingual, intracerebral, intravaginal, transdermal administration or administration by inhalation are in the range of 0.001 milligram to 200 milligrams per kilogram of body weight. Suitable doses of the compounds of the invention for topical administration are in the range of 0.001 milligram to 1 milligram, depending on the area to which the compound is administered. Effective doses may be extrapolated from dose-response curves derived from in vitro or animal model test systems. Such animal models and systems are well known in the art.

The invention also provides pharmaceutical packs or kits comprising one or more containers filled with one or more compounds of the invention. Optionally associated with such container(s) can be a notice in the form prescribed by a governmental agency regulating the manufacture, use or sale of pharmaceuticals or biological products, which notice reflects approval by the agency of manufacture, use or sale for human administration. In a certain embodiment, the kit contains more than one compound of the invention. In another embodiment, the kit comprises a compound of the invention and another lipid-mediating compound, including but not limited to a statin, a thiazolidinedione, or a fibrate.

The compounds of the invention are preferably assayed in vitro and in vivo, for the desired therapeutic or prophylactic activity, prior to use in humans. For example, in vitro assays can be used to determine whether administration of a specific compound of the invention or a combination of compounds of the invention is preferred for lowering fatty acid synthesis. The compounds and compositions of the invention may also be demonstrated to be effective and safe using animal model systems.

Other methods will be known to the skilled artisan and are within the scope of the invention.

The following examples are provided by way of illustration and not limitation.

### 6. SYNTHETIC EXAMPLES

### 6.1 Synthesis of the Compounds of the Invention

A series of long hydrocarbon chain ether-diols was synthesized as described in the schemes below. The side chains connected to the central ether finctionality varied both in the number of methylene spacer units (n=3-5) and in the attached geminal modifying groups (R=Me, Ph), resulting in ether-diols of either the symmetrical (109-111 in Scheme 101; 128 and 131 in Scheme 102) or the unsymmetrical (126, 127, 129, and 130 in Scheme 102) category.

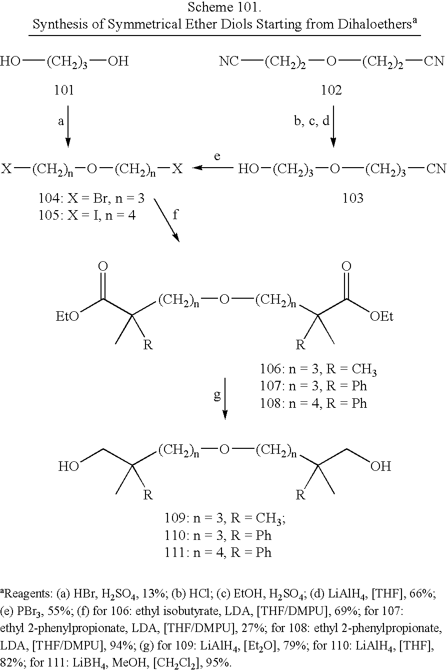

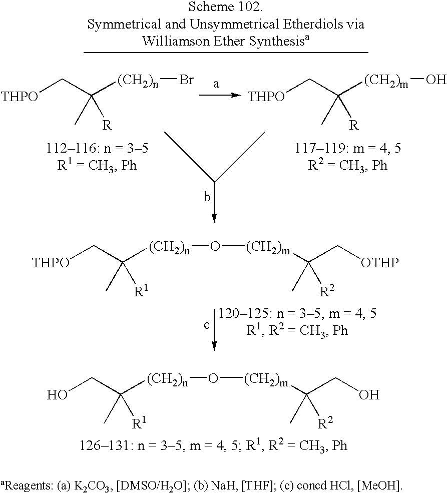

TABLE A
Symmetrical and Unsymmetrical Ether Diols
via Williamson Ether Synthesis

| No. | n | m | R¹ | R² | Yield (%) |
|---|---|---|---|---|---|
| 112 | 3 | — | CH₃ | — | ^{a} |
| 113 | 3 | — | Ph | — | ^{a} |
| 114 | 4 | — | CH₃ | — | ^{a} |
| 115 | 4 | — | Ph | — | ^{a} |
| 116 | 5 | — | CH₃ | — | ^{a} |
| 117 | — | 4 | — | CH₃ | 99^{b} |
| 118 | — | 4 | — | Ph | 38 |
| 119 | — | 5 | — | CH₃ | 83^{b} |
| 120 | 3 | 4 | CH₃ | CH₃ | 63 |
| 121 | 3 | 4 | Ph | Ph | ^{c} |
| 122 | 4 | 4 | CH₃ | CH₃ | ^{c} |
| 123 | 4 | 4 | Ph | CH₃ | ^{c} |
| 124 | 5 | 4 | CH₃ | CH₃ | 34 |
| 125 | 5 | 5 | CH₃ | CH₃ | ^{c} |
| 126 | 3 | 4 | CH₃ | CH₃ | 51 (32^{d}) |
| 127 | 3 | 4 | Ph | Ph | 44^{d} |
| 128 | 4 | 4 | CH₃ | CH₃ | 37^{d} |
| 129 | 4 | 4 | Ph | CH₃ | 24^{d} |
| 130 | 5 | 4 | CH₃ | CH₃ | 90 (31^{d}) |
| 131 | 5 | 5 | CH₃ | CH₃ | 30^{d} |

^{a} Synthesis described in Dasseux, J.-L. H. et al. U.S. Pat. No. 20030078239, 2003; U.S. Pat. Appl. 09/976,938, filed Oct. 11, 2001;
^{b}used without purification for step b;
^{c} directly used for step c;
^{d}overall yield for steps b and c.

Moreover, the aryl-bridged diacid 135 and diol 136 (Scheme 103) as well as the diols 138 and 141 with cyclic ether structures (Scheme 104) were synthesized and examined for comparison. Also included in this study were the ether-diacid 145 (Scheme 105) with γ,γ,γ′,γ′-tetramethyl substitution, the THP-protected derivatives 122 and 146 (Scheme 106), compounds 149 and 150 with a diether structural element (Scheme 107), and finally the hydrocarbon chain analogs 155-157 (Scheme 108).

The synthesis of long hydrocarbon chain ether-diols was accomplished by two different methods. According to the first method (Scheme 101), bis(ω-haloalkyl) ethers were reacted with lithiated ethyl esters and the diesters formed were reduced to the target diols. For n=3, starting dibromo ether 104 was first prepared by condensing 1,3-propanediol (101) with 48% aqueous HBr and concd sulfuric acid (Kamm, O. et al. J. Am. Chem. Soc. 1921, 43, 2228-2230). However, the yield for this reaction was only 13% and 104 was difficult to purify by fractionating distillation. A better overall yield (36%) was obtained when dinitrile 102 was converted via a three-step reaction sequence consisting of saponification (concd HCl) (Pratt, J. A. E. et al. J. Chem. Soc. Perkin Trans 1 1988, 13-22; Samat, A.; Bibout, M. E. M. Heterocycles 1982, 19, 469-472), esterification (EtOH, concd H₂SO₄) (Buchanan, G. W. et al. Can. J. Chem. 2000, 78, 316-321), and reduction (LiAlH₄) to diol 103 (66%) (Buchanan, G. W. et al. Can. J. Chem. 2000, 78, 316-321), which was then transformed to 104 with PBr₃ (55%) (Harrison, G. C. et al. In Organic Syntheses, Collective Volume 3; Homing, E. C., Editor-in-Chief; John Wiley & Sons: New York, N.Y., 1955; pp 370-371). Bromide substitution in 104 with lithio ethyl isobutyrate and lithio ethyl 2-phenylpropionate (Dasseux, J.-L. Het al. US 20030078239, 2003; U.S. patent application Ser. No. 09/976,938, filed Oct. 11, 2001; Yang, H. et al. J. Org. Chem. 1999, 64, 1709-1712) in THF and co-solvent dimethylpropyleneurea (DMPU) gave diesters 106 and 107 in 69% and 27% yield, respectively. Reduction of the esters with lithium aluminum hydride afforded ether-diols 109 (Gleiter, R. et al. Liebigs Ann. 1995, 1655-1661) and 110 (79% and 82%) after purification by distillation or chromatography. For n=4, the same methodology starting with diiodide 105 (Taylor, E. P. J. Chem. Soc. 1952, 142-144) via diester 108 was used and ether-diol 111 was obtained by reduction with lithium borohydride and methanol in dichloromethane (J-L. H. Dasseux et al. U.S. Pat. No. 6,410,802, 2002; U.S. patent application Ser. No. 09/540,740 filed Mar. 31, 2000) (86% over both steps).

According to the second method, symmetrical and unsymmetrical ether-diols 126-131 were prepared via Williamson reaction (Feuer, H. In The Chemistry of the Ether Linkage. Patai, S., Editor; Interscience Publishers, New York, 1967, p. 446; Meerwein, H. In Methoden Der Organischen Chemie (Houben-Weyl). Müller, E., Editor; Georg Thieme Verlag, Stuttgart, 1965, p. 26) of THP-protected bromo alcohols 112-116 (Dasseux, J.-L. Het al. US 20030078239, 2003; U.S. patent application Ser. No. 09/976,938, filed Oct. 11, 2001; Yang, H. et al. J. Org. Chem. 1999, 64, 1709-1712) with the sodium salts of alcohols 117-119 (Scheme 102, Table 101). Therefore, bromides 114-116 were hydrolyzed with K₂CO₃ in a DMSO/water mixture at reflux (Treves, G. R. et al. Chem. Ind. 1971, 544) to afford alcohols 117-119 in varying yields (38-99%). Alcohol 117 was deprotonated with NaH in THF at reflux temperature and condensed with bromo THP-ethers 112 and 116 leading to protected ether intermediates 120 and 124 (60% and 34%), respectively, which were both purified by column chromatography. Deprotection of 120 and 124 with concd HCl in methanol at reflux furnished the two unsymmetrical ether-diols 126 (51%) and 130 (90%). The yields over both steps for these compounds, however, were basically the same (32% and 31%, Table 101). The synthesis of ethers 127-129 and 130 from alcohols 117-119 and bromides 113-116 followed the same protocol, except that the protected intermediates 121-123 and 125 were not purified, but directly deprotected to the final ether-diols. The moderate yields obtained over both steps (24-44%) were in the same range as those with purification of the THP-protected intermediates and the differences in the synthesis of symmetrical (128 and 131) and unsymmetrical (126, 127, 129, 131) ether-diols were not significant.

The synthesis of diaryl ethers 135 and 136 is depicted in Scheme 103. Diphenyl ether 132 (von Schickh, O. Chem. Ber. 1936, 69, 242-244) was brominated with NBS and benzoyl peroxide in CCl₄ and the crude dibromide 133 (Marty, W. Inorg. Chem. 1979, 18, 1246-1250) was reacted with lithio ethyl isobutyrate in THF/DMPU to give diester 134 (43%). Saponification of 134 with KOH in aqueous ethanol led to diacid 135, which was purified by crystallization from hexanes (35%). Reduction of 134 with LiAlH₄ in diethyl ether afforded ether-diol 136 as an oil that had to be purified by repeated chromatography (18% overall yield from 132).

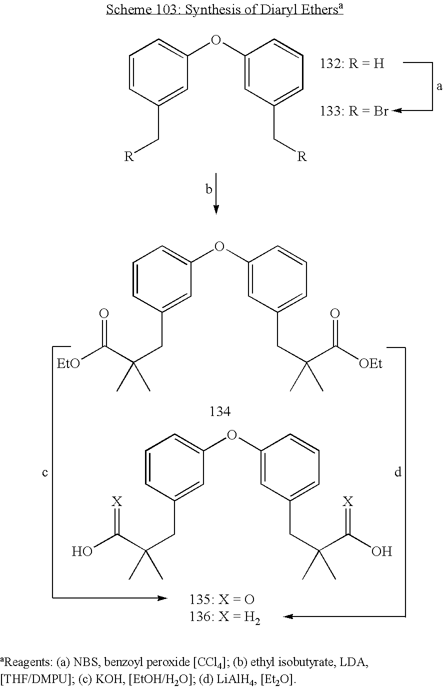

Scheme 104 illustrates the synthesis of cyclic etherdiols 138 and 141. Reaction of the Grignard reagent of bromo THP-ether 112 with freshly prepared succinaldehyde (House, H. O. J. Org. Chem. 1965, 30, 1061-1070; Suzuki, M. Chem. Pharm. Bull. 2001, 49, 29-39) in THF gave diol 137 (92%) (Faulkner, D. J. et al. J. Am. Chem. Soc. 1973, 95, 553-563). The cyclodehydration (Molnar, A. et al. Tetrahedron 1981, 37, 2149-2151) and deprotection to tetrahydrofliran derivative 138 was then accomplished by two different methods: first, 137 was condensed by treatment with p-toluenesulfonic acid in toluene under azeotropic removal (Paquette, L. A. Et al. J. Am. Chem. Soc. 1991, 113, 5072-5073) of the reaction water. Subsequent removal of the THP groups (aq H₂SO₄/MeOH) furnished cyclic ether-diol 138 in moderate yield (35%). Alternatively, 137 was mono-tosylated (1.1 equiv TsCl, py) (Hashimoto, M. et al. J. Org. Chem. 1991, 56, 2299-2311) and then cyclized under basic conditions (Py, HMPA). After deprotection of the terminal alcohols (aq H₂SO₄/MeOH) and purification by chromatography, 138 was obtained in 38% yield. A similar methodology as for the synthesis of 138 was utilized to access tetrahydropyranyl diol 141. Reaction of the Grignard reagent of 112 with glutaric aldehyde (Suzuki, M. Chem. Pharm. Bull. 2001, 49, 29-39) gave compound 139 (55%). Deprotection (aq H₂SO₄/MeOH) furnished tetraol 140 that could conveniently be purified by crystallization from CH₂Cl₂/hexanes (80%). Finally, dehydration of 140 under acidic conditions (pTosOH, toluene, Dean-Stark) led to 141 in 55% yield.

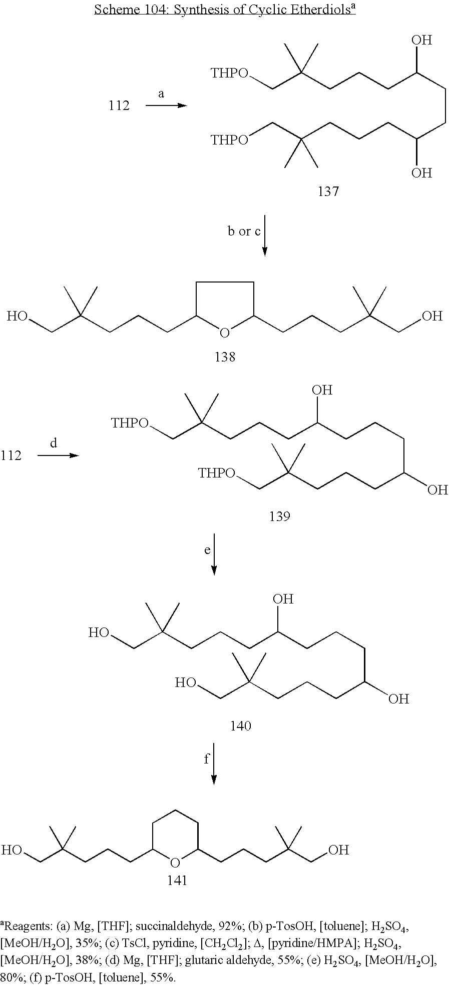

The synthesis of the γ,γ,γ′,γ′-tetramethyl substituted ether-diacid 145 began with the oxidation of diol 128 to dialdehyde 142 using sulfur trioxide pyridine complex and NEt₃ in DMSO (63%, Scheme 105) (Parikh, J. R. Et al. J. Am. Chem. Soc. 1967, 89, 5505-5507). The α,β-unsaturated ester 143 was then prepared by Wittig-Horner reaction of 142 with methyl diethylphosphonoacetate in the presence of sodium hydride in DMF' (Tsuno, T. et al. Tetrahedron 2001, 57, 4831-4840; Shishido, K. et al. Heterocycles 1994, 38, 641-648) Subsequent hydrogenation to 144 (Pd—C)( Shishido, K. et al. Heterocycles 1994, 38, 641-648) followed by saponification of the ester groups (KOH, MeOH/H₂O) (Beckwith, A. L. J. et al. J. Org. Chem. 1988, 53, 1632-1641) furnished the target compound 145 in respectable yield (56% from 142).

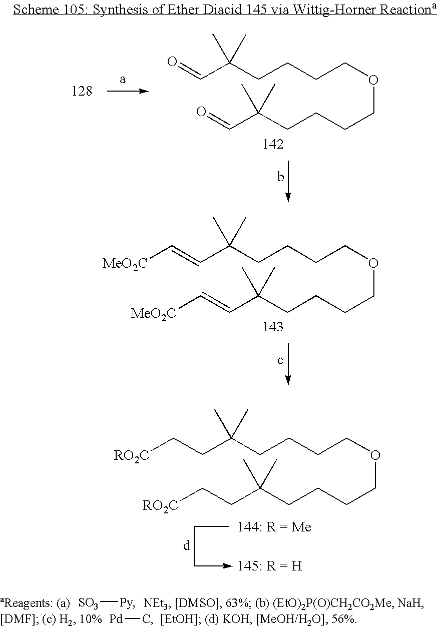

The mono- and bis-THP-ethers 122 and 146 (Scheme 106) were found as trace impurites in the kilogram-scale synthesis of etherdiol 128 (Scheme 102) (J-L. H. Dasseux et al. U.S. Pat. No. 6,410,802, 2002; U.S. patent application Ser. No. 09/540,740 filed Mar. 31, 2000). Therefore, it was important to evaluate their biological properties, which required clean samples of both 1 and 146. Treatment of 128 with 3,4-dihydro-2H-pyran (1 equiv) and catalytic amounts of p-toluenesulfonic acid in CH₂Cl₂ (Ackerley, N. et al. J. Med. Chem. 1995, 38, 1608-1628) produced a mixture of 122, 128, and 146 that was separated by chromatography to yield THP-ethers 122 (21%) and 146 (32%).

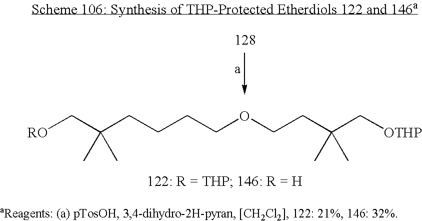

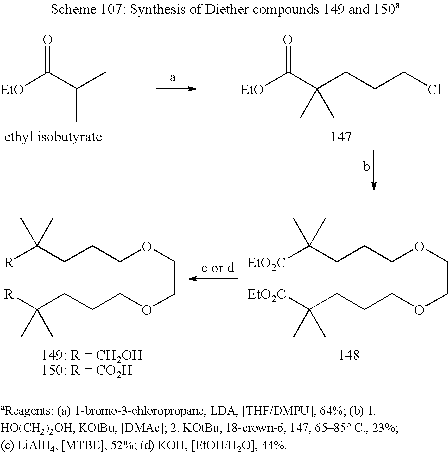

The synthesis of diether-diol 149 and -diacid 150 is shown in Scheme 107. Alkylation of lithiated ethyl isobutyrate with 1-bromo-3-chloropropane in THF/DMPU gave chloro ester 147 (64%). Ethylene glycol was then deprotonated with potassium tert-butoxide (1.5 equiv) in dimethyl acetamide (DMAc) and reacted with 147 (1.5 equiv) at 65° C. (Czech, B. P. et al. J. Org. Chem. 1984, 49, 4805-4810). Further reaction of this mixture with 147 (1.5 equiv), potassium tert-butoxide (1.5 equiv) and catalytic amounts of 18-crown-6 at 65-85° C. afforded diester 148 (23%). Subsequent reduction of 148 with LiAlH₄ in MTBE led to diol 149 (52%), while its saponification (KOH, aq ethanol) produced diacid 150 (44%).

The hydrocarbon chain analogs to the ether compounds described in this work were synthesized here as a reference, by reaction of lithio ethyl isobutyrate with dibromides 151 and 152 in THF/DMPU to furnish diesters 153 and 154 (79% and 94%), respectively (Scheme 8). Reduction of the shorter chain homolog 153 with LiAlH₄ in diethyl ether produced diol 155 (62%), whereas the longer chain homolog 154 was reduced with lithium borohydride and methanol in dichloromethane to 156 (51%) (J-L. H. Dasseux et al. U.S. Pat. No. 6,410,802, 2002; U.S. patent application Ser. No. 09/540,740 filed Mar. 31, 2000). The tetramethyl substituted diacid 157—finally—was synthesized from 154 via ester hydrolysis with potassium hydroxide in aqueous ethanol (69%).

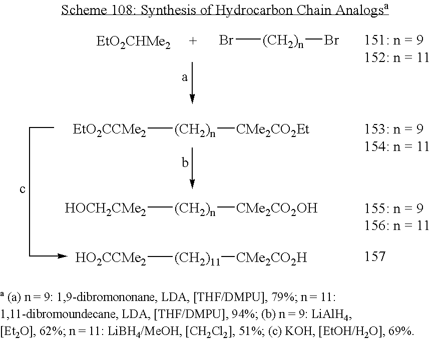

### 6.2 Examples of Illustrative Compounds of the Invention

Bis(3-bromopropyl) ether (104). Under N₂-atmosphere, phosphorus tribromide (7.2 mL, 20.5 g, 75.8 mmol) was added dropwise over 1 h to 103 (Buchanan, G. W. et al. Can. J. Chem. 2000, 78, 316-321) (10.14 g, 75.6 mmol), causing self-heating to reflux. The reaction mixture was stirred overnight at room temperature, then distilled in vacuo to give an oil. This oil was dissolved in CH₂Cl₂ (100 mL), washed with water (100 mL), dried over Na₂SO₄, and concentrated in vacuo, affording 104 (10.9 g, 55%) as a clear, colorless oil. Bp 68-70° C./0.2 mmHg. ¹H NMR (CDCl₃): δ 3.56 (t, 4 H, J=5.8), 3.51 (t, 4H, J=6.4), 2.10 (m, 4H). ¹³C NMR (CDCl₃=77.00 ppm): δ 68.17, 32.68, 30.59.

5-(4-Ethoxycarbonyl-4-methylpentyloxy)-2,2-dimethylpentanoic acid ethyl ester (106). Under nitrogen atmosphere and at −78° C., to a stirred solution of ethyl isobutyrate (6.5 g, 55.9 mmol) in anhydrous THF (30 mL) was added dropwise a solution of lithium diisopropylamide (30 mL, 60.0 mmol, 2.0 M in heptane/THF/ethylbenzene). After 1 h, a solution of 104 (Kamm, O. et al. J. Am. Chem. Soc. 1921, 43, 2228-2230) (6.76 g, 26.0 mmol) and DMPU (2 mL) in THF (15 mL) was added dropwise and the reaction temperature was kept at −78° C. for additional 30 min. The reaction mixture was allowed to warn to room temperature and stirred overnight, then quenched with a mixture of ice (10 g) and concd HCl (10 mL). The product was extracted with diethyl ether (2×30 mL). The combined ether phases were washed with 5% NaHCO₃ solution (30 mL), dried over MgSO₄, and concentrated in vacuo to furnish the crude product (10.9 g), which was distilled in high vacuo to give pure 106 (5.96 g, 69%) as an oil. Bp 115-120° C./0.5 mmHg. ¹H NMR (CDCl₃): δ 4.11 (q, 4H, J=7.0), 3.37 (m, 4H), 1.62-1.43 (m, 8H), 1.25 (t, 4H, J=7.0), 1.17 (s, 12H). ¹³C NMR (CDCl₃=77.00 ppm): δ 177.62, 70.85, 60.06, 41.75, 36.89, 25.14, 24.96, 14.09.

5-(4-Ethoxycarbonyl-4-phenylpentyloxy)-2-methyl-2-phenylpentanoic acid ethyl ester (107). Under N₂-atmosphere, to a stirred solution of 2-phenylpropionic acid ethyl ester (Dasseux, J.-L. H. et al. US 20030078239, 2003; U.S. patent application Ser. No. 09/976,938, filed Oct. 11, 2001) (13.2 g, 74.1 mmol) in anhydrous THF (50 mL) was added dropwise a solution of lithium diisopropylamide (39 mL, 78.0 mmol, 2.0 M in heptane/THF/ethylbenzene) at −78° C. After 1 h, DMPU (3 mL) was added, followed by the dropwise addition of a solution of 104 (9.2 g, 35.4 mmol) in THF (20 mL). After 30 min at −78° C., the reaction mixture was warmed to room temperature and stirred overnight. The reaction mixture was poured into a mixture of ice (30 g) and concd HCl (20 mL) and the product was extracted with diethyl ether (4×75 mL). The combined ether solutions were washed with 5% NaHCO₃ solution (50 mL), and brine (50 mL), dried over MgSO₄, and concentrated in vacuo to furnish the crude product (18.9 g) as a brown oil. Purification by column chromatography (silica; hexanes/ethyl acetate=10/1) gave 7 (4.3 g, 27%) as a yellow oil. ¹H NMR (CDCl₃): δ 7.45-7.10 (m, 10H), 4.11 (q, 4H, J=7.2), 3.45-3.25 (m, 4H), 2.15-1.85 (m, 4H), 1.54 (s, 6H), 1.50-1.35 (m, 4H), 1.17 (t, 6H, J=7.2). ¹³C NMR (CDCl₃=77.00 ppm): δ 176.00, 143.71, 128.23, 126.50, 125.90, 70.89, 60.66, 49.86, 35.66, 25.08, 22.66, 14.02. HRMS (LSIMS, nba): Calcd for C₂₈H₃₉O₅ (MH⁺): 455.2797, found: 455.2796.

6-(5-Ethoxycarbonyl-5-phenylhexyloxy)-2-methyl-2-phenyl-hexanoic acid ethyl ester (108). According to the procedure given for the synthesis of 7, 2-phenylpropionic acid ethyl ester (Yang, H. et al. J. Org. Chem. 1999, 64, 1709-1712.) (35.6 g, 0.20 mol) was deprotonated with lithium diisopropylamide (2.0 M in heptane/THF/ethylbenzene, 105 mL, 0.21 mol) in anhydrous THF (150 mL), then treated with DMPU (18 mL) and 105 (Taylor, E. P. J. Chem. Soc. 1952, 142-144) (38.2 g, 0.10 mol) overnight. The residue obtained after hydrolysis and extraction was purified by flash chromatography (silica; hexanes/ethyl acetate=75/25) affording 108 (45.2 g, 94%) as an oil. ¹H NMR (300 MHz, CDCl₃): δ 7.31-7.20 (m, 10H), 4.11 (q, 4H, J=7.2), 3.34 (t, 4H, J=6.6), 2.12-1.82 (m, 4H), 1.62-1.48 (m, 10H), 1.30-1.12(m, 4H), 1.16 (t, 6H, J=7.2). ¹³C NMR (CDCl₃=77.00 ppm): δ 176.04, 143.99, 128.19, 126.44, 125.85, 70.49, 60.55, 50.09, 38.91, 30.12, 22.61, 21.35, 13.97.

5-(5-Hydroxy-4,4-dimethylpentyloxy)-2,2-dimethylpentan-1-ol (109). Under N₂-atmosphere, to a solution of lithium aluminum hydride in diethyl ether (55 mL, 1.0 M, 55 mmol) was added dropwise a solution of 106 (5.62 g, 17.0 mmol) in anhydrous diethyl ether (25 mL) at such a rate as to prevent the ether from boiling. The mixture was stirred for 30 min, then hydrolyzed by subsequent slow addition of distilled water (30 mL) and 25% sulfuric acid (35 mL). The product was extracted with diethyl ether (5×75 mL). The combined ether extracts were washed with 5% NaHCO₃ solution (2×25 mL), dried over MgSO₄, and concentrated under reduced pressure to give an oil (4.95 g). Purification by vacuum distillation afforded 109 (3.3 g, 79%) as an almost colorless, very viscous oil. Bp 130-140° C./0.5 mmHg (lit. Gleiter, R. et al. Liebigs Ann. 1995, 1655-1661 mp 30-32° C.). ¹H NMR (CDCl₃): δ 3.41 (t, 4H, J=6.3), 3.28 (s, 4H), 3.11 (br s, 2H), 1.60-1.48 (m, 4H), 1.33-1.24 (m, 4H), 0.86 (s, 12H). ¹³C NMR (CDCl₃=77.00 ppm): δ 71.65, 70.55, 34.78, 34.34, 24.06, 23.88. HRMS (CI): Calcd for C₁₄H₃₁O₃ (MH⁺): 247.2273, found: 247.2265. HPLC: Alltima C8 column, 250×4.6 mm, 5μ; 58% acetonitrile, 42% water, flow rate 1.0 mL/min; RI, retention time 4.95 min, 86.3% pure.

5-(5-Hydroxy-4-methyl-4-phenylpentyloxy)-2-methyl-2-phenylpentan-1-ol (110). According to the procedure given for the synthesis of 109, 107 (4.2 g, 9.2 mmol) was reduced with lithium aluminum hydride (1.7 g, 44.8 mmol) in anhydrous diethyl ether (100 mL) at room temperature overnight. Hydrolysis with acid, extraction, and drying afforded pure 110 (2.8 g, 82%) as a colorless oil. ¹H NMR (CDCl₃): δ 7.45-7.10 (m, 10H), 3.70 (d, 2H, J=11.0), 3.58 (d, 2H, J=11.0), 3.28 (t, 4H, J=6.3), 1.90-1.15 (m, 10H), 1.34 (s, 6H). ¹³C NMR (CDCl₃=77.00 ppm): δ 144.75, 128.41, 126.62, 126.11, 71.91, 71.21, 43.09, 34.60, 24.16, 21.87. HRMS (LSIMS, gly): Calcd for C₂₄H₃₅O₃ (MH⁺): 371.2586, found: 371.2581. HPLC: Alltima C8 column, 250×4.6 mm, 5μ; 60% acetonitrile, 40% water, flow rate 1.0 mL/min; RI, retention time 8.50 min, 93.9% pure.

6-(6-Hydroxy-5-methyl-5-phenylhexyloxy)-2-methyl-2-phenylhexan-1-ol (111). Under N₂ atmosphere and at room temperature, to a suspension of lithium borohydride (6.3 g, 289 mmol) in CH₂Cl₂ (210 mL) was added dropwise methanol (8.8 g, 275 mmol) over 30 min. The reaction mixture was heated to reflux and 108 (44.0 g, 91.2 mmol) was added. After refluxing overnight and cooling to room temperature, saturated NH₄Cl solution (100 mL) was added and the layers were separated. The aqueous layer was extracted with CH₂Cl₂ (3×50 mL). The combined organic layers were washed with 2 N HCl (100 mL) and saturated NaCl solution (100 mL), dried over MgSO₄, and concentrated in vacuo. The crude product was purified by flash chromatography (silica; hexanes/ethyl acetate=80/20), affording 111 (34.4 g, 95%) as an oil. ¹H NMR (CDCl₃): δ 7.36-7.14 (m, 10H), 3.67 (d, 2H, J=10.7), 3.52 (d, 2H, J=10.7), 3.27 (t, 4H, J=6.6), 1.82-1.70 (m, 2H), 1.60-1.10 (m, 10H), 1.33 (s, 6H), 1.05-0.95 (m, 2H). ¹³C NMR (CDCl₃=77.00 ppm): δ 144.84, 128.37, 126.63, 126.05, 72.35, 70.57, 43.34, 38.21, 30.25, 21.56, 20.44. HRMS (LSIMS, nba): Calcd for C₂₆H₃₉O₃ (MH⁺): 399.2899, found: 399.2903.

General Procedure for the Hydrolysis of ω-Bromo- to ω-Hydroxyalkyl THP Ethers: 6,6-Dimethyl-7-(tetrahydropyran-2-yloxy)-heptan-1-ol (119). A mixture of 116 (11.0 g, 35.8 mmol), K₂CO₃ (10.0 g, 72.4 mmol), water (100 mL) and DMSO (50 mL) was heated to reflux for 24 h. After cooling to room temperature, the mixture was diluted with water (150 mL) and neutralized by addition of concd HCl (5 mL) and 1 N HCl (15 mL). The solution was extracted with diethyl ether (4×100 mL). The combined organic layers were washed with saturated NH₄Cl solution (100 mL) and saturated NaCl solution (100 mL), dried over MgSO₄, and concentrated in vacuo to furnish 119 (7.3 g, 83%) as a colorless oil, which was used without further purification for the next step. ¹H NMR (CDCl₃): δ 4.55 (m, 1H), 3.84 (m, 1H), 3.61 (t, 2H, J=6.5), 3.51-3.30 (m, 1H), 3.45 (d, 1H, J=9.1), 2.98 (d, 1H, J=9.1), 2.26 (br s, 1H), 1.98-1.40 (m, 8H), 1.40-1.10 (m, 6H), 0.88 (s, 6H). ¹³C NMR (CDCl₃=77.20 ppm): δ 99.17, 76.61, 62.89, 61.95, 39.38, 34.28, 32.87, 30.75, 26.79, 25.66, 24.65, 23.81, 19.50. HRMS (LSIMS, nba): Calcd for C₁₄H₂₉O₃ (MH⁺): 245.2117, found: 245.2119.

5,5-Dimethyl-6-(tetrahydropyran-2-yloxy)-hexan-1-ol (117). According to the method provided for the synthesis of 119, 114 (20.15 g, 68.7 mmol) was treated with K₂CO₃ (19.0 g, 137.7 mmol) in DMSO (100 mL) and water (200 mL) reflux for 17 h. Workup by neutralization, extraction, and drying afforded 117 (15.7 g, 99%) as a yellowish oil, which was used without further purification for the next step. ¹H NMR (CDCl₃): δ 4.54 (t, 1H, J=3.9), 3.84 (m, 1H, 3.62 (t, 2H, J=6.4), 3.50 (m, 1H), 3.48 (d, 1H, J=9.1), 3.00 (d, 1H, J=9.1), 2.14 (s br, 1H), 1.90-1.44 (m, 8H), 1.40-1.22 (m, 4H), 0.89 (s, 6H). ¹³C NMR (CDCl₃=77.22 ppm): δ 99.36, 76.49, 62.77, 62.22, 39.01, 34.33, 33.66, 30.80, 25.67, 24.74, 24.63, 20.14, 19.66.

5-Methyl-5-phenyl-6-(tetrahydropyran-2-yloxy)-hexan-1-ol (118). According to the method provided for the synthesis of 119, 115 (11.14 g, 31.4 mmol) was treated with K₂CO₃ (8.45 g, 61.1 mmol) in DMSO (25 mL) and water (50 mL) under reflux for 22 h. The crude material obtained after neutralization and extraction was purified by flash chromatography (silica; hexanes/ethyl acetate=75/25), affording 118 (3.5 g, 38% yield) as a clear, colorless oil. ¹H NMR (CDCl₃): δ 7.38-7.14 (m, 10H), 4.52 (t, 1H, J=3.4), 4.49 (t, 1H, J=3.4), 3.83 (d, 1H, J=9.3), 3.82 (d, 1H, J=9.3), 3.78-3.60 (m, 2H), 3.54 (t, 4H, J=6.4), 3.48-3.38 (m, 2H), 3.38 (d, 1H, J=9.4), 3.36 (d, 1H, J=9.3), 1.95-1.40 (m, 20H), 1.37 (s, 3H), 1.36 (s, 3H), 1.30-0.95 (m, 4H). Mixture of diastereomers in a ratio of ca. 50/50.

General Procedure for the Williamson Ether Synthesis: 2-{6-[4,4-Dimethyl-5-(tetrahydropyran-2-yloxy)-pentyloxy]-2,2-dimethylhexyloxy}-tetrahydropyran (120). Under Ar-atmosphere, to a suspension of sodium hydride (95%, 0.76 g, 30 mmol) in anhydrous THF (80 mL) was added dropwise 117 (7.93 g, 34.4 mmol) over 10 min at room temperature. The reaction mixture was heated to reflux overnight, before 112 (9.30 g, 33.3 mmol) was added dropwise and heating to reflux was continued for 4 h. After cooling to room temperature, the mixture was hydrolyzed by adding ice (20 g) and saturated NH₄Cl solution (30 mL). The layers were separated and the aqueous layer was extracted with ethyl acetate (3×50 mL). The combined organic layers were washed with saturated NH₄Cl solution (3×50 mL), dried over MgSO₄, and concentrated in vacuo. Purification by flash chromatography (silica gel, ethyl acetate/hexanes=20/80) furnished 120 (9.0 g, 63%) as a yellowish oil. ¹H NMR (CDCl₃): δ 4.49 (m, 2H), 3.79 (m, 2H), 3.39 (m, 8H), 2.94 (m, 2H), 1.90-1.38 (m, 14H), 1.28-1.16 (m, 8H), 0.84 (s, 12H). ¹³C NMR (CDCl₃=77.23 ppm): δ 99.23, 76.68, 76.58, 71.95, 71.04, 62.03, 39.35, 35.56, 34.40, 34.20, 30.83, 25.75, 24.71, 24.63, 20.78, 19.60. HRMS (LSIMS, nba): Calcd for C₂₅H₄₇O₅ [(M−2H)+H⁺]: 427.3423, found: 427.3428.

2-{7-[5,5-Dimethyl-6-(tetrahydropyran-2-yloxy)-hexyloxy]-2,2-dimethylheptyloxy}-tetrahydropyran (124). According to the procedure provided for 120, 117 (7.9 g, 34.3 mmol) was successively treated with sodium hydride (95%, 0.76 g, 30.1 mmol) and 116 (10.2 g, 33.2 mmol) in anhydrous THF (80 mL). After aqueous workup, the crude was purified by flash chromatography (silica, hexanes/ethyl acetate=10/1, then 4/1), affording 124 (5.2 g, 34%) as a yellowish oil. ¹H NMR (CDCl₃): δ 4.50 (m, 2H), 3.74 (m, 2H), 3.34 (m, 8H), 2.92 (m, 2H), 1.74-1.21 (m, 26H), 0.81 (s, 12H). ¹³C NMR (CDCl₃): δ 99.15, 76.60, 71.01, 61.91, 39.44, 39.31, 34.31, 30.79, 29.93, 27.27, 25.72, 24.67, 23.91, 20.74, 19.54. HRMS (LSIMS, nba): Calcd for C₂₇H₅₁O₅ [(M−2H)+H⁺]: 455.3736, found: 455.3732.

General Procedure for the Deprotection of THP Ethers:. 6-(5-Hydroxy-4,4-dimethylpentyloxy)-2,2-dimethylhexan-1-ol (126). A solution of 120 (8.0 g, 18.7 mmol) in MeOH (80 mL) and concd HCl (8 mL) was heated to reflux for 4 h, then poured into ice-water (40 mL). The solution was neutralized with saturated NaHCO₃ solution (100 mL) and extracted with ethyl acetate (3×100 mL). The combined organic layers were washed with saturated NH₄Cl solution (100 mL) and brine (100 mL), dried over Na₂SO₄, and concentrated in vacuo. The residue (7.4 g) was purified by chromatography (silica gel; ethyl acetate/hexanes=20/80) to furnish 126 (2.5 g, 51%) as a colorless oil. ¹H NMR (CDCl₃): δ 3.35 (m, 4H), 3.23 (s, 4H), 2.54 (br, 2H), 1.47 (m, 4H), 1.22 (m, 6H), 0.79 (s, 12H). ¹³C NMR (CDCl₃=77.23 ppm): δ 71.77, 71.43, 71.07, 70.98, 38.25, 35.18, 35.06, 34.68, 30.45, 24.23, 20.63. HRMS (LSIMS, gly): Calcd for C₁₅H₃₃O₃ (MH⁺): 261.2430, found: 261.2413. HPLC: Alltima C8 column, 250×4.6 mm, 5μ; 50% acetonitrile, 50% water, flow rate 1.0 mL/min; RI, retention time 7.43 min, 96.4% pure.

7-(6-Hydroxy-5,5-dimethylhexyloxy)-2,2-dimethylheptan-1-ol (130). According to the method given for the synthesis of 126, 124 (5.0 g, 10.9 mmol) was heated to reflux in MeOH (60 mL) and concd HCl (6 mL) for 4 h. Extractive workup gave a crude product that was purified by chromatography (silica; ethyl acetate/hexanes=20/80), affording 130 (2.85 g, 90%) as a colorless oil. ¹H NMR (CDCl₃): δ 3.34 (m, 4H), 3.21 (s, 4H), 2.26 (br, 2H), 1.60-1.40 (m, 4H), 1.34-1.10 (m, 10H), 0.78 (s, 12H). ¹³C NMR (CDCl₃): δ 71.83, 71.67, 71.06, 70.86, 38.68, 38.42, 35.17, 30.53, 29.73, 27.22, 24.02, 23.76, 20.58. HRMS (LSIMS, gly): Calcd for C₁₇H₃₇O₃ (MH⁺): 289.2743, found: 289.2739. HPLC: Alltima C8 column, 250×4.6 mm, 5μ; 60% acetonitrile, 40% water, flow rate 1.0 mL/min; RI, retention time 7.63 min, 95.7% pure.

General Procedure for the Williamson Ether Synthesis followed by THP Deprotection: 6-(5-Hydroxy-4-methyl-4-phenylpentyloxy)-2-methyl-2-phenylhexan-1-ol (127). Under Ar atmosphere, to a suspension of sodium hydride (60%, 600 mg, 15 mmol) in anhydrous THF (100 mL) was added dropwise a solution of 118 (3.3 g, 11.3 mmol) in anhydrous THF (25 mL). After 30 min stirring at room temperature, the mixture was heated to reflux for 1 h, cooled to room temperature, and a solution of 133 (Dasseux, J.-L. H. et al. US 20030078239, 2003; U.S. patent application Ser. No. 09/976,938, filed Oct. 11, 2001) (3.8 g, 11.1 mmol) in anhydrous THF (25 mL) was added dropwise. The reaction mixture was heated to reflux for 22 h, and hydrolyzed by addition of ice (100 g) and saturated NH₄Cl solution (200 mL). The mixture was extracted with ethyl acetate (3×300 mL). The combined organic layers were washed with saturated NH₄Cl solution (3×300 mL), dried over MgSO₄, and concentrated in vacuo to give 121 (10.6 g). A solution of this residue in MeOH (200 mL) and concd HCl (20 mL) was heated to reflux for 6 h. The reaction mixture was diluted with water (50 mL) and the methanol was evaporated under reduced pressure. The solution was extracted with CH₂Cl₂ (4×100 mL). The combined organic layers were washed with saturated NaHCO₃ solution (3×400 mL), dried over MgSO₄, and evaporated to give the crude product (6.5 g). Purification by flash chromatography (silica; hexanes/ethyl acetate=50/50) afforded 127 (1.88 g, 44%) as a colorless oil. ¹H NMR (CDCl₃): δ 7.36-7.15 (m, 10H), 3.68 (m, 2H), 3.55 (m, 2H), 3.27 (m, 4H), 1.85-1.30 (m, 10H), 1.36 (s, 3H), 1.33 (s, 3H). ¹³C NMR (CDCl₃): δ 145.07, 144.97, 128.61, 126.85, 126.31, 126.28, 72.52, 72.24, 71.40, 70.75, 43.55, 43.33, 38.38, 34.84, 30.44, 24.36, 22.03, 21.84, 20.70. HRMS (LSIMS, gly): Calcd for C₂₅H₃₇O₃ (MH⁺): 385.2743, found: 385.2749. HPLC: Alltima C18/cation column, 250×4.6 mm, 5μ; 60% acetonitrile, 40% water, flow rate 1.0 mL/min; RI, retention time 9.10 min, 90.1% pure.

6-(6-Hydroxy-5-methyl-5-phenylhexyloxy)-2,2-dimethyl-hexan-1-ol (129). According to the procedure provided for the synthesis of 127, 117 (10.57 g, 45.9 mmol) was treated with sodium hydride (95%, 1.01 g, 40.0 mmol) and 115 (14.95 g, 42.1 mmol) in anhydrous THF (105 mL). After workup by hydrolysis and extraction, the crude intermediate 123 (21.8 g) was heated to reflux in methanol (80 mL) and concd HCl (8 mL) overnight. After extractive workup, the volatile impurities were distilled off (195° C./0.5 mmHg) and the residue was purified by column chromatography (silica; CH₂Cl₂/acetone=15:1) to furnish 129 (3.2 g, 24%) as an oil. ¹H NMR (CDCl₃): δ 7.38-7.16 (m, 5H), 3.70 (dd, 1H, J=10.7, 4.4), 3.55 (dd, 1H, J=10.7, 7.7), 3.42-3.18 (m, 6H), 1.76 (m, 2H), 1.64-0.95 (m, 15H), 0.85 (s, 6H). ¹³C NMR (CDCl₃=77.00 ppm): δ 144.86, 128.31, 126.60, 125.98, 72.23, 71.58, 70.69, 70.58, 43.32, 38.20, 34.97, 30.35, 30.22, 23.88, 21.62, 20.42. HRMS (LSIMS, gly): Calcd for C₂₁H₃₇O₃ (MH⁺): 337.2743, found: 337.2751. HPLC: Alltima C18 column, 250×4.6 mm, 5μ; 70% acetonitrile, 30% 0.05 M KH₂PO₄, flow rate 1.2 mL/min; UV, retention time 5.83 min, 94.8% pure.

7-(7-Hydroxy-6,6-dimethylheptyloxy)-2,2-dimethylheptan-1-ol (131). According to the procedure provided for the synthesis of 127, 119 (1.83 g, 7.5 mmol) was treated with NaH (60% w/w dispersion in mineral oil, 0.6 g, 15 mmol) and 116 (2.3 g, 7.5 mmol) in anhydrous THF (50 mL). The residue obtained after extractive workup was heated to reflux in methanol (20 mL) and concd HCl (2 mL) for 4 h. Workup and purification by column chromatography (silica; hexanes/ethyl acetate=10/1 to 3/1) afforded 131 (0.68 g, 30%) as a yellow oil. ¹H NMR (CDCl₃): δ 3.40 (t, 4H, J=6.6), 3.31 (s, 4H), 1.71-1.50 (m, 6H), 1.40-1.17 (m, 12H), 0.86 (s, 12H). ¹³C NMR (CDCl₃=77.00 ppm): δ 71.90, 70.90, 38.60, 34.98, 29.66, 27.15, 23.84, 23.66. HRMS (LSIMS, gly): Calcd for C₁₈H₃₉O₃ (MH⁺): 303.2899, found: 303.2907. HPLC: Alltima C18 column, 250×4.6 mm, 5μ; 60% acetonitrile, 40% water, flow rate 1.0 mL/min; RI, retention time 15.73 min, 91.8% pure.

6-(6-Hydroxy-5,5-dimethylhexyloxy)-2,2dimethylhexan-1-ol (128). Under N₂ atmosphere, sodium hydride (60% w/w dispersion in mineral oil, 150 g, 3.75 mol) was washed with hexanes (3×0.5 L) and anhydrous THF (3×0.5 L), then suspended in THF (2 L). A solution of 117 (496 g, 2.15 mol) in THF (1.5 L) was added and the mixture stirred for 30 min at room temperature. After heating to 60° C. for 17 h, the suspension was cooled to 0° C. and a solution of 114 (639 g, 2.18 mol) in THF (1.5 L) was added dropwise, keeping the internal temperature below 35° C. The mixture was heated to reflux for 10 h, stirred at room temperature for 17 h, and hydrolyzed by addition of ice (0.5 L) and saturated NH₄Cl solution (1.5 L). The layers were separated and the aqueous layer was extracted with ethyl acetate (2 L, 2×0.5 L). The combined organic phases were washed with saturated NH₄Cl solution (2×0.6 L), dried over Na₂SO₄ and concentrated in vacuo to give 122 as a yellow, oily residue. A solution of this residue in MeOH (2 L) and concd HCl (0.3 L) was heated to reflux for 48 h. The reaction mixture was cooled to room temperature, diluted with water (1 L), and neutralized with saturated NaHCO₃ solution (1.1 L). The mixture was extracted with ethyl acetate (3×0.7 L). The combined organic extracts were washed with saturated NH₄Cl solution (0.5 L) and saturated NaCl solution (0.5 L), dried over Na₂SO₄, and concentrated in vacuo. The volatiles were removed by distillation under high vacuo at 35-70° C./5 mmHg. The residue was dissolved in MeOH (0.5 L) and concd HCl (50 mL) and heated to reflux for 17 h. The residual oil obtained after extractive workup as above was distilled in high vacuo, affording 128 (216 g, 37%) as a colorless oil. Bp 155-159° C./0.03 mmHg; 160-162° C./0.15 mmHg. ¹H NMR (CDCl₃): δ 3.42 (t, 4H, J=6.8), 3.32 (s, 4H), 1.88 (br, 2H), 1.55 (m, 4H), 1.40-1.30 (m, 8H), 0.86 (s, 12H). ¹³C NMR (CDCl₃=77.00 ppm): δ 71.58, 70.70, 38.16, 34.99, 30.33, 23.90, 20.43. HRMS (LSIMS, nba): Calcd for C₁₆H₃₅O₃ (MH⁺): 275.2586, found: 275.2568. HPLC: Alltima C8 column, 250×4.6 mm, 5μ; 55% acetonitrile, 45% water, flow rate 1.0 mL/min; RI, retention time 8.83 min, 98.0% pure.

3-{3-[3-(2-Ethoxycarbonyl-2-methylpropyl)-phenoxy]-phenyl}-2,2-dimethylpropionic acid ethyl ester (134). To a solution of 132 (von Schickh, O. Chem. Ber. 1936, 69, 242-244) (4.72 g, 23.8 mmol) in CCl₄ (20 mL) were added N-bromo-succinimide (10.6 g, 59.6 mmol) and benzoyl peroxide (75 mg, 0.3 mmol) under N₂ atmosphere. The suspension was heated to reflux for 26 h. The succinimide was removed by filtration and washed with CCl₄ (20 mL). The filtrate was concentrated in vacuo and dried in high vacuo to furnish crude 133 (Marty, W. Inorg. Chem. 1979, 18, 1246-1250) (9.3 g) as a viscous oil. A solution of lithium diisopropylamide (2.0 M in THF/heptane/ethylbenzene, 75 mL, 150 mmol) was added dropwise to a solution of ethyl isobutyrate (18.2 g, 156.6 mmol) in anhydrous THF (200 mL) at −78° C. under N₂ atmosphere. The reaction mixture was stirred for 30 min before the solution of crude 133 (9.3 g) in anhydrous THF (100 mL) was added dropwise, followed by addition of DMPU (30 mL). After 10 min, the reaction mixture was allowed to warm to room temperature and saturated NH₄Cl solution (250 mL) was added. The layers were separated and the aqueous layer was extracted with ethyl acetate (2×200 mL). The combined organic layers were washed with 1 N HCl (200 mL) and saturated NaCl solution (150 mL), dried over MgSO₄, and concentrated in vacuo. The residue was purified by flash chromatography (silica, hexanes/ethyl acetate=95/5) to give 134 (4.40 g, 43%) as a viscous oil. ¹H NMR (CDCl₃): δ 7.20 (t, 2H, J=7.8), 6.84 (m, 4H) 6.76 (t, 2H, J=1.8), 4.06 (q, 4H, J=7.1), 2.82 (s, 4H) 1.20 (t, 6H, J=7.1), 1.17 (s, 12H). ¹³C NMR (CDCl₃): δ 177.40, 157.10, 140.15, 129.29 125.21, 120.77, 117.00, 60.58, 46.23, 43.62, 25.17, 14.31. HRMS (LSIMS, nba): Calcd for C₂₆H₃₅O₅ (MH⁺): 427.2484, found: 427.2443.

3-{3-[3-(2-Carboxy-2-methylpropyl)-phenoxy]-phenyl}-2,2-dimethylpropionic acid (135). A solution of 134 (4.3 g, 10.1 mmol) and KOH (85%, 2.0 g, 30.3 mmol) in ethanol (15 mL) and water (15 mL) was heated to reflux for 4 h. The ethanol was removed in vacuo. The solution was acidified with concd HCl (3 mL) and 1 N HCl (10 mL) to pH 1 and extracted with CH₂Cl₂ (3×50 mL). The combined organic layers were washed with saturated NaCl solution (50 mL), dried over MgSO₄, concentrated in vacuo, and dried in high vacuo to give 135 (3.30 g, 88%) as a viscous, yellowish oil. This material was crystallized from hexanes at 0° C., filtered, washed with cold hexanes (3×5 mL), and dried in vacuo over NaOH to furnish 135 (1.32 g, 35%) as an off-white powder. Mp 84-85° C. ¹H NMR (CDCl₃): δ 14.0-10.0 (br, 2H), 7.27 (t, 2H, J=7.9), 6.99 (m, 2H), 6.59 (s, 2H), 2.76 (s, 4H), 1.25 (s, 12H). “³C NMR (CDCl₃): δ 184.25, 157.21, 139.71, 129.58, 125.67, 118.77, 118.75, 47.79, 44.10, 25.07. HRMS (LSIMS, CI): Calcd for C₂₂H₂₇O₅ (MH⁺): 371.1858, found: 371.1837. HPLC: Alltima phenyl column, 250×4.6 mm, 5μ; 60% acetonitrile, 40% 0.05 M KH₂PO₄, flow rate 1.2 mL/min; RI, retention time 6.67 min, 92.5% pure.

3-{3-[3-(3-Hydroxy-2,2-dimethylpropyl)-phenoxy]-phenyl}-2,2-dimethylpropan-1-ol (136). According to the procedure described for the synthesis of 134, 132 (von Schickh, O. Chem. Ber. 1936, 69, 242-244) (41.2 g, 0.21 mol) was reacted with N-bromo-succinimide (80.9 g, 0.45 mol) and benzoyl peroxide (750 mg, 3.1 mmol) in CCl₄ (170 mL), affording crude 133 (Marty, W. Inorg. Chem. 1979, 18, 1246-1250) (80.2 g) as a viscous oil. Treatment of this dibromide with lithium diisopropylamide (2.0 M, 333 mL, 0.67 mol) and ethyl isobutyrate (77.8 g, 0.67 mol) in anhydrous THF (200 mL) and DMPU (50 mL) led to crude 134 (102.0 g) as a yellow oil. A portion of this diester (20.0 g) in anhydrous THF (100 mL) was added dropwise to a solution of lithium aluminum hydride in diethyl ether (1 M in diethyl ether, 117 mL, 117 mmol) over 45 min at room temperature under N₂ atmosphere. The reaction mixture was stirred for 20 h and then carefully hydrolyzed with water (20 mL). The pH was adjusted to 1 with 25% H₂SO₄ (200 mL) and concd H₂SO₄ (25 mL). The solution was extracted with diethyl ether (2×200 mL). The combined organic phases were washed with saturated NaHCO₃ solution (100 mL), saturated NH₄Cl solution, and saturated NaCl solution (100 mL), dried over MgSO₄, and concentrated in vacuo to give the crude product (16.3 g). This residue was purified repeatedly by flash chromatography (silica, first: hexanes/ethyl acetate=70/30; second: CHCl₃/MeOH=98/2; third: toluene/ethyl acetate=80/20) to afford 136 (3.24 g, 18%) as a colorless, very viscous oil. ¹H NMR (CDCl₃): δ 7.28-7.12 (m, 2H), 6.96-6.78 (m, 6H), 3.28 (s, 4H), 2.53 (s, 4H), 2.23 (s, 2H) 0.86 (s, 12H). ¹³C NMR (CDCl₃): δ 156.98, 141.00, 129.08, 125.54, 121.02, 116.45, 71.07, 44.64, 36.53, 24.15. HRMS (LSIMS, gly): Calcd for C₂₂H₃₁O₃ (MH⁺): 343.2273, found: 343.2257. HPLC: Alltima C8 column, 250×4.6 mm, 5μ; 60% acetonitrile, 40% water, flow rate 1.2 mL/min; RI, retention time 8.19 min, 93.4% pure.

1,14-Bis(tetrahydropyran-2-yloxy)-2,2,13,13-tetramethyltetradecan-6,9-diol (137). A mixture of 2,5-dimethoxytetrahydrofuran (26.4 g, 0.2 mol) and 0.6 N HCl (160 mL) was stirred at room temperature for 1.5 h. The mixture was neutralized with NaHCO₃ (8.4 g, 0.10 mol) and extracted with CH₂Cl₂ (3×50 mL). The aqueous phase was acidified with concd HCl (10 mL), stirred for 1.5 h, neutralized with NaHCO₃ (10.1 g), and extracted with CH₂Cl₂ (3×50 mL). This sequence of acidification, neutralization, and extraction was repeated two more times. The combined organic extracts were dried over MgSO₄ and the solvent was distilled off under atmospheric pressure. Distillation of the residue under reduced pressure gave succinaldehyde (Suzuki, M. Chem. Pharm. Bull. 2001, 49, 29-39) (5.71 g, 33%) as a foul smelling, colorless liquid (bp 75-77° C./15 mmHg. Lit. (House, H. O. J. Org. Chem. 1965, 30, 1061-1070) bp 55-60° C./12 mmHg). Under N₂-atmosphere, to a suspension of Mg powder (3.65 g, 0.15 mol) in anhydrous THF (100 mL) was added dropwise a solution of 112 (Dasseux, J.-L. H. et al. US 20030078239, 2003; U.S. patent application Ser. No. 09/976,938, filed Oct. 11, 2001) (27.9 g, 0.10 mol) in THF (100 mL). The reaction mixture was heated to reflux for 2 h. Under cooling with an ice-bath, a solution of freshly distilled succinaldehyde (3.44 g, 40.0 mmol) in THF (30 mL) was added dropwise and the reaction mixture was stirred at room temperature overnight. The solution was decanted from excess Mg and poured into aqueous saturated NH₄Cl solution (300 mL). After acidification to pH 1-2 with 2 N HCl, the reaction mixture was extracted with diethyl ether (2×100 mL). The combined organic extracts were washed with saturated NaCl solution (100 mL), dried over MgSO₄, and concentrated in vacuo to give a residue that was purified by flash column chromatography (silica, ethyl acetate/hexanes=25/75, then 50/50), affording 137 (18.0 g, 92%) as an almost colorless, very viscous oil. ¹H NMR (CDCl₃): δ 4.54-4.50 (m, 2H), 3.89-3.82 (m, 2H), 3.66 (br s, 2H), 3.48 (pseudo-t, 4H, J=9.6), 2.99 (dd, 2H, J=9.1, 3.5), 2.60 (br s, 2H), 1.90-1.20 (m, 28H), 0.90-0.88 (m, 12H). ¹³C NMR (CDCl₃=77.00 ppm): δ 99.38, 99.15, 76.40, 76.14, 72.14, 71.67, 71.29, 62.39, 62.05, 39.19, 38.77, 38.30, 38.17, 34.18, 33.35, 30.74, 30.64, 25.51, 24.93, 24.65, 24.48, 24.37, 20.04, 19.74, 19.51. HRMS (LSIMS, gly): Calcd for C₂₈H₅₅O₆ (MH⁺): 487.399, found: 487.3995.

5-[5-(5-Hydroxy-4,4-dimethylpentyl)-tetrahydrofuran-2-yl]-2,2-dimethylpentan-1-ol (138). Method A: A solution of 137 (6.18 g, 12.7 mmol) and p-toluenesulfonic acid monohydrate (0.3 g, 1.6 mmol) in toluene (300 mL) was heated under reflux with azeotropic removal of water for 3 h. The reaction mixture was cooled to room temperature and concentrated under reduced pressure. The residue was dissolved in methanol (100 mL) and 3 N sulfuric acid (30 mL) and stirred at room temperature overnight. The methanol was distilled under reduced pressure and the aqueous phase was extracted with ethyl acetate (3×75 mL). The combined organic extracts were washed with water (75 mL) and saturated NaCl solution (75 mL), dried over MgSO₄, and concentrated under reduced pressure. The residue was purified by column chromatography (twice; silica, ethyl acetate/hexanes=75/25, then 50/50) to give 138 (1.35 g, 35%) as a light-yellow oil. Method B: Under N₂-atmosphere, a solution of 137 (3.79 g, 7.8 mmol), tosyl chloride (1.64 g, 8.6 mmol), and pyridine (1.0 mL, 12.4 mmol) in dichloromethane (40 mL) was stirred at room temperature for 21 h. The solvent was removed under reduced pressure, and the residue was dissolved in pyridine (3 mL) and HMPA (5 mL). The mixture was heated to 70-75° C. for 4 h, cooled to room temperature, diluted with water (100 mL) and extracted with CH₂Cl₂ (3×50 mL). The organic extracts were washed with 2 N HCl (until the washings were acidic) and with water, and dried over MgSO₄. The residue obtained after solvent removal was dissolved in methanol (40 mL) and aqueous sulfuric acid (2 mL concd H₂SO₄/5 mL water) and stirred at room temperature overnight. The methanol was removed under reduced pressure. The residue was diluted with water (40 mL) and extracted with ethyl acetate (3×50 mL). The combined organic layers were washed with brine, dried over MgSO₄, and concentrated in vacuo. The residue was purified by column chromatography (silica, ethyl acetate/hexanes=25/75, then 50/50) to give 138 (0.9 g, 38%, mixture of diastereomers in a ratio of ca. 60/40) as a yellow oil. ¹H NMR (CDCl₃): δ 3.96 (m, 2H), 3.86 (m, 2H), 3.30 (m, 8H), 2.41 (s br, 4H), 2.05-1.91 (m, 4H), 1.57-1.18 (m, 28H), 0.86 (s, 12H), 0.85 (s, 12H). ¹³C NMR (CDCl₃=77.00 ppm): δ 79.20, 78.53, 71.13, 37.99, 36.69, 36.48, 35.02, 32.16, 31.12, 24.23, 24.00, 23.95, 20.47. HRMS (LSIMS, gly): Calcd for C₁₈H₃₇O₃ (MH⁺): 301.2743, found: 301.2743. HPLC: Alltima C8 column, 250×4.6 mm, 5μ; 50% acetonitrile, 50% water, flow rate 1.0 mL/min; RI, retention time 11.58 min, 53.3%, retention time 12.00 min, 43.8%; combined: 97.1% pure.

1,15-Bis(tetrahydropyran-2-yloxy)-2,2,14,14-tetramethylpentadecan-6,10-diol (139). An aqueous solution of glutaric aldehyde (25 mL, 50% w/w) was extracted with CH₂Cl₂ (4×50 mL). The organic extracts were dried over MgSO₄ and the solvent was removed by distillation under atmospheric pressure. The residue was distilled in vacuo to give glutaric dialdehyde (7.97 g, 64%, bp 65-66° C./5 mmHg. Lit. bp 68-69° C./25 mmHg) as a malodorous, colorless liquid. According to the procedure given for the synthesis of 136, the Grignard reagent prepared from 112 (Dasseux, J.-L. H. et al. US 20030078239, 2003; U.S. patent application Ser. No. 09/976,938, filed Oct. 11, 2001) (36.9 g, 0.13 mol) and Mg powder (4.8 g, 0.20 mol) in anhydrous THF was reacted with a solution of freshly distilled glutaric aldehyde (6.0 g, 60 mmol) in THF. After workup and concentration, the residue was purified by flash column chromatography (silica, ethyl acetate/hexanes=1/5 to 1/1) to afford 139 (16.67 g, 55%) as an almost colorless, very viscous oil. ¹H NMR (CDCl₃): δ 4.53 (m, 2H), 3.85 (m, 2H), 3.63 (br s, 2H), 3.48 (pseudo-t, 4H, J=8.6), 3.00 (d, 1H, J=9.1), 2.99 (d, 1H, J=9.1), 1.90-1.20 (m, 32H), 0.90 (s, 6H), 0.89 (s, 6H). ¹³C NMR (CDCl₃=77.00 ppm): δ 99.34, 99.15, 76.26, 71.76, 71.47, 62.29, 62.03, 39.24, 38.93, 38.24, 37.38, 34.22, 30.66, 25.53, 24.83, 24.65, 24.44, 21.73, 19.99, 19.83, 19.68, 19.51. HRMS LSIMS, gly): Calcd for C₂₉H₅₇O₆ (MH⁺): 501.4155, found: 501.4152.

2,2,14,14-Tetramethylpentadecane-1,6,10,15-tetraol (140). A solution of 139 (5.75 g, 11.5 mmol) in methanol (100 mL) and diluted aqueous sulfuric acid (1 mL concd H₂SO₄/9 mL water) was stirred at room temperature for 5 h. After dilution with water (20 mL), the methanol was removed under reduced pressure. The obtained aqueous phase was extracted with ethyl acetate (3×50 mL). The combined organic extracts were washed with water (30 mL) and brine (30 mL). The combined aqueous extracts were saturated with NaCl and reextracted with ethyl acetate (3×50 mL). The organic phases were washed with water (20 mL) and brine (20 mL). Saturation with NaCl and reextraction of the aqueous layer was repeated, and the combined organic extracts were dried over MgSO₄. After solvent removal under reduced pressure, the residue was dissolved in the minimal amount of CH₂Cl₂, treated with hexanes for 15 min, and crystallized at room temperature. The crystals were filtered and washed with hexanes to afford 140 (3.06 g, 80%) as a white solid. Mp 85-86° C. ¹H NMR (DMSO-d₆): δ 4.40 (t, 2H, J=5.3), 4.20 (d, 2H, J=5.5), 3.40-3.30 (m, 2H), 3.06 (d, 4H, J=5.3), 1.50-1.05 (m, 18H), 0.77 (s, 12H). ¹³C NMR (DMSO-d₆): δ 69.87, 69.72, 38.36, 37.52, 34.83, 24.10, 21.67, 19.72. HRMS (LSIMS, gly): Calcd for Cl₉H₄₁O₄ (MH⁺): 333.3005, found: 333.2997.

5-[6-(5-Hydroxy-4,4-dimethylpentyl)-tetrahydropyran-2-yl]-2,2-dimethylpentan-1-ol (141). A suspension of 140 (3.06 g, 9.2 mmol) and p-toluenesulfonic acid monohydrate (0.59 g, 3.1 mmol) in toluene (350 mL) was heated to reflux under azeotropic water removal for 7 h. The solvent was evaporated and the residual oil was purified by column chromatography (silica, first: ethyl acetate/hexanes=1/5 to 1/3; second: ethyl acetate/hexanes=1/3), affording 141 (1.58 g, 55%) as a very viscous, colorless oil. ¹H NMR (CDCl₃): δ 3.69 (br s, 2H), 3.31 (s, 4H), 2.10-2.00 (m, 2H), 1.75-1.50 (m, 6H), 1.50-1.18 (m, 12H), 0.86 (s, 12H). ¹³C NMR (CDCl₃=77.00 ppm): δ 71.50, 70.61, 38.51, 35.02, 34.03, 30.28, 24.10, 23.91, 20.02, 18.72. HRMS (LSIMS, gly): Calcd for C₁₉H₃₉O₃ (MH⁺): 315.2899, found: 315.2899. HPLC: Alltima C8 column, 250×4.6 mm, 5μ; 50% acetonitrile, 50% water, flow rate 1.0 mL/min; RI, retention time 14.80 min, 88.3% pure.

6-(5,5-Dimethyl-6-oxohexyloxy)-2,2-dimethylhexanal (142). Under N₂-atmosphere, to a solution of 128 (1.63 g, 5.9 mmol) and freshly distilled triethylamine (2.8 mL) in anhydrous DMSO (10 mL) was added a solution of sulfur trioxide pyridine complex (3.3 g, 20.7 mmol) in anhydrous DMSO (10 mL) at room temperature. The mixture was stirred for 4 h and additional triethylamine (5.6 mL) and sulfur trioxide pyridine complex (3.3 g, 20.7 mmol) was added. The mixture was stirred at room temperature overnight, poured into ice water (100 mL), and stirred for 20 min. The mixture was extracted with diethyl ether (4×30 mL) and the combined organic layers were washed with 10% citric acid (2×20 mL), water (2×20 mL), and saturated NaHCO₃ solution (2×20 mL). Drying over MgSO₄, concentration under reduced pressure, and purification by column chromatography (silica; hexanes/ethyl acetate=5/1 to 3/1) afforded 142 (1.0 g, 63%) as a colorless oil, which should be used as soon as possible for the next step. ¹H NMR (CDCl₃): δ 9.45 (s, 2H), 3.38 (t, 4H, J=6.4), 1.62-1.38 (m, 8H), 1.38-1.14 (m, 4H), 1.05 (s, 1 2H). ¹³C NMR (CDCl₃=77.00 ppm): δ 206.17, 70.36, 45.65, 36.89, 30.08, 21.11, 20.90. HRMS (LSIMS, gly): Calcd for C₁₆H₃₁O₃ (MH⁺): 271.2273, found: 271.2279.

4,4,14,14-Tetramethyl-9-oxaheptadecane-1,17-dioic acid (145). To a solution of methyl diethylphosphonoacetate (12.0 g, 57.1 mmol) in anhydrous DMF (60 mL) was added sodium hydride (60% w/w dispersion in mineral oil, 3.3 g, 82.5 mmol) at room temperature under nitrogen atmosphere, resulting in an exothermic reaction. This mixture was stirred for 30 min, 142 (7.4 g, 24.7 mmol) was added, and stirring was continued overnight. The mixture was hydrolyzed by addition of deionized water (100 mL) and extracted with diethyl ether (4×50 mL). The combined organic layers were dried over MgSO₄ and concentrated in vacuo at 40-50° C. to give crude 143 (11.2 g) as an oil, which was used for the next step without further purification [¹H NMR (CDCl₃): δ 6.91 (d, 2H, J=16.1), 5.71 (d, 2H, J=16.1), 3.73 (s, 6H), 3.36 (t, 4H, J=6.4), 1.65-1.15 (m, 12H), 1.04 (s, 12H). ¹³C NMR (CDCl₃=77.00 ppm): δ 167.54, 158.54, 117.32, 70.68, 51.41, 42.10, 36.75, 30.26, 26.25, 21.28. HRMS (LSIMS, gly): Calcd for C₂₂H₃₉O₅ (MH⁺): 383.2797, found: 383.2789]. A portion of crude 143 (2.0 g) was hydrogenated under elevated H₂ pressure (38 psi) on 10% Pd/C (0.5 g) in EtOH (50 mL) for 20 h. The catalyst was removed by filtration through Celite (1 cm bed) and washed with some EtOH. The filtrate was concentrated under reduced pressure to give crude 144 (1.76 g) as a colorless oil [¹H NMR (CDCl₃): δ 3.66 (s, 6H), 3.40 (t, 4H, J=6.4), 2.25 (m, 4H), 1.63-1.45 (m, 6H), 1.40-1.10 (m, 10H), 0.85 (s, 12H). ³C NMR (CDCl₃=77.00 ppm): δ 174.67, 70.70, 51.38, 41.44, 36.34, 32.27, 30.43, 29.28, 26.60, 20.51. HRMS (LSIMS, nba): Calcd for C₂₂H₄₃O₅ (MH⁺): 387.3110, found: 387.3116]. A solution of 144 (1.76 g) and KOH (85%, 1.5 g, 22.7 mmol) in methanol (50 mL) and deionized water (10 mL) was stirred at room temperature overnight under N₂ atmosphere. Methanol was removed under reduced pressure and the residue was diluted with deionized water (50 mL). The solution was extracted with diethyl ether (4×20 mL) and the ethereal layers were discarded. The aqueous layer was acidified with 2 N HCl (11 mL) to pH 2 and extracted with diethyl ether (4×20 mL). The combined organic layers were washed with saturated NaCl solution (20 mL), dried over MgSO₄, and concentrated and dried in vacuo to afford 145 (0.89 g, 56%) as a colorless oil. ¹H NMR (CDCl₃): δ 9.8-8.8 (br, 2H), 3.42 (t, 4H, J=6.4), 2.29 (m, 4H), 1.67-1.40 (m, 8H), 1.40-1.08 (m, 8H), 0.86 (s, 12H). ¹³C NMR (CDCl₃=77.00 ppm): δ 180.59, 70.75, 41.45, 36.11, 32.37, 30.41, 29.42, 26.71, 20.55. HRMS (LSIMS, gly): Calcd for C₂₀H₃₉O₅ (MH⁺): 359.2797, found: 359.2788. HPLC: Inertsil ODS2 column, 250×4.6 mm, 5μ; 50% acetonitrile, 50% water, flow rate 1.0 mL/min; RI, retention time 23.22 min, 93.6% pure.

2-{6-[5,5-Dimethyl-6-(tetrahydropyran-2-yloxy)-hexyloxy]-2,2-dimethylhexyloxy}-tetrahydropyran (122) and 6-[5,5-Dimethyl-6-(tetrahydropyran-2-yloxy)-hexyloxy]-2,2-dimethylhexan-1-ol (146). Under N₂-atmosphere and at 0° C., to a solution of 128 (85.3 g, 0.31 mol) and p-toluenesulfonic acid hydrate (0.35 g, 1.8 mmol) in CH₂Cl₂ (400 mL) was added dropwise 3,4-dihydro-2H-pyran (26.3 g, 0.31 mol) over 1.5 h. The reaction mixture was stirred at room temperature for 20 h and concentrated in vacuo. The residue was purified twice by column chromatography (silica; CH₂Cl₂/acetone=95/5) to afford 122 (29.0 g, 21%) and 146 (35.7 g, 32%) as colorless oils. 122: ¹H NMR (CDCl₃): δ 4.47 (t, 2H, J=3.3), 3.77 (m, 2H), 3.44 (m, 2H), 3.39 (d, 2H, J=9.1), 3.33 (t, 4H, J=6.6), 2.91 (d, 2H, J=9.1), 1.81-1.40 (m, 16H), 1.30-1.19 (m, 8H), 0.82 (s, 6H), 0.81 (s, 6H). ¹³C NMR (CDCl₃=77.00 ppm): δ 99.03, 76.47, 70.87, 61.80, 39.14, 34.18, 30.62, 30.60, 25.53, 24.48, 24.41, 20.55, 19.37. HRMS (LSIMS, nba): Calcd for C₂₆H₄₉O₅ (M−H⁺): 441.3567, found: 441.3610. HPLC: Alltima phenyl column, 250×4.6 mm, 5μ; 70% acetonitrile, 30% water, flow rate 1.0 mL/min; RI, retention time 7.40 min, 93.5% pure. Anal. (C₂₆H₅₀O₅): C, H. 146: ¹H NMR (CDCl₃): δ 4.53 (t, 1H, J=3.3), 3.88-3.78 (m, 1H), 3.52-3.44 (m, 1H), 3.45 (d, 1H, J=9.1), 3.41 (t, 2H, J=6.5), 3.39 (t, 2H, J=6.5), 3.30 (s br, 2H), 2.99 (d, 1H, J=9.1), 1.90-1.40 (m, 12H), 1.40-1.20 (m, 7H), 0.89 (s, 3H), 0.88 (s, 3H), 0.84 (s, 6H). ¹³C NMR (CDCl₃=77.00 ppm): δ 98.48, 76.02, 70.94, 70.48, 70.38, 61.25, 38.84, 38.11, 34.65, 33.83, 30.24, 30.19, 25.22, 24.21, 24.17, 23.61, 20.22, 20.16, 18.92. HRMS (LSIMS, nba): Calcd for C₂₁H₄₂O₄ (M+1): 359.3161, found: 359.3161. HPLC: Alltima phenyl column, 250×4.6 mm, 5μ; 70% acetonitrile, 30% water, flow rate 1.0 mL/min; RI, retention time 5.05 min, 93.6% pure.

5-Chloro-2,2-dimethylpentanoic acid ethyl ester (147). To a solution of ethyl isobutyrate (130 g, 1.13 mol) and DMPU (5 mL) in THF (160 mL) was added a solution of lithium diisopropylamide (790 mL, 2 M in THF/heptane, 1.58 mol) at −50 to −78° C. The mixture was stirred for 1 h at −78° C. 1-Bromo-3-chloropropane (250 g, 1.58 mol) was added and the mixture was stirred overnight, gradually warming to room temperature. The reaction mixture was poured into a mixture of aqueous hydrochloric acid (6 N, 250 mL), water (500 mL) and ice (500 g) and diluted with saturated NH₄Cl solution (400 mL). The solution was extracted with MTBE (250 mL, 2×150 mL). The combined organic layers were washed with saturated NaCl solution (200 mL), dried over MgSO₄, and concentrated under vacuum to give 147 (248 g) as a colorless oil. Distillation under vacuum furnished pure product (140 g, 64%, bp 73-75° C./2 mmHg) as a colorless oil. ¹H NMR (CDCl₃): δ 4.14 (q, 2H, J=7.1 Hz), 3.53 (t, 2H, J=6.1 Hz), 1.74-1.69 (m, 4H), 1.27 (t, 3H, J=7.1 Hz), 1.21 (s, 6H). ¹³C NMR (CDCl₃): δ 177.2, 60.2, 45.1, 41.7, 37.8, 28.3, 25.1, 14.2. HRMS (EI): Calcd for C₉H₁₇O₂Cl (M⁺): 192.0917, found: 192.0915.

5-[2-(4-Ethoxycarbonyl-4-methylpentyloxy)-ethoxy]-2,2-dimethylpentanoic acid ethyl ester (148). To a solution of ethylene glycol (13.6 g, 220 mmol) in anhydrous N,N′-dimethylacetamide (DMAc, 250 mL) was added potassium tert-butoxide (40 g, 360 mmol) under nitrogen atmosphere. The mixture was stirred at 80° C. for 18 h. A solution of 147 (70 g, 363 mmol) in DMAc (30 mL) and 18-crown-6 (0.35 g, 1.3 mmol) was added and the mixture was stirred at 65° C. for 30 h. Second portions of potassium tert-butoxide (40 g, 360 mmol) and, 3 h later, of 147 (70 g, 363 mmol) were added and stirring was continued, first at 65° C. for 63 h and then at 85° C. for 28 h. The reaction mixture was poured into ice water (1300 mL) and the crude product was extracted with MTBE (4×250 mL). The combined organic layers were washed with saturated NaHCO₃ solution (100 mL) and saturated NaCl solution (100 mL), and dried over anhydrous MgSO₄. The solution was evaporated to yield the crude product as a colorless oil (112 g). The crude product (110 g) was subjected to column chromatography on silica gel eluting with hexanes/ethyl acetate (9:1) to give 148 (19.1 g, 23%) as a colorless oil. ¹H NMR (CDCl₃): δ 4.16 (q, 4H, J=7.1 Hz), 3.61 (s, 4H), 3.49 (t, 4H, J=6.7 Hz), 1.61-1.58 (m, 8H), 1.29 (t, 6H, J=7.1 Hz), 1.22 (s, 12H). ¹³C NMR (CDCl₃): δ 177.6, 71.5, 69.9, 60.1, 41.8, 36.8, 25.1, 25.0, 14.1. HRMS (EI): Calcd for C₂₀H₃₈O₆ (M⁺): 374.2668, found: 374.2664.

5-[2-(5-Hydroxy-4,4-dimethylpentyloxy)-ethoxy]-2,2-dimethylpentan-1-ol (149). Under Ar atmosphere, LiAlH₄ (4.55 g, 120 mmol) was added in portions to MTBE (200 mL) and the suspension was stirred at room temperature for 1 h. A solution of 148 (11.2 g, 30 mmol) in MTBE (40 mL) was added slowly. The mixture was heated to 45° C. for 3 h, then stirred at room temperature for 60 h. The excess of LiAlH₄ was destroyed by dropwise addition of a solution of ethyl acetate (50 mL) in MTBE (50 mL) at 0-10° C. The mixture was stirred at room temperature for 1 h, then hydrolyzed with aqueous hydrochloric acid (6 N, 100 mL) and water (50 mL). The solution was extracted with ethyl acetate (3×100 mL). The combined organic layers were washed with saturated NaCl solution (2×200 mL), dried over MgSO₄, and concentrated under vacuum to give the crude product (9.0 g) as a colorless oil. This residue was subjected to column chromatography on silica using hexanes/ethyl acetate (2:1, then 1:1) as eluent to afford 149 (4.52 g, 52%) as a colorless oil. ¹H NMR (CDCl₃): δ 3.63 (br s, 2H), 3.58 (s, 4H), 3.46 (t, 4H, J=6.5 Hz), 3.27 (s, 4H), 1.59-1.54 (m, 4H), 1.31-1.21 (m, 4H), 0.87 (s, 6H), 0.86 (s, 6H). ¹³C NMR (CDCl₃): δ 72.0, 70.7, 69.8, 34.7, 34.3, 24.0, 23.9. HRMS (LSIMS, nba): Calcd for C₁₆H₃₅O₄ (MH⁺): 291.2535, found 291.2534. HPLC (Alltima C-18, 4.6 mm×250 mm, acetonitrile/water=60/40, flow rate 1.0 mL/min, 35° C., RI detection at 254 nm, retention time 5.37 min): 99.1% pure.

5-[2-(4-Carboxy-4-methylpentyloxy)-ethoxy]-2,2-dimethylpentanoic acid (150). A solution of 148 (15.1 g, 40.3 mmol) and potassium hydroxide (85%, 9.4 g, 142.0 mmol) in ethanol (28 mL) and water (12 mL) was heated to reflux for 4 h. Most of the ethanol was evaporated under reduced pressure. The residue was diluted with water (50 mL). The solution was extracted with diethyl ether (50 mL) and the ether extracts were discarded. The aqueous solution was acidified with aqueous 6 N HCl to pH 1, extracted with MTBE (3×50 mL) and dichloromethane (2×50 mL). The combined organic layers were washed with sat. NaCl (50 mL), dried over MgSO₄, and concentrated in vacuum to get the crude product (12.0 g) as a pale yellow solid. The residue was subjected to column chromatography (silica gel, hexanes:ethyl acetate=2:1, 1:1) to give 150 (5.6 g, 44%) as a colorless oil, which solidified upon standing to give a white solid. Mp 60-61° C. ¹H NMR (CDCl₃): δ 11.93 (br s, 2H), 3.60 (s, 4H), 3.49 (br s, 4H), 1.61-1.60 (m, 8H), 1.21 (s, 12H). ¹³C NMR (CDCl₃): δ 184.4, 71.5, 70.0, 41.8, 36.7, 25.1, 24.9. HRMS (LSIMS, gly): Calcd for C₁₆H₃₁O₆ (MH⁺): 319.2121, found: 319.2117. HPLC (Luna C-18, 4.6 mm×250 mm, acetonitrile/25 mM aq KH₂PO₄ (pH 3)=55/45, 25° C., RI detection at 224 nm, flow rate 1.0 mL/min, retention time 5.03 min): 99.3% pure.

2,2,12,12-Tetramethyltridecanedioic acid diethyl ester (153). Under N₂ atmosphere and at −78° C., a solution of lithium diisopropylamide (2 M in heptane/THF/ethylbenzene, 52.5 mL, 105 mmol) was added dropwise to a solution of ethyl isobutyrate (17.4 g, 150 mmol) in THF (50 mL). The mixture was stirred for 1 h and 1,9-dibromononane (151, 14.3 g, 50 mmol) was added, followed by DMPU (4.4 g, 34.3 mmol). The mixture was stirred for 30 min and the cooling bath was removed. After 30 min, the THF was evaporated under reduced pressure. The residue was diluted with saturated NH₄Cl solution (300 mL) and extracted with ethyl acetate (3×100 mL). The combined organic layers was washed with brine (200 mL), 5% aqueous HCl (100 mL) and saturated NaHCO₃ solution (50 mL), and dried over Na₂SO₄. The solvent was removed under reduced pressure and the residue was distilled in high vacuo to give 153 (14.0 g, 79%) as an oil. Bp 150-151° C./0.1 mmHg. ¹H NMR (CDCl₃): δ (ppm): 4.08 (q, J=7.2, 4H), 1.48-0.98 (m, 18H), 1.21 (t, J=7.2, 6H), 1.12 (s, 12H). ¹³C NMR (CDCl₃): δ (ppm): 178.1, 60.0, 42.1, 40.7, 30.0, 29.4, 25.1, 24.8, 14.2.

2,2,14,14-Tetramethylpentadecanedioic acid diethyl ester (154). A solution of lithium diisopropylamide (1.8 M in hexanes/THF/ethylbenzene, 103.8 mL, 187 mmol) was added to a solution of ethyl isobutyrate (22.0 g, 189 mmol) in THF (150 mL) at −78° C. under N₂ atmosphere. After 1 h, 1,11-dibromoundecane (151, 20.0 g, 63.7 mmol) in THF (20 mL) was added, followed by DMPU (5.0 g, 39.0 mmol). The mixture was stirred for 1 h, then allowed to warm to room temperature and stirred overnight. The THF was evaporated and ethyl acetate (200 mL) and a saturated solution of NH₄Cl (20 mL) were added. The organic layer was separated, washed with saturated NaHCO₃ solution (200 mL) and brine (100 mL), and dried over MgSO₄. The solvent was evaporated and the residue was purified by column chromatography (silica gel, EtOAc:hexanes=1:30) to give 154 (23.1 g, 94%) as a colorless oil. ¹H NMR (CDCl₃): δ (ppm): 4.07 (q, J=7.2, 4H), 1.49-1.44 (m, 4H), 1.24-1.18 (m, 18H), 1.12 (s, 12H). ¹³C NMR (CDCl₃): δ (ppm): 178.0, 60.0, 42.1, 40.7, 30.1, 29.5, 29.4, 25.1, 24.8, 14.2. HRMS (LSIMS, nba): Calcd for C₂₃H₄₅O₄ [M+1]⁺: 385.3318, found: 385.3312. HPLC: 97.4% pure.

2,2,12,12-Tetramethyl-1,13-tridecanediol (155). Under N₂ atmosphere, to a stirred suspension of LiAlH₄ (0.80 g, 21.1 mmol) in diethyl ether (20 mL) was added dropwise a solution of 153 (6.35 g, 17.8 mmol) in diethyl ether (15 mL) at room temperature. The reaction mixture was heated to reflux for 3 h, cooled with an ice bath and carefully hydrolyzed by addition of water (10 mL) and aqueous 2 N HCl (5 mL). The layers were separated and the aqueous layer was extracted with diethyl ether (3×40 mL). The combined organic extracts were dried over Na₂SO₄ and concentrated in vacuo. The residue was distilled in high vacuo to afford 155 (3.0 g, 62%) as an oil, which solidified upon standing. Bp 150-151° C./0.08 mmHg. Mp 52-54° C. ¹H NMR (CDCl₃): δ (ppm): 3.29 (s, 4H), 1.50 (s, 2H), 1.23 (m, 18H), 0.85 (s, 12H). ¹³C NMR (CDCl₃): δ (ppm): 72.0, 38.7, 35.0, 30.5, 29.6, 23.8. HRMS (LSIMS, nba): Calcd for C₁₇H₃₇O₂ (MH⁺): 273.2794, found: 273.2796. HPLC: 97.2% pure. Anal. (C₁₇H₃₆O₂): C, H.

2,2,14,14-Tetramethylpentadecane-1,15-diol (156). Under N₂ atmosphere, methanol (4.2 g, 131.1 mmol) was added dropwise to a stirred suspension of LiBH₄ (2.9 g, 133.1 mmol) in methylene chloride (200 mL), followed by addition of 154 (17.0 g, 44.2 mmol). The reaction mixture was heated to reflux overnight. Water (80 mL) and saturated aqueous NH₄Cl solution (80 mL) were added. The organic phase was separated and the aqueous layer was extracted with methylene chloride (2×50 mL). The organic solutions were combined, washed with saturated aqueous NaHCO₃ solution (50 mL) and brine (80 mL), and dried over MgSO₄. The solvent was partially evaporated, EtOAc (10 mL) was added and the formed crystals were filtered, affording 156 (6.8 g, 51%). Mp 45-47° C. ¹H NMR (CDCl₃): δ (ppm): 3.31 (br s, 4H), 1.71 (br s, 2H), 1.27-1.23 (m, 22H), 0.86 (s, 12H). ¹³C NMR (CDCl₃): δ (ppm): 72.0, 38.7, 35.0, 30.6, 29.6, 23.8. HRMS (LSIMS, nba): Calcd for C₁₉H₄₁O₂ [M+1]⁺: 301.3107, found: 301.3106. HPLC: 100% pure.

2,2,14,14-Tetramethylpentadecanedioic acid (157). A solution of 154 (11.0 g, 28.6 mmol) and KOH (85%, 4.8 g, 72.7 mmol) in ethanol (70 mL) and water (30 mL) was heated to reflux for 20 h. The ethanol was evaporated in vacuo and the remaining mixture was diluted with water (100 mL). After acidification with dilute aqueous HCl, crystals formed, which were filtered and dissolved in ethyl acetate. The solution was dried over MgSO₄ and reduced in volume. The crystals formed were filtered and dried to give 157 (6.5 g, 69%). Mp 95-96° C. ¹H NMR (CDCl₃): δ (ppm): 1.57-1.52 (m, 4H), 1.30-1.25 (m, 18H), 1.20 (s, 12H). ¹³C NMR (CDCl₃): δ (ppm): 185.3, 42.2, 40.7, 30.0, 29.5, 29.5, 29.4, 25.0, 24.9. HRMS (LSIMS, gly): Calcd for C₁₉H₃₇O₄ [M+1]⁺: 329.2692, found: 329.2678.

### 6.3 Large Scale Synthesis of Illustrative Compounds of the Invention

The synthesis of bis(6-hydroxy-5,5-dimethylhexyl)ether [Compound 1], an illustrative compound of the invention was performed on kg-scale. The method involved six synthetic steps with an overall yield of ca. 20%, producing a material of >98% purity.

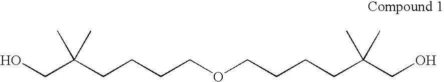

In attempts the synthesis of 1 (Scheme 1) departed from 4,4′-dichlorobutyl ether (4), initially prepared via treatment of THF with phosphorus oxychloride and concentrated sulfuric acid, later available commercially. Substitution of chloride in 4 by iodide led to diiodobutyl ether 5, which was further reacted with lithio ethyl isobutyrate in THF/HMPA to generate 6. Subsequent reduction of the ester groups with lithium aluminum hydride provided 1.

Method A for Synthesis of Illustrative Compounds of the Invention

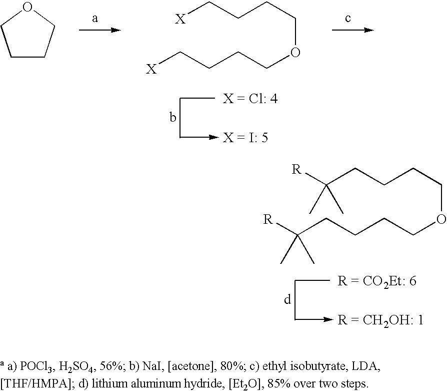

The Williamson reaction is one of the most commonly used methods for the construction of the ether linkage. Accordingly, the synthesis of 1 from two appropriately protected building blocks (i.e., 10 and 11 in Scheme 2) was achieved.

Method B: Synthesis of Bis(6-hydroxy-5,5-dimethylhexyl)ether (1)

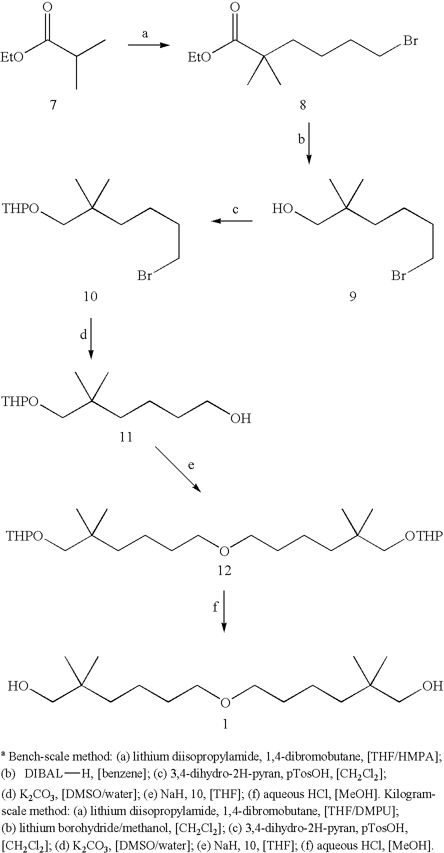

In the first bench-scale runs, ethyl isobutyrate (7) was deprotonated with lithium diisopropylamide (LDA) solution in THF at −78° C. and reacted with 1,4-dibromobutane under addition of HMPA as co-solvent to give bromoester 8 in 70% yield after distillation. Selective reduction of the ester in 8 with diisobutylaluminum hydride (DIBAL-H, 1 M in hexanes) in benzene solution at 50-60° C. furnished bromo alcohol 9 (82%),ⁱ which was subsequently protected with 3,4-dihydro-2H-pyran in the presence of catalytic amounts of p-toluenesulfonic acid in dichloromethane to afford THP-ether 10 (97%). Hydrolysis of 10 with K₂CO₃ in a refluxing mixture of DMSO and waterⁱⁱ gave alcohol 11 (85%), concluding the synthesis of both building blocks required for the Williamson reaction. While first attempts to condense 10 with 11 using various metal/solvent systems at room temperature or reflux with or without addition of crown ethers were unsuccessful, the conversion to protected ether 12 was easily achieved when sodium hydride (3 equiv) in THF was used. Deprotection of crude 12 with 1 M HCl in acetone for 3 days at room temperature provided 1, which was purified by column chromatography (42% yield over both steps).

In the reduction of 8 to 9, care was taken to replace benzene with a non-carcinogenic, preferably non-flammable solvent and to make the reaction more volume-efficient. Although 1.5 M solutions of DEBAL-H in toluene are available, this reagent supplies only 1 mol hydride/mol reagent. Reductions with lithium aluminum hydride or sodium borohydride on the other hand were not chemoselective and the reactions were not reproducible. The same result was obtained, when trimethyl borate was used as a catalyst. An alternate reagent with the capability to reduce esters is lithium borohydride. Several experiments were conducted in order to evaluate its suitability for the reduction of 8. Reactions performed in CH₂Cl₂ or THF at room temperature were too slow; by heating the THF solution to reflux significant amounts of side-products were produced, most likely due to substitution of bromide. However, addition of methanol (1 equiv) to LiBH₄ (150 mol %) in diethyl ether at reflux temperature led to a fast and selective reduction of 8 to 9. It was then shown that the highly flammable Et₂O could be safely replaced with CH₂Cl₂ without affecting the outcome of this reaction, furnishing 9 in high yield (95%) and purity (over 90%) without the need for further purification.

The next two steps could be scaled-up without significant modifications. The THP-protection to 10 with p-toluenesulfonic acid and 3,4-dihydro-2H-pyran gave good yields and purities. For purification of 10, the reaction solution was simply filtered through aluminum oxide for removal of the acid catalyst, a process which was amenable to scale-up. Similarly, the ensuing hydrolysis of bromide 10 to alcohol 11 was performed under the same conditions as described for preliminary small scale experiments (i.e., reaction with 2 equiv K₂CO₃ in a DMSO/water mixture at reflux), and the product was obtained in good yield and sufficient purity to be used as crude material in the next step.

The last two steps of the synthesis were further investigated. The excess of sodium hydride (introduced as a 60% w/w dispersion with mineral oil) used in the Williamson reaction was reduced from three to two equivalents without having an effect on the yield. A smaller excess—however—led to incomplete formation of 12. Since mineral oil from the NaH dispersion contaminated the final product due to co-distillation, it was decided to use dry NaH (95%) in this reaction. The removal of the THP groups in 12 was better effected with 1 M HCl in refluxing methanol, as compared to the originally used HCl in acetone at room temperature. This change led to a shorter reaction time and less side reactions. For example, reacting 10 (1 equiv) and 11 (1 equiv) with NaH (2 equiv) in THF for 10 h at reflux and 17 h at 20-25° C. followed by deprotection with aqueous HCl in refluxing methanol (2 d) and purification by distillation produced 1 in 46% yield and 98% purity (GC).

Although all intermediates in Scheme 2 are liquids, only the first one (8) as well as the final product ESP24232 (1) required purification by distillation. The purities of all the other intermediates were suitable for their use in the synthesis and the obtained yields were satisfactory. Therefore, the procedure was considered adequate for transfer to larger scale.

6.3.1 Kilogram-Scale Synthesis of Illustrative Compounds of the Invention

Further scale-up was performed in steps, aimed first at the production of three 400-g batches of (1) in order to test the robustness and efficiency of the developed method. Surprisingly, in one of the earlier batches of the hydrolysis step (starting with 1900 g of 10), 1115 g of alcohol 11 was isolated in only 51% purity. An investigation into this failure revealed, that this procedure was quite sensitive to the heating conditions: on this scale approximately 2 days at reflux temperature are required to achieve complete conversion; if this heating process was conducted in a discontinued manner, with stops and cool-downs between heating stages, significant amounts of side-products 13 (ca. 20%) and 14 (ca. 8%) were formed (FIG. 2).

Sideproducts Formed During Synthesis of 11.

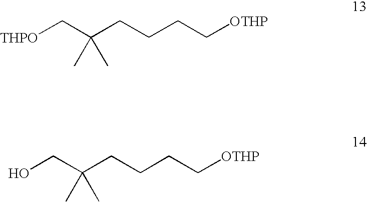

In the following batches the formation of 13 and 14 was avoided by continuously heating the well-stirred reaction mixture to reflux. Starting with the production of ca. 2.9 kg of 8, all three scheduled 400-g runs were completed successfully and produced 1 in quantities of 385 g, 428 g, and 594 g and with purities of 97.6%, 97.6%, and 99.2% (HPLC), respectively. As these results were deemed sufficient for further scale-up, the production of three 1-kg batches of (1) was initiated.

Table B gives an overview of the scale-up campaign of three 1-kg batches of (1). Except for the first run (batch no. A1), the yields and purity results for the alkylation step to 8 varied little and were quite consistent. In the last five experiments (A2-A6) purities ranged from 97.5% to 98.7% (GC) and yields from 63.0% to 65.0%. Since distillation fractions with purities >95% (GC) were retained and combined, the slight deviation both in purity and yield of experiment A1 is explainable by different choices made during the distillation process. In batch no. A6 the amount of co-solvent DMPU was reduced by ca. 25% compared to the previous runs without having a substantial influence on yield or purity.

TABLE B

| batch | | SM wt. | P wt. | purity | | precursor |
|---|---|---|---|---|---|---|
| no. | conversion | (kg) | (kg) | (%) | yield (%) | batch |
| A1 | 7 → 8 | 2.64 | 3.54 | 95.7^{a} | 65.3 | N/A |
| A2 | | 2.81 | 3.75 | 98.6^{a} | 65.0 | |
| A3 | | 2.81 | 3.74 | 98.3^{a} | 64.5 | |
| A4 | | 2.81 | 3.73 | 98.2^{a} | 63.4 | |
| A5 | | 2.81 | 3.73 | 98.7^{a} | 64.6 | |
| A6 | | 2.81 | 3.70 | 97.5^{a} | 64.1^{d} | |
| B1 | 8 → 9 | 7.27 | 6.18 | 97.7^{a} | 105.4^{c,g} | A1, A3 |
| B2 | | 7.48 | 6.25 | 93.7^{a} | 101.9^{c,e} | A2, A4 |
| B3 | | 7.32 | 6.34 | 92.4^{a} | 106.0^{c} | A5, A6 |
| C1 | 9 → 10 | 6.25 | 9.88 | 93.0^{a} | 121.5^{c} | B2 |
| C2 | | 5.10 | 8.20 | 94.6^{a} | 122.3^{c} | B1 |
| C3 | | 5.97 | 7.89 | 94.9^{a} | 101.9^{c,f} | B3 |
| D1 | 10 → 11 | 5.09 | 3.33 | 96.7^{b} | 89.0 | C1 |
| D2 | | 4.56 | 3.08 | 80.4^{b} | 90.9 | C2 |
| D3 | | 4.31 | 3.22 | 88.2^{b} | 99.9 | C3 |
| E1 | 11 → 12 → 1 | 2.68 | 1.17 | 98.6^{b} | 43.2 | C1, D1 |
| E2 | | 2.62 | 1.10 | 99.3^{b} | 40.5 | C2, D2 |
| E3 | | 2.80 | 1.13 | 98.3^{b} | 41.6 | C3, D3 |

^{a}determined by GC-FID;
^{b}determined by RP-HPLC;
^{c}weight yield >100% caused by incompletely removed solvents from reaction or extraction;
^{d}amount of DMPU was reduced by ca. 25% compared to batches A1-A5;
^{e}134 mol % LiBH₄ was used instead of 150 mol %;
^{f}dried for additional 24 h in high vacuo under stirring;
^{g}distilled before use in next step because of accidental contamination with Syltherm heat-transfer oil;
SM = starting material;
P = product.

In the reduction step to bromo alcohol 9 (batch nos. B1-B3), weight yields and purities varied by a greater margin (weight yields from 101.9% to 106.0%; purities from 92.4% to 97.7%).ⁱⁱⁱ Since this compound is a viscous oil, the solvent (CH₂Cl₂) could not be completely removed^{iv} and calculated weight yields >100% were observed. However as the reaction solvent in the next step was also CH₂Cl₂, additional solvent removal efforts seemed to be unnecessary. Moreover in this reaction step, a reduction of the excess of LiBH₄ used (134 mol % instead of 150 mol %) appeared not to affect the outcome (B2). As in entries B1-B3, complete removal of the solvent CH₂Cl₂ was not achieved in the synthesis of 10 either, because of the high viscosity of this oil (C1, C2).^{iv} In experiment C3, 10 was dried in high vacuo under stirring for 24 h in addition to the usual drying procedure, but the reduced amount of residual CH₂Cl₂ had no apparent positive effect in the following steps.

During the scale-up campaign, the most significant fluctuation in purities (80.4-96.7%) and yields (89.0-99.9%) was registered in the synthesis of alcohol 11 (batch nos. D1-D3, Table 1). More consistent results were again obtained in the final conversion of 11 via 12 to 1 (Scheme 2, Table 1). After the Williamson reaction, the crude intermediate 12 was directly deprotected with concd HCl in methanol to 1, which was further purified by distillation (see Experimental Section for details). However, the product obtained in this manner still contained impurities 12 and 15 in the range of 5-7 percent. Therefore, a second deprotection protocol employing the same conditions, concd HCl in refluxing methanol, was performed. After workup and additional fractional distillation in high vacuo, kg-quantities of target compound 1 (ESP24232) having HPLC purities from 98.3 to 99.3% were obtained in yields ranging from 40.5 to 43.2% (over two steps). Moreover, the overall yield for the described six-step synthesis proved to be quite reproducible (E1: 20.0%; E2: 19.4%; E3: 19.4%).

6.3.2 Experimental Data of Illustrative Compounds of the Invention

Chemical reagents were purchased from Sigma-Aldrich or Lancaster and were used without further purification. ACS grade solvents from Fisher Scientific or Mallinckrodt were routinely used for chromatographic purifications and extractions. ¹H NMR spectra were recorded at 300 MHz and ¹³C NMR spectra at 75 MHz and ambient temperature on Varian NMR spectrometers. Chemical shifts for proton NMR are given in parts per million downfield from an internal tetramethylsilane standard and ¹³C chemical shifts are calibrated on the CDCl₃ resonance at 77.23 ppm, unless otherwise specified. Coupling constants (J) are given in Hz. The purity of target compounds was analyzed by HPLC using Shimadzu HPLC systems combined with UV and/or RI detection.

Ethyl 6-bromo-2,2-dimethylhexanoate (8), [Batch No. A6]. In a 100-L glass reactor, a solution of ethyl isobutyrate (7, 3230 mL, 24.2 mol) and THF (5.3 L) was cooled to −38!° C. under stirring and Ar atmosphere. A solution of lithium duisopropylamide (2.0 M in THF/heptane, 11.5 L, 23.0 mol) was added dropwise over 135 min, while maintaining the temperature between −45 and −35° C. After 1 h, 1,4-dibromobutane (4020 mL, 33.7 mol) was added dropwise over 20 min, followed directly by addition of DMPU (420 mL, 3.5 mol) over 2 h, adjusting the addition rates in such a way as to keep the internal reactor temperature between −45 and −35° C. The reaction mixture was stirred for 1 h under cooling and then allowed to slowly warm to room temperature over the next 15 h. The mixture was hydrolyzed with saturated NH₄Cl solution (8.5 L) and water (5 L) and the layers were separated. The aqueous phase was extracted with ethyl acetate (2.2 L). The organic solutions were combined and washed with saturated NaCl solution (7.5 L), aqueous 1 N HCl solution (3.8 L), saturated NaHCO₃ solution (7.5 L), and saturated NaCl solution (2. L). The organic layer was dried over anhydrous MgSO₄ (550 g) and concentrated under reduced pressure. The residue (8178 g) was distilled in vacuum (bp 72-75° C./0.4-0.6 mmHg; lit. 86° C./0.2 mmHg) to give 8 (3700 g, 97.5% pure by GC, 64.1% yield) as an oil. ¹H NMR (CDCl₃): δ (ppm): 4.13 (q, J=7.1 Hz, 2H), 3.40 (t, J=6.8 Hz, 2H), 1.85 (m, 2H), 1.60-1.45 (m, 2H), 1.40-1.30 (m, 2H), 1.25 (t, J=7.1 Hz, 3H), 1.20 (s, 6H). ¹³C NMR (CDCl₃): δ (ppm): 177.3, 60.0, 41.8, 39.4, 33.2, 32.9, 24.9, 23.3, 14.0. GC: 97.5% pure.

6-Bromo-2,2-dimethylhexanol (9) [Batch No. B3]. A 100-L glass reacto was charged with dichloromethane (44 L) and lithium borohydride (95%, 984 g, 42.9 mol) under Ar atmosphere at room temperature. Under stirring methanol (1377 g, 43.0 mol) was added dropwise over 4 h 50 min at a rate that maintained the temperature in the vessel below 28-32° C. A solution of 8 (98.1%, 7318 g, 28.6 mol) in dichloromethane (2.2 L) was added over 3 h 25 min at a rate that maintained a gentle reflux. The mixture was heated to reflux for 15 h, cooled to 0° C., and carefully hydrolyzed by addition of crushed ice (8 kg). Cold, saturated NH₄Cl solution (8.8 L) was added slowly and the mixture was stirred until the effervescence ceased. Water (16 L) was added, the layers were separated, and the aqueous layer was extracted with dichloromethane (2×6.6 L). The organic layers were combined and washed with saturated NH₄Cl solution (2×6.6 L) and saturated NaCl solution (6.6 L). The solution was dried over anhydrous MgSO₄ (1.1 kg) and concentrated under reduced pressure, affording 9 (6338 g, 92.4% pure by GC, 106% weight yield) as an oil, which was used without further purification for the next step. ¹H NMR (CDCl₃): δ (ppm): 3.43 (t, 2H, J=6.8 Hz), 3.33 (s, 2H), 1.85 (m, 2H), 1.61 (br, 1H), 1.48-1.30 (m, 2H), 1.28-1.22 (m, 2H), 0.88 (s, 6H). ¹³C NMR (CDCl₃): δ (ppm): 71.8, 37.7, 35.1, 34.1, 33.6, 23.9, 22.6.

2-(6-Bromo-2,2-dimethylhexyloxy)-tetrahydropyran (10) [Batch No. C₃]. In a 100-L glass reactor, a solution of 9 (92.4%, 5973 g, 26.4 mol) and p-toluenesulfonic acid mono-hydrate (98.5%, 30.1 g, 158 mmol) in dichloromethane (36 L) was cooled to 1° C. under stirring and Ar atmosphere. 3,4-Dihydro-2H-pyran (97%, 2885 g, 33.3 mol) was added dropwise over 4 h at a rate that maintained the temperature below 5° C. The mixture was stirred for 18 h and divided into three equal portions. Each portion was filtered through aluminum oxide (Brockmann Type I, activated, neutral, 3 kg each), which was washed with dichloromethane (2 L each). The combined organic filtrates were concentrated under reduced pressure and dried in high vacuo (1-2 mmHg) for 24 h, affording 10 (94.9% by GC, 7891 g, 101.9% weight yield) as an oil, which was used without farther purification for the next step. ¹H NMR (CDCl₃): δ (ppm): 4.55 (t, 1H, J=3.3 Hz), 3.84 (m, 1H), 3.50 (m, 1H), 3.47 (d, 1H, J=9.0 Hz), 3.42 (t, 2H. J=6.7 Hz), 2.99 (d, 1H, J=9.0 Hz), 1.88 (m, 2H), 1.75-1.23 (m, 10H), 0.91 (s, 3H), 0.90 (s, 3H). ³C NMR (CDCl₃): δ (ppm): 99.4, 76.6, 62.2, 38.6, 34.4, 34.2, 33.9, 30.9, 25.8, 24.8, 24.7, 22.9, 19.7.

5,5-Dimethyl-6-(tetrahydropyran-2-yloxy)-hexan-1-ol (11) [Batch No. D3]. In a 100-L glass reactor, to a solution of 10 (94.9%, 4309 g, 14.0 mol) in DMSO (12.9 L) was added a solution of potassium carbonate (99.5%, 3856 g, 27.9 mol) in water (25.8 L). The mixture was heated to a gentle reflux for 48 h, cooled to room temperature, and diluted with water (13.95 L). The mixture was neutralized by addition of aqueous 5 M HCl (5.6 L) and extracted with dichloromethane (4×4.65 L). The combined organic solutions were washed with saturated NH₄Cl solution (3×4.65 L), dried over anhydrous MgSO₄ (465 g), and concentrated under reduced pressure to give 11 (88.2% by GC, 3222 g, 99.9% yield) as an oil, which was used without further purification for the next step. ¹H NMR (CDCl₃): δ (ppm): 4.54 (t, 1H, J=3.9 Hz), 3.84 (m, 1H), 3.62 (t, 2H, J=6.4 Hz), 3.50 (m, 1H), 3.48 (d, 1H, J=9.1 Hz), 3.00 (d, 1H, J=9.1 Hz), 2.14 (s br, 1H), 1.90-1.44 (m, 8H), 1.40-1.22 (m, 4H), 0.89 (s, 6H). ¹³C NMR (CDCl₃): δ (ppm): 99.4, 76.5, 62.8, 62.2, 39.0, 34.3, 33.7, 30.8, 25.7, 24.7, 24.6, 20.1, 19.7.

Bis(6-hydroxy-5,5-dimethylhexyl)ether (1) (ESP24232) [Batch No. E3]. A 100-L glass reactor was charged with anhydrous THF (25.7 L) under Ar atmosphere. After careful addition of sodium hydride (95%, 504 g, 19.95 mol), 11 (81.9%, 2801 g, 9.9 mol) was added under stirring over 1 h. The mixture was heated to reflux for 24 h and then cooled to room temperature. Bromide 10 (94.9%, 3056 g, 9.9 mol) was added over 20 min and the mixture was heated to reflux for 28 h. After cooling to room temperature, the mixture was hydrolyzed by addition of crushed ice (3 kg) and cold, saturated NH₄Cl solution (10 L). The mixture was extracted with ethyl acetate (9.9 L, 2×3.3 L). The combined organic layers were washed with saturated NH₄Cl solution (3×6 L) and concentrated in vacuo. The residue (12, 5254 g) was dissolved in methanol (18.7 L) and concentrated, aqueous HCl (1.87 L) and heated to reflux for 6 h. The mixture was cooled to room temperature and crushed ice (4.4 kg) was added. The pH of the solution was adjusted to 6.5 by addition of saturated NaHCO₃ solution (22 L). The phases were separated and the aqueous layer was extracted with ethyl acetate (12 L, 2×6 L). The combined organic phases were washed with saturated NH₄Cl solution (4.7 L) and saturated NaCl solution (4.7 L), dried over anhydrous Na₂SO₄ (600 g), and concentrated in vacuo to give crude 1 (47.4% pure by GC, 3575 g). The crude material was divided into two portions (2084 g and 1491 g), which were distilled separately in vacuo (Vigreux column, 30 cm×24 mm, vacuum-jacketed, heated to ca. 40° C.; reflux ratio adjusted to ca. 8/1). The fractions distilling at 158-163° C./0.03-0.05 Torr and 151-167° C./0.1-0.3 Torr, respectively, were combined, affording 1 of higher purity (95.4% pure by GC, 1586 g). This material was dissolved in methanol (1 L) and concentrated, aqueous HCl (100 mL) and heated to reflux for 3 h. The mixture was cooled to room temperature and crushed ice (1 kg) was added. The pH was adjusted to 7 by addition of saturated NaHCO₃ solution (600 mL). The mixture was extracted with ethyl acetate (3×1 L). The combined organic layers were washed with saturated NH₄Cl solution (2×1 L) and saturated NaCl solution (1 L), dried over anhydrous Na₂SO₄ (200 g), and concentrated in vacuo. The residue (1595 g) was divided into two portions (1000 g and 595 g), which were distilled separately in vacuo (Vigreux column, 30 cm×24 mm, vacuum-jacketed, heated to ca. 40° C.; reflux ratio adjusted to ca. 8/1). The fractions distilling at 150-160° C./0.03-0.1 Torr and 150-152° C./0.07-0.1 Torr, respectively, were combined, affording 1 (1130 g, 98.3% pure by HPLC, 41.6% yield) as a colorless, viscous oil. ¹H NMR (CDCl₃): δ (ppm): 3.42 (t, J=6.8 Hz, 4H), 3.20 (s, 4H), 2.80 (br s, 2H), 1.48 (quint, J=6.8 Hz, 4H), 1.10-1.30 (m, 8H), 0.76 (s, 12H). ¹³C NMR (CDCl₃): δ (ppm): 71.1, 70.6, 38.1, 34.8, 30.2, 23.8, 20.3. HRMS (FAB, POS, nba): Calcd for C₁₆H₃₅O₃ (MH⁺): 275.2586, found: 275.2568. HPLC (Alltima C₈, 5μ, 250 mm×4.6 mm, acetonitrile/water=58/42, flow rate 1.0 mL/min, RI detection, retention time 7.08 min): 98.3% pure. Anal. Calcd. for C₁₆H₃₄O₃: C, 70.02; H, 12.49. Found: C, 69.73; H, 12.66 (for batch no. E1).

2-{6-[5,5-Dimethyl-6-(tetrahydropyran-2-yloxy)-hexyloxy]-2,2-dimethylhexyloxy}-tetrahydropyran (12) and 6-[5,5-Dimethyl-6-(tetrahydropyran-2-yloxy)-hexyloxy]-2,2-dimethylhexan-1-ol (15). Under N₂-atmosphere and at 0° C., to a solution of 1 (85.3 g, 0.31 mol) andp-toluenesulfonic acid hydrate (0.35 g, 1.8 mmol) in CH₂Cl₂ (400 mL) was added dropwise 3,4-dihydro-2H-pyran (26.3 g, 0.31 mol) over 1.5 h. The reaction mixture was stirred at room temperature for 20 h and concentrated in vacuo. The residue was purified twice by column chromatography (silica; CH₂Cl₂/acetone=95/5) to afford 12 (29.0 g, 21%) and 15 (35.7 g, 32%) as colorless oils. 12: ¹H NMR (CDCl₃): δ 4.47 (t, 2H, J=3.3), 3.77 (m, 2H), 3.44 (m, 2H), 3.39 (d, 2H, J=9.1), 3.33 (t, 4H, J=6.6), 2.91 (d, 2H, J=9.1), 1.81-1.40 (m, 16H), 1.30-1.19 (m, 8H), 0.82 (s, 6H), 0.81 (s, 6H). ¹³C NMR (CDCl₃=77.00 ppm): δ 99.03, 76.47, 70.87, 61.80, 39.14, 34.18, 30.62, 30.60, 25.53, 24.48, 24.41, 20.55, 19.37. HRMS (LSIMS, nba): Calcd for C₂₆H₄₉O₅ (M−H⁺): 441.3567, found: 441.3610. HPLC: Alltima phenyl column, 250×4.6 mm, 5μ; 70% acetonitrile, 30% water, flow rate 1.0 mL/min; RI, retention time 7.40 min, 93.5% pure. Anal. Calcd. for C₂₆H₅₀O₅: C, 70.54; H, 11.38. Found: C, 70.80; H, 11.49. 15: ¹H NMR (CDCl₃): δ 4.53 (t, 1H, J=3.3), 3.88-3.78 (m, 1H), 3.52-3.44 (m, 1H), 3.45 (d, 1H, J=9.1), 3.41 (t, 2H, J=6.5), 3.39 (t, 2H, J=6.5), 3.30 (s br, 2H), 2.99 (d, 1H, J=9.1), 1.90-1.40 (m, 12H), 1.40-1.20 (m, 7H), 0.89 (s, 3H), 0.88 (s, 3H), 0.84 (s, 6H). ¹³C NMR (CDCl₃=77.00 ppm): δ 98.48, 76.02, 70.94, 70.48, 70.38, 61.25, 38.84, 38.11, 34.65, 33.83, 30.24, 30.19, 25.22, 24.21, 24.17, 23.61, 20.22, 20.16, 18.92. HRMS (LSIMS, nba): Calcd for C₂₁H₄₂O₄ (M+1): 359.3161, found: 359.3161. HPLC: Alltima phenyl column, 250×4.6 mm, 5μ; 70% acetonitrile, 30% water, flow rate 1.0 mL/min; RI, retention time 5.05 min, 93.6% pure. Anal. Calcd. for C₂₁H₄₂O₄: C, 70.34; H, 11.81. Found: C, 70.32; H, 11.91.

2,2,7,7-Tetramethyloctane-1,8-diol (16). Mp 120-122° C. (lit.: 122° C.). ¹H NMR (CDCl₃): δ (ppm): 3.33 (s, 4H), 1.45 (br, 2H), 1.22 (m, 8H), 0.85 (s, 12H). ¹³C NMR (CDCl₃): δ (ppm): 72.18, 38.86, 35.23, 24.95, 24.08.

### 7. BIOLOGICAL ASSAYS

### 7.1 Effects of Illustrative Compounds of the Invention on Non HDL Cholesterol, HDL Cholesterol, Triglyceride Levels, Glycemic Control indicators and Body Weight Control in Obese Female Zucker Rats

In a number of different experiments, illustrative compounds of the invention are administered daily at a dose of up to 100 mg/kg to chow fed obese female Zucker rats for fourteen days in the morning by oral gavage in 1.5% carboxymethylcellulose/0.2% Tween 20 or 20% ethanol/80% polyethylene glycol (dosing vehicles). Animals are weighed daily. Animals are allowed free access to rodent chow and water throughout the study except on days of blood sampling where food is restricted for six hours prior to blood sampling. Blood glucose is determined after the 6 hour fast in the afternoon without anesthesia from a tail vein. Serum is also prepared from pretreatment blood samples subsequently obtained from the orbital venous plexus (with O₂/CO₂ anesthesia) and following the fourteenth dose at sacrifice from the heart following O₂/CO₂ anesthesia. Serums are assayed for lipoprotein cholesterol profiles, triglycerides, total cholesterol, Non HDL cholesterol, HDL cholesterol, the ratio of HDL cholesterol to that of Non HDL cholesterol, insulin, non esterified fatty acids, and beta hydroxy butyric acid. The percent body weight gain and the ratio of liver to body weight is also determined. These are shown as absolute values or as a percent change of the pretreatment values in Table C.

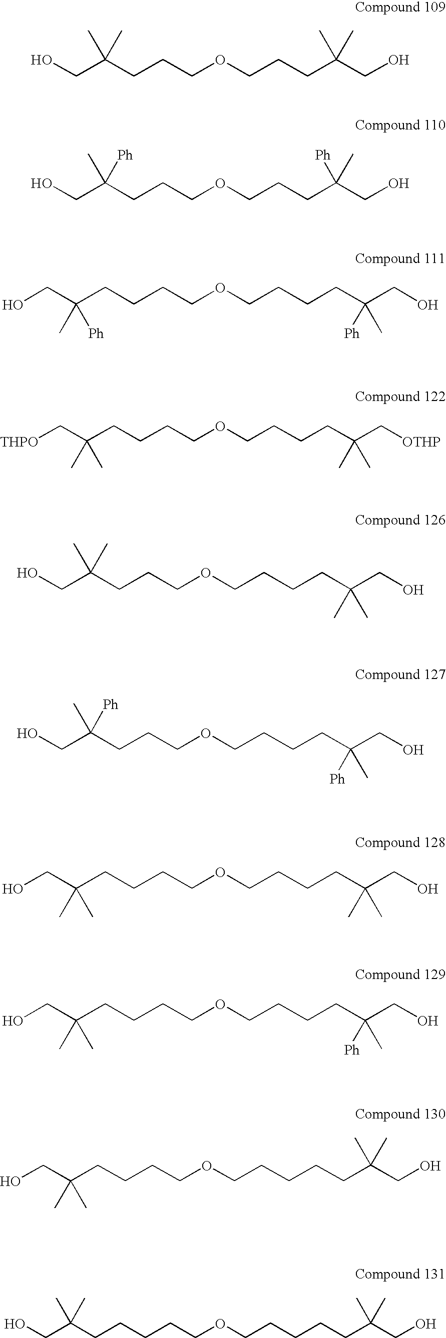

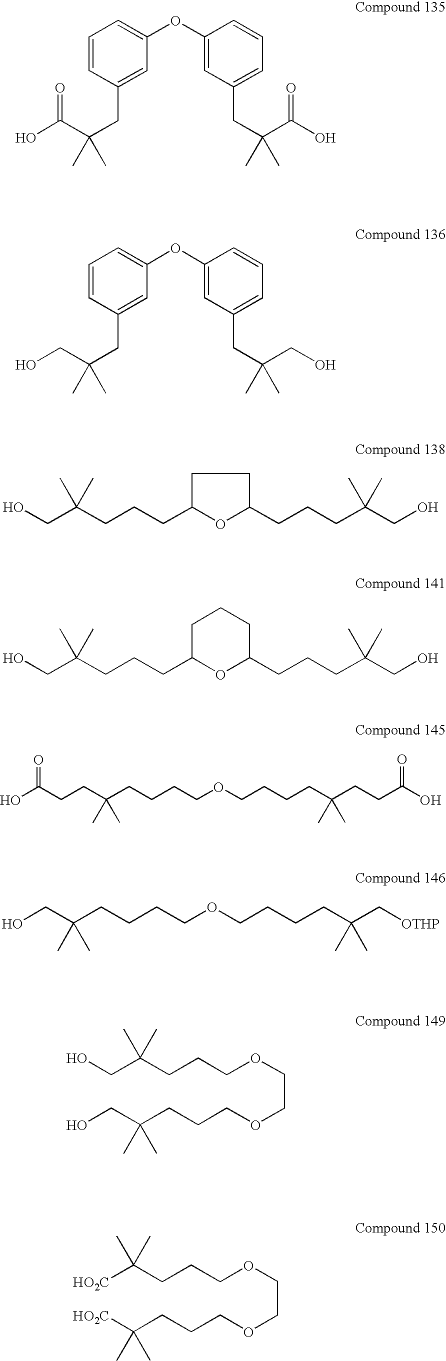

TABLE C
Examples of effects of oral daily treatment of obese female
Zucker rats with compounds of the invention for fourteen days.
Values are the percent change from study prebleed.

| | | Dose | | | Non- | | | |
|---|---|---|---|---|---|---|---|---|
| Compound | Week | (mg/kg/day) | TG | TC | HDL-C | HDL-C | Glucose | Insulin |
| Compound 110 | 1 | 100 | −27 | −4 | −32 | 7 | 12 | −2 |
| | 2 | 100 | −18 | −4 | −26 | 5 | 10 | −6 |
| Compound 111 | 1 | 30 | −14 | −17 | −33 | −4 | | −37 |
| | 2 | 30 | −7 | −20 | −1 | −28 | −18 | −36 |
| Compound 122 | 1 | 100 | −1 | 42 | 46 | 43 | −7 | −18 |
| | 2 | 100 | 22 | 79 | 92 | 79 | 9 | −20 |
| Compound 126 | 1 | 100 | −63 | 119 | −28 | 630 | 68 | 240 |
| | 2 | 100 | −58 | 161 | −17 | 786 | 9 | 145 |
| Compound 127 | 1 | 30 | 0 | 3 | | −2 | 5 | 3 |
| | 2 | 30 | 21 | 2 | 45 | −20 | −3 | 0 |
| Compound 128 | 1 | 30 | −52 | 131 | −14 | 174 | 2 | 48 |
| | 2 | 30 | −39 | 224 | 59 | 349 | 44 | 48 |
| Compound 129 | 1 | 100 | −61 | −6 | −55 | 26 | 14 | 2 |
| | 2 | 100 | −45 | 26 | −13 | 52 | 4 | 6 |
| Compound 130 | 1 | 30 | −66 | 41 | −26 | 71 | 6 | 25 |
| | 2 | 30 | −47 | 77 | 17 | 105 | 6 | 13 |
| Compound 131 | 1 | 100 | −91 | 85 | −79 | 292 | −6 | 40 |
| | 2 | 100 | −88 | 106 | −57 | 289 | −5 | −1 |
| Compound 135 | 1 | 70 | −45 | 3 | −53 | 63 | −13 | −43 |
| | 2 | 70 | −52 | −7 | −41 | 31 | −16 | 1 |
| Compound 136 | 1 | 90 | 13 | 1 | −12 | 22 | −20 | −57 |
| | 2 | 90 | 18 | 9 | 23 | −4 | −3 | −45 |
| Compound 138 | 1 | 97 | −49 | 128 | 11 | 287 | 35 | 43 |
| | 2 | 97 | −13 | 74 | 31 | 140 | 10 | 69 |
| Compound 141 | 1 | 100 | −74 | 28 | −70 | 75 | −14 | −2 |
| | 2 | 100 | −47 | 70 | −31 | 127 | −9 | 10 |
| Compound 145 | 1 | 100 | −80 | −3 | −50 | 13 | −1 | −34 |
| | 2 | 100 | −73 | 21 | −30 | 39 | 16 | −45 |
| Compound 149 | 1 | 30 | −37 | −7 | −48 | 37 | −20 | −40 |
| | 2 | 30 | −30 | −7 | −27 | 17 | −27 | −26 |

### 7.2 Effects of Illustrative Compounds of the Invention on the in Vitro Lipid Synthesis in Isolated Hepatocytes

Compounds were tested for inhibition of lipid synthesis in primary cultures of rat hepatocytes. Male Sprague-Dawley rats were anesthetized with intraperitoneal injection of sodium pentobarbital (80 mg/kg). Rat hepatocytes were isolated essentially as described by the method of Seglen (Seglen, P. O. Hepatocyte suspensions and cultures as tools in experimental carcinogenesis. J. Toxicol. Environ. Health 1979, 5, 551-560). Hepatocytes were suspended in Dulbecco's Modified Eagles Medium containing 25 mM D-glucose, 14 mM HEPES, 5 mM L-glutamine, 5 mM leucine, 5 mM alanine, 10 mM lactate, 1 mM pyruvate, 0.2% bovine serum albumin, 17.4 mM non-essential amino acids, 20% fetal bovine serum, 100 nM insulin and 20 μg/mL gentamycin) and plated at a density of 1.5×10⁵ cells/cm² on collagen-coated 96-well plates. Four hours after plating, media was replaced with the same media without serum. Cells were grown overnight to allow formation of monolayer cultures. Lipid synthesis incubation conditions were initially assessed to ensure the linearity of [1-¹⁴C]-acetate incorporation into hepatocyte lipids for up to 4 hours. Hepatocyte lipid synthesis inhibitory activity was assessed during incubations in the presence of 0.25 μCi [1-¹⁴C]-acetate/well (final radiospecific activity in assay is 1 Ci/mol) and 0, 1, 3, 10, 30, 100 or 300 μM of compounds for 4 hours. At the end of the 4-hour incubation period, medium was discarded and cells were washed twice with ice-cold phosphate buffered saline and stored frozen prior to analysis. To determine total lipid synthesis, 170 μl of MicroScint-E® and 50 μl water was added to each well to extract and partition the lipid soluble products to the upper organic phase containing the scintillant. Lipid radioactivity was assessed by scintillation spectroscopy in a Packard TopCount NXT. Lipid synthesis rates were used to determine the IC₅₀s of the compounds that are presented in Table D.

TABLE D
Effect of Compounds on Lipid Synthesis
in Primary Rat Hepatocytes.

95% Confidence
Interval

| Compound | IC₅₀ (μM) | Lower | Upper | r² |
|---|---|---|---|---|
| 110 | 143 | 106.7 | 194 | .944 |
| 111 | 106 | 66.4 | 168 | .987 |
| 122 | 20 | 8.9 | 45.2 | .978 |
| 126 | 17 | 10.6 | 27.8 | .989 |
| 128 | 10 | 9.3 | 11.9 | .999 |
| 129 | 4.7 | 1.8 | 12.2 | .971 |
| 130 | 8.4 | 3.1 | 22.6 | .965 |
| 131 | 3.8 | 2.4 | 6.0 | .992 |
| 135 | 52 | 32.3 | 86.1 | .889 |
| 136 | 98 | | | .901 |
| 138 | 9.0 | | | .986 |
| 141 | 39 | 14.5 | 106 | .986 |
| 145 | 22 | 2.5 | 200 | .989 |

The present invention is not to be limited in scope by the specific embodiments disclosed in the examples which are intended as illustrations of a few aspects of the invention and any embodiments which are functionally equivalent are within the scope of this invention. Indeed, various modifications of the invention in addition to those shown and described herein will become apparent to those skilled in the art and are intended to fall within the appended claims.

What is claimed is:

1. A compound of a formula I: 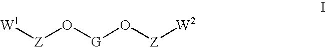
or a pharmaceutically acceptable salt, hydrate, solvate, or a mixture thereof, wherein:
(a) each occurrence of Z is independently (CH₂)ₘ, (CH═CH)ₜ, or phenyl, where each occurrence of m and t is an independent integer ranging from 1 to 9;
(b) G is (CH₂)ₓ, CH₂CH═CHCH₂, CH═CH, CH₂₋phenyl-CH₂, or phenyl, where x is 2, 3, or 4;
(c) W¹ and W² are independently C(R¹)(R²)(CH₂)ₙ₋Y, V, C(R³)(R⁴)—(CH₂)_{c-}C(R⁵)(R⁶)—(CH₂)ₙ₋Y, or C(R¹)(R²)—(CH₂)_{c-}V where c is 1 or 2 and n is an integer ranging from 0 to 4;
(d) each occurrence of R¹ and R² is independently (C₁₋C₆)alkyl, (C₂₋C₆)alkenyl, (C₂₋C₆)alkynyl, phenyl, benzyl, or, R¹ and R² and the carbon to which they are both attached are taken together to form a (C₃-C₇)cycloakyl group;
(e) each occurrence of R³ and R⁴ is independently H, (C₁₋C₆)alkyl, (C₂₋C₆)alkenyl, (C₂₋C₆)alkynyl, phenyl, benzyl, R³ and R⁴ and the carbon to which they are both attached are taken together to form a (C₃-C₇)cycloakyl group;
(f) R⁵ is H, (C₁₋C₆)alkyl, (C₂₋C₆)alkenyl, (C₂₋C₆)alkynyl, (C₁₋C₆)alkoxy, phenyl, benzyl, Cl, Br, CN, NO₂, or CF₃;
(g) R⁶ is OH, (C₁₋C₆)alkyl, (C₂₋C₆)alkenyl, (C₂₋C₆)alkynyl, (C₁₋C₆)alkoxy, phenyl, benzyl, Cl, Br, CN, NO₂, or CF₃;
(h) V is 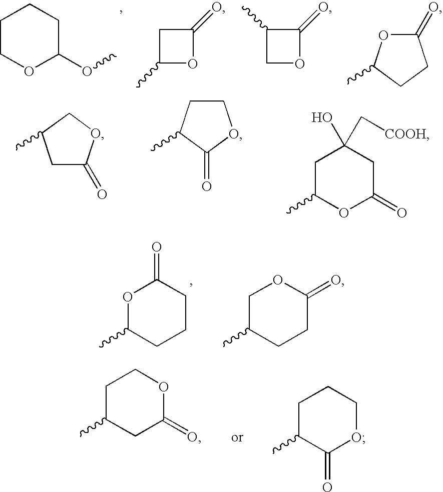
(i) each occurrence of Y is independently (C₁₋C₆)alkyl, OH, COOH, CHO, COOR⁷, SO₃H, 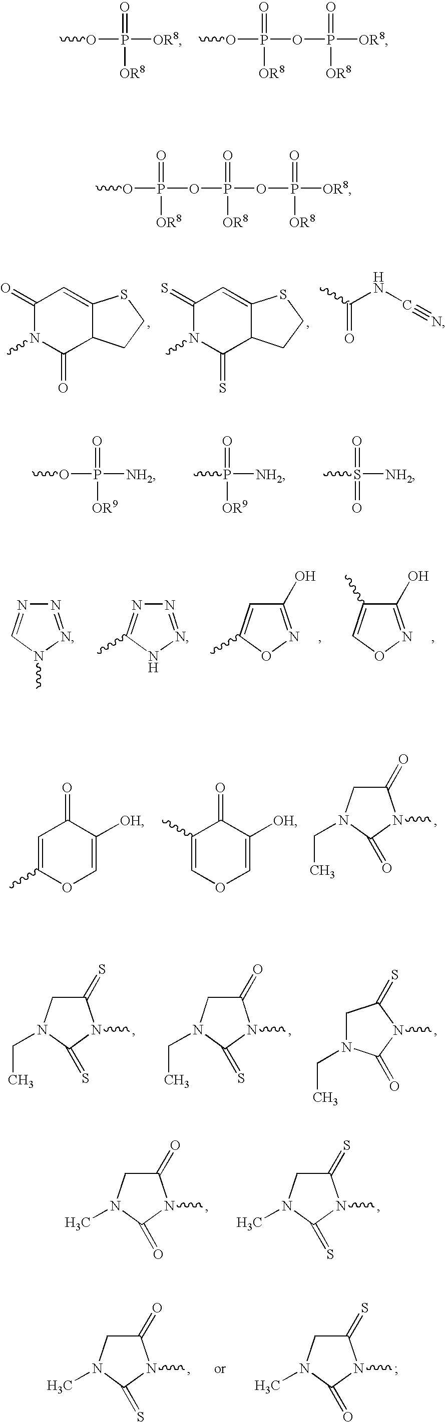
(j) R⁷ is (C₁₋C₆)alkyl, (C₂₋C₆)alkenyl, (C₂₋C₆)alkynyl, phenyl, or benzyl and is unsubstituted or substituted with one or more halo, OH, (C₁₋C₆)alkoxy, or phenyl groups;
(k) each occurrence of R⁸ is independently H, (C₁₋C₆)alkyl, (C₂₋C₆)alkenyl, or (C₂₋C₆)alkynyl and is unsubstituted or substituted with one or two halo, OH, (C₁₋C₆)alkoxy, or phenyl groups;
(l) each occurrence of R⁹ is independently H, (C₁₋C₆)alkyl, (C₂₋C₆)alkenyl, or (C₂₋C₆)alkynyl; and
(m) with the provisos:
(i) that when G is (CH₂)ₓ, then W¹ and W² cannot both be C(R¹)(R²)—CHO or cannot both be 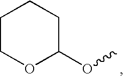
(ii) that when G is phenyl, then W¹ and W² cannot:
both be C(R¹)(R²)—COOH,
both be C(R¹)(R²)—CH₂OH,
both be C(R¹)(R²)—COOR⁷,
both be (CH₂)₃₋C(H)(OH)—CH₂OH,
both be (CH₂)₂₋C(H)(OH)—CH₂OH,
both be C(R¹)(R²)—CHO, or
both be 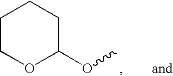
(iii) that when every occurrence of Z is phenyl, then W¹ and W² cannot both be C(R¹)(R²)—OH.

2. The compound of claim 1, wherein W¹ and W² are independently C(R¹)(R²)(CH₂)ₙ₋Y, V, C(R³)(R⁴)—(CH₂)_{c-}C(R⁵)(R⁶)—Y, or C(R¹)(R²)—(CH₂)_{c-}V.

3. The compound of claim 1, wherein W¹ and W² are independently C(R¹)(R²)(CH₂)ₙ₋Y, V, or C(R¹)(R²)—(CH₂)_{c-}V.

4. The compound of claim 1, wherein W¹ and W² are independent C(R¹)(R²)(CH₂)ₙ₋Y groups.

5. The compound of claim 1, wherein W¹ is C(R¹)(R²)(CH₂)ₙ₋Y.

6. The compound of claim 1, wherein W¹ is V.

7. The compound of claim 1, wherein W¹ is C(R³)(R⁴)—(CH₂)_{c-}C(R⁵)(R⁶)—Y.

8. The compound of claim 1, wherein W¹ is C(R¹)(R²)—(CH₂)_{c-}V.

9. The compound of claim 4, wherein each occurrence of Y is independently OH, COOR⁷, or COOH.

10. The compound of claim 4, wherein each occurrence of Y is independently OH or COOH.

11. The compound of claim 1, wherein m is an integer ranging from 1 to 4 and t is 1.

12. The compound of claim 1, wherein R⁶ is OH, (C₁₋C₆)alkyl, (C₂₋C₆)alkenyl, (C₂₋C₆)alkynyl, (C₁₋C₆)alkoxy, phenyl, benzyl, Cl, or Br.

13. A compound of a formula Ia: 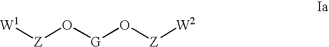
or a pharmaceutically acceptable salt, hydrate, solvate, or a mixture thereof, wherein:
(a) each occurrence of Z is independently (CH₂)ₘ or (CH═CH)ₜ, where each occurrence of m and t is an independent integer ranging from 1 to 9;
(b) G is (CH₂)ₓ, CH₂CH═CHCH₂, or CH═CH, where x is 2, 3, or 4;
(c) W¹ and W² are independently C(R¹)(R²)(CH₂)ₙ₋Y, V, or C(R¹)(R²)—(CH₂)_{c-}V, where c is 1 or 2 and n is an integer ranging from 0 to 4;
(d) each occurrence of R¹ and R² is independently (C₁₋C₆)alkyl, (C₂₋C₆)alkenyl, (C₂₋C₆)alkynyl, phenyl, benzyl, or R¹ and R² and the carbon to which they are both attached are taken together to form a (C₃-C₇)cycloakyl group;
(e) V is 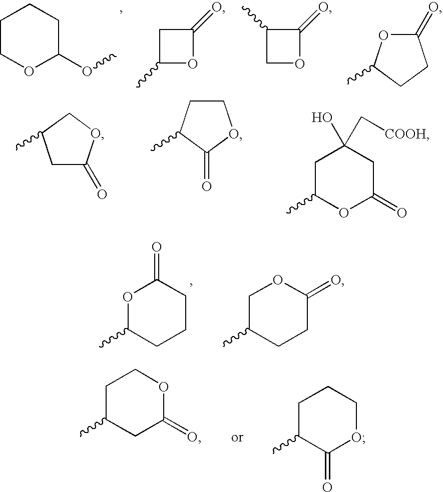
(f) each occurrence of Y is independently OH, COOH, CHO, COOR⁷, SO₃H, 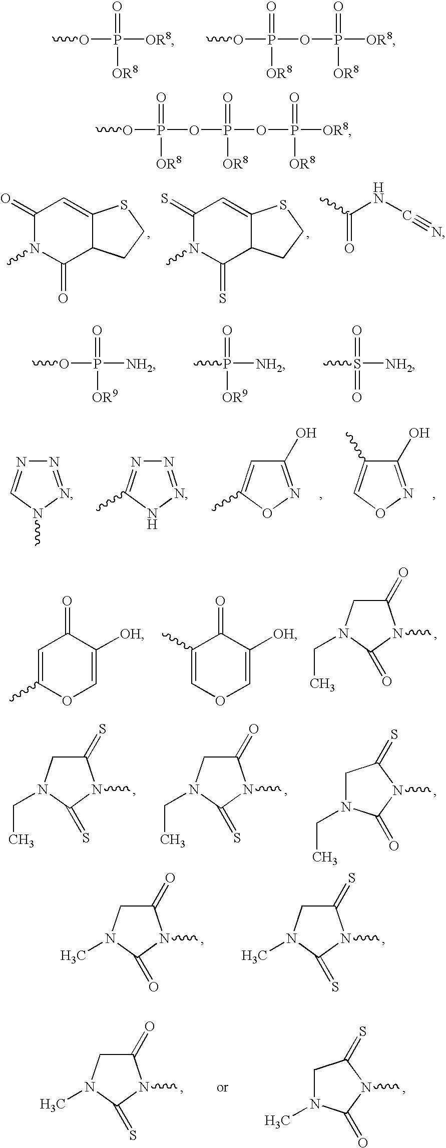
(g) R⁷ is (C₁₋C₆)alkyl, (C₂₋C₆)alkenyl, (C₂₋C₆)alkynyl, phenyl, or benzyl and is unsubstituted or substituted with one or more halo, OH, (C₁₋C₆)alkoxy, or phenyl groups;
(h) each occurrence of R⁸ is independently H, (C₁₋C₆)alkyl, (C₂₋C₆)alkenyl, or (C₂₋C₆)alkynyl and is unsubstituted or substituted with one or two halo, OH, (C₁₋C₆)alkoxy, or phenyl groups;
(i) each occurrence of R⁹ is independently H, (C₁₋C₆)alkyl, (C₂₋C₆)alkenyl, or (C₂₋C₆)alkynyl; and
(j) with the proviso that when G is(CH₂)ₓ, then W¹ and W² cannot both be C(R¹)(R²)—CHO or cannot both be

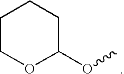

14. The compound of claim 13, wherein W¹ and W² are independent C(R¹)(R²)(CH₂)ₙ₋Y groups.

15. The compound of claim 13, wherein W¹ is C(R¹)(R²)(CH₂)ₙ₋Y.

16. The compound of claim 13, wherein W¹ is V.

17. The compound of claim 13, wherein W¹ is C(R¹)(R²)—(CH₂)_{c-}V.

18. The compound of claim 14, wherein each occurrence of Y is independently OH, COOR⁷, or COOH.

19. The compound of claim 14, wherein each occurrence of Y is independently OH or COOH.

20. The compound of claim 13, wherein m is an integer ranging from 1 to 4 and t is 1.

21. A compound of the formula Ib 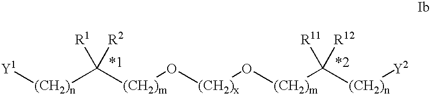
or a pharmaceutically acceptable salt, hydrate, solvate, or a mixture thereof, wherein:
(a) each occurrence of m is independently an integer ranging from 1 to 9;
(b) each occurrence of n is an independent integer ranging from 0 to 4;
(c) x is 2, 3, or 4;
(d) each occurrence of R¹ and R² is independently (C₁-C₆)alkyl, (C₂-C₆)alkenyl, (C₂-C₆)alkynyl, phenyl, benzyl, or R¹ and R² and the carbon to which they are both attached are taken together to form a (C₃-C₇)cycloakyl group;
(e) each occurrence of R¹¹ and R¹² and the carbon to which they are both attached are taken together to form a (C₃-C₇)cycloakyl group;
(f) Y¹ and Y² are independently (C₁-C₆)alkyl, OH, COOH, CHO, COOR⁷, SO₃H, 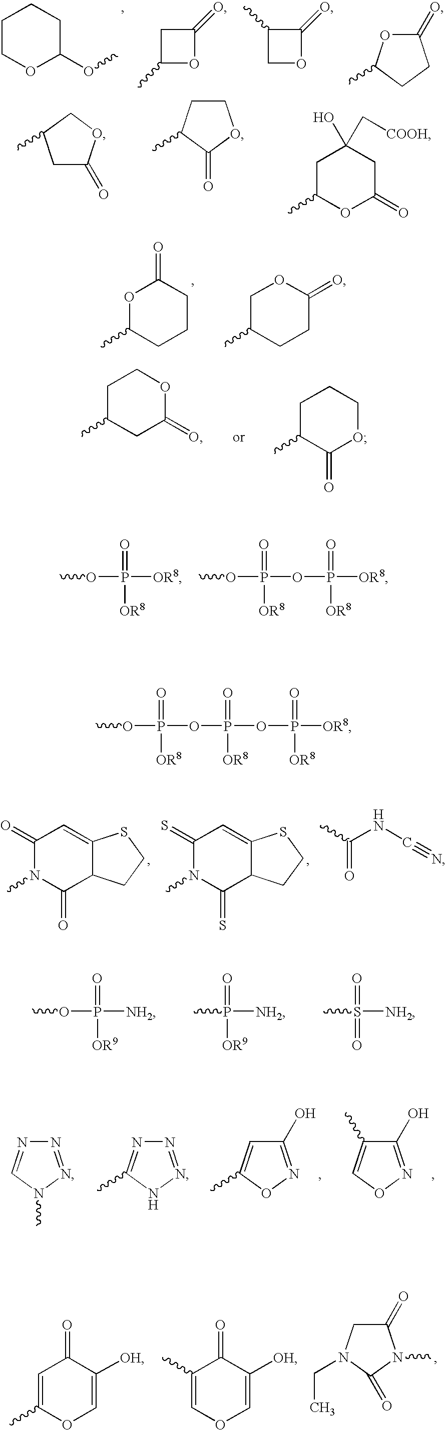 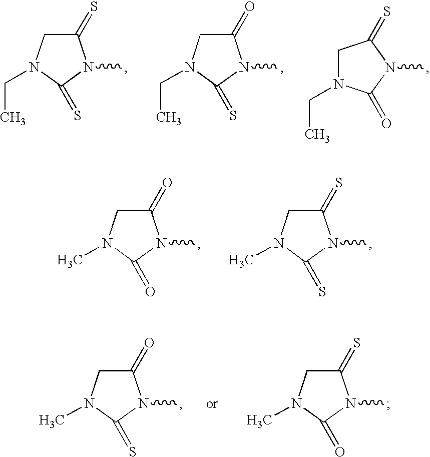
(g) R⁷ is (C₁₋C₆)alkyl, (C₂₋C₆)alkenyl, (C₂₋C₆)alkynyl, phenyl, or benzyl and is unsubstituted or substituted with one or more halo, OH, (C₁₋C₆)alkoxy, or phenyl groups;
(h) each occurrence of R⁸ is independently H, (C₁₋C₆)alkyl, (C₂₋C₆)alkenyl, or (C₂₋C₆)alkynyl and is unsubstituted or substituted with one or two halo, OH, (C₁₋C₆)alkoxy, or phenyl groups;
(i) each occurrence of R⁹ is independently H, (C₁₋C₆)alkyl, (C₂₋C₆)alkenyl, or (C₂₋C₆)alkynyl; and
(j) with the proviso that both occurrences of Y cannot both be CHO.

22. The compound of claim 21, wherein each occurrence of Y is independently OH, COOR⁷, or COOH.

23. The compound of claim 21, wherein each occurrence of Y is independently OH or COOH.

24. The compound of claim 22, wherein each occurrence of R¹ or R² is independently (C₁₋C₆)alkyl group.

25. The compound of claim 22, wherein each occurrence of R¹ or R² is methyl.

26. A compound of the formula Ic 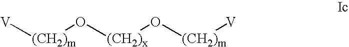
or a pharmaceutically acceptable salt, hydrate, solvate, or a mixture thereof, wherein:
(a) each occurrence of m is an independent integer ranging from 1 to 9;
(b) x is 2, 3, or 4;
(c) V is

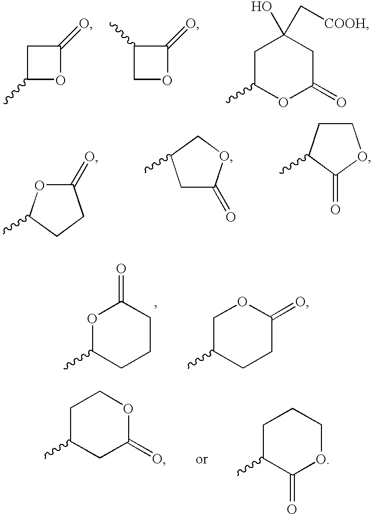

27. A compound of a formula Id: 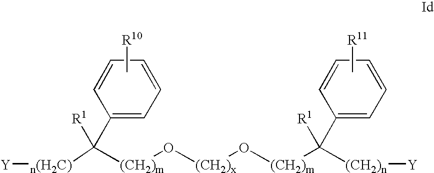
or a pharmaceutically acceptable salt, hydrate, solvate, or a mixture thereof, wherein
(a) each occurrence of m is independently an integer ranging from 1 to 9;
(b) each occurrence of n is an independent integer ranging from 0 to 4;
(c) x is 2, 3, or 4;
(d) each occurrence of R¹ is independently (C₁₋C₆)alkyl, (C₂₋C₆)alkenyl, (C₂₋C₆)alkynyl, phenyl, or benzyl;
(e) each occurrence of Y is (C₁₋C₆)alkyl, OH, COOH, CHO, COOR⁷, SO₃H, 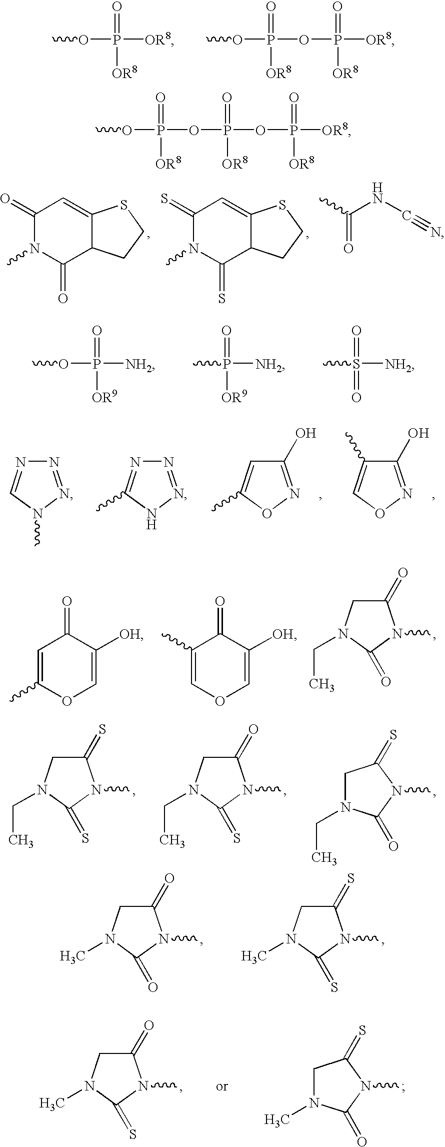
(f) R⁷ is H, (C₁₋C₄)alkyl, phenyl, or benzyl, and is substituted or unsubstituted with one or more halo, OH, (C₁-C₆)alkoxy, or phenyl groups;
(g) each occurrence of R⁸ is independently H, (C₁₋C₆)alkyl, (C₂₋C₆)alkenyl, or (C₂₋C₆)alkynyl and is unsubstituted or substituted with one or two halo, OH, (C₁₋C₆)alkoxy, or phenyl groups;
(h) each occurrence of R⁹ is independently H, (C₁₋C₆)alkyl, (C₂₋C₆)alkenyl, or (C₂₋C₆)alkynyl;
(i) R¹⁰ and R¹¹ are independently H, halogen, (C₁₋C₄)alkyl, (C₁₋C₄)alkoxy, (C₆)aryl, (C₆)aryloxy, CN, or NO₂, N(R⁷)₂.

28. A compound of the formula:
I-114 4-[3-(3-carboxy-3-methyl-butoxy)-propoxy]-2,2-dimethyl-butyric acid,
I-297 5-[2-(5-hydroxy-4,4-dimethyl-pentyloxy)-ethoxy]-2,2-dimethyl-pentan-1-ol,
IV-1 3-{3-[3-(2-Carboxy-2-methyl-propyl)-phenoxy]-phenyl}-2,2-dimethyl-propionic acid,
IV-2 1-{3-[3-(2-hydroxy-2-methyl-propyl)-phenoxy]-phenyl}-2-methyl-propan-2-ol,
or a pharmaceutically acceptable salt, hydrate, solvate, clathrate, enantiomer, diastereomer, racemate, or a mixture of stereoisomer thereof.

29. A compound of the formula:
I-1: 4-[2-(3-hydroxy-3-methyl-butoxy)-ethoxy]-2-methyl-butan-2-ol;
I-2: 4-[2-(4-hydroxy-3,3-dimethyl-butoxy)-ethoxy]-2,2-dimethyl-butan-1-ol;
I-3: 4-[2-(3-carboxy-3-methyl-butoxy)-ethoxy]-2,2-dimethyl-butyric acid;
I-4: 4-[2-(3,3-dimethyl-4-oxo-butoxy)-ethoxy]-2,2-dimethyl-butanal;
I-5: 4-[2-(3-methoxycarbonyl-3-methyl-butoxy)-ethoxy]-2,2-dimethyl-butyric acid methyl ester;
I-6: 2,2-dimethyl-4-[2-(3-methyl-3-phenoxycarbonyl-butoxy)-ethoxy]-butyric acid phenyl ester;
I-7: benzyl-2,2,2′,2′-tetramethyl-4,4′-[ethylenebis(oxadiyl)]dibutryrate;
I-8: 2,2′-dimethyl-4,4′-[ethylenebis(oxadiyl)]dibutane-2-sulfonic acid;
I-9: phosphoric acid mono-{3-[2-(3,3-dimethyl-butoxy)-ethoxy]-1,1-dimethyl-propyl}ester;
I-10: 1-ethyl-3-(3-{2-[3-(4,6-dioxo-2,3,3a,6-tetrahydro-4H-thieno[3,2-c]pyridin-5-yl))-3-methyl-butoxy]-ethoxy}-1,1-dimethyl-propyl)-4,6-dioxo-2,3,3a,6-tetrahydro-4H-thieno[3,2-c]pyridin-5-yl-4,6-dione;
I-11: 1-ethyl-3-(3-{2-[3-(4,6-dithioxo-2,3,3a,6-tetrahydro-4H-thieno[3,2-c]pyridin-5-yl))-3-methyl-butoxy]-ethoxy}-1,1-dimethyl-propyl)-4,6-dioxo-2,3,3a,6-tetrahydro-4H-thieno[3,2-c]pyridin-5-yl-4,6-dithione;
I-12: 2,2-dimethyl-4-[2-(3-methyl-3-cyanocarbamoyl-butoxy)-ethoxy]-N-cyano-butyramide;
I-13: phosphoradimic acid mono-(3-{2-[3-(amino-hydroxy-phosphoryloxy)-3-methyl-butoxy]-ethoxy}-1,1-dimethyl-propyl)ester;
I-14: {1,1-dimethyl-3-[2-(3-methyl-3-phosphonamido-butoxy)-ethoxy]-propyl}-phosphonic acid amide;
I-15: 1-{3-[2-(3-methyl-3-{(1H)-tetrazol-1-yl}-butoxy)-ethoxy]-1,1-dimethyl-propyl}-1H-tetrazole;
I-16: 5-{3-[2-(3-methyl-3-{(1H)tetrazol-5-yl}-butoxy)-ethoxy]-1,1-dimethyl-propyl}-1H-tetrazole;
I-17: 1-ethyl-3-(3-{2-[3-(3-ethyl-2,5-dithioxo-imidazolidin-1-yl)-3-methyl-butoxy]-ethoxy}-1,1-dimethyl-propyl)-imidazolidine-2,4-dione;
I-18: 1-ethyl-3-(3-{2-[3-(3-ethyl-2,5-dioxo-imidazolidin-1-yl)-3-methyl-butoxy]-ethoxy}-1,1-dimethyl-propyl)-imidazolidine-2,4-dione;
I-19: 1-ethyl-3-(3-{2-[3-(3-ethyl-2-oxo-5-thioxo-imidazolidin-1-yl)-3-methyl-butoxy]-ethoxy}-1,1-dimethyl-propyl)-imidazolidine-2-thioxo-4-one;
I-20: 1-ethyl-3-(3-{2-[3-(3-ethyl-5-oxo-2-thioxo-imidazolidin-1-yl)-3-methyl-butoxy]-ethoxy}-1,1-dimethyl-propyl)-imidazolidine-4-thioxo-2-one;
I-21: 1-{3-[2-(3-methyl-3-(3-methyl-isoxazol-5-yl)-butoxy)-ethoxy]-1,1-dimethyl-propyl}-5-isoxazole;
I-22: 1-{3-[2-(3-methyl-3-(3-methyl-isoxazol-4-yl)-butoxy)-ethoxy]-1,1-dimethyl-propyl}-4-isoxazole;
I-23: 3-{3-[2-(3-methyl-3-(5-hydroxy-pyran-3-yl-4-one)-butoxy)-ethoxy]-1,1-dimethyl-propyl}-5-hydroxy-pyran-4-one;
I-24: 2-{3-(2-[3-methyl-3-(5-hydroxy-pyran-2-yl-4-one)-butoxy]-ethoxy)]-1,1-dimethyl-propyl}-5-hydroxy-pyran-4-one;
I-25: 2-{3-[2-(3-methyl-3-(5-hydroxy-pyran-3-yl-4-one)-butoxy]-ethoxy}-1,1-dimethyl-propyl}-5-hydroxy-pyran-4-one;
I-26: 1-(2-tetrahydropyranyloxy)-2-{2-[2-(2-tetrahydropyranyloxy)-ethoxy]-ethoxy}ethane;
I-27: 4-{2-[2-(4-oxetan-2-one)-propoxy-ethoxy]-ethyl}-oxetan-2-one;
I-28: 3-{2-[2-(3-oxetan-2-one)-propoxy-ethoxy]-ethyl}-oxetan-2-one;
I-29: 5-{2-[2-(5-dihydro-furan-2-one)-propoxy-ethoxy]-ethyl}-dihydro-furan-2-one;
I-30: 4-{2-[2-(4-dihydro-furan-2-one)-propoxy-ethoxy]-ethyl}-dihydro-furan-2-one;
I-31: 3-{2-[2-(3-dihydro-furan-2-one)-propoxy-ethoxy]-ethyl}-dihydro-furan-2-one;
I-32: 2-{2-[2-(2-{2-[4-(carboxy-methyl)-4-hydroxy-6-oxo-tetrahydro-pyran-2-yl]-ethoxy}-ethoxy)-ethyl]-4-hydroxy-6-oxo-tetrahydro-pyran-4-yl}-acetic acid;
I-33: 2,2′-[ethylenebis(oxadiyl)]diethane-6-d-valerolactone;
I-34: 2,2′-[ethylenebis(oxadiyl)]diethane-5-d-valerolactone;
I-35: 2,2′-[ethylenebis(oxadiyl)]diethane-4-d-valerolactone;
I-36: 2,2′-[ethylenebis(oxadiyl)]diethane-3-d-valerolactone;
I-37: 3,3,3′,3′-tetramethyl-5,5′-[ethylenebis(oxadiyl)]dipentanol;
I-38: 3,3,3′,3′-tetramethyl-5,5′-[ethylenebis(oxadiyl)]dipentanoic acid;
I-39: 3,3,3′,3′-tetramethyl-5,5′-[ethylenebis(oxadiyl)]dipentanal;
I-40: methyl-3,3,3′,3′-tetramethyl-5,5′-[ethylenebis(oxadiyl)]dipentanoate;
I-41: phenyl-3,3,3′,3′-tetramethyl-5,5′-[ethylenebis(oxadiyl)]dipentanoate;
I-42: benzyl-3,3,3′,3′-tetramethyl-5,5′-[ethylenebis(oxadiyl)]dipentanoate;
I-43: 4,4,4′,4′-tetramethyl-6,6′-[ethylenebis(oxadiyl)]dihexanol;
I-44: 4,4,4′,4′-tetramethyl-6,6′-[ethylenebis(oxadiyl)]dihexanoic acid;
I-45: 4,4,4′,4′-tetramethyl-6,6′-[ethylenebis(oxadiyl)]dihexanal;
I-46: methyl-4,4,4′,4′-tetramethyl-6,6′-[ethylene-(oxadiyl)]-dihexanoate;
I-47: phenyl-4,4,4′,4′-tetramethyl-6,6′-[ethylenebis(oxadiyl)]dihexanoate;
I-48: benzyl-4,4,4′,4′-tetramethyl-6,6′-[ethylenebis(oxadiyl)]dihexanoate;
I-49: 2,2,2′,2′-tetramethyl-4,4′-[ethylenebis(oxadiyl)]dibutane sulfonic acid;
I-50: phosphoric acid mono-{4-[2-(3,3-dimethyl-4-phosphonooxy-butoxy)-ethoxy]-2,2-dimethyl-butyl}ester;
I-51: 5-{4-[2-(3,3-dimethyl-4-(5-(3,3a-dihydro-2H-thieno-[3,2-c]pyridine-4,6-dioxo)pentyloxy)-ethoxy]-2,2-dimethyl-butyl}-3,3a-dihydro 3,3a-dihydro-2H-thieno-[3,2-c]pyridine-4,6-dione;
I-52: 5-{4-[2-(3,3-dimethyl-4-(5-(3,3a-dihydro-2H-thieno-[3,2-c]pyridine-4,6-dithioxo)pentyloxy)-ethoxy]-2,2-dimethyl-butyl}-3,3a-dihydro3,3a-dihydro-2H-thieno-[3,2-c]pyridine-4,6-dithione;
I-53: 5-[2-(3,3-dimethyl-4-cyanocarbamoyl-butoxy)-ethoxy]-3,3-dimethyl-N-cyano-pentanoic acid-amide;
I-54: phosphoramidic acid mono-(4-{2-[4-(amino-hydroxy-phosphoryloxy)-3,3-dimethyl-butoxy]-ethoxy}-2,2-dimethyl-butyl)ester;
I-55: {4-[2-(3,3-dimethyl-4-phosphonamido-butoxy)-ethoxy]-2,2-dimethyl-butyl}-phosphonamide;
I-56: 1-{4-[2-(3,3-dimethyl-5-{1H-tetrazol-1-yl}-butoxy)-ethoxy]-2,2-dimethyl-butyl}-1H-tetrazole;
I-57: 5-{4-[2-(3,3-dimethyl-5-{1H-tetrazol-5-yl}-butoxy)-ethoxy]-2,2-dimethyl-butyl}-1H-tetrazole;
I-58: 5-{4-[2-(3,3-dimethyl-5-{3-hydroxy-isoxazol-5-yl}-butoxy)-ethoxy]-2,2-dimethyl-butyl}-3-hydroxy-isoxazole;
I-59: 4-{4-[2-(3,3-dimethyl-5-{3-hydroxy-isoxazol-4-yl}-butoxy)-ethoxy]-2,2-dimethyl-butyl}-3-hydroxy-isoxazole;
I-60: 2-{4-[2-(3,3-dimethyl-5-{5-hydroxy-pyran-4-oxo-3-yl}-butyloxy)-ethoxy]-2,2-dimethyl-butyl}-5-hydroxy-pyran-4-one;
I-61: 2-{4-[2-(3,3-dimethyl-5-{5-hydroxy-pyran-4-oxo-2-yl}-butyloxy)-ethoxy]-2,2-dimethyl-butyl}-5-hydroxy-pyran-4-one;
I-62: 3-{4-[2-(3,3-dimethyl-5-{5-hydroxy-pyran-4-oxo-3-yl}-butyloxy)-ethoxy]-2,2-dimethyl-butyl}-5-hydroxy-pyran-4-one;
I-63: 1-ethyl-3-(4-{2-[4-(3-ethyl-2,5-dithioxo-imidazolidin-1-yl)-3,3-dimethyl-butoxy]-ethoxy}-2,2-dimethyl-butyl)-imidazolidine-2,4-dione;
I-64: 1-ethyl-3-(4-{2-[4-(3-ethyl-2,5-dioxo-imidazolidin-1-yl)-3,3-dimethyl-butoxy]-ethoxy}-2,2-dimethyl-butyl)-imidazolidine-2,4-dione;
I-65: 1-ethyl-3-(4-{2-[4-(3-ethyl-2-thioxo-4-oxo-imidazolidin-1-yl)-3,3-dimethyl-butoxy]-ethoxy}-2,2-dimethyl-butyl)-imidazolidine-2-thioxo-4-one;
I-66: 1-ethyl-3-(4-{2-[4-(3-ethyl-2-oxo-4-thioxo-imidazolidin-1-yl)-3,3-dimethyl-butoxy]-ethoxy}-2,2-dimethyl-butyl)-imidazolidine-2- oxo-4-thione;
I-67: 3,3,3′,3′-tetramethyl-5,5′-[ethylenebis(oxadiyl)]dipentane sulfonic acid;
I-68: phosphoric acid mono-{1,1-dimethyl-3-[2-(3-methyl-3-phosphonooxy-butoxy)-ethoxy]-propyl}ester;
I-69: 5-(5-{2-[3,3-dimethyl-5-(4,6-dioxo-2,3,3a,6-tetrahydro-4h-thieno-[3,2-c]pyridin-5-yl)-pentyloxy]-ethoxy}-3,3-dimethyl-pentyl)-3,3a-dihydro-2H-thieno[3,2-c]pyridine-4,6-dione;
I-70: 5-(5-{2-[3,3-dimethyl-5-(4,6-dithioxo-2,3,3a,6-tetrahydro-4h-thieno[3,2-c]pyridin-5-yl)-pentyloxy]-ethoxy}-3,3-dimethyl-pentyl)-3,3a-dihydro-2H-thieno[3,2-c]pyridine-4,6-dione;
I-71: 6-[2-(3,3-dimethyl-5-cyano-carbamoyl-butoxy)-ethoxy]-4,4-dimethyl-N-cyano-hexanoic acid-amide;
I-72: phosphoramidic acid mono-(5-{2-[5-(amino-hydroxy-phosphoryloxy)-3,3-dimethyl-pentyloxy]-ethoxy}-3,3-dimethyl-pentyl)ester;
I-73: {5-[2-(3,3-dimethyl-5-phosphonamido-pentyloxy)-ethoxy]-3,3-dimethyl-pentyl}-phosphonamide;
I-74: 1-{[2-(3,3-dimethyl-5-tetrazol-1-yl-pentyloxy)-ethoxy]-3,3-dimethyl-pentyl}-1H-tetrazole;
I-75: 5-{5-[2-(3,3-dimethyl-5-tetrazol-1-yl-pentyloxy)-ethoxy]-3,3-dimethyl-pentyl}-1H-tetrazole;
I-76: 5-{5-[2-(3,3-dimethyl-5-{3-hydroxy-isoxazol-5-yl}-pentyloxy)-ethoxy]-3,3-dimethyl-pentyl}-isoxazol-3-ol;
I-77: 4-{5-[2-(3,3-dimethyl-5-{3-hydroxy-isoxazol-4-yl}-pentyloxy)-ethoxy]-3,3-dimethyl-pentyl}-isoxazol-3-ol;
I-78: 3-{5-[2-(5-{5-hydroxy-4-oxo-4H-pyran-2-yl}-3,3-dimethyl-pentyloxy)-3,3-dimethyl-pentyl]-5-hydroxy-pyran-4-one;
I-79: 2-{5-[2-(5-{5-hydroxy-4-oxo-4H-pyran-2-yl}-3,3-dimethyl-pentyloxy)-3,3-dimethyl-pentyl]-5-hydroxy-pyran-4-one;
I-80: 3-{5-[2-(5-{5-hydroxy-4-oxo-4H-pyran-3-yl}-3,3-dimethyl-pentyloxy)-3,3-dimethyl-pentyl]-5-hydroxy-pyran-4-one;
I-81: 1-ethyl-3-(5-{2-[5-(3-ethyl-2,5-dithioxo-imidazolidin-1-yl)-3,3-dimethyl-pentyloxy]-ethoxy}-3,3-dimethyl-pentyl)-imidazolidine-2,4-dione;
I-82: 1-ethyl-3-(5-{2-[5-(3-ethyl-2,5-dioxo-imidazolidin-1-yl)-3,3-dimethyl-pentyloxy]-ethoxy}-3,3-dimethyl-pentyl)-imidazolidine-2,4-dione;
I-83: 1-ethyl-3-(5-{2-[5-(1-ethyl-2-thioxo-5-oxo-imidazolidin-3-yl)-3,3-dimethyl-pentyloxy]-ethoxy}-3,3-dimethyl-pentyl)-imidazolidine-2-thioxo-4-one;
I-84: 1-ethyl-3-(5-{2-[5-(1-ethyl-2-oxo-5-thioxo-imidazolidin-3-yl)-3,3-dimethyl-pentyloxy]-ethoxy}-3,3-dimethyl-pentyl)-imidazolidine-2-oxo-4-thione;
I-85: 4-[4-(3-hydroxy-3-methyl-butoxymethyl)-benzyloxy]-2-methyl-butan-2-ol;
I-86: 4-[4-(4-hydroxy-3,3-dimethyl-butoxymethyl)-benzyloxy]-2,2-dimethyl-butan-1-ol;
I-87: 4-[4-(3-carboxyl-3,3-dimethyl-butoxymethyl)-benzyloxy]-2,2-dimethyl-butyric acid;
I-88: 4-[4-(4-hydroxy-3,3-dimethyl-butoxymethyl)-benzyloxy]-2,2-dimethyl-butanal;
I-89: 4-[4-(3,3-dimethyl-3-carboxymethyl-butoxymethyl)-benzyloxy]-2,2-dimethyl-butyric acid methyl ester;
I-90: 2,2-dimethyl-4-[4-(3-methyl-3-phenoxycarbonyl-butoxymethyl)-benzyloxy]-butyiic acid phenyl ester;
I-91: 4-[4-(3-benzyloxycarbonyl-3-methyl-butoxymethyl)-benzyloxy]-2,2-dimethyl-butyric acid benzyl ester;
I-92: 2,2′-dimethyl-4,4′-[vinylbis(oxadiyl)]dibutane-2-sulfonic acid;
I-93: phosphoric acid mono-{1,1-dimethyl-3-[4-(3-methyl-3-phosphonooxy-butoxymethyl)-benzyloxy]-propyl}ester;
I-94: 2,2′-dimethyl-4,4′-[vinylbis(oxadiyl)]dibutanol;
I-95: 4-[2-(4-hydroxy-3,3-dimethyl-butoxy)-vinyloxy]-2,2-dimethyl-butan-1-ol;
I-96: 4-[2-(3-carboxyl-3,3-dimethyl-butoxy)-vinyloxy]-2,2-dimethyl-butyric acid;
I-97: 4-[2-(4-hydroxy-3,3-dimethyl-butoxy)-vinyloxy]-2,2-dimethyl-butanal;
I-98: 4-[2-(3,3-dimethyl-3-carboxymethyl-3-butoxy)-vinyloxy]-2,2-dimethyl-butyric acid methyl ester;
I-99: 2,2-dimethyl-4-[2-(3-methyl-3-phenoxycarbonyl-butoxy)-vinyloxy]-butyric acid phenyl ester;
I-100: 2,2-dimethyl-4-[2-(3-methyl-3-benzyloxycarbonyl-butoxy)-vinyloxy]-butyric acid benzyl ester;
I-101: 4-[2-(3,3-dimethyl-3-sulfono-butoxy)-vinyloxy]-2-methyl-butane-2-sulfonic acid;
I-102: phosphoric acid mono-{3-[2-(3,3-dimethyl-butoxy)-vinyloxy]-1,1-dimethyl-propyl}ester;
I-103: 4-[4-(3-hydroxy-3-methyl-butoxy)-phenoxy]-2-methyl-butan-2-ol;
I-104: 4-[4-(4-hydroxy-3,3-dimethyl-butoxy)-phenoxy]-2,2-dimethyl-butan-1-ol;
I-105: 4-[4-(3-carboxyl-3,3-dimethyl-butoxy)-phenoxy]-2,2-dimethyl-butyric acid;
I-106: 4-[4-(4-hydroxy-3,3-dimethyl-butoxy)-phenoxy]-2,2-dimethyl-butanal;
I-107: 4-[4-(3,3-dimethyl-3-carboxymethyl-butoxy)-phenoxy]-2,2-dimethyl-butyric acid methyl ester;
I-108: 2,2-dimethyl-4-[4-(3-methyl-3-phenoxycarbonyl-butoxy)-phenoxy]-butyric acid phenyl ester;
I-109: 4-[4-(3-benzyloxycarbonyl-3-methyl-butoxy)-phenoxy]-2,2-dimethyl-butyric acid benzyl ester;
I-110: 4-[4-(3,3-dimethyl-3-sulfono-butoxy)-phenoxy]-2-methyl-butane-2-sulfonic acid;
I-111: 4-[4-(3,3-dimethyl-3-oxyphosphono-butoxy)-phenoxy]-2-methyl-butane-2-oxyphosphoric acid;
I-112: 4-[3-(3-hydroxy-3-methyl-butoxy)-propoxy]-2-methyl-butan-2-ol;
I-113: 4-[3-(4-hydroxy-3,3-dimethyl-butoxy)-propoxy]-2,2-dimethyl-butan-1-ol;
I-114: 4-[3-(3-carboxy-3-methyl-butoxy)-propoxy]-2,2-dimethyl-butyric acid;
I-115: 4-[3-(3,3-dimethyl-4-oxo-butoxy)-propoxy]-2,2-dimethyl-butanal;
I-116: 4-[3-(3-Methoxycarbonyl-3-methyl-butoxy)-propoxy]-2,2-dimethyl-butyric acid methyl ester;
I-117: 4-[3-(3,3-dimethyl-4-oxo-5-phenyl-pentyloxy)-propoxy]-2,2-dimethyl-butyric acid phenyl ester;
I-118: 4-[3-(3-benzyloxycarbonyl-3-methyl-butoxy)-propoxy]-2,2-dimethyl-butyric acid benzyl ester;
I-119: 2-methyl-4-[3-(3-methyl-3-sulfo-butoxy)-propoxy]-butane-2-sulfonic acid;
I-120: phosphoric acid mono-{1,1-dimethyl-3-[3-(3-methyl-3-phosphonooxy-butoxy)-propoxy]-propyl}ester;
I-121: 1-ethyl-3-(3-{3-[3-(4,6-dioxo-2,3,3a,6-tetrahydro-4H-thieno[3,2-c]pyridin-5-yl))-3-methyl-butoxy]-propoxy}-1,1-dimethyl-propyl)-4,6-dioxo-2,3,3a,6-tetrahydro-4H-thieno [3,2-c]pyridin-5-yl-4,6-dione;
I-122: 1-ethyl-3-(3-{3-[3-(4,6-dithioxo-2,3,3a,6-tetrahydro-4H-thieno[3,2-c]pyridin-5-yl))-3-methyl-butoxy]-propoxy}-1,1-dimethyl-propyl)-4,6-dioxo-2,3,3a,6-tetrahydro-4H-thieno[3,2-c]pyridin-5-yl-4,6-dithione;
I-123: 2,2-dimethyl-4-[3-(3-methyl-3-cyano-carbamoyl-butoxy)-propoxy]-N-cyano-butyric acid-amide;
I-124: phosphoramidic acid mono-(3-{3-[3-(amino-hydroxy-phosphoryloxy)-3-methyl-butoxy]-propoxy}-1,1-dimethyl-propyl)ester;
I-125: {1,1-dimethyl-3-[3-(3-(methyl-3-phosponamido-butoxy)-propoxy]-propyl}-phosphonamide;
I-126: 1-{3-[3-(3-methyl-3-tetrazol-1-yl-butoxy)-propoxy]-1,1-dimethyl-propyl}-1H-tetrazole;
I-127: 5-{3-[3-(3-methyl-3-tetrazol-5-yl-butoxy)-propoxy]-1,1-dimethyl-propyl}-(1H)-tetrazole;
I-128: 5-{3-[3-(3-methyl-3-(3-methyl-isoxazol-5-yl)-butoxy)-propoxy]-1,1-dimethyl-propyl}-3-methyl-isoxazole;
I-129: 4-{3-[3-(3-methyl-3-(3-methyl-isoxazol-4-yl)-butoxy)-propoxy]-1,1-dimethyl-propyl}-3-methyl-isoxazole;
I-130: 3-{3-[3-(3-methyl-3-(5-hydroxy-pyran-3-yl-4-one)-butoxy)-propoxy]-1,1-dimethyl-propyl}-5-hydroxy-pyran-4-one;
I-131: 2-{3-[3-(3-methyl-3-(5-hydroxy-pyran-2-yl-4-one)-butoxy)-propoxy]-1,1-dimethyl-propyl}-5-hydroxy-pyran-4-one;
I-132: 3-{3-[3-(3-methyl-3-(5-hydroxy-pyran-2-yl-4-one)-butoxy)-propoxy]-1,1-dimethyl-propyl}-5-hydroxy-pyran-4-one;
I-133: 1-ethyl-3-(3-{3-[3-(3-ethyl-2,5-dithioxo-imidazolidin-1-yl)-3-methyl-butoxy]-propoxy}-1,1-dimethyl-propyl)-imidazolidine-2,4-dithione;
I-134: 1-ethyl-3-(3-{3-[3-(3-ethyl-2,5-dioxo-imidazolidin-1-yl)-3-methyl-butoxy]-propoxy}-1,1-dimethyl-propyl)-imidazolidine-2,4-dithione;
I-135: 1-ethyl-3-(3-{3-[3-(3-ethyl-2,5-dioxo-imidazolidin-1-yl)-3-methyl-butoxy]-propoxy}-1,1-dimethyl-propyl)-imidazolidine-2,4-dione;
I-136: 1-ethyl-3-(3-{3-[3-(3-ethyl-2-thioxo-5-oxo-imidazolidin-1-yl)-3-methyl-butoxy]-propoxy}-1,1-dimethyl-propyl)-imidazolidine-2-thioxo-4-one;
I-137: 1-ethyl-3-(3-{3-[3-(3-ethyl-2-oxo-5-thioxo-imidazolidin-1-yl)-3-methyl-butoxy]-propoxy}-1,1-dimethyl-propyl)-imidazolidine-2-oxo-4-thione;
I-138: 1-(2-tetrahydropyranyloxy)-2-{2-[2-(2-tetrahydropyranyloxy)-ethoxy]-propoxy}ethane;
I-139: 4-{2-[3-(oxetan-4-yl-2-one)-propoxy-propoxy]-ethyl}-oxetan-2-one;
I-140: 3-{2-[3-(oxetan-3-yl-2-one)-propoxy-propoxy]-ethyl}-oxetan-2-one;
I-141: 5-{2-[3-(dihydro-furan-5-yl-2-one)-propoxy-propoxy]-ethyl}-dihydro-furan-2-one;
I-142: 4-{2-[3-(dihydro-furan-4-yl-2-one)-propoxy-propoxy]-ethyl}-dihydro-furan-2-one;
I-143: 3-{2-[3-(dihydro-furan-3-yl-2-one)-propoxy-propoxy]-ethyl}-dihydro-furan-2-one;
I-144: [2-(2-{3-[2-(4-carboxymethyl-4-hydroxy-6-oxo-tetrahydro-pyran-2-yl)-ethoxy]-propoxy}-ethyl)-4-hydroxy-6-oxo-tetrahydro-pyran-4-yl]-acetic acid;
I-145: 2,2′-[propylenebis(oxadiyl)]diethane-6-d-valerolactone;
I-146: 2,2′-[propylenebis(oxadiyl)]diethane-5-d-valerolactone;
I-147: 2,2′-[propylenebis(oxadiyl)]diethane-4-d-valerolactone;
I-148: 2,2′-[propylenebis(oxadiyl)]diethane-3-d-valerolactone;
I-149: 5-[3-(5-hydroxy-3,3-dimethyl-pentyloxy)-propoxy]-3,3-dimethyl-pentan-1-ol;
I-150: 5-[3-(4-carboxy-3,3-dimethyl-butoxy)-propoxy]-3,3-dimethyl-pentanoic acid;
I-151: 5-[3-(3,3-dimethyl-5-oxo-pentyloxy)-propoxy]-3,3-dimethyl-pentanal;
I-152: 5-[3-(4-methoxycarbonyl-3,3-dimethyl-butoxy)-propoxy]-3,3-dimethyl-pentanoic acid methyl ester;
I-153: 5-[3-(3,3-dimethyl-4-phenoxycarbonyl-butoxy)-propoxy]-3,3-dimethyl-pentanoic acid phenyl ester;
I-154: 5-[3-(4-benzyloxycarbonyl-3,3-dimethyl-butoxy)-propoxy]-3,3-dimethyl-pentanoic acid benzyl ester;
I-155: 4-[3-(3,3-dimethyl-pentyloxy)-propoxy]-2,2-dimethyl-butane-1-sulfonic acid;
I-156: phosphoric acid mono-{4-[3-(3,3-dimethyl-4-phosphonooxy-butoxy)-propoxy]-2,2-dimethyl-butyl}ester;
I-157: 5-{4-[3-(3,3-dimethyl-4-(5-(3,3a-dihydro-2H-thieno-[3,2-c]pyridine-4,6-dioxo)pentyloxy)-propoxy]-2,2-dimethyl-butyl}-3,3a-dihydro3,3a-dihydro-2H-thieno-[3,2-c]pyridine-4,6-dione;
I-158: 5-{4-[3-(3,3-dimethyl-4-(5-(3,3a-dihydro-2H-thieno-[3,2-c]pyridine-4,6-dithioxo)pentyloxy)-propoxy]-2,2-dimethyl-butyl}-3,3a-dihydro-3,3a-dihydro-2H-thieno-[3,2-c]pyridine-4,6-dithione;
I-159: 5-[3-(3,3-dimethyl-4-cyano-carbamoyl-butoxy)-propoxy]-3,3-dimethyl-n-cyano-pentanoic acid-amide;
I-160: phosphoramidic acid mono-(5-{2-[4-(amino-hydroxy-phosphoryloxy)-3,3-dimethyl-butoxy]-ethoxy}-2,2-dimethyl-pentyl)ester;
I-161: {4-[3-(3,3-dimethyl-4-phosponamido-butoxy)-propoxy]-2,2-dimethyl-butyl}-phosphonamide;
I-162: 1-{4-[3-(3,3-dimethyl-5-(1H-tetrazol-1-yl)-butoxy)-propoxy]-2,2-dimethyl-butyl}-1H-tetrazole;
I-163: 5-{4-[3-(3,3-dimethyl-5-(1H-tetrazol-5-yl)-butoxy)-propoxy]-2,2-dimethyl-butyl}-1H-tetrazole;
I-164: 5-{4-[3-(3,3-dimethyl-5-(3-hydroxy-isoxazol-5-yl)-butoxy)-propoxy]-2,2-dimethyl-butyl}-3-hydroxy-isoxazole;
I-165: 4-{4-[3-(3,3-dimethyl-5-(3-hydroxy-isoxazol-4-yl)-butoxy)-propoxy]-2,2-dimethyl-butyl}-3-hydroxy-isoxazole;
I-166: 2-{4-[3-(3,3-dimethyl-4-{5-hydroxy-pyran-4-oxo-3-yl}-butyloxy)-propoxy]-2,2-dimethyl-butyl}-5-hydroxy-pyran-4-one;
I-167: 2-{4-[3-(3,3-dimethyl-4-{5-hydroxy-pyran-4-oxo-2-yl}-butyloxy)-propoxy]-2,2-dimethyl-butyl}-5-hydroxy-pyran-4-one;
I-168: 3-{4-[3-(3,3-dimethyl-4-{5-hydroxy-pyran-4-oxo-3-yl}-butyloxy)-propoxy]-2,2-dimethyl-butyl}-5-hydroxy-pyran-4-one;
I-169: 1-ethyl-3-(4-{3-[4-(3-ethyl-2,5-dithioxo-imidazolidin-1-yl)-3,3-dimethyl-butoxy]-propoxy}-2,2-dimethyl-butyl)-imidazolidine-2,4-dione;
I-170: 1-ethyl-3-(4-{3-[4-(3-ethyl-2,5-oxo-imidazolidin-1-yl)-3,3-dimethyl-butoxy]-propoxy}-2,2-dimethyl-butyl)-imidazolidine-2,4-dione;
I-171: 1-ethyl-3-(4-{3-[4-(3-ethyl-2-thioxo-5-oxo-imidazolidin-1-yl)-3,3-dimethyl-butoxy]-propoxy}-2,2-dimethyl-butyl)-imidazolidine-2-thioxo-4-one;
I-172: 1-ethyl-3-(4-{3-[4-(3-ethyl-2-oxo-5-thioxo-imidazolidin-1-yl)-3,3-dimethyl-butoxy]-propoxy}-2,2-dimethyl-butyl)-imidazolidine-2-oxo-4-thione;
I-173: 5-[3-(4-hydroxy-4-methyl-pentyloxy)-propoxy]-2-methyl-pentan-2-ol;
I-174: 5-[3-(5-hydroxy-4,4-dimethyl-pentyloxy)-propoxy]-2,2-dimethyl-pentan-1-ol;
I-175: 5-[3-(4-carboxy-4-methyl-pentyloxy)-propoxy]-2,2-dimethyl-pentanoic acid;
I-176: 5-[3-(4,4-dimethyl-5-oxo-pentyloxy)-propoxy]-2,2-dimethyl-pentanal;
I-177: 5-[3-(4-methoxycarbonyl-4-methyl-pentyloxy)-propoxy]-2,2-dimethyl-pentanoic acid methyl ester;
I-178: 5-[3-(4,4-dimethyl-5-oxo-6-phenyl-hexyloxy)-propoxy]-2,2-dimethyl-pentanoic acid phenyl ester;
I-179: 4-{3-[1-(2-benzyloxycarbonyl-2-methyl-propyl)-vinyloxy]-propoxy}-2,2-dimethyl-pent-4-enoic acid benzyl ester;
I-180: 2-methyl-5-[3-(4-methyl-4-sulfo-pentyloxy)-propoxy]-pentane-2-sulfonic acid;
I-181: phosphoric acid mono-{1,1-dimethyl-4-[3-(4-methyl-4-phosphonooxy-pentyloxy)-propoxy]-butyl}ester;
I-182: 5-(5-{3-[3,3-dimethyl-5-(4,6-dioxo-2,3,3a,6-tetrahydro-4h-thieno-[3,2-c]pyridin-5-yl)-pentyloxy]-propoxy}-3,3-dimethyl-pentyl)-3,3a-dihydro-2H-thieno[3,2-c]pyridine-4,6-dione;
I-183: 5-(5-{3-[3,3-dimethyl-5-(4,6-dithioxo-2,3,3a,6-tetrahydro-4h-thieno-[3,2-c]pyridin-5-yl)-pentyloxy]-propoxy}-3,3-dimethyl-pentyl)-3,3a-dihydro-2H-thieno[3,2-c]pyridine-4,6-dithione;
I-184: 5-{3-[4-N-cyano-carbamoyl-4-methyl-pentyloxy)-propoxy]-2,2-dimethyl-N-cyano-pentanoic acid-amide;
I-185: phosphoramidic acid mono-[3-(3-{1-[2-(amino-hydroxy-phosphoryloxy)-2-methyl-propyl]-vinyloxy}-propoxy)-1,1-dimethyl-but-3-enyl]ester;
I-186: {1,1-dimethyl-4-[3-(4-methyl-4-phosphonamido-pentyloxy)-propoxy]-butyl}-phosphonamide;
I-187: 1-{4-[3-(4-{1H-tetrazol-1-yl}-4-methyl-pentyloxy)-propoxy]-1,1-dimethyl-butyl}-1H-tetrazol;
I-188: 5-{4-[3-(4-{1H-tetrazol-5-yl}-4-methyl-pentyloxy)-propoxy]-1,1-dimethyl-butyl}-1H-tetrazole;
I-189: 5-{4-[3-(4-{3-methyl-isoxazol-5-yl}-4-methyl-pentyloxy)-propoxy]-1,1-dimethyl-butyl}-3-methyl-isoxazole;
I-190: 4-{4-[3-(4-{3-methyl-isoxazol-4-yl}-4-methyl-pentyloxy)-propoxy]-1,1-dimethyl-butyl}-3-methyl-isoxazole;
I-191: 3-{4-[3-(4-{5-hydroxy-4-oxo-pyran-3-yl}-4-methyl-pentyloxy)-propoxy]-1,1-dimethyl-butyl}-5-hydroxy-pyran-4-one;
I-192: 2-{4-[3-(4-{5-hydroxy-4-oxo-pyran-2-yl}-4-methyl-pentyloxy)-propoxy]-1,1-dimethyl-butyl}-5-hydroxy-pyran-4-one;
I-193: 1-ethyl-3-(4-{3-[4-(3-ethyl-2,5-dithioxo-imidazolidin-1-yl)-4-methyl-pentyloxy]-propoxy}-1,1-dimethyl-butyl)-imidazolidine-2,4-dione;
I-194: 1-ethyl-3-(4-{3-[4-(3-ethyl-2,5-dioxo-imidazolidin-1-yl)-4-methyl-pentyloxy]-propoxy}-1,1-dimethyl-butyl)-imidazolidine-2,4-dione;
I-195: 1-ethyl-3-(4-{3-[4-(3-ethyl-5-oxo-2-thioxo-imidazolidin-1-yl)-4-methyl-pentyloxy]-propoxy}-1,1-dimethyl-butyl)-imidazolidine-4-oxo-2-thione;
I-196: 1-ethyl-3-(4-{3-[4-(3-ethyl-2-oxo-5-thioxo-imidazolidin-1-yl)-4-methyl-pentyloxy]-propoxy}-1,1-dimethyl-butyl)-imidazolidine-2-oxo-4-thione;
I-197: 2-{3-[3-(3-{tetrahydro-pyran-2-yl}-propoxy)-propoxy]-propoxy}-tetrahydro-pyran;
I-198: 4-{3-[3-(3-{oxetan-2-one-4-yl}propoxy)-propoxy]-propyl}-oxetan-2-one;
I-199: 3-{3-[3-(3-{oxetan-2-one-3-yl}propoxy)-propoxy]-propyl}-oxetan-2-one;
I-200: 5-{3-[3-(3-{dihydro-furan-2-one-5-yl}-propoxy)-propoxy]-propyl}-dihydro-furan-2-one;
I-201: 4-{3-[3-(3-{dihydro-furan-2-one-4-yl}-propoxy)-propoxy]-propyl}-dihydro-furan-2-one;
I-202: 3-{3-[3-(3-{dihydro-furan-2-one-3-yl}-propoxy)-propoxy]-propyl}-dihydro-furan-2-one;
I-203: {2-[3-(3-{3-[4-carboxymethyl-4-hydroxy-6-oxo-tetrahydro-pyran-2-yl)-propoxy}-propoxy)-propyl]-4-hydroxy-6-oxo-tetrahydro-pyran-4-yl}-acetic acid;
I-204: 6-{3-[3-(3-{dihydro-pyran-2-one-6-yl}-propoxy)-propoxy]-propyl}-dihydro-pyran-2-one;
I-205: 5-{3-[3-(3-{dihydro-pyran-2-one-5-yl}-propoxy)-propoxy]-propyl}-dihydro-pyran-2-one;
I-206: 4-{3-[3-(3-{dihydro-pyran-2-one-4-yl}-propoxy)-propoxy]-propyl}-dihydro-pyran-2-one;
I-207: 3-{3-[3-(3-{dihydro-pyran-2-one-3-yl}-propoxy)-propoxy]-propyl}-dihydro-pyran-2-one;
I-208: 6-[3-(6-hydroxy-4,4-dimethyl-hexyloxy)-propoxy]-3,3-dimethyl-hexan-1-ol;
I-209: 6-[3-(5-carboxy-4,4-dimethyl-pentyloxy)-propoxy]-3,3-dimethyl-hexanoic acid;
I-210: 6-[3-(4,4-dimethyl-6-oxo-hexyloxy)-propoxy]-3,3-dimethyl-hexanal;
I-211: 6-[3-(5-methoxycarbonyl-4,4-dimethyl-pentyloxy)-propoxy]-3,3-dimethyl-hexanoic acid methyl ester;
I-212: 6-[3-(4,4-dimethyl-5-phenoxycarbonyl-pentyloxy)-propoxy]-3,3-dimethyl-hexanoic acid cyclohexyl ester;
I-213: 6-[3-(5-benzyloxycarbonyl-4,4-dimethyl-pentyloxy)-propoxy]-3,3-dimethyl-hexanoic acid benzyl ester;
I-214: 5-[3-(4,4-dimethyl-5-sulfo-pentyloxy)-propoxy]-2,2-dimethyl-pentane-1-sulfonic acid;
I-215: 5-[3-(4,4-dimethyl-5-phospo-pentyloxy)-propoxy]-2,2-dimethyl-pentane-1-phosphonic acid;
I-216: 5-{5-[3-(5-{3,3a-dihydro-2H-thieno[3,2-c]pyridine-4,6-dione-5-yl}-4,4-dimethyl-pentyloxy)-propoxy]-2,2-dimethyl-3-pentyl}-3,3a-dihydro-2H-thieno[3,2-c]pyridine-4,6-dione;
I-217: 5-{5-[3-(5-{3,3a-dihydro-2H-thieno[3,2-c]pyridine-4,6-dithione-5-yl}-4,4-dimethyl-pentyloxy)-propoxy]-2,2-dimethyl-3-pentyl}-3,3a-dihydro-2H-thieno[3,2-c]pyridine-4,6-dithione;
I-218: 6-[3-(5-cyano-carbamoyl-4,4-dimethyl-pentyloxy)-propoxy]-3,3-dimethyl-N-cyano-hexanoic acid-amide;
I-219: phosphoramidic acid mono-(6-{2-[5-(amino-hydroxy-phosphoryloxy)-4,4-dimethyl-pentyloxy]-ethoxy}-2,2-dimethyl-hexyl)ester;
I-220: {5-[³-(4,4-dimethyl-5-phosphonamido-pentyloxy)-propoxy]-2,2-dimethyl-pentyl}-phosphonamide;
I-221: 1-{5-[3-(5-{1H-tetrazol-1-yl}-4,4-dimethyl-pentyloxy)-propoxy]-2,2-dimethyl-pentyl}-1H-tetrazole;
I-222: 5-{5-[3-(5-{1H-tetrazol-5-yl}-4,4-dimethyl-pentyloxy)-propoxy]-2,2-dimethyl-pentyl}-1H-tetrazole;
I-223: 5-{5-[3-(5-{3-hydroxy-isoxazol-5-yl}-4,4-dimethyl-pentyloxy)-propoxy]-2,2-dimethyl-pentyl}-3-hydroxy-isoxazole;
I-224: 4-{5-[3-(5-{3-hydroxy-isoxazol-4-yl}-4,4-dimethyl-pentyloxy)-propoxy]-2,2-dimethyl-pentyl}-3-hydroxy-isoxazole;
I-225: 2-{5-[3-(5-{5-hydroxy-4-oxo-pyran-3-yl}-4,4-dimethyl-pentyloxy)-propoxy]-2,2-dimethyl-pentyl}-5-hydroxy-pyran-4-one;
I-226: 2-{5-[3-(5-{5-hydroxy-4-oxo-pyran-2-yl}-4,4-dimethyl-pentyloxy)-propoxy]-2,2-dimethyl-pentyl}-5-hydroxy-pyran-4-one;
I-227: 3-{5-[3-(5-{5-hydroxy-4-oxo-pyran-3-yl}-4,4-dimethyl-pentyloxy)-propoxy]-2,2-dimethyl-pentyl}-5-hydroxy-pyran-4-one;
I-228: 3-{4-[3-(5-{3-ethyl-2,5-dithioxo-imidazolidin-1-yl}-4,4-dimethyl-pentyloxy)-propoxy]-2,2-dimethyl-butyl}-1-ethyl-imidazolidine-2,4-dithione;
I-229: 3-{4-[3-(5-{3-ethyl-2,5-dioxo-imidazolidin-1-yl}-4,4-dimethyl-pentyloxy)-propoxy]-2,2-dimethyl-butyl}-1-ethyl-imidazolidine-2,4-dione;
I-230: 3-{4-[3-(5-{3-ethyl-5-oxo-2-thioxo-imidazolidin-1-yl}-4,4-dimethyl-pentyloxy)-propoxy]-2,2-dimethyl-butyl}-1-ethyl-imidazolidine-4-oxo-2-thione;
I-231: 3-{4-[3-(5-{3-ethyl-5-oxo-2-thioxo-imidazolidin-1-yl}-4,4-dimethyl-pentyloxy)-propoxy]-2,2-dimethyl-butyl}-1-ethyl-imidazolidine-2-oxo-4-thione;
I-232: 6-[3-(5-hydroxy-5-methyl-hexyloxy)-propoxy]-2-methyl-hexan-2-ol;
I-233: 6-[3-(6-hydroxy-5,5-dimethyl-hexyloxy)-propoxy]-2,2-dimethyl-hexan-1-ol;
I-234: 6-[3-(5-carboxy-5-methyl-hexyloxy)-propoxy]-2,2-dimethyl-hexanoic acid;
I-235: 6-[3-(5,5-dimethyl-6-oxo-hexyloxy)-propoxy]-2,2-dimethyl-hexanal;
I-236: 6-[3-(5-methoxycarbonyl-5-methyl-hexyloxy)-propoxy]-2,2-dimethyl-hexanoic acid methyl ester;
I-237: 6-[3-(5,5-dimethyl-6-oxo-7-phenyl-heptyloxy)-propoxy]-2,2-dimethyl-hexanoic acid phenyl ester;
I-238: 6-[3-(5-benzyloxycarbonyl-5-methyl-hexyloxy)-propoxy]-2,2-dimethyl-hexanoic acid benzyl ester;
I-239: 2-methyl-6-[3-(5-methyl-5-sulfo-hexyloxy)-propoxy]-hexane-2-sulfonic acid;
I-240: phosphoric acid mono-{1,1-dimethyl-5-[3-(5-methyl-5-phosphonooxy-hexyloxy)-propoxy]-pentyl}ester;
I-241: 5-(5-{3-[4-(4,6-dioxo-hexahydro-thieno[3,2-c]pyridin-5-yl)-4-methyl-pentyloxy]-propoxy}-1,1-dimethyl-pentyl)-3,3a-dihydro-2H-thieno-[3,2-c]pyridine-4,6-dione;
I-242: 5-(5-{3-[4-(4,6-dithioxo-hexahydro-thieno[3,2-c]pyridin-5-yl)-4-methyl-pentyloxy]-propoxy}-1,1-dimethyl-pentyl)-3,3a-dihydro-2H-thieno[3,2-c]pyridine-4,6-dithione;
I-243: 6-[3-(4-N-cyano-carbamoyl-4-methyl-pentyloxy)-propoxy]-2,2-dimethyl-N-cyano-hexanoic acid-amide;
I-244: phosphoramidic acid mono-(5-{3-[5-(amino-hydroxy-phosphoryloxy)-5-methyl-hexyloxy]-propoxy}-1,1-dimethyl-pentyl) ester;
I-245: {1,1-dimethyl-5-[3-(5-methyl-5-phosphonamido-hexyloxy)-propoxy]-pentyl}-phosphonamide;
I-246: 1-{5-[3-(5-{1H-tetrazol-1-yl}-5-methyl-hexyloxy)-propoxy]-1,1-dimethyl-pentyl}-1H-tetrazole;
I-247: 5-{5-[3-(5-{1H-tetrazol-5-yl}-5-methyl-hexyloxy)-propoxy]-1,1-dimethyl-pentyl}-1H-tetrazole;
I-248: 5-{5-[3-(5-{3-hydroxy-isoxazol-5-yl}-5-methyl-hexyloxy)-propoxy]-1,1-dimethyl-pentyl}-3-hydroxy-isoxazole;
I-249: 4-{5-[3-(5-{3-hydroxy-isoxazol-4yl}-5-methyl-hexyloxy)-propoxy]-1,1-dimethyl-pentyl}-3-hydroxy-isoxazole;
I-250: 3-{5-[3-(5-{5-hydroxy-4-oxo-pyran-3-yl}-5-methyl-hexyloxy)-propoxy]-1,1-dimethyl-pentyl}-5-hydroxy-pyran-4-one;
I-251: 2-{5-[3-(5-{5-hydroxy-4-oxo-pyran-2-yl}-5-methyl-hexyloxy)-propoxy]-1,1-dimethyl-pentyl}-5-hydroxy-pyran-4-one;
I-252: 1-ethyl-3-(5-{3-[5-(3-ethyl-2,5-dithioxo-imidazolidin-1-yl)-5-methyl-hexyloxy]-propoxy}-2,2-dimethyl-hexyl)-imidazolidine-2,4-dione;
I-253: 1-ethyl-3-(5-{3-[5-(3-ethyl-2,5-dioxo-imidazolidin-1-yl)-5-methyl-hexyloxy]-propoxy}-2,2-dimethyl-hexyl)-imidazolidine-2,4-dione;
I-254: 1-ethyl-3-(5-{3-[5-(3-ethyl-2-thioxo-5-oxo-imidazolidin-1-yl)-5-methyl-hexyloxy]-propoxy}-2,2-dimethyl-hexyl)-imidazolidine-4-oxo-2-thione;
I-255: 1-ethyl-3-(5-{3-[5-(3-ethyl-5-thioxo-2-oxo-imidazolidin-1-yl)-5-methyl-hexyloxy]-propoxy}-2,2-dimethyl-hexyl)-imidazolidine-2-oxo-4-thione;
I-256: 2-{4-[3-(4-{tetrahydro-pyran-2-yl}-butoxy)-propoxy]-butoxy}-tetrahydro-pyran;
I-257: 4-{4-[3-(4-{oxetan-2-one-4-yl}-butoxy)-propoxy]-butyl}-oxetan-2-one;
I-258: 3-{4-[3-(4-{oxetan-2-one-3-yl}-butoxy)-propoxy]-butyl}-oxetan-2-one;
I-259: 5-{4-[3-(4-{tetrahydro-furan-2-one-5-yl}-butoxy)-propoxy]-butyl}-tetrahydro-furan-2-one;
I-260: 4-{4-[3-(4-{tetrahydro-furan-2-one-4-yl}-butoxy)-propoxy]-butyl}-tetrahydro-furan-2-one;
I-261: 3-{4-[3-(4-{tetrahydro-furan-2-one-3-yl}-butoxy)-propoxy]-butyl}-tetrahydro-furan-2-one;
I-262: [2-(4-{3-[4-(4-carboxymethyl-4-hydroxy-6-oxo-tetrahydro-pyran-2-yl)-butoxy]-propoxy}-butyl)-4-hydroxy-6-oxo-tetrahydro-pyran-4-yl)-acetic acid;
I-263: 6-{4-[3-(4-{tetrahydro-pyran-2-one-6-yl}-butoxy)-propoxy]-butyl}-tetrahydro-pyran-2-one;
I-264: 5-{4-[3-(4-{tetrahydro-pyran-2-one-5-yl}-butoxy)-propoxy]-butyl}-tetrahydro-pyran-2-one;
I-265: 4-{4-[3-(4-{tetrahydro-pyran-2-one-4-yl}-butoxy)-propoxy]-butyl}-tetrahydro-pyran-2-one;
I-266: 3-{4-[3-(4-{tetrahydro-pyran-2-one-3-yl}-butoxy)-propoxy]-butyl}-tetrahydro-pyran-2-one;
I-267: 7-[3-(7-hydroxy-5,5-dimethyl-heptyloxy)-propoxy]-3,3-dimethyl-heptan-1-ol;
I-268: 7-[3-(6-carboxy-5,5-dimethyl-hexyloxy)-propoxy]-3,3-dimethyl-heptanoic acid;
I-269: 7-[3-(5,5-dimethyl-6-oxo-hexyloxy)-propoxy]-3,3-dimethyl-heptanal;
I-270: 7-[3-(6-methoxycarbonyl-5,5-dimethyl-hexyloxy)-propoxy]-3,3-dimethyl-heptanoic acid methyl ester;
I-271: 7-[3-(5,5-dimethyl-6-phenoxycarbonyl-hexyloxy)-propoxy]-3,3-dimethyl-heptanoic acid phenyl ester;
I-272: 7-[3-(6-benzyloxycarbonyl-5,5-dimethyl-hexyloxy)-propoxy]-3,3-dimethyl-heptanoic acid benzyl ester;
I-273: 6-[3-(5,5-dimethyl-6-sulfo-hexyloxy)-propoxy]-2,2-dimethyl-hexane-1-sulfonic acid;
I-274: phosphoric acid mono-{6-[3-(5,5-dimethyl-6-phosphonooxy-hexyloxy)-propoxy]-2,2-dimethyl-hexyl}-ester;
I-275: 5-(6-{3-[6-(4,6-dioxo-hexahydro-thieno[3,2-c]pyridin-5-yl)-5,5-dimethyl-hexyloxy]-propoxy}-2,2-dimethyl-hexyl)-3,3a-dihydro-2H-thieno[3,2-c]pyridine-4,6-dione;
I-276: 5-(5-{3-[4-(4,6-dithioxo-hexahydro-thieno[3,2-c]pyridin-5-yl)-4-methyl-pentyloxy]-propoxy}-1,1-dimethyl-pentyl)-3,3a-dihydro-2H- thieno [3,2-c]pyridine-4,6-dithione;
I-277: 7-[3-(6-N-cyano-carbamoyl-5,5-dimethyl-hexyloxy)-propoxy]-3,3-dimethyl-N-cyano-heptanoic acid-amide;
I-278: phosphoramidic acid mono-{7-[2-(6-{amino-hydroxy-phosphoryloxy}-5,5-dimethyl-hexyloxy)-ethoxy]-2,2-dimethyl-heptyl}ester;
I-279: {6-[3-(5,5-dimethyl-6-phosphonamido-hexyloxy)-propoxy]-2,2-dimethyl-hexyl}-phosphonamide;
I-280: 1-{6-[3-(6-{1H-tetrazol-1-yl}-5,5-dimethyl-hexyloxy)-propoxy]-2,2-dimethyl-hexyl}-1H-tetrazole;
I-281: 5-{6-[3-(6-{1H-tetrazol-5-yl}-5,5-dimethyl-hexyloxy)-propoxy]-2,2-dimethyl-hexyl}-1H-tetrazole;
I-282: 5-{6-[3-(6-{3-hydroxy-isoxazol-5-yl}-5,5-dimethyl-hexyloxy)-propoxy]-2,2-dimethyl-hexyl}-3-hydroxy-isoxazole;
I-283: 4-{6-[3-(6-{3-hydroxy-isoxazol-4-yl}-5,5-dimethyl-hexyloxy)-propoxy]-2,2-dimethyl-hexyl}-3-hydroxy-isoxazole;
I-284: 2-{6-[3-(6-{5-hydroxy-4-oxo-pyran-3-yl}-5,5-dimethyl-hexyloxy)-propoxy]-2,2-dimethyl-hexyl}-5-hydroxy-pyran-4-one;
I-285: 2-{6-[3-(6-{5-hydroxy-4-oxo-pyran-2-yl}-5,5-dimethyl-hexyloxy)-propoxy]-2,2-dimethyl-hexyl}-5-hydroxy-pyran-4-one;
I-286: 3-{6-[3-(6-{5-hydroxy-4-oxo-pyran-3-yl}-5,5-dimethyl-hexyloxy)-propoxy]-2,2-dimethyl-hexyl}-5-hydroxy-pyran-4-one;
I-287: 1-ethyl-3-(6-{3-[6-(3-ethyl-2,5-dithioxo-imidazolidin-1-yl)-5,5-dimethyl-hexyloxy]-propoxy}-2,2-dimethyl-hexyl)- imidazolidine-2,4-dione;
I-288: 1-ethyl-3-(6-{3-[6-(3-ethyl-2,5-dioxo-imidazolidin-1-yl)-5,5-dimethyl-hexyloxy]-propoxy}-2,2-dimethyl-hexyl)-imidazolidine-2,4-dione;
I-289: 1-ethyl-3-(6-{3-[6-(3-ethyl-5-oxo-2-thioxo-imidazolidin-1-yl)-5,5-dimethyl-hexyloxy]-propoxy}-2,2-dimethyl-hexyl)-imidazolidine-4-oxo-2-thione;
I-290: 1-ethyl-3-(6-{3-[6-(3-ethyl-2-oxo-5-thioxo-imidazolidin-1-yl)-5,5-dimethyl-hexyloxy]-propoxy}-2,2-dimethyl-hexyl)-imidazolidine-2-oxo-4-thione;
I-291: 6-[3-(5-carboxy-5-methyl-hexyloxymethyl)-benzyloxy]-2,2-dimethyl-hexanoic acid;
I-292: 6-[3-(5-carboxy-5-methyl-hexyloxymethyl)-benzyloxy]-2,2-dimethyl-hexan-1-ol;
I-293: 6-[3-(6-hydroxy-5,5-dimethyl-hexyloxymethyl)-benzyloxy]-2,2-dimethyl-hexan-1-ol;
I-294: 5-[3-(4-carboxy-4-methyl-pentyloxymethyl)-benzyloxy]-2,2-dimethyl-pentanoic acid;
I-295: 5-[3-(4-carboxy-4-methyl-pentyloxymethyl)-benzyloxy]-2,2-dimethyl-hexan-1-ol;
I-296: 5-[3-(5-hydroxy-4,4-dimethyl-pentyloxyrnethyl)-benzyloxy]-2,2-dimethyl-pentan-1-ol; or
I-297: 5-[2-(5-hydroxy-4,4-dimethyl-pentyloxy)-ethoxy]-2,2-dimethyl-pentan-1-ol.

30. A compound of the formula II: 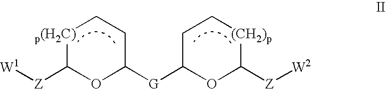
or a pharmaceutically acceptable salt, hydrate, solvate, or a mixture thereof, wherein:
(a) each occurrence of Z is independently (CH₂)ₘ, (CH═CH)ₜ, or phenyl, where each occurrence of m and t are independent integers ranging from 1 to 5;
(b) G is (CH₂)ₓ, CH₂CH═CHCH₂, CH═CH, CH₂₋phenyl-CH₂, or phenyl, where x is an integer ranging from 1 to 4;
(c) W¹ and W² are independently C(R¹)(R²)(CH₂)ₙ₋Y, V, or C(R¹)(R²)—(CH₂)_{c-}V where c is 1 or 2 and n is an integer ranging from 0 to 4;
(d) each occurrence of R¹ and R² is independently (C₁₋C₆)alkyl, (C₂₋C₆)alkenyl, (C₂₋C₆)alkynyl, phenyl, or benzyl;
(e) V is 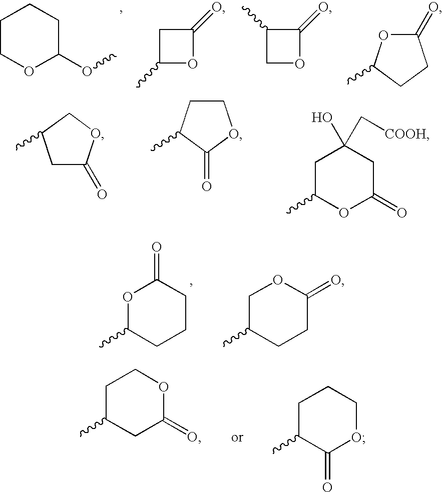
(f) each occurrence of Y is independently OH, COOH, CHO, COOR⁷, SO₃H, 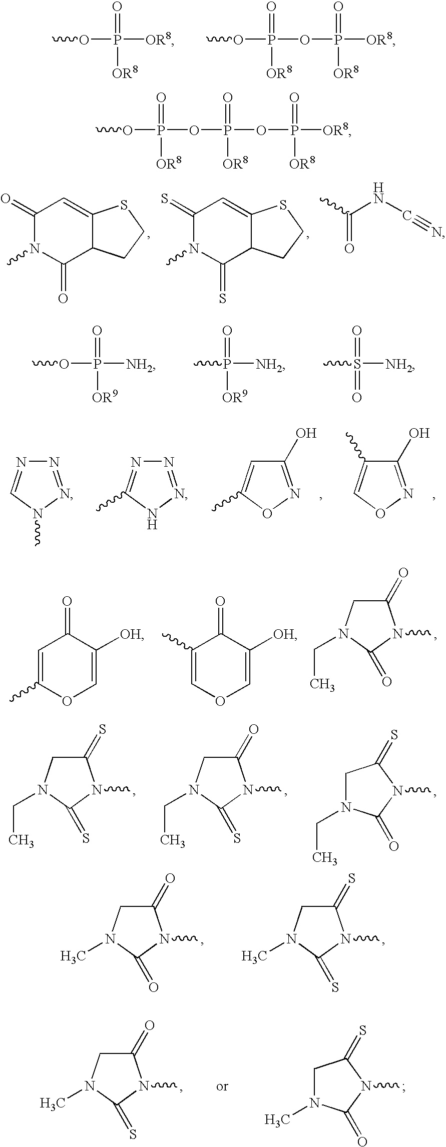
(g) R⁷ is (C₁₋C₆)alkyl, (C₂₋C₆)alkenyl, (C₂₋C₆)alkynyl, phenyl, or benzyl and is unsubstituted or substituted with one or more halo, OH, (C₁₋C₆)alkoxy, or phenyl groups;
(h) each occurrence of R⁸ is independently H, (C₁₋C₆)alkyl, (C₂₋C₆)alkenyl, or (C₂₋C₆)alkynyl and is unsubstituted or substituted with one or two halo, OH, (C₁₋C₆)alkoxy, or phenyl groups;
(i) each occurrence of R⁹ is independently H, (C₁₋C₆)alkyl, (C₂₋C₆)alkenyl, or (C₂₋C₆)alkynyl; and
(j) each occurrence of p is independently 0 or 1 where the broken line represents an optional presence of 1 or 2 additional carbon-carbon bonds that when present complete 1 or 2 carbon-carbon double bonds.

31. The compound of claim 30, wherein W¹ and W² are independent C(R¹)(R²)(CH₂)ₙ₋Y groups and each occurrence of Y is independently OH, COOR⁷, or COOH.

32. The compound of claim 30, wherein W¹ is C(R¹)(R²)(CH₂)ₙ₋Y.

33. The compound of claim 30, wherein W¹ is V.

34. The compound of claim 30, wherein W¹ is C(R¹)(R²)—(CH₂)_{c-}V.

35. The compound of claim 30, wherein p is 0.

36. The compound of claim 30, wherein p is 1.

37. The compound of claim 30, wherein t is 1.

38. A compound of the formula Ia: 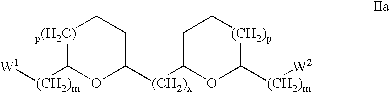
or a pharmaceutically acceptable salt, hydrate, solvate, or a mixture thereof, wherein:
(a) each occurrence of m is independently an integer ranging from 1 to 5;
(b) x is an integer ranging from 1 to 4;
(c) W¹ and W² are independently C(R¹)(R²)(CH₂)ₙ₋Y, V, or C(R¹)(R²)—(CH₂)_{c-}V where c is 1 or 2 and n is an integer ranging from 0 to 4;
(d) each occurrence of R¹ and R² is independently (C₁₋C₆)alkyl, (C₂₋C₆)alkenyl, (C₂₋C₆)alkynyl, phenyl, or benzyl;
(e) V is 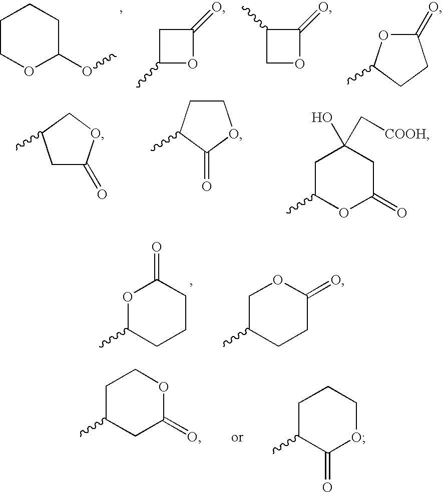
(f) Y is OH, COOH, CHO, COOR⁷, SO₃H, 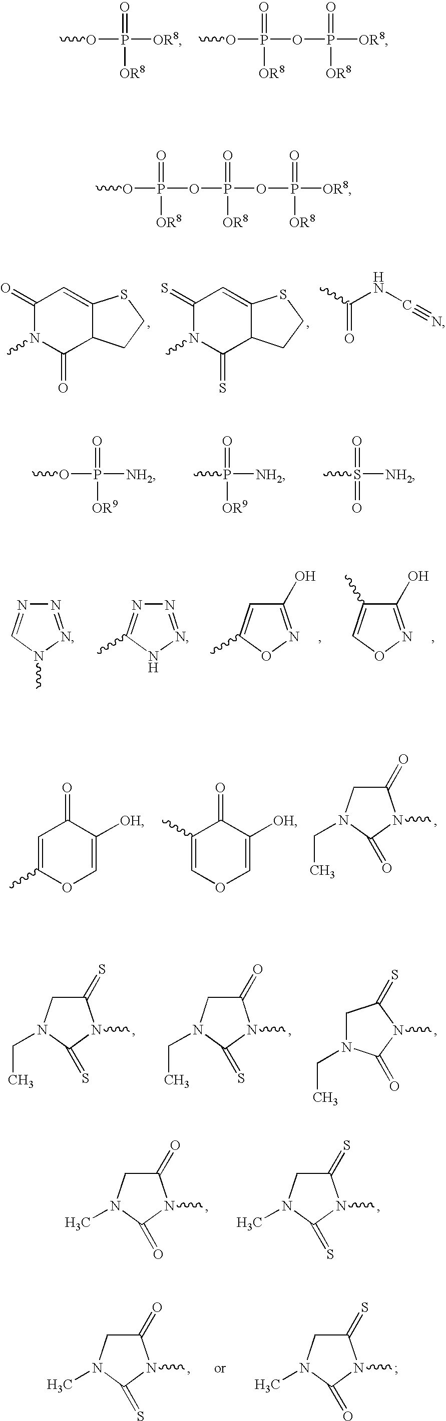
(g) R⁷ is (C₁₋C₆)alkyl, (C₂₋C₆)alkenyl, (C₂₋C₆)alkynyl, phenyl, or benzyl and is unsubstituted or substituted with one or more halo, OH, (C₁₋C₆)alkoxy, or phenyl groups;
(h) each occurrence of R⁸ is independently H, (C₁₋C₆)alkyl, (C₂₋C₆)alkenyl, or (C₂₋C₆)alkynyl and is unsubstituted or substituted with one or two halo, OH, (C₁₋C₆)alkoxy, or phenyl groups;
(i) each occurrence of R⁹ is independently H, (C₁₋C₆)alkyl, (C₂₋C₆)alkenyl, or (C₂₋C₆)alkynyl; and
(j) each occurrence of p is independently 0 or 1.

39. The compound of claim 38, wherein W¹ and W² are independent C(R¹)(R²)(CH₂)ₙ₋Y groups and each occurrence of Y is independently OH, COOR⁷, or COOH.

40. The compound of claim 38, wherein W¹ is C(R¹)(R²)(CH₂)ₙ₋Y.

41. The compound of claim 38, wherein W¹ is V.

42. The compound of claim 38, wherein W¹ is C(R¹)(R²)—(CH₂)_{c-}V.

43. The compound of claim 38, wherein p is 0.

44. The compound of claim 38, wherein p is 1.

45. A compound of the formula:
II-1: 5-(6-{3-[6-(5-hydroxy-4,4-dimethyl-pentyl)-tetrahydro-pyran-2-yl]-propyl}-tetrahydro-pyran-2-yl)-2,2-dimethyl-pentan-1-ol;
II-2: 5-(6-{3-[6-(5-hydroxy-4,4-dimethyl-pentyl)-tetrahydro-pyran-2-yl]-propyl}-tetrahydro-pyran-2-yl)-2,2-dimethyl-pentanoic acid;
II-3: 5-(6-{3-[6-(4-carboxyl-4,4-dimethyl-pentyl)-tetrahydro-pyran-2-yl]-propyl}-tetrahydro-pyran-2-yl)-2,2-dimethyl-pentanoic acid;
II-4: 5-(6-{3-[6-(5-hydroxy-4,4-dimethyl-pentyl)-4-oxo-pyran-2-yl]-propyl}-4-oxo-pyran-2-yl)-2,2-dimethyl-pentan-1-ol;
II-5: 5-(6-{3-[6-(5-hydroxy-4,4-dimethyl-pentyl)-4-oxo-pyran-2-yl]-propyl-4-oxo-pyran-2-yl)-2,2-dimethyl-pentanoic acid;
II-6: 5-(6-{3-[6-(4-carboxyl-4,4-dimethyl-pentyl)-4-oxo-pyran-2-yl]-propyl}-4-oxo-pyran-2-yl)-2,2-dimethyl-pentanoic acid;
II-7: 6-(6-{3-[6-(6-hydroxy-5,5-dimethyl-hexyl)-tetrahydro-pyran-2-yl]-propyl}-tetrahydro-pyran-2-yl)-2,2-dimethyl-hexan-1-ol;
II-8: 6-(6-{3-[6-(6-hydroxy-5,5-dimethyl-hexyl)-tetrahydro-pyran-2-yl]-propyl}-tetrahydro-pyran-2-yl)-2,2-dimethyl-hexanoic acid;
II-9: 6-(6-{3-[6-(5-carboxyl-5,5-dimethyl-hexyl)-tetrahydro-pyran-2-yl]-propyl}-tetrahydro-pyran-2-yl)-2,2-dimethyl-hexanoic acid;
II-10: 6-(6-{3-[6-(6-hydroxy-5,5-dimethyl-hexyl)-4-oxo-pyran-2-yl]-propyl}-4-oxo-pyran-2-yl)-2,2-dimethyl-hexan-1-ol;
II-11: 6-(6-{3-[6-(6-hydroxy-5,5-dimethyl-hexyl)-4-oxo-pyran-2-yl]-propyl}-4-oxo-pyran-2-yl)-2,2-dimethyl-hexanoic acid;
II-12: 6-(6-{3-[6-(5-carboxyl-5,5-dimethyl-hexyl)-4-oxo-pyran-2-yl]-propyl}-4-oxo-pyran-2-yl)-2,2-dimethyl-hexanoic acid;
II-13: 6-(6-{2-[6-(6-hydroxy-5,5-dimethyl-hexyl)-tetrahydro-pyran-2-yl]-vinyl}-tetrahydro-pyran-2-yl)-2,2-dimethyl-hexan-1-ol;
II-14: 6-(6-{2-[6-(6-hydroxy-5,5-dimethyl-hexyl)-tetrahydro-pyran-2-yl]-vinyl}-tetrahydro-pyran-2-yl)-2,2-dimethyl-hexanoic acid;
II-15: 6-(6-{2-[6-(5-carboxyl-5,5-dimethyl-hexyl)-tetrahydro-pyran-2-yl]-vinyl}-tetrahydro-pyran-2-yl)-2,2-dimethyl-hexanoic acid;
II-16: 6-(6-{2-[6-(6-hydroxy-5,5-dimethyl-hexyl)-4-oxo-pyran-2-yl]-vinyl}-4-oxo-pyran-2-yl)-2,2-dimethyl-hexan-1-ol;
II-17: 6-(6-{2-[6-(6-hydroxy-5,5-dimethyl-hexyl)-4-oxo-pyran-2-yl]-vinyl}-4-oxo-pyran-2-yl)-2,2-dimethyl-hexanoic acid;
II-18: 6-(6-{2-[6-(5-carboxyl-5,5-dimethyl-hexyl)-4-oxo-pyran-2-yl]-vinyl}-4-oxo-pyran-2-yl)-2,2-dimethyl-hexanoic acid;
II-19: 5-(6-{2-[6-(5-hydroxy-4,4-dimethyl-pentyl)-tetrahydro-pyran-2-yl]-vinyl}-tetrahydro-pyran-2-yl)-2,2-dimethyl-pentan-1-ol;
II-20: 5-(6-{2-[6-(5-hydroxy-4,4-dimethyl-pentyl)-tetrahydro-pyran-2-yl]-vinyl}-tetrahydro-pyran-2-yl)-2,2-dimethyl-pentanoic acid;
II-21: 5-(6-{2-[6-(4-carboxyl-4,4-dimethyl-pentyl)-tetrahydro-pyran-2-yl]-vinyl}-tetrahydro-pyran-2-yl)-2,2-dimethyl-pentanoic acid;
II-22: 5-(6-{2-[6-(5-hydroxy-4,4-dimethyl-pentyl)-4-oxo-pyran-2-yl]-vinyl}-4-oxo-pyran-2-yl)-2,2-dimethyl-pentan-1-ol;
II-23: 5-(6-{2-[6-(5-hydroxy-4,4-dimethyl-pentyl)-4-oxo-pyran-2-yl]-vinyl}-4-oxo-pyran-2-yl)-2,2-dimethyl-pentanoic acid;
II-24: 5-(6-{2-[6-(4-carboxyl-4,4-dimethyl-pentyl)-4-oxo-pyran-2-yl]-vinyl}-4-oxo-pyran-2-yl)-2,2-dimethyl-pentanoic acid;
II-25: 6-(6-{2-[6-(6-hydroxy-5,5-dimethyl-hexyl)-tetrahydro-pyran-2-yl]-phenyl}-tetrahydro-pyran-2-yl)-2,2-dimethyl-hexan-1-ol;
II-26: 6-(6-{2-[6-(6-hydroxy-5,5-dimethyl-hexyl)-tetrahydro-pyran-2-yl]-phenyl}-tetrahydro-pyran-2-yl)-2,2-dimethyl-hexanoic acid;
II-27: 6-(6-{2-[6-(5-carboxyl-5,5-dimethyl-hexyl)-tetrahydro-pyran-2-yl]-phenyl}-tetrahydro-pyran-2-yl)-2,2-dimethyl-hexanoic acid;
II-28: 5-(6-{2-[6-(5-hydroxy-4,4-dimethyl-pentyl)-4-oxo-pyran-2-yl]-phenyl}-4-oxo-pyran-2-yl)-2,2-dimethyl-pentan-1-ol;
II-29: 5-(6-{2-[6-(5-hydroxy-4,4-dimethyl-pentyl)-4-oxo-pyran-2-yl]-phenyl}-4-oxo-pyran-2-yl)-2,2-dimethyl-pentanoic acid;
II-30: 5-(6-{2-[6-(4-carboxyl-4,4-dimethyl-pentyl)-4-oxo-pyran-2-yl]-phenyl}-4-oxo-pyran-2-yl)-2,2-dimethyl-pentan-1-ol;
II-31: 5-(6-{2-[6-(5-hydroxy-4,4-dimethyl-pentyl)-tetrahydro-pyran-2-yl]-phenyl}-tetrahydro-pyran-2-yl)-2,2-dimethyl-pentan-1-ol;
II-32: 5-(6-{2-[6-(5-hydroxy-4,4-dimethyl-pentyl)-4-oxo-pyran-2-yl]-phenyl}-4-oxo-pyran-2-yl)-2,2-dimethyl-pentanoic acid;
II-33: 5-(6-{2-[6-(4-carboxyl-4,4-dimethyl-pentyl)-4-oxo-pyran-2-yl]-phenyl}-4-oxo-pyran-2-yl)-2,2-dimethyl-pentanoic acid;
II-34: 6-(6-{2-[6-(6-hydroxy-5,5-dimethyl-hexyl)-4-oxo-pyran-2-yl]-phenyl}-4-oxo-pyran-2-yl)-2,2-dimethyl-hexan-1-ol;
II-35: 6-(6-{2-[6-(6-hydroxy-5,5-dimethyl-hexyl)-4-oxo-pyran-2-yl]-phenyl}-4-oxo-pyran-2-yl)-2,2-dimethyl-hexanoic acid;
II-36: 6-(6-{2-[6-(5-carboxyl-5,5-dimethyl-hexyl)-4-oxo-pyran-2-yl]-phenyl}-4-oxo-pyran-2-yl)-2,2-dimethyl-hexanoic acid;
II-37: 5-(5-{3-[5-(5-hydroxy-4,4-dimethyl-pentyl)-tetrahydro-furan-2-yl]-propyl}-tetrahydro-furan-2-yl)-2,2-dimethyl-pentan-1-ol;
II-38: 5-(5-{3-[5-(5-hydroxy-4,4-dimethyl-pentyl)-tetrahydro-furan-2-yl]-propyl}-tetrahydro-furan-2-yl)-2,2-dimethyl-pentanoic acid;
II-39: 5-(5-{3-[5-(4-carboxyl-4,4-dimethyl-pentyl)-tetrahydro-furan-2-yl]-propyl}-tetrahydro-furan-2-yl)-2,2-dimethyl-pentanoic acid;
II-40: 5-(5-{3-[5-(5-hydroxy-4,4-dimethyl-pentyl)-furan-2-yl]-propyl}-furan-2-yl)-2,2-dimethyl-pentan-1-ol;
II-41: 5-(5-{3-[5-(5-hydroxy-4,4-dimethyl-pentyl)-furan-2-yl]-propyl}-furan-2-yl)-2,2-dimethyl-pentanoic acid;
II-42: 5-(5-{3-[5-(4-carboxyl-4,4-dimethyl-pentyl)-furan-2-yl]-propyl}-furan-2-yl)-2,2-dimethyl-pentanoic acid;
II-43: 6-(5-{3-[5-(6-hydroxy-5,5-dimethyl-hexyl)-tetrahydro-furan-2-yl]-propyl}-tetrahydro-furan-2-yl)-2,2-dimethyl-hexan-1-ol;
II-44: 6-(5-{3-[5-(6-hydroxy-5,5-dimethyl-hexyl)-tetrahydro-furan-2-yl]-propyl}-tetrahydro-furan-2-yl)-2,2-dimethyl-hexanoic acid;
II-45: 6-(5-{3-[5-(5-carboxyl-5,5-dimethyl-hexyl)-tetrahydro-furan-2-yl]-propyl}-tetrahydro-furan-2-yl)-2,2-dimethyl-hexanoic acid;
II-46: 6-(5-{3-[5-(6-hydroxy-5,5-dimethyl-hexyl)-furan-2-yl]-propyl}-furan-2-yl)-2,2-dimethyl-hexan-1-ol;
II-47: 6-(5-{3-[5-(6-hydroxy-5,5-dimethyl-hexyl)-furan-2-yl]-propyl}-furan-2-yl)-2,2-dimethyl-hexanoic acid;
II-48: 6-(5-{3-[5-(5-carboxyl-5,5-dimethyl-hexyl)-furan-2-yl]-propyl}-furan-2-yl)-2,2-dimethyl-hexanoic acid;
II-49: 6-(5-{2-[5-(6-hydroxy-5,5-dimethyl-hexyl)-tetrahydro-furan-2-yl]-vinyl}-tetrahydro-furan-2-yl)-2,2-dimethyl-hexan-1-ol;
II-50: 6-(5-{2-[5-(6-hydroxy-5,5-dimethyl-hexyl)-tetrahydro-furan-2-yl]-vinyl}-tetrahydro-furan-2-yl)-2,2-dimethyl-hexanoic acid;
II-51: 6-(5-{2-[5-(5-carboxyl-5,5-dimethyl-hexyl)-tetrahydro-furan-2-yl]-vinyl}-tetrahydro-furan-2-yl)-2,2-dimethyl-hexanoic acid;
II-52: 6-(5-{2-[5-(6-hydroxy-5,5-dimethyl-hexyl)-furan-2-yl]-vinyl}-furan-2-yl)-2,2-dimethyl-hexan-1-ol;
II-53: 6-(5-{2-[5-(6-hydroxy-5,5-dimethyl-hexyl)-furan-2-yl]-vinyl}-furan-2-yl)-2,2-dimethyl-hexanoic acid;
II-54: 6-(5-{2-[5-(5-carboxyl-5,5-dimethyl-hexyl)-furan-2-yl]-vinyl}-furan-2-yl)-2,2-dimethyl-hexanoic acid;
II-55: 5-(5-{2-[5-(5-hydroxy-4,4-dimethyl-pentyl)-tetrahydro-furan-2-yl]-vinyl}-tetrahydro-furan-2-yl)-2,2-dimethyl-pentan-1-ol;
II-56: 5-(5-{2-[5-(5-hydroxy-4,4-dimethyl-pentyl)-tetrahydro-furan-2-yl]-vinyl}-tetrahydro-furan-2-yl)-2,2-dimethyl-pentanoic acid;
II-57: 5-(5-{2-[5-(4-carboxyl-4,4-dimethyl-pentyl)-tetrahydro-furan-2-yl]-vinyl}-tetrahydro-furan-2-yl)-2,2-dimethyl-pentanoic acid;
II-58: 5-(5-{2-[5-(5-hydroxy-4,4-dimethyl-pentyl)-furan-2-yl]-vinyl}-furan-2-yl)-2,2-dimethyl-pentan-1-ol;
II-59: 5-(5-{2-[5-(5-hydroxy-4,4-dimethyl-pentyl)-furan-2-yl]-vinyl}-furan-2-yl)-2,2-dimethyl-pentanoic acid;
II-60: 5-(5-{2-[5-(4-carboxyl-4,4-dimethyl-pentyl)-furan-2-yl]-vinyl}-furan-2-yl)-2,2-dimethyl-pentanoic acid;
II-61: 6-(5-{2-[5-(6-hydroxy-5,5-dimethyl-hexyl)-tetrahydro-furan-2-yl]-phenyl}-tetrahydro-furan-2-yl)-2,2-dimethyl-hexan-1-ol;
II-62: 6-(5-{2-[5-(6-hydroxy-5,5-dimethyl-hexyl)-tetrahydro-furan-2-yl]-phenyl}-tetrahydro-furan-2-yl)-2,2-dimethyl-hexanoic acid;
II-63: 6-(5-{2-[5-(5-carboxyl-5,5-dimethyl-hexyl)-tetrahydro-furan-2-yl]-phenyl}-tetrahydro-furan-2-yl)-2,2-dimethyl-hexanoic acid;
II-64: 5-(5-{2-[5-(5-hydroxy-4,4-dimethyl-pentyl)-tetrahydro-furan-2-yl]-phenyl}-tetrahydro-furan-2-yl)-2,2-dimethyl-pentan-1-ol;
II-65: 5-(5-{2-[5-(5-hydroxy-4,4-dimethyl-pentyl)-tetrahydro-furan-2-yl]-phenyl}-tetrahydro-furan-2-yl)-2,2-dimethyl-pentanoic acid;
II-66: 5-(5-{2-[5-(4-carboxyl-4,4-dimethyl-pentyl)-tetrahydro-furan-2-yl]-phenyl}-tetrahydro-furan-2-yl)-2,2-dimethyl-pentanoic acid;
II-67: 5-(5-{2-[5-(5-hydroxy-4,4-dimethyl-pentyl)-furan-2-yl]-phenyl}-furan-2-yl)-2,2-dimethyl-pentan-1-ol;
II-68: 5-(5-{2-[5-(5-hydroxy-4,4-dimethyl-pentyl)-furan-2-yl]-phenyl}-furan-2-yl)-2,2-dimethyl-pentanoic acid;
II-69: 5-(5-{2-[5-(4-carboxyl-4,4-dimethyl-pentyl)-furan-2-yl]-phenyl}-furan-2-yl)-2,2-dimethyl-pentanoic acid;
II-70: 6-(5-{2-[5-(6-hydroxy-5,5-dimethyl-hexyl)-furan-2-yl]-phenyl}-filran-2-yl)-2,2-dimethyl-hexan-1-ol;
II-71: 6-(5-{2-[5-(6-hydroxy-5,5-dimethyl-hexyl)-furan-2-yl]-phenyl}-furan-2-yl)-2,2-dimethyl-hexanoic acid;
II-72: 6-(5-{2-[5-(5-carboxyl-5,5-dimethyl-hexyl)-furan-2-yl]-phenyl}-furan-2-yl)-2,2-dimethyl-hexanoic acid;
II-73: 5-(6-{2-[6-(5-hydroxy-4,4-dimethyl-pentyl)-tetrahydro-pyran-2-yl]-ethyl}-tetrahydro-pyran-2-yl)-2,2-dimethyl-pentan-1-ol;
II-74: 5-(6-{2-[6-(5-hydroxy-4,4-dimethyl-pentyl)-tetrahydro-pyran-2-yl]-ethyl}-tetrahydro-pyran-2-yl)-2,2-dimethyl-pentanoic acid;
II-75: 5-(6-{2-[6-(4-carboxyl-4,4-dimethyl-pentyl)-tetrahydro-pyran-2-yl]-ethyl}-tetrahydro-pyran-2-yl)-2,2-dimethyl-pentanoic acid;
II-76: 5-(6-{2-[6-(5-hydroxy-4,4-dimethyl-pentyl)-4-oxo-pyran-2-yl]-ethyl}-4-oxo-pyran-2-yl)-2,2-dimethyl-pentan-1-ol;
II-77: 5-(6-{2-[6-(5-hydroxy-4,4-dimethyl-pentyl)-4-oxo-pyran-2-yl]-ethyl}-4-oxo-pyran-2-yl)-2,2-dimethyl-pentanoic acid;
II-78: 5-(6-{2-[6-(4-carboxyl-4,4-dimethyl-pentyl)-4-oxo-pyran-2-yl]-ethyl}-4-oxo-pyran-2-yl)-2,2-dimethyl-pentanoic acid;
II-79: 6-(5-{2-[5-(6-hydroxy-5,5-dimethyl-hexyl)-tetrahydro-furan-2-yl]-ethyl}-tetrahydro-furan-2-yl)-2,2-dimethyl-hexan-1-ol;
II-80: 6-(5-{2-[5-(6-hydroxy-5,5-dimethyl-hexyl)-tetrahydro-furan-2-yl]-ethyl}-tetrahydro-furan-2-yl)-2,2-dimethyl-hexanoic acid;
II-81: 6-(5-{2-[5-(5-carboxyl-5,5-dimethyl-hexyl)-tetrahydro-furan-2-yl]-ethyl}-tetrahydro-furan-2-yl)-2,2-dimethyl-hexanoic acid;
II-82: 6-(5-{2-[5-(6-hydroxy-5,5-dimethyl-hexyl)-furan-2-yl]-ethyl}-furan-2-yl)-2,2-dimethyl-hexan-1-ol;
II-83: 6-(5-{2-[5-(6-hydroxy-5,5-dimethyl-hexyl)-furan-2-yl]-ethyl}-furan-2-yl)-2,2-dimethyl-hexanoic acid; or
II-84: 6-(5-{2-[5-(5-carboxyl-5,5-dimethyl-hexyl)-furan-2-yl]-ethyl}-furan-2-yl)-2,2-dimethyl-hexanoic acid.

46. A compound of the formula III: 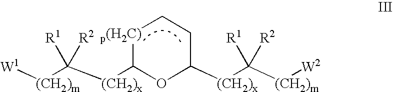
or a pharmaceutically acceptable salt, hydrate, solvate, or a mixture thereof, wherein:
(a) each occurrence of R¹ and R² is independently (C₁₋C₆)alkyl, (C₂₋C₆)alkenyl, (C₂₋C₆)alkynyl, phenyl, or benzyl; or R¹, R², and the carbon to which they are both attached are taken together to form a (C₃₋C₇)cycloalkyl group;
(b) each occurrence of m is an independent integer ranging from 0 to 4;
(c) each occurrence of x is independently 2 or 3;
(d) W¹ and W² are independently OH, C(O)OH, CHO, OC(O)R⁷, C(O)OR⁷, SO₃H, 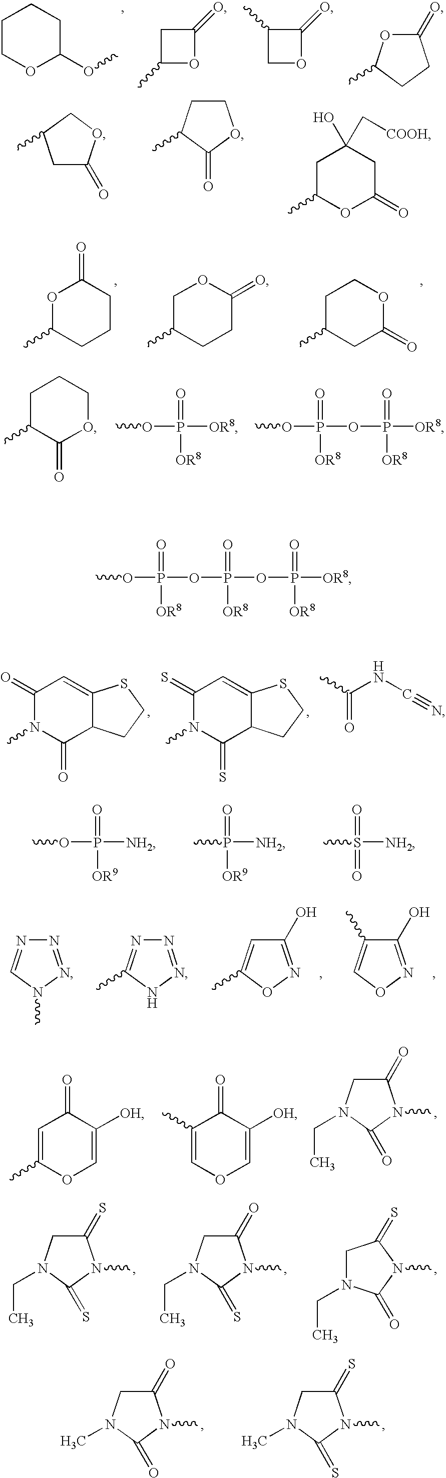 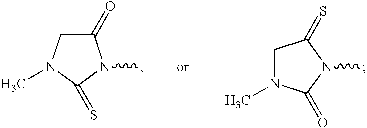
(e) R⁷ is (C₁₋C₆)alkyl, (C₂₋C₆)alkenyl, (C₂₋C₆)alkynyl, phenyl, or benzyl and is unsubstituted or substituted with one or more halo, OH, (C₁₋C₆)alkoxy, or phenyl groups;
(f) each occurrence of R⁸ is independently H, (C₁₋C₆)alkyl, (C₂₋C₆)alkenyl, or (C₂₋C₆)alkynyl and is unsubstituted or substituted with one or two halo, OH, (C₁₋C₆)alkoxy, or phenyl groups;
(g) each occurrence of R⁹ is independently H, (C₁₋C₆)alkyl, (C₂₋C₆)alkenyl, or (C₂₋C₆)alkynyl; and
(h) p is 0 or 1 where the broken line represents an optional presence of 1 or 2 additional carbon-carbon bonds that when present complete 1 or 2 carbon-carbon double bonds.

47. The compound of claim 46, wherein W¹ and W² are independently OH, COOR⁷, or COOH.

48. The compound of claim 46, wherein p is 0.

49. The compound of claim 46, wherein p is 1.

50. The compound of claim 46, wherein the broken line is absent.

51. The compound of claim 46, wherein each occurrence of R¹ and R² is independently (C₁₋C₆)alkyl, (C₂₋C₆)alkenyl, (C₂₋C₆)alkynyl, phenyl, or benzyl.

52. A compound of the of formula IV: 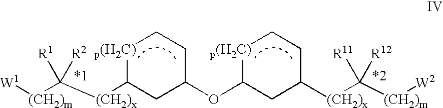
or a pharmaceutically acceptable salt, hydrate, solvate, or a mixture thereof, wherein:
(a) each occurrence of R¹ and R² is independently (C₁-C₆)alkyl, (C₂-C₆)alkenyl, (C₂-C₆)alkynyl, phenyl, benzyl, or R¹ and R² and the carbon to which they are both attached are taken together to form a (C₃-C₇)cycloakyl group;
(b) each occurrence of R¹¹ and R¹² is independently H, (C₁-C₆)alkyl, (C₂-C₆)alkenyl, (C₂-C₆)alkynyl, phenyl, benzyl, or R¹ and R² and the carbon to which they are both attached are taken together to form a (C₃-C₇)cycloakyl group;
(c) each occurrence of m is independently an integer ranging from 0 to 6;
(d) each occurrence of x is independently and integer from 0 to 4;
(e) W¹ and W² are independently (C₁-C₆)alkyl, OH, C(O)OH, CHO, OC(O)R⁷, C(O)OR⁷, SO₃H, 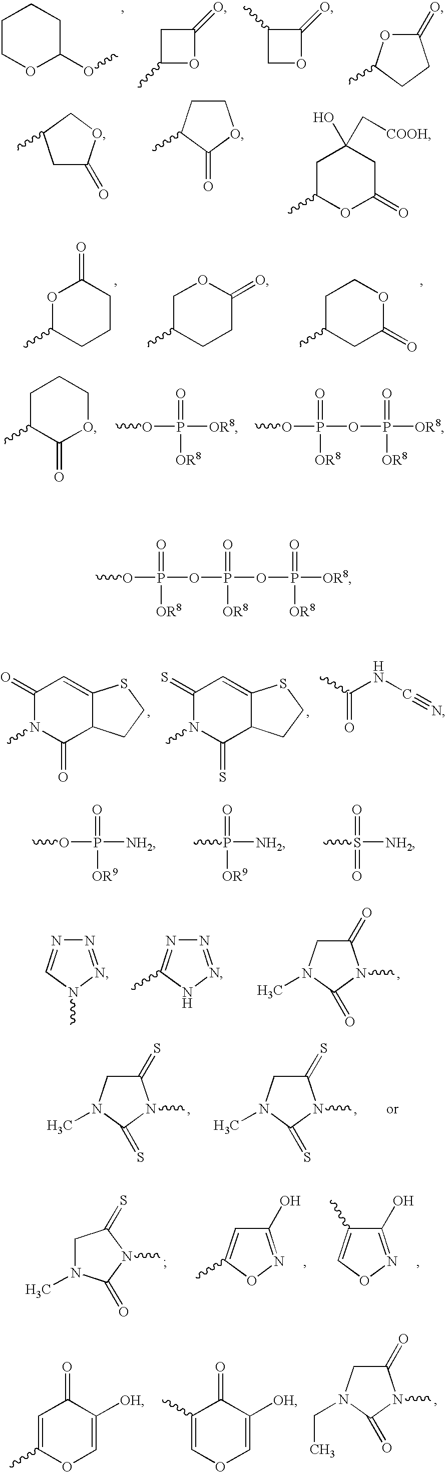 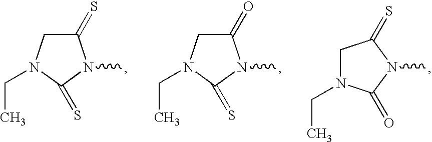
(f) R⁷ is (C₁₋C₆)alkyl, (C₂₋C₆)alkenyl, (C₂₋C₆)alkynyl, phenyl, or benzyl and is unsubstituted or substituted with one or more halo, OH, (C₁₋C₆)alkoxy, or phenyl groups;
(g) each occurrence of R⁸ is independently H, (C₁₋C₆)alkyl, (C₂₋C₆)alkenyl, or (C₂₋C₆)alkynyl and is unsubstituted or substituted with one or two halo, OH, (C₁₋C₆)alkoxy, or phenyl groups;
(h) each occurrence of R⁹ is independently H, (C₁₋C₆)alkyl, (C₂₋C₆)alkenyl, or (C₂₋C₆)alkynyl; and
(a) (i) each occurrence of p is independently 0 or 1 where the broken line represents an optional presence of 1, 2, or 3 additional carbon-carbon bonds that when present form a cycloalkenyl group, a cyclodienyl group, or a phenyl group.

53. The compound of claim 52, wherein W¹ and W² are independently OH, COOR⁷, or COOH.

54. The compound of claim 52, wherein each occurrence of R¹ and R² is independently (C₁₋C₆)alkyl, (C₂₋C₆)alkenyl, (C₂₋C₆)alkynyl, phenyl, or benzyl.

55. The compound of claim 52, wherein p is 0.

56. The compound of claim 52, wherein p is 1.

57. The compound of claim 52, wherein the broken line is absent.

58. The compound of claim 52, having the formula:

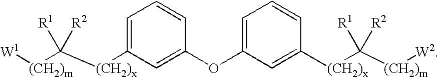

59. The compound of claim 52, having the formula:

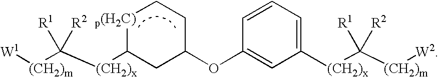

60. A pharmaceutical composition comprising a compound of claim 1, 13, 21, 26, 27, 28, 29, 30, 38, 45, 46, or 52 or a pharmaceutically acceptable salt, hydrate, solvate, clathrate, enantiomer, diastereomer, racemate, or a mixture of stereoisomers thereof and a pharmaceutically acceptable vehicle, excipient, or diluent and a pharmaceutically acceptable vehicle, excipient, or diluent.

61. A pharmaceutical composition comprising one or more of the following compounds:
I-114 4-[3-(3-carboxy-3-methyl-butoxy)-propoxy]-2,2-dimethyl-butyric acid,
I-297 5-[2-(5-hydroxy-4,4-dimethyl-pentyloxy)-ethoxy]-2,2-dimethyl-pentan-1-ol,
IV-1 3-{3-[3-(2-Carboxy-2-methyl-propyl)-phenoxy]-phenyl}-2,2-dimethyl-propionic acid,
IV-2 1-{3-[3-(2-hydroxy-2-methyl-propyl)-phenoxy]-phenyl}-2-methyl-propan-2-ol,
or a pharmaceutically acceptable salt, hydrate, solvate, clathrate, enantiomer, diastereomer, racemate, or a mixture of stereoisomers thereof and a pharmaceutically acceptable vehicle, excipient, or diluent.

62. A method for treating or preventing a cardiovascular disease in a patient, comprising administering to a patient in need of such treatment or prevention a therapeutically effective amount of a compound of claim 1, 13, 21, 26, 27, 28, 29, 30, 38, 45, 46, or 52 or a pharmaceutically acceptable salt, hydrate, solvate, or a mixture thereof.

63. A method for treating or preventing a dyslipidemia in a patient, comprising administering to a patient in need of such treatment or prevention a therapeutically effective amount of a compound of claim 1, 13, 21, 26, 27, 28, 29, 30, 38, 45, 46, or 52 or a pharmaceutically acceptable salt, hydrate, solvate, or a mixture thereof.

64. A method for treating or preventing a dyslipoproteinemia in a patient, comprising administering to a patient in need of such treatment or prevention a therapeutically effective amount of a compound of claim 1, 13, 21, 26, 27, 28, 29, 30, 38, 45, 46, or 52 or a pharmaceutically acceptable salt, hydrate, solvate, or a mixture thereof.

65. A method for treating or preventing a disorder of glucose metabolism in a patient, comprising administering to a patient in need of such treatment or prevention a therapeutically effective amount of a compound of claim 1, 13, 21, 26, 27, 28, 29, 30, 38, 45, 46, or 52 or a pharmaceutically acceptable salt, hydrate, solvate, or a mixture thereof.

66. A method for treating or preventing hypertension in a patient, comprising administering to a patient in need of such treatment or prevention a therapeutically effective amount of a compound of claim 1, 13, 21, 26, 27, 28, 29, 30, 38, 45, 46, or 52 or a pharmaceutically acceptable salt, hydrate, solvate, or a mixture thereof.

67. A method for treating or preventing renal disease in a patient, comprising administering to a patient in need of such treatment or prevention a therapeutically or prophylactically effective amount of a compound of claim 1, 13, 21, 26, 27, 28, 29, 30, 38, 45, 46, or 52 or a pharmaceutically acceptable salt, hydrate, solvate, or a mixture thereof.

68. A method for treating or preventing cancer in a patient, comprising administering to a patient in need of such treatment or prevention a therapeutically or prophylactically effective amount of a compound of claim 1, 13, 21, 26, 27, 28, 29, 30, 38, 45, 46, or 52 or a pharmaceutically acceptable salt, hydrate, solvate, or a mixture thereof

69. A method for treating or preventing inflammation in a patient, comprising administering to a patient in need of such treatment or prevention a therapeutically or prophylactically effective amount of a compound of claim 1, 13, 21, 26, 27, 28, 29, 30, 38, 45, 46, or 52 or a pharmaceutically acceptable salt, hydrate, solvate, or a mixture thereof

70. A method for treating or preventing impotence in a patient, comprising administering to a patient in need of such treatment or prevention a therapeutically or prophylactically effective amount of a compound of comprising administering to a patient in need of such treatment or prevention a therapeutically or prophylactically effective amount of a compound of claim 1, 13, 21, 26, 27, 28, 29, 30, 38, 45, 46, or 52 or a pharmaceutically acceptable salt, hydrate, solvate, or a mixture thereof

71. A method for treating or preventing a neurodegenerative disease or disorder in a patient, comprising administering to a patient in need of such treatment or prevention a therapeutically or prophylactically effective amount of a compound of claim 1, 13, 21, 26, 27, 28, 29, 30, 38, 45, 46, or 52 or a pharmaceutically acceptable salt, hydrate, solvate, or a mixture thereof

72. A method of inhibiting hepatic fatty acid synthesis in a patient, comprising administering to a patient in need of such treatment or prevention a therapeutically or prophylactically effective amount of a compound of claim 1, 13, 21, 26, 27, 28, 29, 30, 38, 45, 46, or 52 or a pharmaceutically acceptable salt, hydrate, solvate, or a mixture thereof.

73. A method of inhibiting sterol synthesis in a patient, comprising administering to a patient in need of such treatment or prevention a therapeutically or prophylactically effective amount of a compound of claim 1, 13, 21, 26, 27, 28, 29, 30, 38, 45, 46, or 52 or a pharmaceutically acceptable salt, hydrate, solvate, or a mixture thereof.

74. A method of treating or preventing metabolic syndrome disorders in a patient, comprising administering to a patient in need of such treatment or prevention a therapeutically or prophylactically effective amount of a compound of claim 1, 13, 21, 26, 27, 28, 29, 30, 38, 45, 46, or 52 or a pharmaceutically acceptable salt, hydrate, solvate, or a mixture thereof.

75. A method of treating or preventing a disease or disorder that is capable of being treated or prevented by increasing HDL levels, which comprises administering to a patient in need of such treatment or prevention a therapeutically effective amount of a compound of claim 1, 13, 21, 26, 27, 28, 29, 30, 38, 45, 46, or 52 or a pharmaceutically acceptable salt, hydrate, solvate, or a mixture thereof.

76. A method of treating or preventing a disease or disorder that is capable of being treated or prevented by lowering LDL levels, which comprises administering to such patient in need of such treatment or prevention a therapeutically effective amount of a compound of claim 1, 13, 21, 26, 27, 28, 29, 30, 38, 45, 46, or 52 or a pharmaceutically acceptable salt, hydrate, solvate, or a mixture thereof

77. A pharmaceutical composition comprising a compound of claim 1, 11, 28, 29, 30, 31, 32, 33, 34, or 35 and a pharmaceutically acceptable vehicle, excipient, or diluent which is administered in combination with a statin.
